(19) United States
(12) Patent Application Publication
(10) Pub. No.: US 2026/0209215 A1
(43) Pub. Date: Jul. 23, 2026

(54) GLYCINE DERIVATIVES WITH P2X4 RECEPTOR-BLOCKING ACTIVITY AS DIAGNOSTICS AND FOR THE TREATMENT OF PAIN, INFLAMMATION, CANCER, AND OTHER P2X4 RECEPTOR-RELATED DISEASES

(71) Applicant: RHEINISCHE-FRIEDRICH-WILHELMS-UNIVERSITÄT BONN, Bonn (DE)

(72) Inventors: Christa E. MÜLLER, Bonn (DE); Victor Hernandez OLMOS, Bonn (DE); Ahmed ELGOKHA, Bonn (DE); Ali EL-TAYEB, Bonn (DE); Mahmoud RASHED, Bonn (DE); Jörg HOCKEMEYER, Bonn (DE); Enas MALIK, Bonn (DE); Samer ALSHAIBANI, Bonn (DE); Anton IVANOV, Bonn (DE); Federica ROSI, Bonn (DE); Svenja WERNER, Bonn (DE); Thanigaimalai PILLAIYAR, Bonn (DE); Aliaa ABDELRAHMAN, Bonn (DE); Vigneshwaran NAMASIVAYAM, Bonn (DE)

(21) Appl. No.: 19/140,808

(22) PCT Filed: Jun. 20, 2023
(86) PCT No.: PCT/EP2023/087016

§ 371 Date: Jun. 18, 2025

(30) Foreign Application Priority Data
Dec. 20, 2022 (EP) 22214883.5

(51) Int. Cl. C07D 417/12; A61K 31/4045; A61K 31/41; A61K 31/4245; A61K 31/433; A61K 31/437; A61K 31/438; A61K 31/4439; A61K 31/4745; A61K 31/496; A61K 31/5377; A61K 31/5517; A61K 31/553; C07D 401/12; C07D 403/12; C07D 405/12; C07D 405/14; C07D 413/12; C07D 417/14; C07D 471/04; C07D 471/06; C07D 487/06; C07D 491/107; C07D 498/06

(52) U.S. Cl. CPC C07D 417/12; A61K 31/4045; A61K 31/41; A61K 31/4245; A61K 31/433; A61K 31/437; A61K 31/438; A61K 31/4439; A61K 31/4745; A61K 31/496; A61K 31/5377; A61K 31/5517; A61K 31/553; C07D 401/12; C07D 403/12; C07D 405/12; C07D 405/14; C07D 413/12; C07D 417/14; C07D 471/04; C07D 471/06; C07D 487/06; C07D 491/107; C07D 498/06

(57) ABSTRACT

The invention relates to glycine derivatives with P2X4 receptor-blocking activity for the treatment of pain, inflammation, cancer, and other P2X4 receptor-related diseases.

This application claims the priority of European patent application no. 22 214 883.5.

The role of purine nucleotides (ATP, ADP, AMP and adenosine) in neurotransmission took a long time to be understood and accepted. Geoffrey Burnstock showed in 1972 that ATP (adenosine triphosphate) was a neurotransmitter in non-adrenergic, non-cholinergic nerves in the gut. Later, ATP was demonstrated to be a co-transmitter in sympathetic and parasympathetic nerves. Now, it is widely recognized that ATP is a co-transmitter in all nerve types of the peripheral and central nervous systems. Enzymes, termed ecto-nucleotidases, break down ATP to ADP, AMP and adenosine. Adenosine, acting through purinergic P1 receptors (A₁, A_{2A}, A_{2B}, A₃) has been considered a stop signal, showing, e.g. sedative, anti-convulsive, analgesic, anti-inflammatory, and immunosuppressive properties. On the other hand, ATP, an endogenous nociceptive and pro-inflammatory substance, acts on purinergic P2 receptors. Seven P2 receptor subtypes exist which are ligand-gated ion channels (“ionotropic” P2X receptors: P2X1-7), activated by ATP. A sub-family of 8 P2 receptors are G-protein-coupled receptors (“metabotropic”) P2Y_{1, 2, 4, 6, 11, 12, 13, 14} receptors, some of which are activated by ATP or ADP, others by uracil nucleotides. The functions of the P2 receptors are intensely researched and have been reviewed in detail. A remarkable functional aspect of ATP receptors, of particular therapeutic importance, is that they play a crucial role in the interaction between neurons and glia in the central nervous system (CNS). Purinergic signaling has been shown to be involved in plenty of disorders of the CNS and the periphery, and purinergic receptors have been advanced as targets for various indications.

The present invention is focused on antagonists of the P2X4 receptor, an ionotropic P2 receptor activated by ATP, which has a pathophysiologic and therapeutic role in chronic neuropathic pain, chronic inflammatory pain, neuroprotection, cancer, and other pathological conditions (see e.g. Tam, T. H., Salter, M. W. Purinergic signalling in spinal pain processing. Purinergic Signal. 2021, 17, 49-54; and Schmitt, M. et al., Colon tumour cell death causes mTOR dependence by paracrine P2X4 stimulation. Nature 2022).

P2X4Rs are widely expressed in the central nervous system and in the periphery, e.g., in microglia, and on endothelial cells. Peripheral nerve injury leads to microglial activation in the spinal cord which results in increased P2X4R levels. This process leads to neuropathic pain. In contrast, P2X4R knockout mice displayed reduced pain and no development of allodynia. P2X4R antagonists have therefore great potential for the treatment of neuropathic pain. Further potential indications include, but are not limited to spinal cord injury, epilepsy, stroke and acute brain injury, multiple sclerosis, and neurodegenerative diseases such as Parkinson's and Alzheimer's and amyotropic lateral sclerosis (ALS), obesity, obesity-dependent inflammation and artherosclerosis, allergen-induced airway inflammation, allergic asthma and airway remodeling, rheumatoid arthritis, colitis, alcohol-induced liver inflammation and steatohepatitis, various other inflammatory and fibrotic diseases, itch and cutaneous pain, muscle pain, chronic pain, diabetic neuropathy, trigeminal neuralgia, and various other types of pain, pain and depression comorbidity; liver fibrosis, hepatitis virus-induced hepatocellular carcinoma, prostate cancer, gastric cancer, breast cancer, glioma, colon cancer, and various disorders of the central nervous system.

In stark contrast to the situation of P2X3 and P2X7 receptors, there is still a lack of sufficiently powerful compounds to selectively block P2X4 receptor signaling in vivo.

A major step forward has been the discovery of the crystal structure of the ATP-gated P2X4 ionotropic receptor, which has facilitated and accelerated our understanding of the structural requirements for P2X receptors in general (see e.g. Kawate, T., Michel, J. C., Birdsong, W. T., and Gouaux, E., Crystal structure of the ATP-gated P2X4 ion channel in the closed state. Nature, 2009, 460, 592-599; and Young, M. T., P2X receptors: dawn of the post-structure era. TIBS, 2009, 719, 8 Pp).

It has been shown that antidepressants, including paroxetine, other serotonin-specific reuptake inhibitors (SSRIs) and tricyclic antidepressants produce anti-allodynic effects following spinal nerve ligation in rats, independently of serotonin-related mechanisms, but rather correlated to their ability to non-competitively antagonize the P2X4 receptor. Since such antidepressants are clinically used in the treatment of neuropathic pain, this finding validates the P2X4 receptor as a therapeutic target for the treatment of human neuropathic pain.

The P2X4 receptor is present in various CNS areas, on immune cells and on peripheral macrophages. After peripheral nerve or CNS lesions, the P2X4 receptor is upregulated in activated microglia within the CNS. It is the location and upregulation of the P2X4 receptor selectively in CNS spinal and/or supraspinal, injury-induced, activated microglia that links the ATP-gated ion channel P2X4 to pathophysiologic processes underlying persistent and neuropathic pain, traumatic brain injury, cerebral ischemia and spinal cord injury. The link between upregulation of the P2X4 receptor and microglial activation hints to a “neuro-inflammatory” mechanism, the pharmacological attenuation of which is expected to bear therapeutic potential in the treatment of chronic neuropathic pain and in neuroprotection.

Partial damage to a rat peripheral nerve (e. g., the sciatic nerve of the hindlimb), which is typically performed in rodent models of neuropathic pain, produces activation of spinal dorsal horn microglia. Within 1 hour after injury, microglia change into a hypertrophied state with amoeba-like processes, from which diffusible factors, including pro-inflammatory cytokines and marker proteins, are released. This is accompanied by an upregulation of P2X4 receptors located on such activated spinal microglia. In the following 2 to 3 days, spinal microglia proliferate. This neuro-inflammatory process has been implicated in the initiation and maintenance of chronic neuropathic pain. As spinal microglial P2X4 receptors increase within days after peripheral nerve injury, the threshold of the paw withdrawal reflex to a previously neutral tactile stimulus decreases, indicative of the development of central sensitization that induces tactile (mechanical) allodynia. Interestingly, P2X4 receptor-deficient mice show a selective and marked reduction of mechanical allodynia and hyperalgesia in the spinal nerve ligation model of neuropathic pain. Together, these results point to an essential role of the P2X4 receptor in nerve injury-induced chronic tacile allodynia, a hallmark symptom of human neuropathic pain.

Tsuda et al. (2003) first showed that P2X4 receptors induced in activated spinal microglia gate mechanical allodynia after peripheral nerve injury (see Tsuda, M., Shigemoto-Mogami, Y., Koizumi, S., Mizokoshi, A., Kohsaka, S., Salter, M. W., and Inoue, K., P2X₄ receptors induced in spinal microglia gate tactile allodynia after nerve injury. Nature, 2003, 424, 778-783). Tactile allodynia induced by L5 spinal nerve ligation (SNL) could be antagonized by intrathecally administered TNP-ATP, a competitive, nonselective antagonist of P2X receptors, but not by PPADS, an antagonist of P2X1-3, 5, 7, suggesting that this reversal is P2X4 receptor-mediated. Following SNL, upregulation of the P2X4 receptor occurred solely in activated spinal microglia, not in neurons or astroglia. The upregulation of spinal microglial P2X4 receptors within 1 day after SNL matched the development of tactile allodynia. Intrathecal injection of P2X4 antisense or mismatch oligodeoxynucleotide after SNL prevented the upregulation of spinal microglial P2X4, which was associated with suppression of the development of tactile allodynia. Intrathecal injection of ATP-stimulated cultured microglia into intact rats produced upregulation of spinal microglial P2X4 and the development of robust tactile allodynia, which was blocked by TNP-ATP. This result indicates that activation of P2X4 receptors in hyperactive microglia was sufficient to produce tactile allodynia. Moreover, P2X4 receptor upregulation on spinal microglia in the lumbar dorsal horn of the spinal cord was observed in experimental autoimmune neuritis, a rat model of acute inflammatory demyelinating polyradiculopathy, the most common subtype of the Guillain-Barré syndrome. The time-course of spinal P2X4 receptor upregulation was tightly linked to the onset (day 9), peak (day 17-19) and gradual disappearance (until day 37) of neuropathic pain, as measured by means of the mechanical allodynia test. In addition to neuropathic pain, inflammatory pain may also be a therapeutic target for the P2X4 receptor. Thus, following plantar hindpaw injection of formalin in rats, P2X4 receptors are upregulated in the ipsilateral dorsal horn of the spinal cord and the kinetics of this reaction parallel that of spinal microglial activation in the development of formalin-induced long-term hyperalgesia.

Hyperactive microglia are also critical in the pathogenesis underlying neurodegenerative disorders and stroke. In such conditions, as stated above, P2X4 receptors are also upregulated, e.g. after traumatic brain injury, cerebral ischemia and spinal cord injury. This indicates that the P2X4 receptor may be a potential therapeutic target for neuroprotection.

According to further findings, ATP released from microglia that are activated by nerve injury, stimulates, through the P2X4 receptor, the release of brain-derived neurotrophic factor (BDNF), which, in turn, downregulates the neuronal K⁺/Cl⁻cotransporter (KCC2). This causes a switch towards depolarization of the anion/GABA equilibrium potential in spinal lamina I neurons, thereby transforming GABA-related IPSPs into EPSPs and thus yielding hyperexcitability through a deficit in tonic inhibition by GABA-ergic neurons. This mechanism may be responsible for the development of neuronal hyperexcitability in the dorsal horn of the spinal cord (central sensitization), which may underlie chronic neuropathic allodynia and hyperalgesia. It is interesting to note that this BDNF-Trk B receptor mechanism, downregulating KCC2, has also been viewed as a chronic neuroplasticity mechanism involved in the induction and maintenance of neural hyperexcitability associated with kindling, a chronic model of epilepsy. The BDNF-Trk B mechanism apparently acts as a microglia-neuron signaling pathway “kindling” neuropathic pain. Such a neuroplastic mechanism, in which the P2X4 receptor FIGURES as a crucial part, may be important for chronic neurotoxicity/neurodegeneration and, conversely, its modulation may serve as a target for neuroprotection. It has been demonstrated that global ischemia in gerbils is associated with the upregulation of P2X4 receptors, localized to microglia in the hippocampal CA1 and CA1-CA2 transition zone, and that P2X antagonists reduce delayed neuronal death in this model of global ischemia.

Hence, potent and selective P2X4 receptor antagonists may convey neuroprotective properties in cerebral ischemia, traumatic brain injury and spinal cord injury. Further, a neuroprotective/disease modifying approach is of equally prominent importance for the treatment of chronic neuropathic pain (e.g. in diabetes, chemotherapy, HIV and antiretroviral HIV treatment) as well as for epileptogenesis (e.g., in temporal lobe epilepsy, post-traumatic brain injury epileptogenesis).

It is noteworthy that the rat and human P2X4 receptors show 87% identity. In in vitro heterologous expression systems, both have a very similar agonist potency profile, and potent antagonists may be active in both, human and rodents.

The present invention relates to the discovery of potent and selective allosteric antagonists for the human P2X4 receptor. Brain-permeable inhibitors, and also only peripherally active antagonists have been discovered in the same series.

A first aspect of the invention relates to a compound according to general Formula (A), general Formula (B), general Formula (C) and/or general Formula (D), as defined in appended claim 1, or a physiologically acceptable salt thereof:

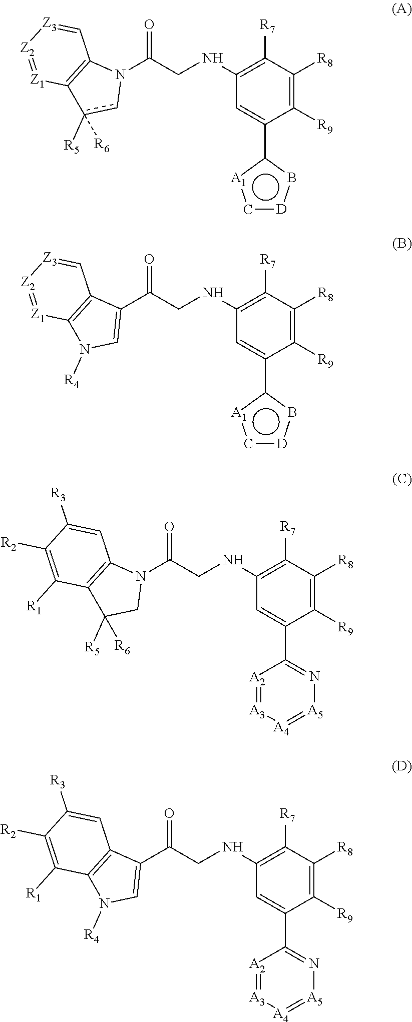

Preferred embodiments are defined in the appended dependent claims.

In a preferred embodiment, in the compounds according to the invention and/or the claims, H is Tritium (³H), and/or F is ¹⁸ F, and/or C is ¹¹C or ¹³C or ¹⁴C, and/or O is ¹⁵O, and/or N is ¹³N, and/or S is ³⁵S, and/or Cl is ³⁶Cl, and/or I is ¹²⁵I or ¹³¹I.

In the combination of different residues, e.g. R₁, R₂ and R₃, and also the combination of residues at substituents thereof such as e.g. —ORₓ, —SRₓ, —S(═O)₁₋₂-Rₓ or —C(═O)ORₓ, a substituent, e.g. Rₓ, can assume different meanings within a substance for two or more residues, e.g. R₁, R₂ and R₃.

For the purposes of the description hydrocarbon residues are divided into aliphatic hydrocarbon residues and aromatic hydrocarbon residues.

Aliphatic hydrocarbon residues are themselves divided into non-cyclic aliphatic hydrocarbon residues (=“aliphatic”) and cyclic aliphatic hydrocarbon residues, i.e. alicyclic hydrocarbon residues (=“cycloaliphatic”). Cycloaliphatic compounds can be monocyclic or multicyclic. Alicyclic hydrocarbon residues (“cycloaliphatic”) comprise both pure aliphatic carbocycles and aliphatic heterocycles, i.e. —unless expressly specified—“cycloaliphatic” comprises pure aliphatic carbocycles (e.g. cyclohexyl), pure aliphatic heterocycles (e.g. piperidyl or piperazyl) and also non-aromatic, multicyclic, possibly mixed, systems (e.g. decalinyl, decahydroquinolinyl).

Aromatic hydrocarbons are themselves divided into carbocyclic aromatic hydrocarbons (=“aryl”) and heterocyclic aromatic hydrocarbons (=“heteroaryl”).

The classification of multicyclic, at least partially aromatic systems preferably depends on whether at least one aromatic ring of the multicyclic system has at least one heteroatom (usually N, O or S) in the ring. If at least one such heteroatom is present in this ring, this is preferably a “heteroaryl” (even if a further carbocyclic aromatic or non-aromatic ring with or without heteroatom is possibly present as additionally present cycle of the multicyclic system); if such a heteroatom is not present in any of the possibly several aromatic rings of the multicyclic system, then this is preferably “aryl” (even if a ring heteroatom is present in a possibly additionally present non-aromatic cycle of the multicyclic system).

Therefore, the following priority in the classification applies within the cyclic substituents: heteroaryl >aryl >cycloaliphatic.

For the purposes of the description monovalent and multivalent, i.e. bivalent, hydrocarbon residues are not distinguished between conceptually, i.e. depending on the context, “C₁₋₈-aliphatic” covers e.g. —C₁₋₈-alkyl, —C₁₋₈-alkenyl and —C₁₋₈-alkinyl, as well as e.g. —C₁₋₈-alkylene-, —C₁₋₈-alkenylene- and C₁₋₈-alkinylene.

Aliphatic means preferably respectively a branched or unbranched, saturated or a mono- or polyunsaturated, unsubstituted or mono- or polysubstituted, aliphatic hydrocarbon residue. Where aliphatic is mono- or polysubstituted, the substituents are selected independently of one another from the group comprising —F, —Cl, —Br, —I, —CN, —NO₂, —CHO, ═O, —Rₓ, —C(═O)Rₓ, —C(═O)H, —C(═O)OH, —C(═O)ORₓ, —C(═O)NH₂, —C(═O)NHRₓ, —C(═O)N(Rₓ)₂, —OH, —ORₓ, —OC(═O)H, —OC(═O)Rₓ, —OC(═O)—ORₓ, —OC(═O)NHRₓ, —OC(═O)N(Rₓ)₂, —SH, -SRₓ, —SO₃H, —S(═O)₁₋₂-Rₓ, —S(═O)₁₋₂NH₂, —NH₂, —NHRₓ, —N(Rₓ)₂, —N⁺(Rₓ)₃, —N⁺(Rₓ)₂ O⁻, —NHC(═O)Rₓ, —NHC(═O)ORₓ, —NHC(═O)NH₂, —NHC(═O)NHRₓ, —NHC(═O)—N(Rₓ)₂, —Si(Rₓ)₃ or —PO(ORₓ)₂.

Thus, “aliphatic” covers acyclic saturated or unsaturated hydrocarbon residues that can be branched or straight-chain, i.e. alkanyls, alkenyls and alkinyls. In this case, alkenyls have at least one C═C double bond and alkinyls have at least one C≡C triple bond. Preferred unsubstituted monovalent aliphatics comprise —CH₃, —CH₂CH₃, —CH₂CH₂CH₃, —CH(CH₃)₂, —CH₂CH₂CH₂CH₃, —CH(CH₃)CH₂CH₃, —CH₂CH(CH₃)₂, —C(CH₃)₃, —CH₂CH₂CH₂—CH₂CH₃ and —CH₂CH₂CH₂CH₂CH₂CH₃; but also —CH═CH₂, —C—CH, —CH₂CH═CH₂, —CH═CHCH₃, —CH₂C—CH, —C—CCH₃ and —CH═CHCH═CH₂. Preferred unsubstituted bivalent aliphatics comprise —CH₂—, —CH₂CH₂—, —CH₂CH(CH₃)—, —CH(CH₃)—CH₂—, —CH₂CH₂CH₂—, —CH(CH₃)CH₂CH₂—, —CH₂CH(CH₃)—CH₂—, —CH₂CH₂CH(CH₃)—, —CH—(CH₂CH₃)CH₂— and —CH₂CH₂—CH₂CH₂—; but also —CH═CH—, —C—C—, —CH₂CH═CH—, —CH═CHCH₂—, —CH₂C—C— and —C—CCH₂—. Preferred substituted monovalent aliphatics comprise —CH₂F, —CHF₂, —CF₃, —CH₂CF₃, —CF₂CF₃, —CH₂OH, —CH₂CH₂OH, —CH₂CHOHCH₃, —CH₂OCH₃, —CH₂CH₂OCH₃ and —CH₂N(CH₃)₂. Preferred substituted bivalent aliphatics comprise —CF₂—, —CF₂CF₂—, —CH₂CHOH—, —CHOHCH₂— and —CH₂CHOHCH₂—·-Methyl-, -ethyl-, -n-propyl- and -n-butyl-are particularly preferred.

Cycloaliphatic means preferably respectively a saturated or a mono- or polyunsaturated, unsubstituted or mono- or polysubstituted, aliphatic (i.e. not aromatic), mono- or multicyclic hydrocarbon residue. The number of ring-carbon atoms preferably lies in the specified range (i.e. a “C₃₋₁₂-cycloaliphatic” preferably has 3, 4, 5, 6, 7, 8, 9, 10, 11 or 12 ring-carbon atoms). For the purposes of the description “C₃₋₁₂-cycloaliphatic” is preferably a cyclic hydrocarbon with 3, 4, 5, 6, 7, 8, 9, 10, 11 or 12 ring-carbon atoms, saturated or unsaturated, but not aromatic, wherein possibly one or two or more carbon atoms are replaced independently of one another by a heteroatom S, N or O. Where cycloalkyl is mono- or polysubstituted, the substituents are selected independently of one another from the group comprising —F, —Cl, —Br, —I, —CN, —NO₂, —CHO, ═O, —Rₓ, —C(═O)Rₓ, —C(═O)H, —C(═O)OH, —C(═O)ORₓ, —C(═O)NH₂, —C(═O)NHRₓ, —C(═O)N(Rₓ)₂, —OH, —ORₓ, —OC(═O)H, —OC(═O)Rₓ, —OC(═O)—ORₓ, —OC(═O)NHRₓ, —OC(═O)N(Rₓ)₂, —SH, -SRₓ, —SO₃H, —S(═O)₁₋₂-Rₓ, —S(═O)₁₋₂NH₂, —NH₂, —NHRₓ, —N(Rₓ)₂, —N⁺(Rₓ)₃, —N⁺(Rₓ)₂ O⁻, —NHC(═O)Rₓ, —NHC(═O)ORₓ, —NHC(═O)NH₂, —NHC(═O)NHRₓ, —NHC(═O)—N(Rₓ)₂, —Si(Rₓ)₃ or —PO(ORₓ)₂.

Preferably, C₃₋₁₂-cycloaliphatic is selected from the group comprising cyclopropyl, cyclobutyl, cyclopentyl, cyclohexyl, cycloheptyl, cyclooctyl, cyclononyl, cyclodecyl, cycloundecyl, cyclododecyl, cyclopentenyl, cyclohexenyl, cycloheptenyl, cyclooctenyl, cyclononenyl, cyclodecenyl, cycloundecenyl, cyclododecenyl, but also tetrahydropyranyl, dioxanyl, dioxolanyl, morpholinyl, piperidinyl, piperazinyl, pyrazolinonyl and pyrrolidinyl.

In association with “aliphatic” or “cycloaliphatic”, “mono- or polysubstituted” is preferably understood to mean the mono- or polysubstitution, e.g. the mono-, di-, tri- or 4-substitution, of one or more hydrogen atoms by —F, —Cl, —Br, —I, —OH, —OC₁₋₈-alkyl, —OC(═O)C₁₋₈-alkyl, —SH, —NH₂, —NHCi_s-alkyl, —N(C₁₋₈-alkyl)₂, —C(═O)OC₁₋₈-alkyl or —C(═O)OH. Particularly preferred substituents are —F, —Cl, —OH, —SH, —NH₂ and —C(═O)OH.

Polysubstituted residues are understood to be those residues that are polysubstituted, e.g. twice or three times either at different or at the same atoms, e.g. three times at the same C-atom, as in the case of —CF₃ or —CH₂CF₃, or at different sites, as in the case of —CH(OH)-CH═CH-CHCl₂. The polysubstitution can occur with the same or with different substituents. A substituent may also be substituted itself. Thus, —O⁻aliphatic also covers —OCH₂CH₂O—CH₂CH₂OH, amongst others. It is preferred if aliphatic or cycloaliphatic is substituted with —F, —Cl, —Br, —I, —CN, —CH₃, —C₂H₅, —NH₂, —NO₂, —SH, —CF₃, —OH, —OCH₃, —OC₂H₅ or —N(CH₃)₂. It is most particularly preferred if aliphatic or cycloaliphatic is substituted with —OH, —OCH₃ or —OC₂H₅.

Aryl preferably respectively independently stands for a carbocyclic ring system with at least one aromatic ring, but without heteroatoms in this ring, wherein the aryl residues can possibly be condensed with further saturated, (partially) unsaturated or aromatic ring systems and each aryl residue can be present in unsubstituted or mono- or polysubstituted form, wherein the aryl substituents are the same or different and can be in any desired and possible position of the aryl. Preferred aryls are phenyl, naphthyl, anthracenyl, phenanthrenyl, fluoroanthenyl, fluoroenyl, indanyl and tetralinyl. Phenyl and naphthyl are particularly preferred. Where aryl is mono- or polysubstituted, the aryl substituents can be the same or different and be in any desired and possible position of the aryl, and are selected independently of one another from the group comprising —F, —Cl, —Br, —I, —CN, —NO₂, —CHO, ═O, —Rₓ, —C(═O)Rₓ, —C(═O)H, —C(═O)OH, —C(═O)ORₓ, —C(═O)NH₂, —C(═O)NHRₓ, —C(═O)—N(Rₓ)₂, —OH, —O(CH₂)₁₋₂O—, —ORₓ, —OC(═O)H, —OC(═O)Rₓ, —OC(═O)ORₓ, —OC(═O)NHRₓ, —OC(═O)N(Rₓ)₂, —SH, -SRₓ, —SO₃H, —S(═O)₁₋₂-Rₓ, —S(═O)₁₋₂NH₂, —NH₂, —NHRₓ, —N(Rₓ)₂, —N⁺(Rₓ)₃, —N⁺(Rₓ)₂ O⁻, —NHC(═O)Rₓ, —NHC(═O)ORₓ, —NH—C(═O)NH₂, —NHC(═O)NHRₓ, —NHC(═O)—N(Rₓ)₂, —Si(Rₓ)₃ and —PO(ORₓ)₂; wherein if necessary N-ring atoms present can be respectively oxidized. Preferred substituted aryls are 2-fluorophenyl, 3-fluorophenyl, 4-fluorophenyl, 2,3-difluorophenyl, 2,4-difluorophenyl, 3,4-difluorophenyl, 2-chlorophenyl, 3-chlorophenyl, 4-chlorophenyl, 2,3-dichlorophenyl, 2,4-dichlorophenyl, 3,4-dichlorophenyl, 2-methoxy-phenyl, 3-methoxy-phenyl, 4-methoxy-phenyl, 2,3-dimethoxy-phenyl, 2,4-dimethoxy-phenyl, 3,4-dimethoxy-phenyl, 2-methyl-phenyl, 3-methyl-phenyl, 4-methyl-phenyl, 2,3-dimethyl-phenyl, 2,4-dimethyl-phenyl and 3,4-dimethyl-phenyl.

Heteroaryl preferably stands for a 5-, 6- or 7-membered cyclic aromatic residue that contains 1, 2, 3, 4 or 5 heteroatoms, wherein the heteroatoms, the same or different, are nitrogen, oxygen or sulphur, and the heterocycle can be unsubstituted or mono- or polysubstituted; wherein in the case of the substitution on the heterocycle, the substituents can be the same or different and can be in any desired and possible position of the heteroaryl; and wherein the heterocycle can also be part of a bi- or polycyclic system. “Heteroaryl” is preferably selected from the group comprising pyrrolyl, indolyl, furyl (furanyl), benzofuranyl, thienyl (thiophenyl), benzothienyl, benzothiadiazolyl, benzooxadiazolyl, benzothiazolyl, benzooxazolyl, benzotriazolyl, benzodioxolanyl, benzodioxanyl, phthalazinyl, pyrazolyl, imidazolyl, thiazolyl, oxazolyl, isoxazoyl, pyridinyl, pyridazinyl, pyrimidinyl, pyrazinyl, pyranyl, indazolyl, purinyl, indolizinyl, quinolinyl, isoquinolinyl, quinazolinyl, carbazolyl, phenazinyl, phenothiazinyl or oxadiazolyl, wherein the bonding can occur via any desirable and possible ring member of the heteroaryl residue. Where heteroaryl is mono- or polysubstituted, the heteroaryl substituents can be the same or different and can be in any desirable and possible position of the heteroaryl, and are selected independently of one another from the group comprising —F, —Cl, —Br, —I, —CN, —NO₂, —CHO, ═O, —Rₓ, —C(═O)Rₓ, —C(═O)H, —C(═O)OH, —C(═O)ORₓ, —C(═O)NH₂, —C(═O)NHRₓ, —C(═O)—N(Rₓ)₂, —OH, —O(CH₂)₁₋₂O—, —ORₓ, —OC(═O)H, —OC(═O)Rₓ, —OC(═O)ORₓ, —OC(═O)NHRₓ, —OC(═O)N(Rₓ)₂, —SH, -SRₓ, —SO₃H, —S(═O)₁₋₂-Rₓ, —S(═O)₁₋₂NH₂, —NH₂, —NHRₓ, —N(Rₓ)₂, —N⁺(Rₓ)₃, —N⁺(Rₓ)₂ O⁻, —NHC(═O)Rₓ, —NHC(═O)ORₓ, —NH—C(═O)NH₂, —NHC(═O)NHRₓ, —NHC(═O)—N(Rₓ)₂, —Si(Rₓ)₃ and —PO(ORₓ)₂; wherein if necessary N-ring atoms present can be respectively oxidized.

Regarding “aryl” or “heteroaryl”, “mono- or polysubstituted” are understood to mean the mono- or polysubstitution, e.g. di-, tri-, 4- or 5-substitution, of one or more hydrogen atoms of the ring system.

Particularly preferred are the (hetero)aryl substituents selected independently of one another from —F, —Cl, —Br, —I, —CN, —CHO, —CO₂H, —NH₂, —NO₂, —NHRₓ, —N(Rₓ)₂, —N⁺(Rₓ)₃, —N⁺(Rₓ)₂ O⁻, —SH, - SRₓ, —OH, -ORₓ, —C(═O)Rₓ, —CO₂Rₓ, —C(═O)NH₂, —C(═O)NHRₓ, —C(═O)N(Rₓ)₂, —S(═O)₁₋₂Rₓ, —S(═O)₂NH₂, —SO₃H, ═O or —Rₓ. Preferred substituents are —F, —Cl, —Br, —I, —OH, —OC₁₋₈-alkyl, —O—C(═O)—C₁₋₈-alkyl, —SH, —NH₂, —NHC₁₋₈-alkyl, —N(C₁₋₈-alkyl)₂, —C(═O)OC₁₋₈-alkyl or —C(═O)OH. Particularly preferred substituents are —F, —Cl, —OH, —SH, —NH₂ and —C(═O)OH.

Unless expressly stated otherwise, residues having more than a single binding partner can be attached in any direction. For example, the residue “—S—(CH₂)—C(═O)—” which is attached to binding partners B₁ and B₂ can be present in either direction, B₁—S—(CH₂)—C(═O)—B₂ or B₁—C(═O)—(CH₂)—S—B₂.

The compounds according to the invention can be present in the form of a single stereoisomer or mixture thereof, the free compounds and/or their physiologically acceptable salts and/or solvates.

The compounds according to the invention can be chiral or achiral, depending on the substitution pattern.

If the compounds according to the invention are chiral, then they are preferably present as racemate or a mixture of stereoisomers or diastereomers or in enriched form of an enantiomer. In a preferred embodiment the enantiomer excess (ee) of the S-enantiomer amounts to at least 50% ee, more preferred at least 75% ee, more preferred at least 90% ee, most preferred at least 95% ee, and in particular at least 99% ee. In another preferred embodiment, the enantiomer excess (ee) of the R-enantiomer amounts to at least 50% ee, more preferred at least 75% ee, more preferred at least 90% ee, most preferred at least 95% ee, and in particular at least 99% ee.

Suitable methods for separating the enantiomers are known to the person skilled in the art. Preparative HPLC on chiral stationary phases and conversion into diastereomeric intermediates can be given as examples. The conversion into diastereomeric intermediates can occur, for example, as salt formation by means of chiral, enantiomer-pure acids. After separation of the diastereomers thus formed, the salt can then be converted into the free base or another salt again.

Unless expressly specified, each reference to the compounds according to the invention covers all isomers in pure form and admixture with one another (e.g. stereoisomers, diastereomers, enantiomers) in any desired mixture ratio.

Unless expressly specified, each reference to the compounds according to the invention covers the free compounds (i.e. the forms that are not present in the form of salt) and all physiologically acceptable salts.

For the purposes of the description, physiologically acceptable salts of the compounds according to the invention are present as salts with anions or acids of the respective compound with inorganic or organic acids, which are physiologically acceptable—in particular on application in humans and/or mammals.

Examples of physiologically acceptable salts of specific acids are salts of: hydrochloric acid, hydrobromic acid, sulphuric acid, methane sulphonic acid, formic acid, acetic acid, oxalic acid, succinic acid, malic acid, tartaric acid, mandelic acid, fumaric acid, lactic acid, citric acid, glutamic acid, saccharinic acid, monomethyl sebacic acid, 5-oxo-proline, hexane-1-sulphonic acid, nicotinic acid, 2-, 3- or 4-aminobenzoic acid, 2,4,6-trimethyl benzoic acid, α-liponic acid, acetylglycine, acetylsalicylic acid, hippuric acid and/or aspartic acid. The hydrochloride, citrate and hemicitrate are particularly preferred.

Physiologically acceptable salts with cations or bases are salts of the respective compound—as anion with at least one, preferably inorganic, cation, which are physiologically acceptable—in particular on application in humans and/or mammals. Particularly preferred are the salts of the alkali and earth alkali metals, also ammonium salts, but in particular (mono-) or (di-) sodium, (mono-) or (di-) potassium, magnesium or calcium salts.

The compounds according to the invention are defined by substituents, e.g. by R₁, R₂ and R₃ (substituents of the first generation), which are themselves possibly substituted (substituents of the second generation). Depending on the definition, these substituents of the substituents can themselves be substituted again (substituents of the third generation). If, for example, R₁=—Rₓ, wherein —Rₓ=—C₁₋₈ aliphatic (substituent of the first generation), then —C₁₋₈-aliphatic can itself be substituted, e.g. with —ORₓ, wherein Rₓ=—aryl (substituent of the second generation). This gives the functional group —C₁₋₈-aliphatic-O-aryl. -Aryl can then in turn be substituted again, e.g. with —Cl (substituent of the third generation). This then gives overall the functional group —C₁₋₈-aliphatic-O-aryl-Cl.

Another aspect of the invention relates to the compounds according to the invention as described above as medicaments.

Another aspect of the invention relates to pharmaceutical compositions or pharmaceutical dosage forms comprising the compounds according to the invention as described above.

Preferably, the pharmaceutical compositions comprise a compound according to the invention as described above, and a pharmaceutically acceptable carrier. The term “pharmaceutically acceptable carrier,” as used herein, means a non-toxic, inert solid, semi-solid or liquid filler, diluent, encapsulating material or formulation auxiliary of any type.

Some examples of materials which may serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as releasing agents, coloring agents, coating agents, sweetening, flavoring and perfuming agents, preservatives and antioxidants may also be present in the composition, according to the judgment of one skilled in the art of formulations.

The pharmaceutical compositions may be administered to subjects (e.g., humans and other mammals) orally, rectally, parenterally, intravaginally, intracisternally, intraperitoneally, topically (as by powders, ointments or drops), bucally, extracorporeally, e.g. by dialysis, or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (polyethylene glycol, propylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions may also contain adjuvants, such as preservative agents, wetting agents, emulsifying agents, and dispersing agents. Prevention of the action of microorganisms may be ensured by various antibacterial and antifungal agents, for example, parabens, phenol, chlorobutanol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug may depend upon its rate of dissolution, which, in turn, may depend upon crystal size and crystalline form. Alternatively, a parenterally administered drug form may be administered by dissolving or suspending the drug in an oil vehicle.

Suspensions, in addition to the active compounds, may contain suspending agents, for example, polyoxyethylene sorbitol, ethoxylated isostearyl alcohols, and sorbitan esters, microcrystalline cellulose, aluminum metahydroxide, bentonite, agar-agar, tragacanth, and mixtures thereof.

If desired, and for more effective distribution, the compounds may be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release may be controlled. Depot injectable formulations also are prepared by entrapping the drug in liposomes or microemulsions which are compatible with body tissues.

The injectable formulations may be sterilized, for example, by filtration through a bacterial-retaining filter or by incorporating sterilizing agents in the form of sterile solid compositions which may be dissolved or dispersed in sterile water or other sterile injectable medium just prior to use.

Injectable preparations, for example, sterile injectable aqueous or oleaginous suspensions may be formulated according to the known art using suitable dispersing or wetting agents, suspending agents and the like. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid are used in the preparation of injectables.

Solid dosage forms for oral administration include, but are not limited to, capsules, tablets, pills, powders, and granules. In such solid dosage forms, one or more compounds is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules may be prepared with coatings and shells such as enteric coatings and other coatings well-known in the pharmaceutical formulating art. They optionally may contain opacifying agents and also may be of a composition that they release the active ingredient(s) only, or preferentially, in a certain part of the intestinal tract in a delayed manner. Examples of materials useful for delaying release of the active agent may include polymeric substances and waxes.

Compositions for rectal or vaginal administration are preferably suppositories which may be prepared by mixing the compounds with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration may include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, and perfuming agents.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds may be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Dosage forms for topical or transdermal administration of a compound include ointments, pastes, creams, lotions, gels, powders, solutions, sprays, inhalants or patches. A desired compound is admixed under sterile conditions with a pharmaceutically acceptable carrier and any needed preservatives or buffers as may be required. Ophthalmic formulation, eardrops, eye ointments, powders and solutions are also contemplated as being within the scope of this disclosure.

The ointments, pastes, creams and gels may contain, in addition to an active compound, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to the compounds, lactose, talc, silicic acid, aluminum hydroxide, calcium silicates and polyamide powder, or mixtures of these substances. Sprays additionally may contain customary propellants such as chlorofluorohydrocarbons.

Compounds also may be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds, stabilizers, preservatives, and the like. The preferred lipids are the natural and synthetic phospholipids and phosphatidylcholines (lecithins) used separately or together. Methods to form liposomes are known in the art.

Dosage forms for topical administration of a compound according to the invention as described above include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also possible. Aqueous liquid compositions may also be useful.

The compounds according to the invention are preferably administered once daily, twice daily, thrice daily or more often to a subject in need thereof.

The compounds according to the invention are preferably administered orally, rectally, intravenously, intramuscularly, intraperitoneally, intrasternally, subcutaneously, by intraarticular injection, by infusion, intravaginally, intracisternally, intraperitoneally, topically, bucally or extracorporeally.

In some cases it is of advantage to treat a patient with a combination of cancer medicaments to achieve the desired remission of cancer cells. The need for such a combination of cancer medicaments, i.e. a combination therapy, particularly arises when cancer cells are or become resistant to conventional cancer medicaments such as e.g. tyrosine-kinase inhibitors. Such a resistance to conventional cancer medicaments has for example been observed in lung cancer cells which may exhibit a resistance to tyrosine-kinase inhibitors after the patient has been treated with tyrosine-kinase inhibitors for a while.

Another aspect of the invention relates to a pharmaceutical composition comprising a combination of
a compound according to general Formula A, general Formula B, general Formula C and/or general Formula D; and
a second pharmacologically active compound.

All aspects of a pharmaceutical composition comprising the compounds according to the invention as described above also apply to the pharmaceutical composition comprising a combination of a compound according to general Formula A, general Formula B, general Formula C and/or general Formula D and a second pharmacologically active compound.

Another aspect of the invention relates to a kit comprising
a first pharmaceutical composition comprising a compound according to general Formula A, general Formula B, general Formula C and/or general Formula D; and
a second pharmaceutical composition comprising a second pharmacologically active compound;
   wherein the first pharmaceutical composition and the second pharmaceutical composition are separate of one another.

Preferably, the first pharmaceutical composition and the second pharmaceutical composition of said kit are for administration through the same route. In another embodiment of the invention, the first pharmaceutical composition is for administration through a different route than the second pharmaceutical composition.

Preferred administration routes for the first and second pharmaceutical composition of said kit are oral, rectal parenteral, intravaginal, intracisternal, intraperitoneal, topical or bucal administration to a patient.

Preferably, the first pharmaceutical composition and the second pharmaceutical composition of said kit are administered to a patient subsequent to each other, wherein
the first pharmaceutical composition is administered first, followed by administration of the second pharmaceutical composition, or
the second pharmaceutical composition is administered first, followed by administration of the first pharmaceutical composition.

It is preferred that the period of time between the administration of the first pharmaceutical composition and the second pharmaceutical composition, or vice versa, is at least 1 hour, at least 2 hours, at least 3 hours, at least 4 hours or at least 5 hours.

More preferred, the period of time between the administration of the first pharmaceutical composition and the second pharmaceutical composition, or vice versa, is at least 1 day, at least 2 days, at least 3 days, at least 4 days, at least 5 days, at least 6 days, at least 7 days or at least 8n days.

More preferred, the period of time between the administration of the first pharmaceutical composition and the second pharmaceutical composition, or vice versa, is at least 2 weeks, at least 3 weeks, at least 4 weeks, at least 5 weeks, at least 6 weeks or at least 7 weeks or at least 8 weeks.

All aspects of a pharmaceutical composition comprising the compounds according to the invention as described above also apply to the first pharmaceutical composition and the second pharmaceutical composition of said kit.

The following examples further illustrate the invention but are not to be construed as limiting its scope.

### Chemistry and the Experimental Details LIST OF THE ABBREVIATIONS

CH₃CN Acetonitrile
Ar Argon gas
APCI Atmospheric pressure chemical ionization
AlCl₃ Aluminium chloride
Aq. Ageous
anhyd. Na₂SO₄ Anhydrous sodium sulfate
AcOH Acetic acid
Ac₂O Acetic anhydride
(Boc)₂O Di-tert-butyl dicarbonate
BINAP 2,2′-Bis(diphenylphosphino)-1,1′-binaphthyl
BBr₃ Boron tribromide
BtOH·H₂O p-Toluenesulfonic acid
CO Carbon monoxide
CuCl Copper monochloride
CDCl₃ Chloroform
Cs₂CO₃ Cesium carbonate
Conc. Concentrated
Cbz Benzyl chloroformate
DCE 1,2-Dichloroethane
DMSO Dimethyl sulfoxide
DCM Dichloromethane
DMF N,N-Dimethylformamide
DAD Diode array detector
DMA N,N-Dimethylacetamide
DAST Diethylaminosulfur trifluoride
DMAC Dimethylacetamide
DMP Dimethyl phthalate
DEAD Diethyl azodicarboxylate
DIAD Diisopropyl azodicarboxylate
4-DMAP 4-Dimethylaminophenol
DIPEA N,N-Diisopropylethylamine
ESI-MS Electrospray ionization mass spectrometry
Et₃SiH Triethylsilane
EtOH Ethanol
EDCI 1-(3-Dimethylaminopropyl)-3-ethylcarbonimide
Eaton's reagent Methanesulfonic acid-phosphorus(V) oxide
Et₂O Diethyl ether
HOBT Hydroxybenzotriazole
EtOAc Ethylacetate
Equiv. Equivalent
FeCl₂ Ferrous chloride
H Hour
HATU O-(7-Azabenzotriazol-1-yl)-N,N,N′,N′-tetramethyluronium-hexafluorphosphat
HPLC High performance liquid chromatography
HRMS High resolution mass spectrometry
HPLC-UV High-performance liquid chromatography-ultraviolet
H₂ Hydrogen gas
H₅IO₆ Orthoperiodic acid
LC-MS Liquid chromatography-mass spectrometry
IPA Isopropylalcohol
INT Intermediate
K₂CO₃ Potassium carbonate
KMnO₄ Potassium permanganate
KOH Potassium hydroxide
LiOH·H₂O Lithium hydroxide monohydrate
L Liter
M⁺ Molecular ions
MeOH Methanol
MeMgBr Methylmagnesium bromide
NaN₃ Sodium azide
NMP N-Methyl-2-pyrrolidone
NBS n-Bromosuccinimide
NaBH₃CN Sodium cyanoborohydride
NaBH₄ Sodium borohydride
NaOH Sodium hydroxide
NaOtBu Sodium tert-butoxide
NMO N-Methylmorpholine N-oxide
NaH Sodiumhydride
NaHCO₃ Sodium bicarbonate
NaSMe Sodium methanethiolate
Na₂CO₃ Sodium carbonate
NH₄Cl Ammonium chloride
NH₄OH Ammonium hydroxide
POCl₃ Phosphorus oxychloride
PE Petroleum ether
PPh₃ Triphenylphosphine
Pd/C Palladium on carbon
Ppm parts per million
Pd(OAc)₂ Palladium(II) acetate
Pd(OH)₂/C Palladium hydroxide on carbon
Pd(PPh₃)₄ Tetrakis(triphenylphosphine)palladium(0)
Rt Room temperature
R_{f} Retension time
RP-18 Reverse phase
Rochelle's salt Potassium sodium tartrate tetrahydrate
sat. Saturated
soln. Solution
SO₂ Sulfur dioxide
SOCl₂ Thionyl chloride
STEP Stepwise
SnCl₂ Tin(II) chloride
SM Starting material
SEM 2-(Trimethylsilyl)ethoxymethyl Ether
TLC Thin layer chromatography
TBAF Tetrabutylammoniumfluoride
T3P 1-Propanephosphonic acid cyclic anhydride
TFA Trifluoroacetic acid
TBAB Tetrabutylammoniumbromide
THF Tetrahydrofuran
TIPS Triisopropylsilyl
Et₃N Triethylamine
t_{R} Retention time
t-BuLi tert-Butyllithium
TMSI Trimethylsilyl iodide
V Volt

### Chemicals and Solvents

Chemicals were purchased from Merck (Darmstadt, Germany), ABCR (Karlsruhe, Germany), or TCI (Eschborn, Germany). Solvents were used of normal and/or HPLC grade. Dry solvents were purchased from Acros Organics and Fisher Scientific GmbH, Schwerte, Germany.

### NMR

Proton NMR (¹H-NMR) spectra were recorded on a Bruker Avance DRX 400 and 500 MHz NMR spectrometer or on a Bruker Avance III 600 MHz NMR spectrometer. The chemical shifts (6) are given in ppm and refer to the chemical shifts of the remaining solvent protons present in chloroform (CDCl₃, 7.26 ppm for ¹H-NMR spectra), in dimethylsulfoxide (DMSO-d₆, 2.50 ppm for ¹H-NMR spectra). Spin multiplicities are given as broad (br), singlet (s), doublet (d), triplet (t), quartet (q), pentet (p); and multiplet (m).

### Chromatography

Thin layer chromatography was performed with pre-coated silica gel plates F₂₅₄ (thickness 0.25 mm) or with reversed-phase silica gel plates 60 RP-18 F₂₅₄ from Merck. The evaluation was performed by UV irradiation (254 nm, 366 nm) or staining (KMnO₄, Ninhydrin). Column chromatography was performed on Silica Gel 60 (35-70 μm) or with the automated flash chromatography system Combiflash Rf 200 (Teledyne ISCO, Nebraska, USA). Some of the final compounds were purified with a reversed phase HPLC system from Knauer (column: C18ec, length x internal diameter 250×20 mm, Nucleodur 100-5). For reactions control, the molecular weight was measured on an Advion Expression L APCI- or ESI-MS in combination with a TLC interface.

### LC-MS

Mass spectra were recorded on a micrOTOF-Q mass spectrometer (Bruker) with electrospray ionization (ESI) coupled with an HPLC Dionex Ultimate 3000 (Thermo Scientific) using an EC50/2 Nucleodur C18 Gravity 3 mm column (Macherey-Nagel). A volume of 1 μL of a sample solution 1.0 mgmL⁻¹ was injected. Mobile phase was water containing 2 mM ammonium acetate/acetonitrile (CH₃CN). Elution was performed from 90:10 up to 0:100 in 9 min, 0:100 for 5 min.

The purity of the compounds was determined by HPLC-UV obtained on a LC-MS instrument (Applied Biosystems API 2000 LC/MS/MS, HPLC Agilent 1100). The compounds were dissolved at a concentration of 1.0 mg mL-1 in CH₃CN (Gradient A) or water containing 2 mM ammonium acetate/methanol (MeOH), Gradient B), and if necessary sonicated to complete dissolving. Then, 10 μL of the substance solution was injected into a Phenomenex Luna C18 HPLC column (50×2.00 mm, particle size 3 μm) and elution performed with a gradient of (water (H₂O)/CH₃CN, Gradient A) or (H₂O/MeOH, Gradient B) from 90:10 up to 0:100 in 10 min and 0:100 for 10 min at a flow rate of 300 μLmin⁻¹, starting the gradient after 1 min. UV absorption was detected from 220 to 400 nm using a diode array detector (DAD). The retention times (t_{R}), purities by area and observed molecular ions (M⁺) are reported.

### TLC-MS

Electrospray ionization mass spectrometry (ESI-MS) was performed on an Advion expressions CMS TLC-ESI-MS coupling system (Advion, Ithaca, NY, USA). The parameters of the ESI positive mode (M⁺) were as follows: capillary temperature 250° C., capillary voltage 180 V, source gas temperature 250° C., ESI voltage 3500 V. The parameters of the ESI negative mode (M⁻) were as follows: capillary temperature 250° C., capillary voltage 180 V, source gas temperature 250° C., ESI voltage 2500 V. The compounds were eluted from the TLC plate with MeOH.

### General Methods of Purification of the Final Examples

Method I:

The corresponding products were purified by column chromatography on silica gel using petroleum ether (PE) or cyclohexane:EtOAc (80:20 to 60:40) or DCM:EtOAc (100:0 to 30:70) to give the desired pure products.

Method II:

The corresponding products were supported in Celite® 503 (from Merck) then were purified by flash column chromatography using Heptane/DCM:EtOAc 100:0 to 30:70 or DCM:MeOH 100:0 to 95:5 as an eluent in gradient over 30 min to afford the pure products. The differences in the purification conditions from methods I and II for specific compounds are reported in the corresponding tables.

### Method III (Using Preparative HPLC)

The corresponding products were dissolved in 5 mL of deionized water and injected into a RP-HPLC column (Knauer 20 mm i.d., Eurospher-100 C18). The column was eluted with a solvent gradient of 0-65% CH₃CN in 50 mM aq. NH₄HCO₃ buffer or H₂O:HCO₂H/CH₃CN:HCO₂H, for 40 min at a flow rate of 10 mL/min. The UV absorption was detected at 254 nm. Fractions were collected, and appropriate fractions were pooled, diluted with water, and lyophilized several times to remove the NH₄HCO₃ buffer, yielding the desired compounds.

### Synthesis Procedures

### Synthesis Pathway A

In the synthetic pathway A, starting material SM1, SM2 or SM3 can be modified before entering to intermediate INT-I. SM1 can be attached to 5-membered heterocyles via Suzuki or Negishi coupling following STEP1. Alternatively, 5-membered heterocycle can be prepared stepwise via STEP2 from SM2 carboxylic acid or amide. The 5-Membered heterocycle can be also prepared stepwise via STEP3 from the corresponding cyano derivatives SM3. The resulted nitro derivatives can be reduced to INT-I aniline using several reductive conditions, the most common of which are tin(II) chloride (SnCl₂), Fe/AcOH, and Zn/AcOH and will be described separately in the upcoming schemes. The alkylation of STEP4 aniline can be done with typical alkylating agentes like alpha halogenated alkyl esters. The hydrolysis of STEP5 ester is performed typically with inorganic bases like LiOH or NaOH. In STEP6 amidation, several amidation methods can be used like EDCI/DIPEA, HOBT/DIPEA but mostly done using mild T3P solution in EtOAc or DMF to afford Formula A. Indoline derivatives INT-IV can be acylated using chloroacetyl chloride to produce INT-V (STEP7). STEP8 aniline (INT-I) alkylation can be done using typical alkylating agentes and INT-V to give Formula A (Scheme 1). This pathway may also further derivatize the final Formula A, which will be discussed separately in the upcoming examples.

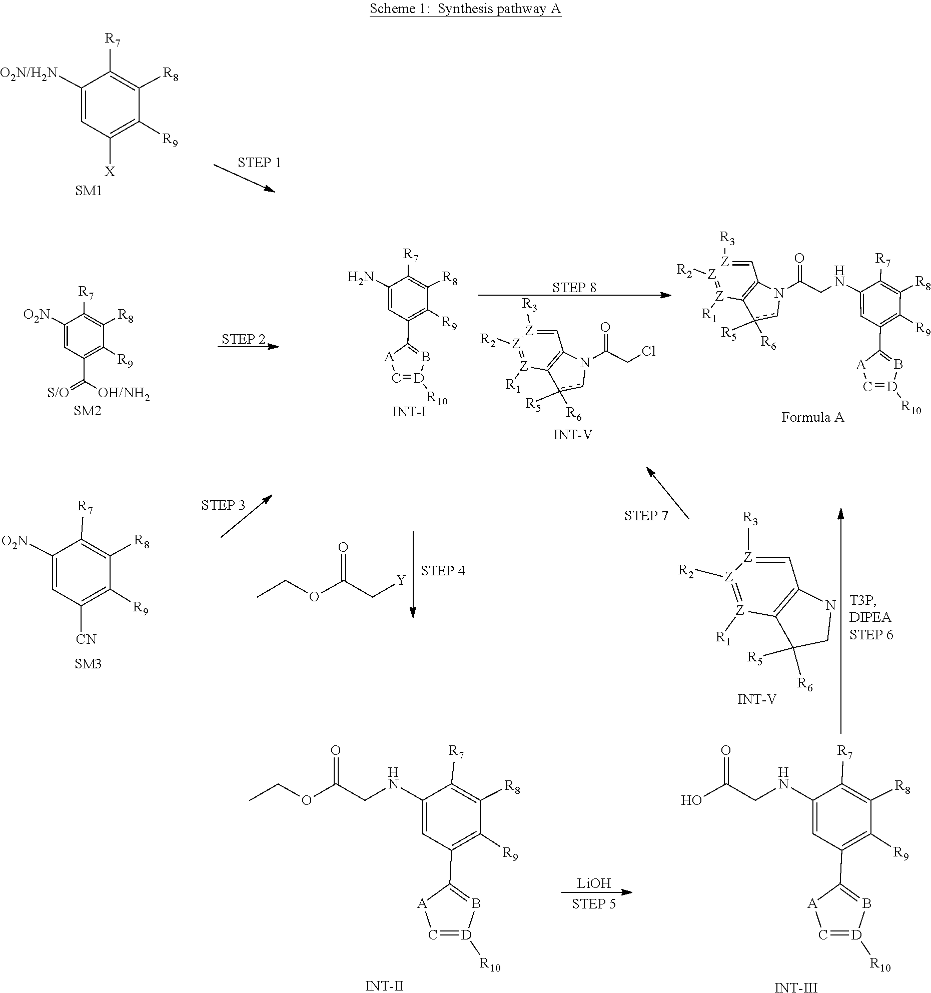

### Synthesis of the Building Blocks and the Intermediate Compounds

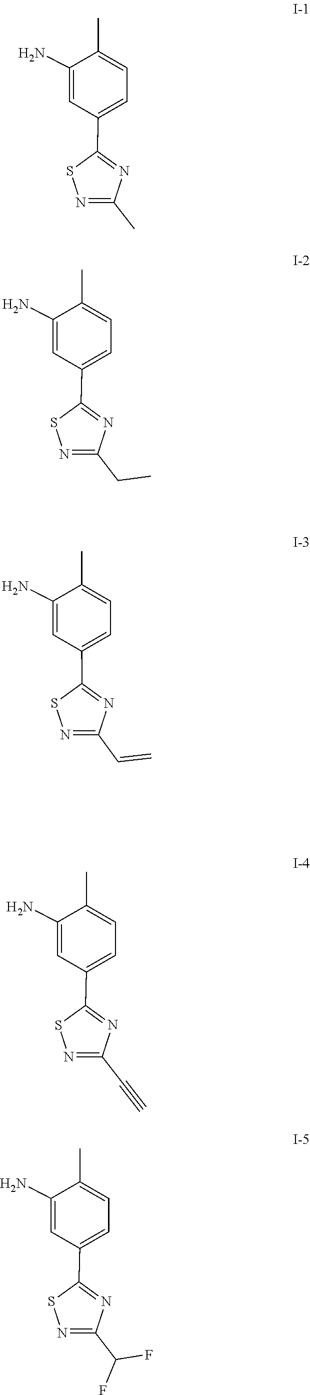 [SMILES: Cc1nsc(-c2ccc(C)c(N)c2)n1 ; CCc1nsc(-c2ccc(C)c(N)c2)n1 ; C=Cc1nsc(-c2ccc(C)c(N)c2)n1 ; C#Cc1nsc(-c2ccc(C)c(N)c2)n1 ; Cc1ccc(-c2nc(C(F)F)ns2)cc1N]

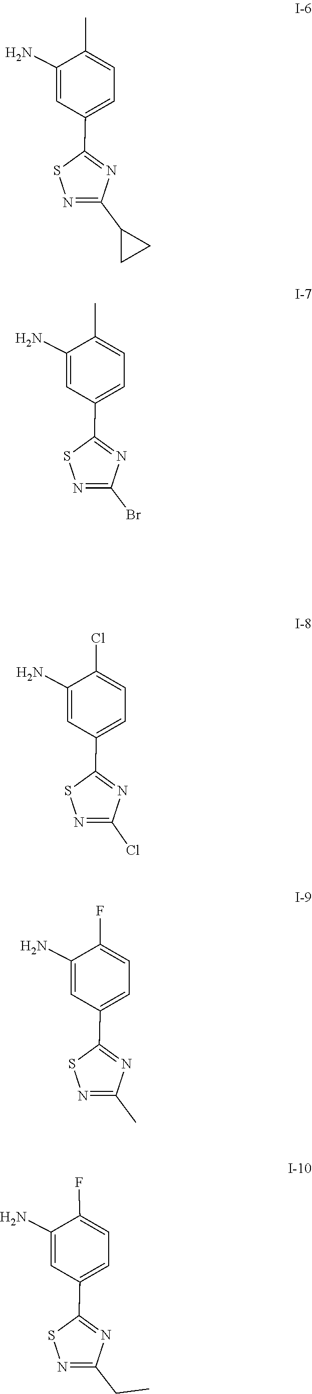 [SMILES: Cc1ccc(-c2nc(C3CC3)ns2)cc1N ; Cc1ccc(-c2nc(Br)ns2)cc1N ; Nc1cc(-c2nc(Cl)ns2)ccc1Cl ; Cc1nsc(-c2ccc(F)c(N)c2)n1 ; CCc1nsc(-c2ccc(F)c(N)c2)n1]

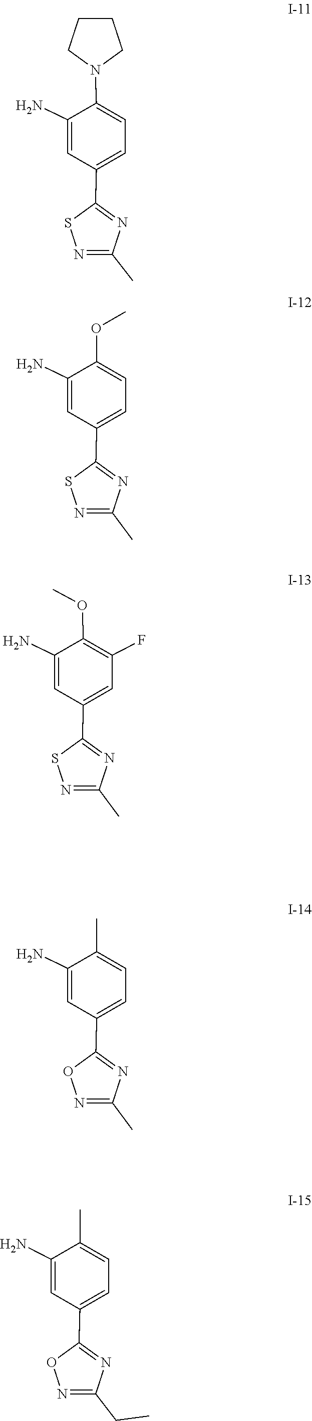

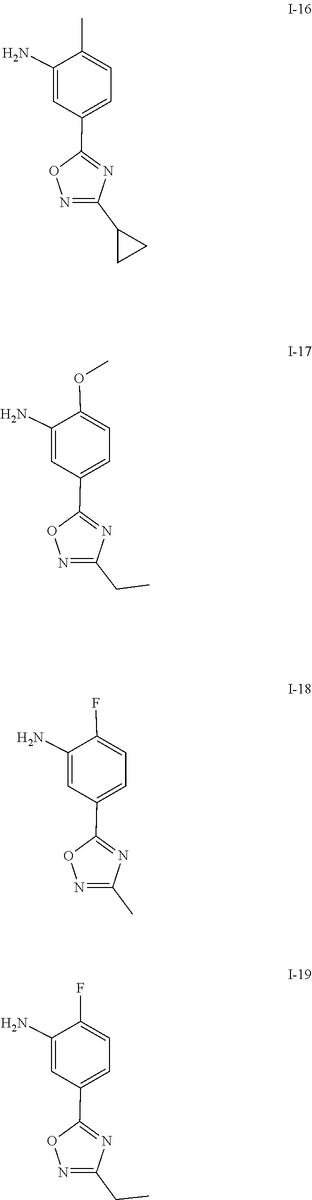 [SMILES: Cc1ccc(-c2nc(C3CC3)no2)cc1N ; CCc1noc(-c2ccc(OC)c(N)c2)n1 ; Cc1noc(-c2ccc(F)c(N)c2)n1 ; CCc1noc(-c2ccc(F)c(N)c2)n1]

### Formulae Part 1: Structures of Intermediates I Used for the Synthesis of the Examples

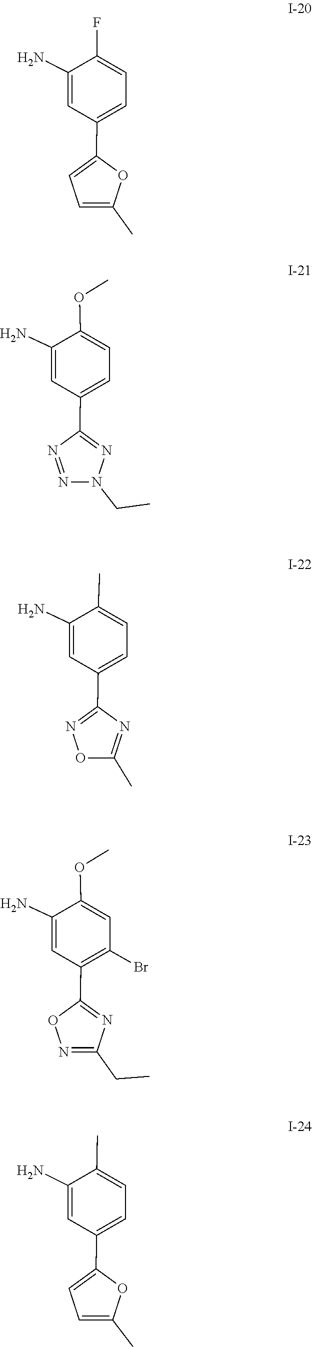 [SMILES: Cc1ccc(-c2ccc(F)c(N)c2)o1 ; CCn1nnc(-c2ccc(OC)c(N)c2)n1 ; Cc1nc(-c2ccc(C)c(N)c2)no1 ; CCc1noc(-c2cc(N)c(OC)cc2Br)n1 ; Cc1ccc(-c2ccc(C)c(N)c2)o1]

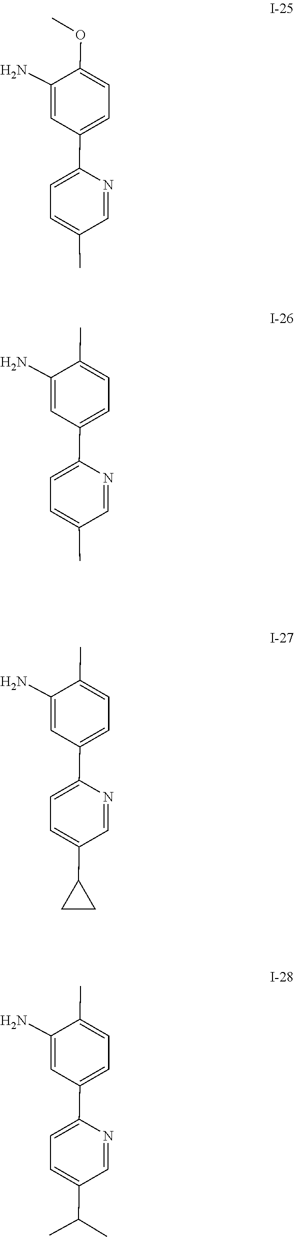 [SMILES: COc1ccc(-c2ccc(C)cn2)cc1N ; Cc1ccc(-c2ccc(C)c(N)c2)nc1 ; Cc1ccc(-c2ccc(C3CC3)cn2)cc1N ; Cc1ccc(-c2ccc(C(C)C)cn2)cc1N]

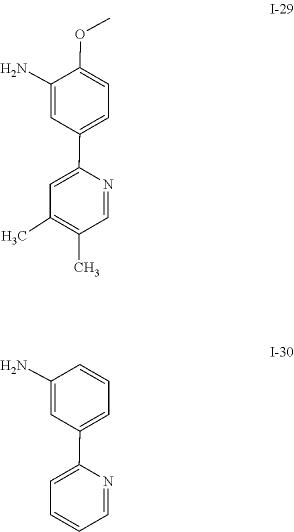 [SMILES: COc1ccc(-c2cc(C)c(C)cn2)cc1N ; Nc1cccc(-c2ccccn2)c1]

### Formulae Part 2: Structures of Intermediates I Used for the Synthesis of the Examples

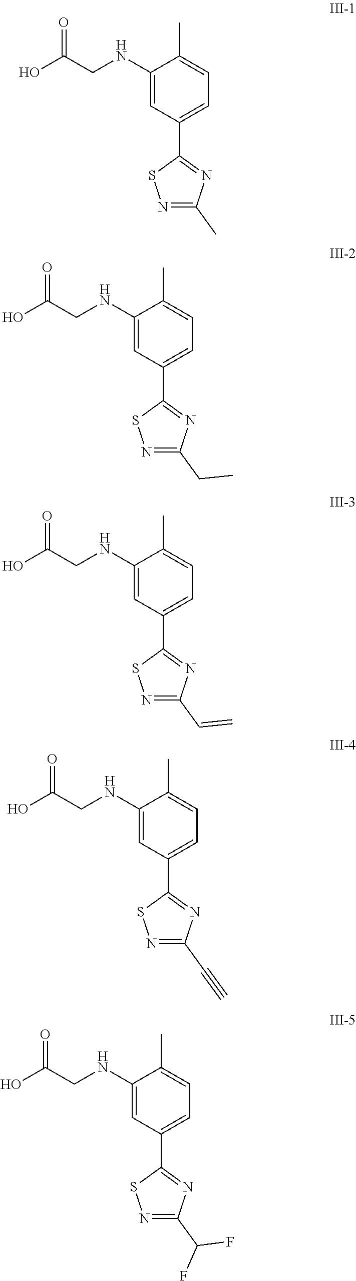

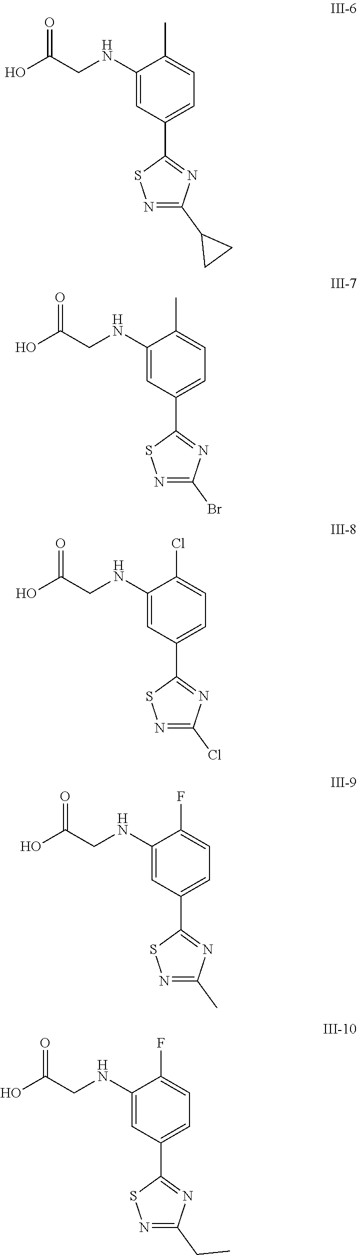

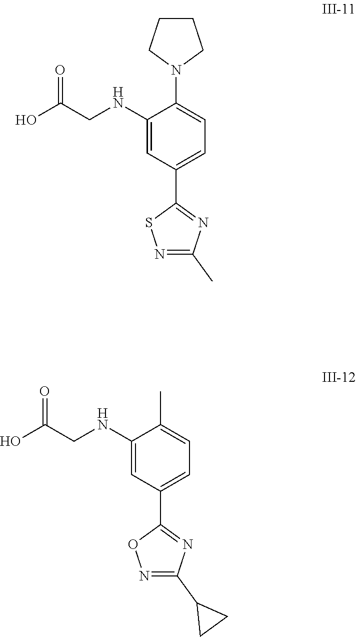 [SMILES: Cc1nsc(-c2ccc(N3CCCC3)c(NCC(=O)O)c2)n1 ; Cc1ccc(-c2nc(C3CC3)no2)cc1NCC(=O)O]

### Formulae Part 3: Structures of Intermediates III Used for the Synthesis of the Examples

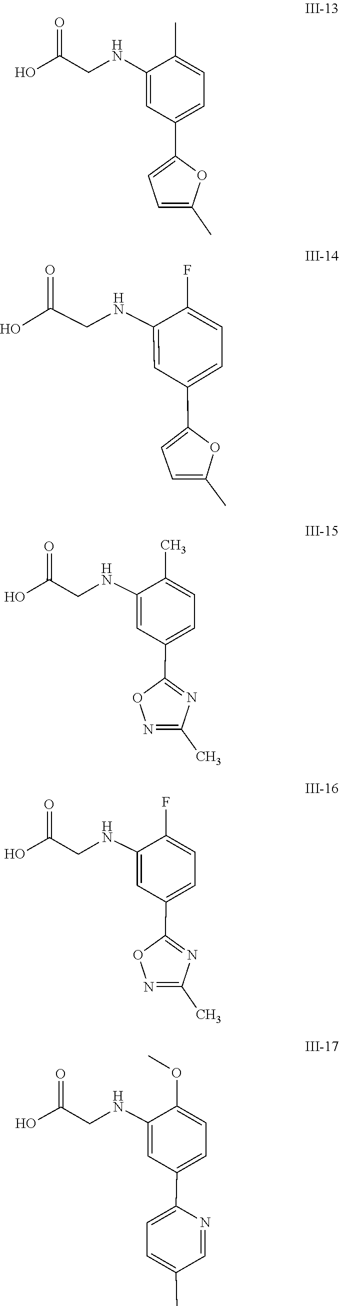

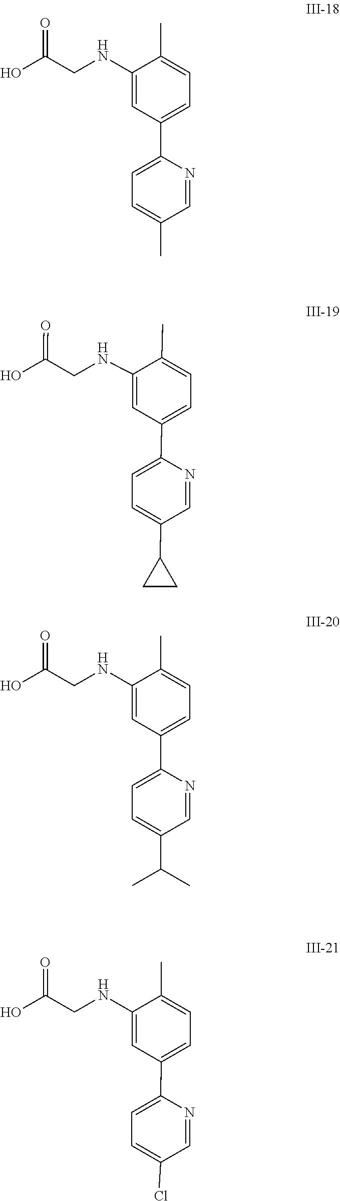 [SMILES: Cc1ccc(-c2ccc(C)c(NCC(=O)O)c2)nc1 ; Cc1ccc(-c2ccc(C3CC3)cn2)cc1NCC(=O)O ; Cc1ccc(-c2ccc(C(C)C)cn2)cc1NCC(=O)O ; Cc1ccc(-c2ccc(Cl)cn2)cc1NCC(=O)O]

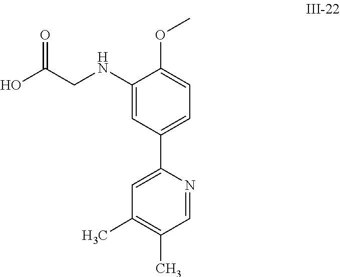 [SMILES: COc1ccc(-c2cc(C)c(C)cn2)cc1NCC(=O)O]

### Formulae Part 4: Structures of Intermediates III Used for the Synthesis of the Examples

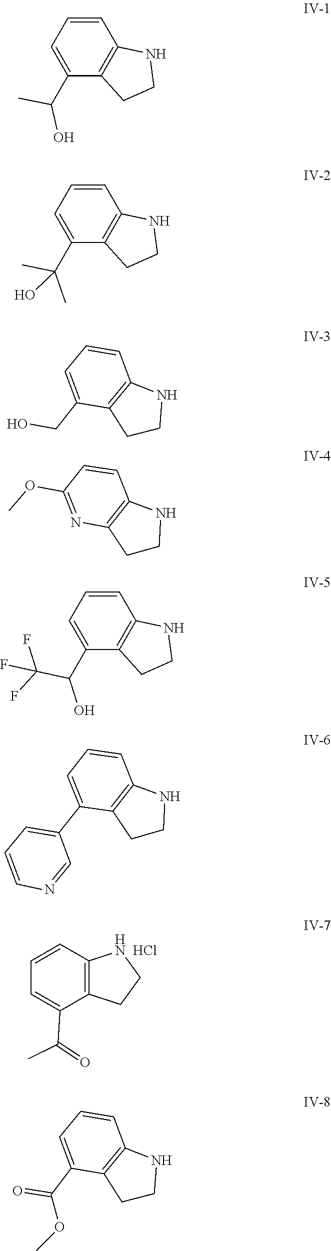

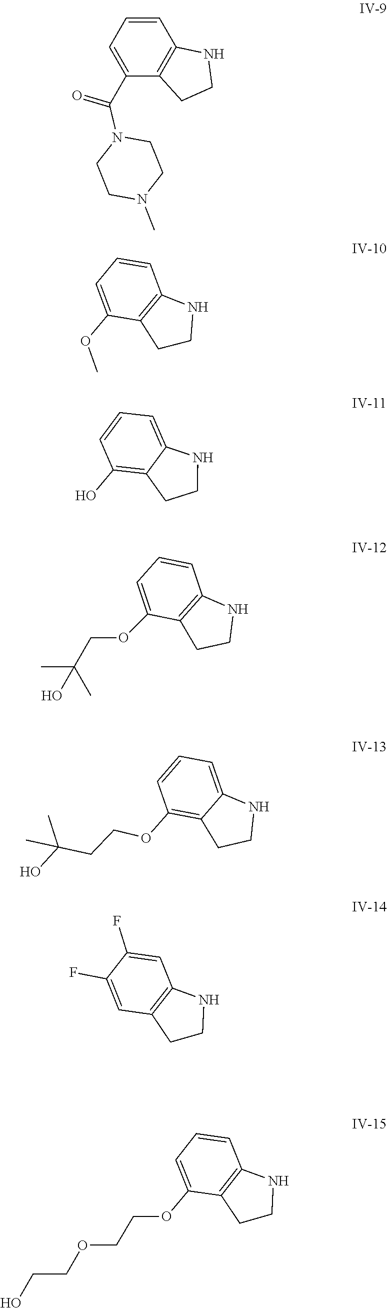

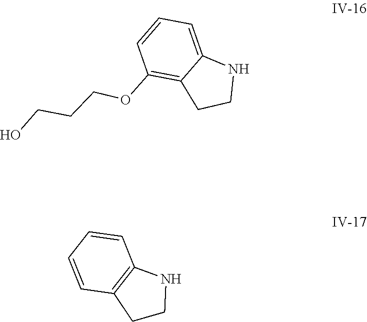 [SMILES: OCCCOc1cccc2c1CCN2 ; c1ccc2c(c1)CCN2]

### Formulae Part 5: Structures of Intermediates IV Used for the Synthesis of the Examples

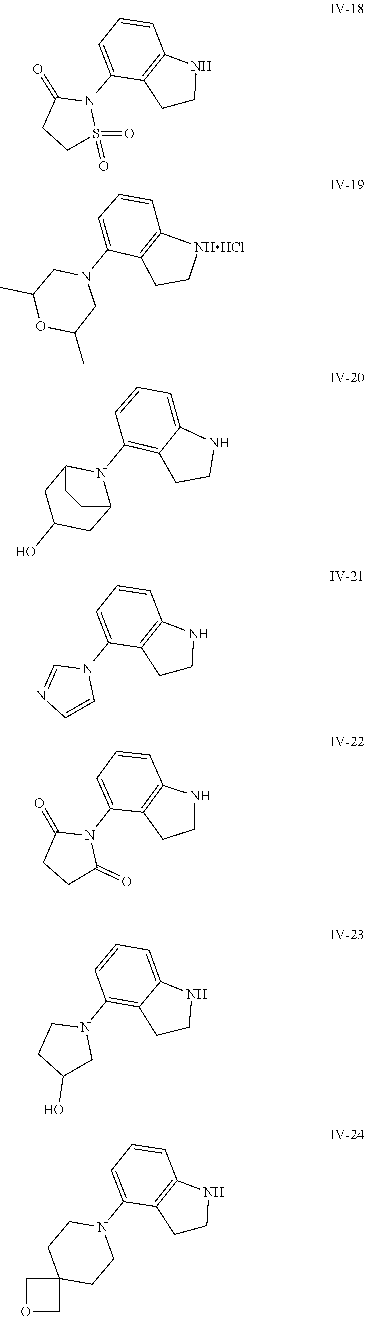

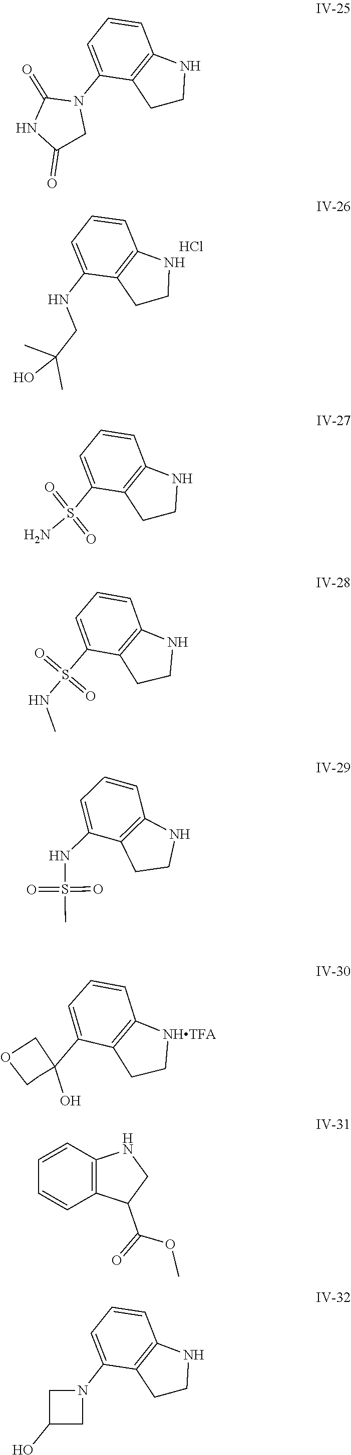

### Formulae Part 6: Structures of Intermediates IV Used for the Synthesis of the Examples

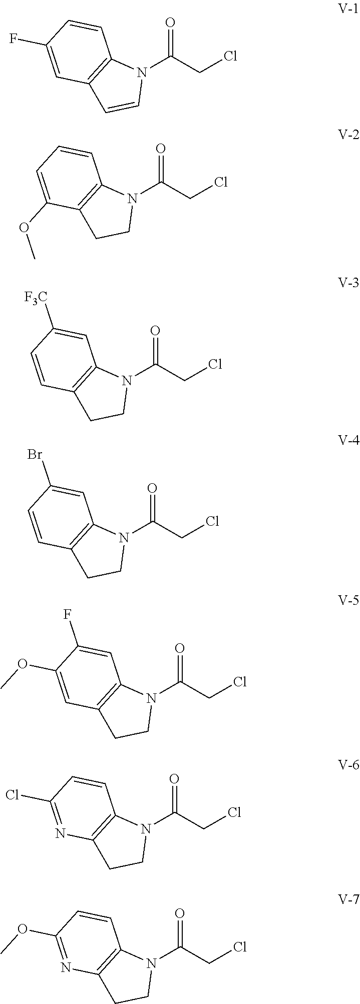

### Formulae Part 7: Structures of Intermediates V Used for the Synthesis of the Examples

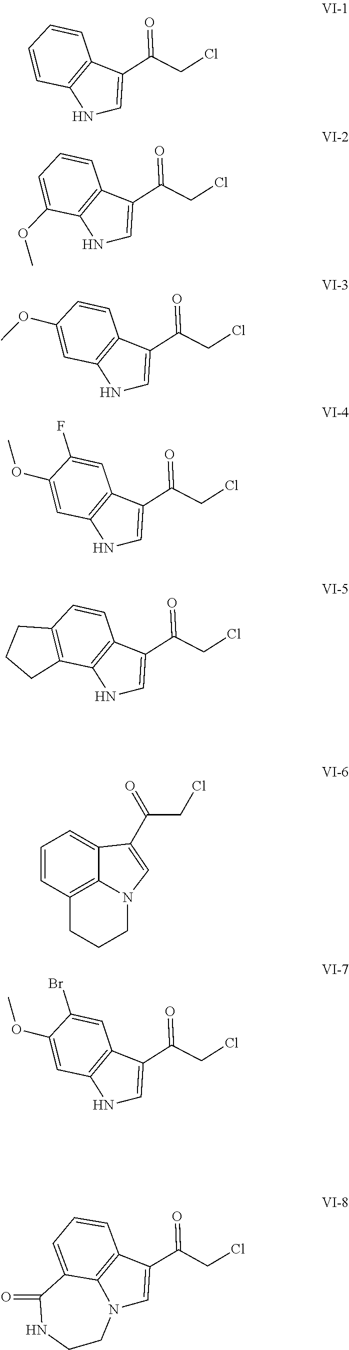

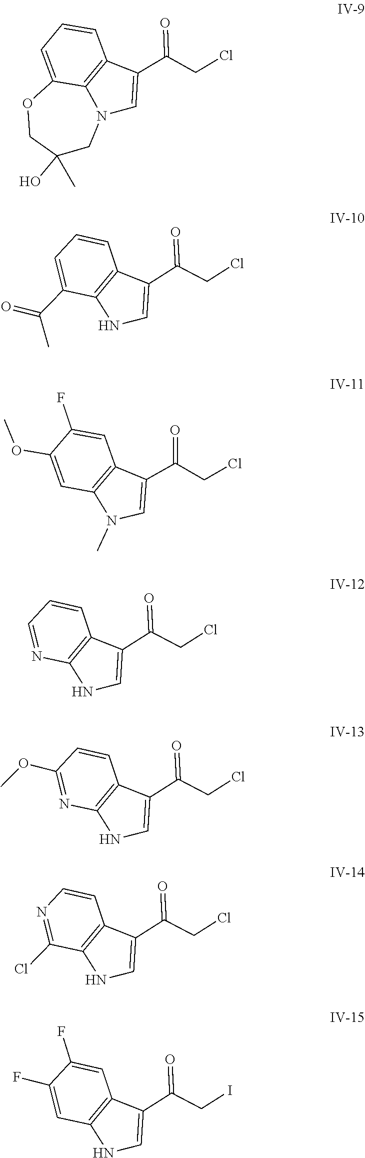

### Formulae Part 8: Structures of Intermediates VI Used for the Synthesis of the Examples

The following examples have been synthesized following Pathway A:

### Synthesis of INT-I, Scheme 1 (Pathway A)

### General Procedure A: Synthesis of Intermediates INT-I-1, INT-I-12, and INT-I-13

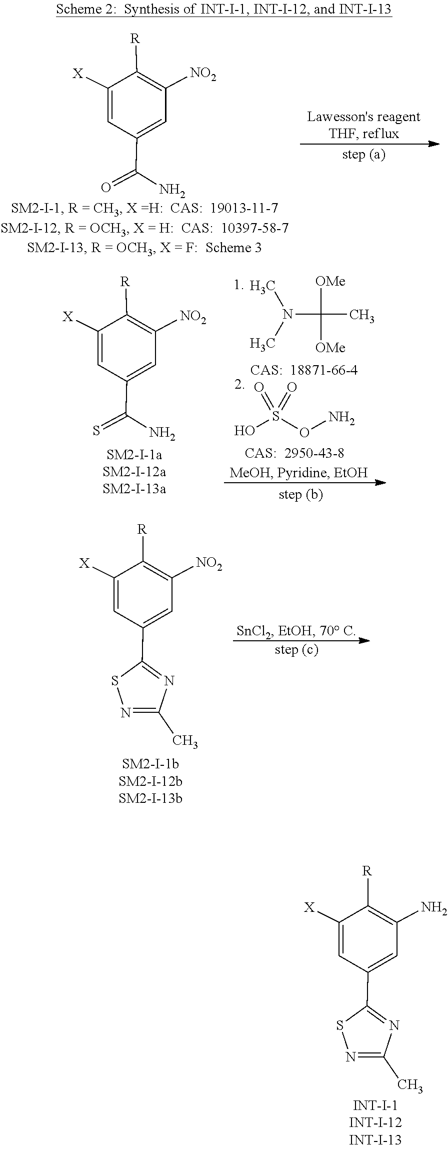

Step (a): Lawesson's reagent (1.2 equiv.) was added to a solution of nitrobenzamide derivatives SM1-I-1, SM1-I-12, and SM1-I-13 (1 equiv.) in dry THF (50 mL) and the resulting solution was refluxed for 18 h. The solvent was evaporated and the residue was treated with a sat. NaHCO₃ soln. (60 mL) and extracted with EtOAc (3×50 mL). The combined organic layers were dried over MgSO₄ and evaporated. Purification was performed using flash chromatography (PE: EtOAc, 1:1 or 100% DCM) to afford the desired compounds SM1-I-1a, SM1-I-12a, and SM1-I-13a as yellow solids.

Step (b): The resulting nitrobenzothioamide derivatives SM1-I-1a, SM1-I-12a, and SM1-I-13a (1 equiv.) was dissolved in N,N-dimethylacetamide dimethylacetal (1.4 mL) and the resulting solution was stirred at rt for 1 h. The volatile materials were removed under reduced pressure at rt. The residue was suspended in EtOH (5 mL), pyridine (2 equiv.) and at rt, was added rapidly a solution of hydroxylamine-O-sulfonic acid (1.1 equiv.) in MeOH (3 mL) and the reaction mixture was stirred at rt for 1 h. The solvents were removed under reduced pressure at rt and the residue was re-dissolved in DCM (70 mL). The resulting mixture was washed with water (H₂O), NaOH 0.1 M (20 mL each), dried over MgSO₄ and concentrated to obtain the crude products SM1-I-1b, SM1-I-12b, and SM1-I-13b as yellow solids that were used in the next step without further purification.

Step (c): To a suspension of the resulted nitro-1,2,4-thiadiazole derivatives SM1-I-1b, SM1-I-12b, and SM1-I-13b (1 equiv.) in EtOH (19 mL) was added SnCl₂ (5 equiv.) and the reaction was heated at 70° C. for 4 h. The resulting solution was poured onto a cold sat. NaHCO₃ soln. and the mixture was stirred for 30 min. The precipitate was filtrated off and washed with water. The solid was heated three times with THF and filtered. The aqueous (aq.) layer was extracted twice with EtOAc. The combined organic layers were dried over MgSO₄ and evaporated to give the desired intermediates INT-I-1, INT-I-12, and INT-I-13.

### Synthesis of 3-Fluoro-4-Methoxybenzamide SM2-I-13

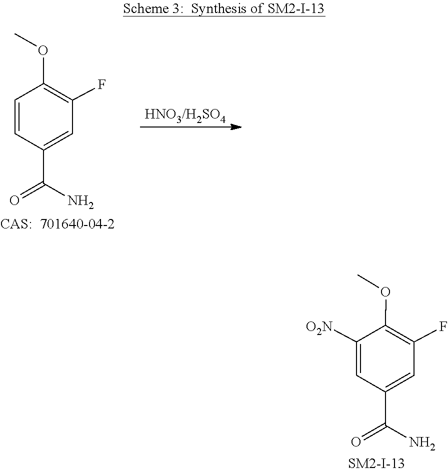

To the mixture of conc. H₂SO₄ (12 mL) and HNO₃ (65%, 10 mL) was added 3-fluoro-4-methoxybenzamide (CAS: 701640-04-2, 66.2 mmol, 10 g, 1 equiv.) slowly in portions at −5° C. The Mixture was stirred at rt for 1 h. The residue was placed in ice (300 mL). The resulting precipitate was filtrated off and washed several times with H₂O (1 L) and dried to give a white-yellow solid. The yield was 6.330 g (99%). LC-MS: (m/z): 215 [M+H]+; purity by HPLC-UV (254 nm)-ESI-MS: 97.2%.

### General Procedure B: Synthesis of Compounds 1-14, 1-17, and I-19

A solution of the corresponding ester SM1-1-14, SM1-I-17, and SM1-I-19 (Scheme 4, 1 equiv.), the corresponding amidine (1.5 equiv.) and K₂CO₃ (2 equiv.) in toluene is heated at reflux with a Dean-Stark system for 18 h. The reaction is quenched by addition of H₂O and extracted with EtOAc (3×50 mL). The combined organic layers were washed with brine, dried over MgSO₄ and evaporated. Pure intermediates I-14, I-17, and I-19 were purified using flash chromatography and/or crystallization (Method I).

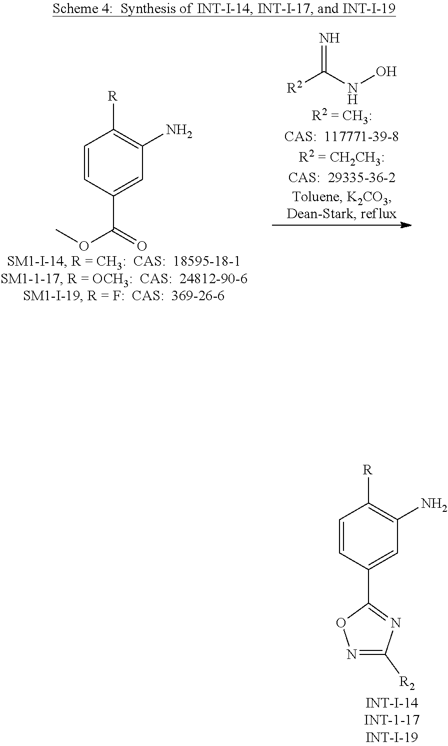

### Synthesis of 5-(2-Ethyl-2H-Tetrazol-5-yl)-2-Methoxyaniline INT-I-21

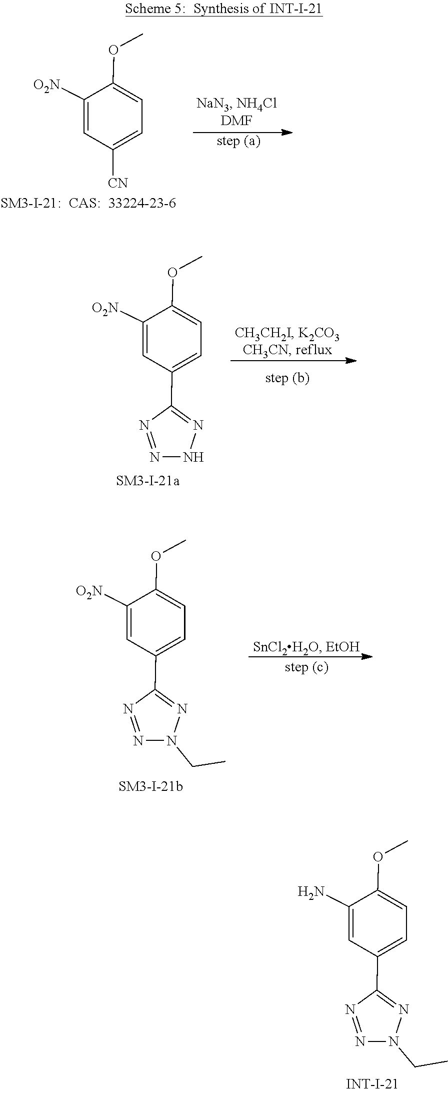 [SMILES: COc1ccc(C#N)cc1[N+](=O)[O-] ; COc1ccc(-c2nn[nH]n2)cc1[N+](=O)[O-] ; CCn1nnc(-c2ccc(OC)c([N+](=O)[O-])c2)n1 ; CCn1nnc(-c2ccc(OC)c(N)c2)n1]

Step (a): A mixture of 4-methoxy-3-nitrobenzonitrile (SM3-I-21, 2 mmol, 1 equiv.), NaN₃ (6 mmol, 3 equiv.) and NH₄Cl (6 mmol, 3 equiv.) in DMF (10 mL) was stirred at 120° C. for 18 h. The reaction mixture was allowed to reach rt and was then poured into water and extracted with EtOAc (2×25 mL). The organic extract was washed with H₂O, dried over MgSO₄, and concentrated in vacuum to give 5-(4-methoxy-3-nitrophenyl)-2H-tetrazole (SM3-I-21a) as a yellow solid. LC-MS: (m/z): 221.1 [M+H]⁺; purity by HPLC-UV (254 nm)-ESI-MS: 91.1% and it was used directly to the next step without further characterization.

Step (b): To a stirred solution of SM3-I-21a (0.125 mmol, 1 equiv.) in 25 mL of CH₃CN was added iodoethane (0.312 mmol, 2.5 equiv.), followed by addition of K₂CO₃ (0.125 mmole, 1 equiv.). The reaction mixture was heated to reflux for 2 h. After this time, the reaction mixture was concentrated under reduced pressure. The residue was taken up in EtOAc and filtered and the filtrate was concentrated under reduced pressure to afford the titled compound (SM3-I-21b) as a yellowish solid. LC-MS: (m/z): 249.9 [M+H]⁺; purity by HPLC-UV (254 nm)-ESI-MS: 81.8% and it was used directly to the next step without further characterization.

Step (c): To a suspension of 2-ethyl-5-(4-methoxy-3-nitrophenyl)-2H-tetrazole (SM3-I-21b; 1 equiv.) in EtOH (50 mL) was added SnCl₂ (5 equiv.) and the reaction was heated at 70° C. for 1 h. The resulting solution was poured onto a cold sat. NaHCO₃ soln. and the mixture was stirred for 30 min. The precipitate was filtrated off and washed with H₂O. The solid was heated two times with THF and filtered. The aqueous layer was extracted twice with EtOAc. The combined organic layers were dried over MgSO₄ and evaporated to give the desired intermediate INT-I-21.

### Synthesis of INT-V, Scheme 1 (Pathway A)

### Synthesis of Intermediate INT-V-1

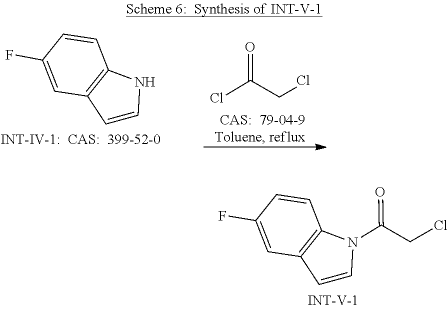 [SMILES: Fc1ccc2[nH]ccc2c1 ; O=C(Cl)CCl ; O=C(CCl)n1ccc2cc(F)ccc21]

A solution of 5-fluoroindole (CAS: 399-52-0, 1 equiv.) in toluene (30 mL) was heated to reflux at 110° C. and was then treated dropwise with chloroacetyl chloride (1.5 equiv.). The reaction mixture was stirred at the same temperature for 18 h. After this, the reaction was allowed to reach rt and the solvent was removed under reduced pressure. Water (50 mL) was then added and the reaction mixture was extracted with EtOAc (3×50 mL). The combined organic layers were washed with sat. NaHCO₃ soln. (2×25 mL), brine (2×25 mL) and was then dried over MgSO₄ and evaporated to dryness to afford INT-V-1.

General Procedure C: Synthesis of Intermediates INT-V-(2-7), Scheme 1, Formulae Part 7 To obtain intermediates INT-V-(2-7), a solution of chloroacetylchloride (1.5 equiv.) in DCM (20 mL) was added dropwise to a stirring solution of the appropriate indoline (scheme 7, 1.0 equiv.) and Et₃N (1.5 equiv.) in DCM (20 mL). The reaction mixture was stirred at room temperature for 30 min. After this time, the solvent was removed under reduced pressure, water (50 mL) was added and the product was then extracted with EtOAc (3×50 mL). The collected organic extracts were washed with brine (3×50 mL), dried over MgSO₄ and evaporated to dryness to afford the desired products INT-V-(2-7).

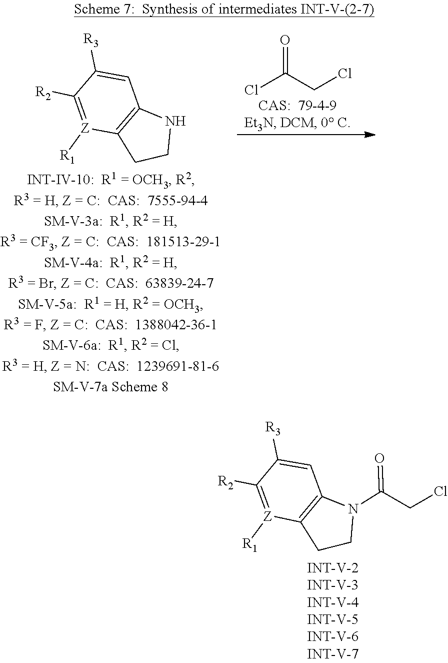

### Synthesis of Compound INT-V-7a

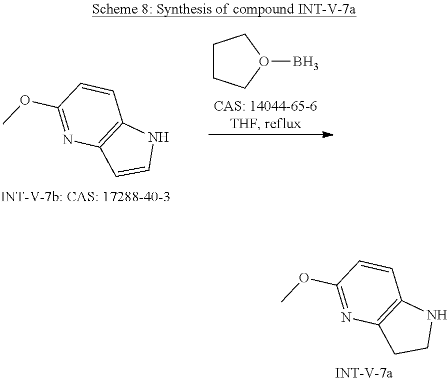

To a solution of 5-methoxy-1H-pyrrolo[3,2-b]pyridine (INT-V-7b, 149 mg, 1 equiv.) in 30 mL of THF, was added borane tetrahydrofuran complex (1 M solution, 6 mL, 6 equiv.) and the mixture was refluxed for 6 h. After allowing the reaction to cool down to rt, MeOH was added slowly until the bubbles were disappeared and the solution was then extracted with EtOAc (2×25 mL). The combined extracts were washed with a sat. NaHCO₃ soln., dried over MgSO₄ and evaporated to afford INT-V-7a. LC-MS: (m/z): 151.08 [M+H]⁺; purity by HPLC-UV (254 nm)-ESI-MS: 78.5% and was used directly to the next step without further purification.

### Synthesis of the of the Final Examples 1-11

### General Procedure D: Synthesis Method of Formula a, Step 8, Scheme 1

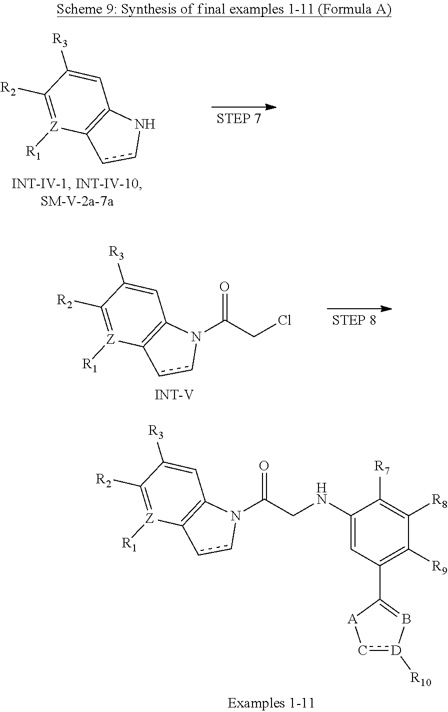

A solution of the appropriate aniline INT-I-1, INT-I-12, INT-I-13, INT-I-14, INT-I-17, INT-I-19, or INT-I-21 (2 equiv.) in 30 mL acetone was mixed with the appropriate acyl(aza)indole/indoline INT-V-1, INT-V-2, INT-V-3, INT-V-4, INT-V-5, INT-V-6, or INT-V-7 (1 equiv.), potassium iodide (KI, 2 equiv.) and N,N-diisopropylethylamine (DIPEA, 3 equiv.). The reaction mixture was heated to reflux for 5-18 h. After the reaction was completed, the mixture was allowed to reach rt and the solvent was removed under reduced pressure. Water (50 mL) was then added and the product was extracted with EtOAc (3×50 mL). The combined organic extracts were washed with 1 M HCl (3×25 mL), then with brine (2×50 mL) and dried over MgSO₄ or Na₂SO₄, filtered, and concentrated under vacuum. The crude was subjected to chromatography purifications on silica gel to obtain the pure products (Method I & II).

TABLE 1
Reaction and purification conditions of the final examples 1-12 synthesized via STEP 8,
scheme 1 (Pathway A)

| | | | | Synthesis | |
|---|---|---|---|---|---|
| | | | | method & | |
| | | | | Difference in | |
| | | INT-V | INT-I | synthesis | Purification method & |
| | | (scheme 1, | (scheme 1, | conditions, | Difference in |
| Example | | Formulae | Formulae | if relevant | Purification conditions |
| Nr. | Strcture | part 7) | part 1) | (Yield %) | if relevant |
| 1 | 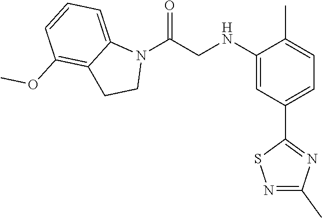 | INT-V-2 | INT-I-1 | (26) | II |
| 2 | 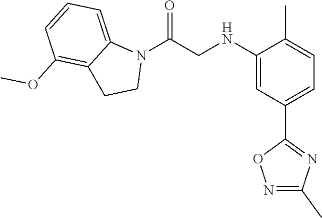 | INT-V-2 | INT-I-14 | D (35) | I |
| 3 | 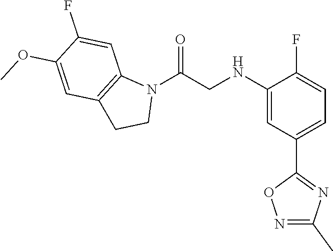 | INT-V-5 | INT-I-19 | D (19) | II |
| 4 | 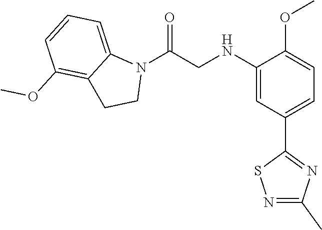 | INT-V-2 | INT-I-12 | D CH₃CN (46) | Water was added and the formed precipitate was filtered off and washed with H₂O_{?}, MeOH and PE. |
| 5 | 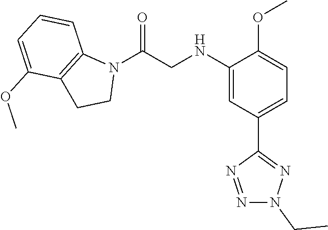 [SMILES: CCn1nnc(-c2ccc(OC)c(NCC(=O)N3CCc4c(OC)cccc43)c2)n1] | INT-V-2 | INT-I-21 | D (28) | I |
| 6 | 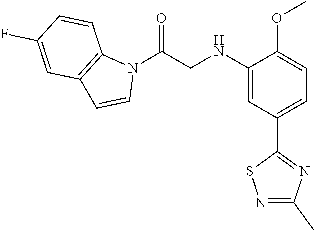 [SMILES: COc1ccc(-c2nc(C)ns2)cc1NCC(=O)n1ccc2cc(F)ccc21] | INT-V-1 | INT-1-12 | D (22.1) | I |
| 7 | 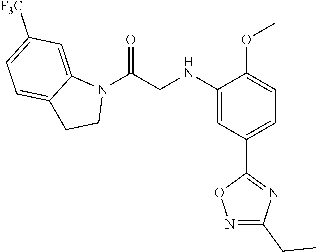 [SMILES: CCc1noc(-c2ccc(OC)c(NCC(=O)N3CCc4ccc(C(F)(F)F)cc43)c2)n1] | INT-V-3 | INT-I-12 | D (30) | I |
| 8 | 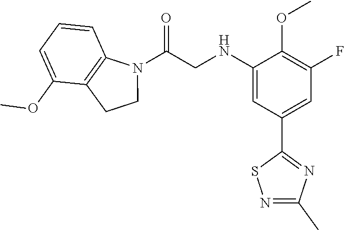 [SMILES: COc1cccc2c1CCN2C(=O)CNc1cc(-c2nc(C)ns2)cc(F)c1OC] | INT-V-2 | INT-1-13 | D (10) | I, then recrystallization with cyclohexane:EtOAc (3:1) |
| 9 | 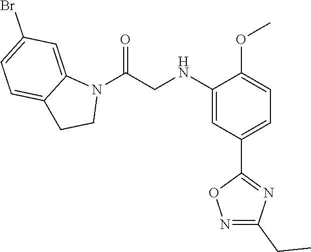 [SMILES: CCc1noc(-c2ccc(OC)c(NCC(=O)N3CCc4ccc(Br)cc43)c2)n1] | INT-V-4 | INT-I-17 | D (39.4) | I |
| 10 | 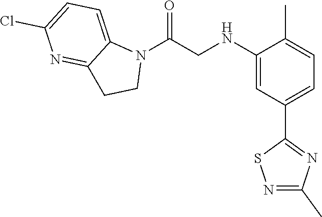 [SMILES: Cc1nsc(-c2ccc(C)c(NCC(=O)N3CCc4nc(Cl)ccc43)c2)n1] | INT-V-6 | INT-I-1 | D (50) | I |
| 11 | 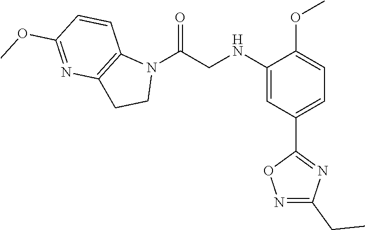 [SMILES: CCc1noc(-c2ccc(OC)c(NCC(=O)N3CCc4nc(OC)ccc43)c2)n1] | INT-V-7 | INT-I-17 | D (22) | I, then recrystallization with cyclohexane:EtOAc (3:1) |

### Synthesis of Compounds INT-III-(1-16), Scheme 1, Formulae Parts 3 and 4

### Synthesis of (1,2,4-thiadiazol-5-yl)phenyl)glycine derivatives INT-III-1, INT-III-2, INT-III-9, INT-III-10, and INT-III-11

### Step (a): General procedure for the synthesis of nitrobenzothioamide derivatives (SM2-I-1a, SM2-I-9a)

To a solution of nitrobenzamide derivatives (1.0 equiv.) in THF was added 2,4-bis(4-methoxyphenyl)-1,3,2,4-dithiadiphosphetane 2,4-disulfide (0.5 equiv.) at 25° C. under N₂ atmosphere. The resulting reaction mixture was stirred at 70° C. for 5 h. After completion of the reaction as indicated by TLC, the reaction mixture was concentrated under reduced pressure. This crude residue was purified by column chromatography using EtOAc: heptane as an eluent to afford the corresponding thioamides.

4-Methyl-3-nitrobenzothioamide (SM2-I-1a): Treatment of 4-methyl-3-nitrobenzamide (SM2-I-1, 90 g, 0.5 mol) with 2,4-bis(4-methoxyphenyl)-1,3,2,4-dithiadiphosphetane 2,4-disulfide (101 g, 0.25 mol) in THF (1 L) for 16 h at 25° C. followed by column chromatography afforded 4-methyl-3-nitrobenzothioamide as (SM2-I-1a) as a yellow solid. Rf(EtOAc: heptane, 5:5)=0.6; yield: 95 g, 96%. MS: m/z 194.99 [M−H]⁺.

4-Fluoro-3-nitrobenzothioamide (SM2-I-9a): Treatment of 4-fluoro-3-nitrobenzamide (SM2-I-9, 9.5 g, 51.6 mmol) with 2,4-bis(4-methoxyphenyl)-1,3,2,4-dithiadiphosphetane 2,4-disulfide (31.2 g, 77.4 mmol) in THF (100 mL) at 70° C. for 1 h followed by column chromatography afforded 4-fluoro-3-nitrobenzothioamide (SM2-I-9a) as a yellow solid. Rf(EtOAc: heptane, 2:8)=0.5; yield: 9.0 g, 87%.

### Step (b): General procedure for the synthesis of N-(1-imino)-3-nitrobenzothioamide (SM2-I-1b, SM2-I-2b, SM2-I-9b, and SM2-I-10b)

To the stirred solution of nitrobenzothioamide derivative (1 equiv.) in EtOAc and the corresponding nitrile derivative (20 equiv.) was purged HCl gas vigorously at 0° C. under N₂ atmosphere for 15 min at an interval of 1 d while the reaction mixture was stirred at 25° C. for 3 d. After completion of the reaction as indicated by TLC, the reaction mixture was concentrated under reduced pressure and the brownish sticky solid product was triturated and washed with EtOAc. The solid residue was dissolved into ice-cold water and basified with sat. NaHCO₃ soln. (pH=8) and the reaction mixture was extracted with EtOAc (3 times). The combined organic layers were dried over anhyd. Na₂SO₄ and concentrated under reduced pressure. This crude residue was forwarded immediately for the next step without further purification.

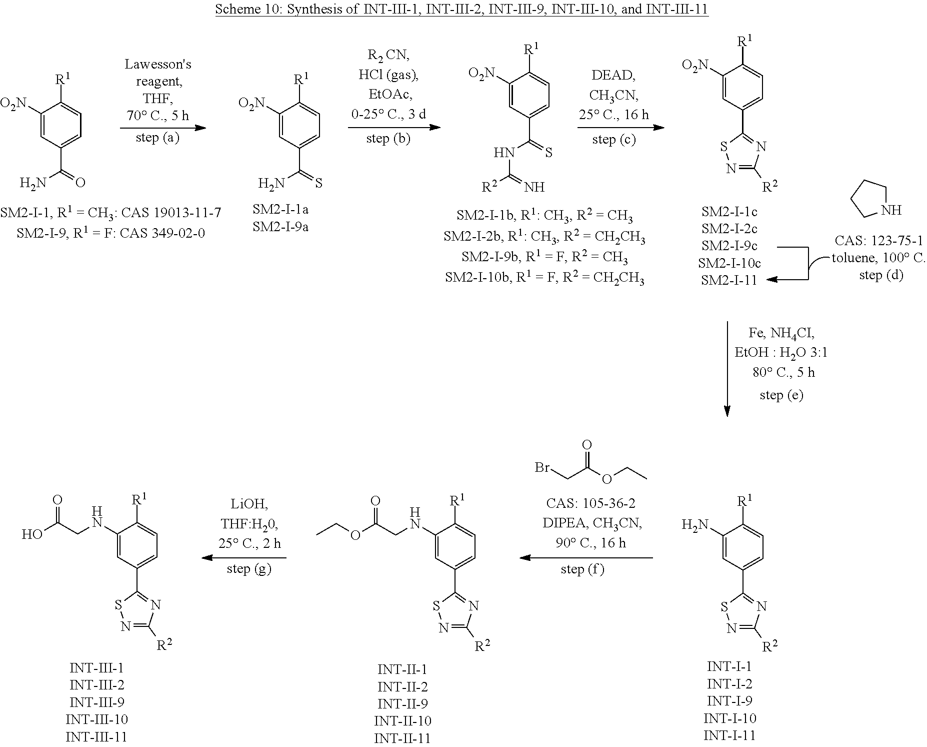

N-(1-Iminoethyl)-4-methyl-3-nitrobenzothioamide (SM2-I-1b): Treatment of 4-methyl-3-nitrobenzothioamide (SM2-I-1a, 100 g, 0.51 mol) with CH₃CN (100 mL) and HCl gas in EtOAc (300 mL) at 25° C. for 2 d followed by basic extraction to afford (SM2-I-1b) as a crude compound which was forwarded to the next step without further purification. Rf(EtOAc: heptane, 5:5)=0.4; yield: 70 g, 58% (crude). MS: m/z 238.02 [M+H]⁺.

N-(1-Iminopropyl)-4-methyl-3-nitrobenzothioamide (SM2-I-2b): Treatment of 4-methyl-3-nitrobenzothioamide (SM2-I-1a, 20 g, 102 mmol) with propiononitrile (35 mL, 510 mmol) and HCl gas in EtOAc (300 mL) at 25° C. for 2 d followed by extraction to afford crude product as a brownish solid. This crude residue SM2-I-2b was forwarded to the next step without further purification. Rf(EtOAc heptane, 3:7)=0.5; yield: 13.0 g, 51% (crude). MS: m/z 251.95 [M+H]⁺.

4-Fluoro-N-(1-iminoethyl)-3-nitrobenzothioamide (SM2-I-9b): Treatment of 4-fluoro-3-nitrobenzothioamide (SM2-I-9a, 10 g, 50 mmol) with CH₃CN (13 mL) and HCl gas in Et₂O (100 mL) at 25° C. for 2 d followed by basic extraction to afford crude productas a red solid. This crude residue SM2-I-9b was forwarded to the next step without further purification. Rf(EtOAc: heptane, 2:8)=0.3; yield: 5.5 g, 46% (crude). MS: m/z 241.99 [M+H]⁺.

4-Fluoro-N-(1-iminopropyl)-3-nitrobenzothioamide (SM2-I-10b): Treatment of 4-fluoro-3-nitrobenzothioamide (SM2-I-9a, 10 g, 50 mmol) with propiononitrile (13.7 mL, 250 mmol) and HCl gas in EtOAc (200 mL) at 25° C. for 2 d followed by basic extraction to afford crude product as a brownish solid. This crude residue SM2-I-10b was forwarded to the next step without further purification. Rf(EtOAc: heptane, 2:8)=0.6; yield: 5.5 g, 43% (crude). MS: m/z 255.99 [M+H]⁺.

Step (c): General procedure for the synthesis of (3-nitrophenyl)-1,2,4-thiadiazole derivatives (SM2-I-1c, SM2-I-2c, SM2-I-9c, and SM2-I-10c) To a stirred solution of N-(1-imino)-4-nitrobenzothioamide (1 equiv.) in CH₃CN was added DEAD (1.1 equiv.) at 0° C. under N₂ atmosphere and the reaction mixture was then stirred at 25° C. for 16 h. After completion of the reaction as indicated by TLC, the reaction mixture was concentrated under reduced pressure. This crude residue was purified by column chromatography to afford the corresponding thiadiazole derivatives.

3-Methyl-5-(4-methyl-3-nitrophenyl)-1,2,4-thiadiazole (SM2-I-1c): Treatment of N-(1-iminoethyl)-3-methyl-4-nitrobenzothioamide (SM2-I-1b, 70 g, 0.29 mol) with DEAD (55 mL, 0.35 mol) in CH₃CN (500 mL) at 25° C. for 16 h followed by purification by column chromatography afforded 3-methyl-5-(4-methyl-3-nitrophenyl)-1,2,4-thiadiazole as (SM2-I-1c) as an off white solid. Rf (EtOAc: heptane, 5:5)=0.6; yield: 40 g, 57%. MS: m/z 236.03 [M+H]⁺.

3-Ethyl-5-(4-methyl-3-nitrophenyl)-1,2,4-thiadiazole (SM2-I-2c): Treatment of N-(1-iminopropyl)-4-methyl-3-nitrobenzothioamide (SM2-I-2b, 17 g, 67.7 mmol) with DIAD (26.6 mL, 135 mmol) in CH₃CN (600 mL) at 25° C. for 2 h followed by purification by column chromatography afforded 3-methyl-5-(4-methyl-3-nitrophenyl)-1,2,4-thiadiazoleas (SM2-I-2c) as a yellow solid. Rf (EtOAc heptane, 3:7)=0.7; yield: 12 g, 71%. MS: (m/z) 250.20 [M+H]⁺.

3-Methyl-5-(4-fluoro-3-nitrophenyl)-1,2,4-thiadiazole (SM2-I-9c): Treatment of 4-fluoro-N-(1-iminoethyl)-3-nitrobenzothioamide (SM2-I-9b, 5.0 g, 27.7 mmol) with DEAD (6.5 mL, 41.5 mmol) in EtOH (40 mL) at 25° C. for 2 h followed by extraction and purification by column chromatography afforded 3-methyl-5-(4-fluoro-3-nitrophenyl)-1,2,4-thiadiazole (SM2-I-9c) as a white solid. Rf (EtOAc: heptane, 2:8)=0.5; yield: 4.0 g, 81%. MS: m/z 240.03 [M+H]⁺.

3-Ethyl-5-(4-fluoro-3-nitrophenyl)-1,2,4-thiadiazole (SM2-I-10c): Treatment of 4-fluoro-N-(1-iminopropyl)-3-nitrobenzothioamide (SM2-I-10b, 5.0 g, 19.6 mmol) with DEAD (6.14 mL, 39.2 mmol) in CH₃CN (40 mL) at 25° C. for 16 h followed by purification by column chromatography afforded 3-ethyl-5-(4-fluoro-3-nitrophenyl)-1,2,4-thiadiazole (SM2-I-10c) as a yellow solid. Rf (EtOAc: heptane, 2:8)=0.7; yield: 4.2 g, 85%. MS: m/z 253.87 [M+H]⁺.

### Step (d): Synthesis of 3-methyl-5-(3-nitro-4-(pyrrolidin-1-yl)phenyl)-1,2,4-thiadiazole (SM2-I-11)

Pyrrolidine (0.308 g, 4.35 mmol) was added to a stirred solution of 5-(4-fluoro-3-nitrophenyl)-3-methyl-1,2,4-thiadiazole (SM2-I-9c, 0.8 g, 3.34 mmol) in toluene (10 mL) under N₂ atmosphere at room temperature for 4 h. After completion of the reaction as indicated by TLC, the reaction mixture was diluted with water and extracted with EtOAc (3×80 mL). The combined organic layers were washed with brine, dried over anhy. Na₂SO₄, filtered and concentrated under reduced pressure to afford SM2-I-11 (0.75 g, 77%) as a yellow solid that used in the next step without any further purification.

### Step (e): General procedure for the synthesis of (1,2,4-thiadiazol-5-yl)aniline derivatives INT-I-1, INT-I-2, INT-I-9, INT-I-10, and INT-I-11

To a stirred solution of (3-nitrophenyl)-1,2,4-thiadiazole (1 equiv.) in EtOH and H₂O (3:1) was added iron powder (7 equiv.) followed by NH₄Cl (7 equiv.) at 25° C. under N₂ atmosphere and the reaction mixture was stirred at 80° C. for 5 h. After completion of the reaction as indicated by TLC, the reaction mixture was concentrated, diluted with EtOAc and filtered through celite bed. Filtrate was extracted with EtOAc (3 times). The combined organic layers were washed with brine, dried over anhy. Na₂SO₄, filtered and concentrated under reduced pressure. This crude residue was triturated with DCM and heptane to afford the corresponding anilines.

2-Methyl-5-(3-methyl-1,2,4-thiadiazol-5-yl)aniline (INT-I-1): Treatment of 3-methyl-5-(3-methyl-4-nitrophenyl)-1,2,4-thiadiazole (SM2-I-1c, 40 g, 0.17 mol) with iron powder (47 g, 0.85 mol) in the presence of NH₄Cl (45 g, 0.85 mol) in MeOH: H₂O (4:1, 500 mL) at 90° C. for 5 h followed by work-up afforded 2-methyl-5-(3-methyl-1,2,4-thiadiazol-5-yl)aniline (INT-I-1) as an off white solid. Rf (EtOAc: heptane, 5:5)=0.4; yield: 30 g, 88% (crude). MS: m/z 206.03 [M+H]⁺.

5-(3-Ethyl-1,2,4-thiadiazol-5-yl)-2-methylaniline (INT-I-2): Treatment of 3-ethyl-5-(4-methyl-3-nitrophenyl)-1,2,4-thiadiazole (SM2-I-2c, 12 g, 48.2 mmol) with iron powder (13.3 g, 241 mmol) NH₄Cl (12.7 g, 241 mmol) in EtOH: H₂O (5:1, 120 mL) at 100° C. for 3 h followed by work-up and trituration afforded 2-methyl-4-(3-methyl-1,2,4-thiadiazol-5-yl)aniline (INT-I-2) as an off white solid. R_{f}(EtOAc: heptane, 5:5)=0.4; yield: 10 g, 95%. MS: m/z 220.14 [M+H]⁺.

2-Fluoro-5-(3-methyl-1,2,4-thiadiazol-5-yl)aniline (INT-I-9): Treatment of 3-methyl-5-(4-fluoro-3-nitrophenyl)-1,2,4-thiadiazole (SM2-I-9c, 4.0 g, 16.7 mmol) with iron powder (4.6 g, 83.7 mmol) and NH₄Cl (4.4 g, 83.7 mmol) in EtOH: H₂O (3:1, 40 mL) at 80° C. for 2 h followed by work-up and trituration afforded 2-fluoro-5-(3-methyl-1,2,4-thiadiazol-5-yl)aniline (INT-I-9) as a dark grey liquid. R_{f}(EtOAc: heptane, 1:9)=0.3; yield: 2.8 g, 80% (crude). MS: m/z 209.97 [M+H]⁺.

5-(3-Ethyl-1,2,4-thiadiazol-5-yl)-2-fluoroaniline (INT-I-10): Treatment of 3-ethyl-5-(4-fluoro-3-nitrophenyl)-1,2,4-thiadiazole (SM2-I-10c, 3.2 g, 12.6 mmol) with iron powder (3.4 g, 63.2 mmol) and NH₄Cl (3.3 g, 63.2 mmol) in EtOH: H₂O (4:1, 25 mL) at 80° C. for 2 h followed by work-up and trituration afforded 5-(3-ethyl-1,2,4-thiadiazol-5-yl)-2-fluoroaniline (INT-I-10) as awhite solid. R_{f} (EtOAc: heptane, 2:8)=0.4; yield: 2.1 g, 75%. MS: m/z 224.13 [M+H]

5-(3-Methyl-1,2,4-thiadiazol-5-yl)-2-(pyrrolidin-1-yl)aniline (INT-I-11): Treatment of 3-methyl-5-(3-nitro-4-(pyrrolidin-1-yl)phenyl)-1,2,4-thiadiazole (SM2-I-11, 0.75 g, 2.58 mmol) with iron powder (0.72 g, 12.93 mmol) in the presence of NH₄Cl (0.69 g, 12.93 mmol) in MeOH: H₂O (2:1, 15 mL) at 90° C. for 4 h followed by work-up afforded 5-(3-methyl-1,2,4-thiadiazol-5-yl)-2-(pyrrolidin-1-yl)aniline (INT-I-11) as an yellow solid. yield: 600 mg, 88% (crude).

### Step (f): General procedure for the synthesis of (1,2,4-thiadiazol-5-yl)phenyl)glycinate derivatives INT-II-1, INT-II-2, INT-II-9, INT-II-10, and INT-II-11

To a stirred solution of (1,2,4-thiadiazol-5-yl)aniline derivative (1.0 equiv.) in DMF were added DIPEA (2.0 equiv.) and ethyl 2-bromoacetate (1.1 equiv.) under N₂ atmosphere. This reaction mixture was stirred at 100° C. for 16 h. After completion of the reaction as indicated by TLC, reaction mixture was diluted with ice cold water and extracted with EtOAc (3 times). The combined organic layer was washed with ice cold brine and dried over anhy. Na₂SO₄, filtered and concentrated under reduced pressure. This crude residue was purified by column chromatography to afford the corresponding ethyl carboxylates.

Ethyl (2-methyl-5-(3-methyl-1,2,4-thiadiazol-5-yl)phenyl)glycinate (INT-II-1): Treatment of 2-methyl-5-(3-methyl-1,2,4-thiadiazol-5-yl)aniline (INT-I-1, 6.5 g, 31.7 mmol) with ethyl 2-bromoacetate (3.5 mL, 31.7 mmol) and DIPEA (11 mL, 63 mmol) in DMF (70 mL) at 100° C. for 16 h followed by work-up and trituration afforded ethyl (2-methyl-5-(3-methyl-1,2,4-thiadiazol-5-yl)phenyl)glycinate (INT-II-1) as an off white solid. R_{f}(EtOAc: heptane, 3:7)=0.6; yield: 5.1 g, 55%. MS: m/z 292.01 [M+H]⁺.

Ethyl (5-(3-ethyl-1,2,4-thiadiazol-5-yl)-2-methylphenyl)glycinate (INT-II-2): Treatment of 5-(3-ethyl-1,2,4-thiadiazol-5-yl)-2-methylaniline (INT-I-2, 5.0 g, 22.8 mmol) with ethyl 2-bromoacetate (7.57 mL, 68.5 mmol) and DIPEA (19.9 mL, 114 mmol) in DMF (50 mL) at 100 C for 16 h followed by work-up and purification afforded ethyl (5-(3-ethyl-1,2,4-thiadiazol-5-yl)-2-methylphenyl)glycinate (INT-II-2). R_{f}(EtOAc:heptane, 3:7)=0.6; yield: 5 g, 72%. MS: m/z 306.14 [M+H]⁺.

Ethyl (2-fluoro-5-(3-methyl-1,2,4-thiadiazol-5-yl)phenyl)glycinate (INT-II-9): Treatment of 2-fluoro-5-(3-methyl-1,2,4-thiadiazol-5-yl)aniline (INT-I-9, 2.8 g, 13.4 mmol) with ethyl 2-bromoacetate (4.4 mL, 40.2 mmol) and DIPEA (23.3 mL, 134 mmol) in DMF (20 mL) at 120° C. for 16 h followed by work-up and purification afforded ethyl (2-fluoro-5-(3-methyl-1,2,4-thiadiazol-5-yl)phenyl)glycinate (INT-II-9) as an off white solid. R_{f}(EtOAc: heptane, 2:8)=0.5; yield: 2.6 g, 66%. MS: m/z 295.99 [M+H]⁺.

Ethyl (5-(3-ethyl-1,2,4-thiadiazol-5-yl)-2-fluorophenyl)glycinate (INT-II-10): Treatment of 5-(3-ethyl-1,2,4-thiadiazol-5-yl)-2-fluoroaniline (INT-I-10, 2.1 g, 9.42 mmol) with ethyl 2-bromoacetate (3.1 mL, 28.3 mmol) and DIPEA (16.4 mL, 94.2 mmol) in DMF (10 mL) at 100 C for 16 h followed by work-up and purification afforded ethyl (5-(3-ethyl-1,2,4-thiadiazol-5-yl)-2-fluorophenyl)glycinate (INT-II-10). R_{f}(EtOAc: heptane, 1:9)=0.4; yield: 1.8 g, 62%. MS: m/z 310.03 [M+H]⁺.

Ethyl (5-(3-methyl-1,2,4-thiadiazol-5-yl)-2-(pyrrolidin-1-yl)phenyl)glycinate (INT-II-11): Treatment of 5-(3-methyl-1,2,4-thiadiazol-5-yl)-2-(pyrrolidin-1-yl)aniline (INT-I-11, 600 mg, 2.29 mmol) with ethyl 2-bromoacetate (0.5 mL, 4.59 mmol) and DIPEA (1.89 mL, 11.45 mmol) in DMF (70 mL) at 100° C. for 16 h followed by work-up and trituration afforded ethyl (5-(3-methyl-1,2,4-thiadiazol-5-yl)-2-(pyrrolidin-1-yl)phenyl)glycinate (INT-II-11) as an yellow solid. Yield: 600 g, 75%.

### Step (g): General Procedure for the Synthesis of (1,2,4-Thiadiazol-5-yl)Phenyl)Glycine Derivatives INT-III-1, INT-III-2, INT-III-9, INT-III-10 and INT-III-11

To a stirred solution of (1,2,4-thiadiazol-5-yl)phenyl)glycinate derivative (1.0 equiv.) in MeOH: THF: H₂O (1:2:1), was added lithium hydroxide monohydrate (LiOH. H₂O, 3.0 equiv.) at 0° C. The resulting reaction mixture stirred at 25° C. for 2 h. After completion of the reaction as indicated by TLC, the reaction mixture was concentrated under reduced pressure and diluted with H₂O. The aq. layer was acidified with sat. citric acid soln. (pH=5-6) at 0° C., resulting precipitation of the product. The solid product was dried under high reduced pressure to afford the corresponding acid as a white solid.

2-Methyl-5-(3-methyl-1,2,4-thiadiazol-5-yl)phenyl)glycine (INT-III-1): Treatment of ethyl (2-methyl-5-(3-methyl-1,2,4-thiadiazol-5-yl)phenyl)glycinate (INT-II-1, 5.1 g, 17.5 mmol) with LiOH. H₂O (2.2 g, 52.6 mmol) in mixture of MeOH: THF: H₂O (1:2:1, 80 mL) at 25° C. for 2 h followed by acidification to afford 2-methyl-5-(3-methyl-1,2,4-thiadiazol-5-yl)phenyl)glycine (INT-III-1) as a white solid. R_{f} (MeOH: EtOAC, 5:95)=0.3; yield: 4.4 g, 93%. MS: m/z 264.13 [M+H]⁺.

(5-(3-Ethyl-1,2,4-thiadiazol-5-yl)-2-methylphenyl)glycine (INT-III-2): Treatment of ethyl (5-(3-ethyl-1,2,4-thiadiazol-5-yl)-2-methylphenyl)glycinate (INT-II-2, 2.0 g, 6.55 mmol) with LiOH. H₂O (0.82 g, 19.6 mmol) in MeOH: THF: H₂O (1:2:1, 30 mL) at 25° C. for 1 h followed by acidification to to afford (5-(3-ethyl-1,2,4-thiadiazol-5-yl)-2-methylphenyl)glycine (INT-III-2) as a white solid. R_{f} (EtOAc: heptane, 8:2)=0.1; yield: 1.6 g, 88%. MS: m/z 278.19 [M+H]⁺. ¹H-NMR (400 MHz; DMSO-d₆): δ 12.65 (s, 1H), 7.16 (s, 2H), 6.91 (s, 1H), 5.54 (br s, 1H) 3.94 (s, 2H), 2.96 (q, 2H), 2.17 (s, 3H), 1.32 (t, 3H).

(2-Fluoro-5-(3-methyl-1,2,4-thiadiazol-5-yl)phenyl)glycine (INT-III-9): Treatment of ethyl (2-fluoro-5-(3-methyl-1,2,4-thiadiazol-5-yl)phenyl)glycinate (INT-II-9, 2.5 g, 8.47 mmol) with LiOH. H₂O (1.06 g, 25.4 mmol) in MeOH: THF: H₂O (1:2:1, 30 mL) at 25° C. for 2 h followed by acidificationto afford (2-fluoro-5-(3-methyl-1,2,4-thiadiazol-5-yl)phenyl)glycine (INT-III-9) as a white solid. R_{f} (MeOH: EtOAc, 5:95)=0.1; yield: 2.0 g, 88%. MS: m/z 267.99 [M+H]⁺.

(5-(3-Ethyl-1,2,4-thiadiazol-5-yl)-2-fluorophenyl)glycine (INT-III-10): Treatment of ethyl (5-(3-ethyl-1,2,4-thiadiazol-5-yl)-2-fluorophenyl)glycinate (INT-II-10, 2.4 g, 7.76 mmol) with LiOH. H₂O (0.98 g, 23.30 mmol) in EtOH: THF: H₂O (1:2:1, 4 mL) at 25° C. for 2 h followed by acidification to afford (5-(3-ethyl-1,2,4-thiadiazol-5-yl)-2-fluorophenyl)glycine (INT-III-10) as a white solid. R_{f} (EtOAc: heptane, 80:20)=0.1; yield: 1.6 g, 73%. MS: m/z 282.15 [M+H]⁺.

(5-(3-Methyl-1,2,4-thiadiazol-5-yl)-2-(pyrrolidin-1-yl)phenyl)glycine (INT-III-11): Treatment of ethyl (5-(3-methyl-1,2,4-thiadiazol-5-yl)-2-(pyrrolidin-1-yl)phenyl)glycinate (INT-II-11, 600 mg, 1.73 mmol) with LiOH. H₂O (228 mg, 5.2 mmol) in mixture of EtOH: THF: H₂O (5:10:2, 17 mL) at 25° C. for 1 h followed by acidification to afford (5-(3-methyl-1,2,4-thiadiazol-5-yl)-2-(pyrrolidin-1-yl)phenyl)glycine (INT-III-11) as a yellow solid. Yield: 600 mg, 88%.

### Synthesis of (2-methyl-5-(3-vinyl-1,2,4-thiadiazol-5-yl)phenyl)glycine (INT-III-3)

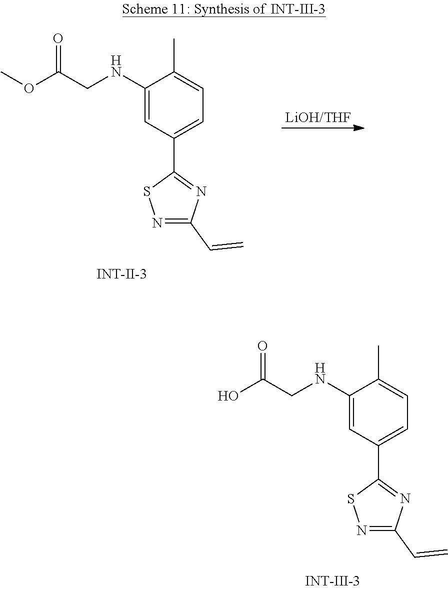 [SMILES: C=Cc1nsc(-c2ccc(C)c(NCC(=O)OC)c2)n1 ; C=Cc1nsc(-c2ccc(C)c(NCC(=O)O)c2)n1]

To a solution of methyl (2-methyl-5-(3-vinyl-1,2,4-thiadiazol-5-yl)phenyl)glycinate (INT-II-3, 50 mg, 0.17 mmol) in MeOH: THF: H₂O (5 mL, 7:2:1) was added LiOH. H₂O (15 mg, 0.34 mmol) and stirred at 25° C. for 2 h. Upon completion of the reaction, it was concentrated at 35° C. under reduced pressure. The resulting crude material was dissolved in water (2 mL) and acidified with 5% citric acid solution. The solid obtained was filtered, washed with water (5 mL) and dried to get 40 mg of ((2-methyl-5-(3-vinyl-1,2,4-thiadiazol-5-yl)phenyl)glycine (INT-III-3) as a brown solid that used in the next step without further purification. MS: m/z 276.0 [M+H]⁺.

### Synthesis of (5-(3-ethynyl-1,2,4-thiadiazol-5-yl)-2-methylphenyl)glycine (INT-III-4)

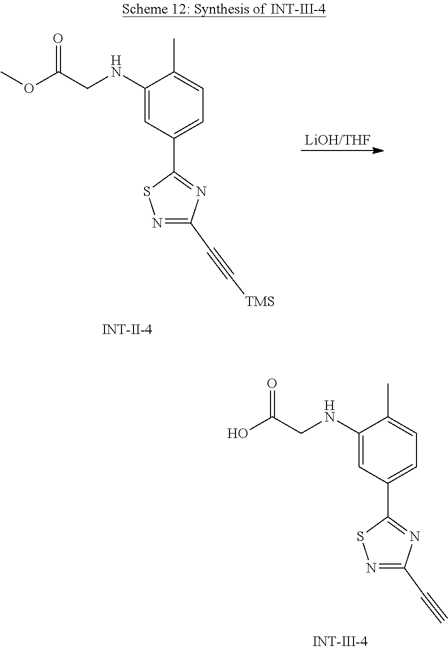 [SMILES: COC(=O)CNc1cc(-c2nc(C#C[Si](C)(C)C)ns2)ccc1C ; C#Cc1nsc(-c2ccc(C)c(NCC(=O)O)c2)n1]

To a solution of methyl (2-methyl-5-(3-((trimethylsilyl)ethynyl)-1,2,4-thiadiazol-5-yl)phenyl)glycinate (INT-II-4, 200 mg, 0.53 mmol) in MeOH: THF: H₂O (10 mL, 7:2:1) was added LiOH. H₂O (44 mg, 1.07 mmol) and it was stirred at 25° C. for 2 h. Upon completion of the reaction, it was concentrated at 35° C. under reduced pressure. The resulting crude material was dissolved in H₂O (2 mL) and it was acidified with 5% citric acid solution. The solid obtained was filtered, washed with H₂O (10 mL) and dried to get 100 mg of (5-(3-ethynyl-1,2,4-thiadiazol-5-yl)-2-methylphenyl)glycine (INT-III-4) as a yellow solid that used in the next step without any further purification.

### Synthesis of (5-(3-(difluoromethyl)-1,2,4-thiadiazol-5-yl)-2-methylphenyl) glycine (INT-III-5)

Step (a): To a stirred solution of 4,4,5,5-tetramethyl-2-(4-methyl-3-nitrophenyl)-1,3,2-dioxaborolane (SM2-I-7a, 10 g, 38 mmol) in DMF (110 mL) were added 3-bromo-5-chloro-1,2,4-thiadiazole (7.52 g, 38 mmol), potassium phosphate (20.1 g, 95 mmol) and the reaction mixture was degassed with nitrogen for 5 min. After 5 min [1,1′-bis(diphenylphosphino)ferrocene]dichlopalladium dichloromethane complex (3.10 g, 3.80 mmol) was added and the reaction mixture was stirred for 30 min at 100° C. After completion of the reaction as indicated by TLC, the reaction mixture was diluted with ice cold H₂O and extracted with EtOAc (2×500 mL), the combined organic layer was washed with brine (3×200 mL), dried over anhyd. Na₂SO₄ and concentrated under reduced pressure. This crude residue was purified by column chromatography using (EtOAc: heptane, 12:88) as an eluent to afford 3-bromo-5-(4-methyl-3-nitrophenyl)-1,2,4-thiadiazole (SM2-I-5a) as a white solid. R_{f}(EtOAc: heptane, 10:90)=0.3; yield: 6.2 g, 55%. MS: m/z 297.98 [M−H]⁺. ¹H-NMR (400 MHz; DMSO-d₆): δ 8.58 (s, 1H), 8.28 (d, 1H), 7.74 (d, 1H), 2.61 (s, 3H).

Step (b): To a stirred solution of 3-bromo-5-(4-methyl-3-nitrophenyl)-1,2,4-thiadiazole (SM2-I-5a, 6.0 g; 20 mmol) in EtOH (90 mL) was added potassium acetate (5.9 g, 60 mmol) and the reaction mixture was degassed under nitrogen for 5 min. After 5 min, [1,1′-bis(diphenylphosphino)-ferrocene]dichloropalladium (II) dichloromethane complex (2.4 g, 13 mmol) was added and the reaction mixture was stirred at 80° C. under 50 psi atm. pressure of CO gas for 16 h. After completion of the reaction as indicated by TLC, the reaction mixture was concentrated under reduced pressure and water (50 mL) was added. The aq. layer was extracted with EtOAc (2×100 mL), the combined organic layer was washed with brine, dried over Na₂SO₄ and concentrated under reduced pressure. This crude residue was purified by column chromatography using (EtOAc: heptane, 15:85) as an eluent to afford ethyl 5-(3-amino-4-methylphenyl)-1,2,4-thiadiazole-3-carboxylate (SM2-I-5b) as a solid. R_{f}(EtOAc: heptane, 30:70)=0.4; yield: 1.45 g, 28%. MS: m/z 264.12 [M+H]⁺.

Step (c): To a stirred solution of ethyl 5-(3-amino-4-methylphenyl)-1,2,4-thiadiazole-3-carboxylate (SM2-I-5b, 1.4 g, 5.3 mmol), 4-dimethylaminopyridine (1.9 g, 16 mmol) in CH₃CN (20 mL) were added boc-anhydride (2.4 mL, 10.6 mmol) at 0° C. and the reaction mixture was stirred at 25° C. for 6 h. After completion of the reaction as indicated by TLC, the reaction mixture was concentrated under reduced pressure and diluted by water (15 mL) and extracted with EtOAc (2×30 mL). The combined organic layer was washed with brine, dried over Na₂SO₄ and concentrated under reduced pressure. This crude residue was purified by column chromatography using (EtOAc: heptane, 22:88) as an eluent to afford ethyl 5-(3-((tert-butoxycarbonyl)amino)-4-methylphenyl)-1,2,4-thiadiazole-3-carboxylate (SM2-I-5c). R_{f}(EtOAc: heptane, 5:5)=0.4; yield: 0.83 g, 43%. MS: m/z 364.08 [M+H]⁺.

Step (d): To a stirred solution of ethyl ethyl 5-(3-((tert-butoxycarbonyl)amino)-4-methylphenyl)-1,2,4-thiadiazole-3-carboxylate (SM2-I-5c, 0.75 g, 2.06 mmol) in EtOH (10 mL) were added sodium borohydride (157 mg, 4.13 mmol) at 0° C. and the reaction mixture was stirred at 25° C. for 2 h. After completion of the reaction as indicated by TLC, the reaction mixture was concentrated under reduced pressure, diluted by water and extracted by EtOAc (2×30 mL). The combined organic layer was washed with brine, dried over Na₂SO₄ and concentrated under under reduced pressure. This crude residue was purified by column chromatography using (EtOAc: heptane, 30:70) as an eluent to afford tert-butyl(5-(3-(hydroxymethyl)-1,2,4-thiadiazol-5-yl)-2-methylphenyl) carbamate (SM2-I-5d). R_{f} (EtOAc heptane, 5:5)=0.3; yield: 0.45 g, 68%. MS: m/z 322.21 [M+H]⁺.

Step (e): To a stirred solution of tert-butyl (5-(3-(hydroxymethyl)-1,2,4-thiadiazol-5-yl)-2-methylphenyl)carbamate (SM2-I-5d, 0.6 g, 1.87 mmol) in DCM (10 mL) was added DMP (1.58 g, 3.73 mmol) at 0° C. and the reaction mixture was stirred at 25° C. for 16 h. After completion of the reaction as indicated by TLC, the reaction mixture was filtered and the filtrate was concentrated under reduced pressure, diluted with H₂O (10 mL) and extracted by EtOAc (3×30 mL). The combined organic layer was washed with brine, dried over Na₂SO₄, concentrated under under reduced pressure. This crude residue was purified by silica gel column chromatography using (EtOAc: heptane, 20:80) as an eluent to afford tert-butyl (5-(3-formyl-1,2,4-thiadiazol-5-yl)-2-methylphenyl)carbamate (SM2-I-5e). R_{f} (EtOAc: heptane, 50:50)=0.5; yield: 0.52 g, 87%. MS: m/z 320.11 [M+H]⁺.

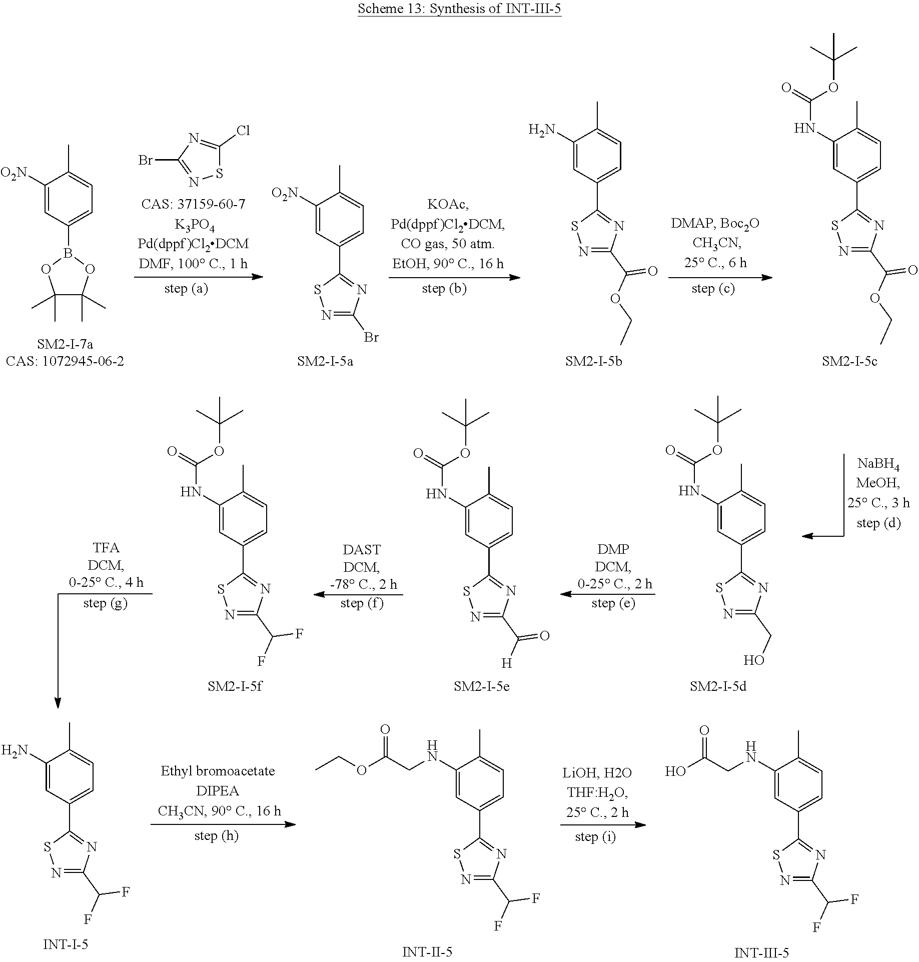

Step (f): To a stirred solution of tert-butyl (5-(3-formyl-1,2,4-thiadiazol-5-yl)-2-methylphenyl)carbamate (SM2-I-5e, 0.5 g, 1.57 mmol) in DCM (10 mL) were added DAST (0.62 mL, 4.70 mmol) at −70° C. and the reaction mixture was kept for stirring at 0-25° C. for 1 h. After completion of the reaction as indicated by TLC, the reaction mixture was quenched with NaHCO₃ soln. and extracted with DCM (3×60 mL). The combined organic layer was dried over Na₂SO₄ and concentrated under reduced pressure. This crude residue was purified by column chromatography using (EtOAc: heptane, 18:82) as an eluent to afford tert-butyl (5-(3-(difluoromethyl)-1,2,4-thiadiazol-5-yl)-2-methylphenyl)carbamate (SM2-I-5f). R_{f}(EtOAc: heptane, 40:60)=0.7; yield: 0.49 g, 92%. MS: m z 342.06 [M−H]⁺.

Step (g): To a stirred solution of tert-butyl (5-(3-(difluoromethyl)-1,2,4-thiadiazol-5-yl)-2-methylphenyl)carbamate (SM2-I-5f, 0.46 g, 1.35 mmol) in DCM (8 mL) were added TFA (1.04 mL, 13.5 mmol) at 0° C. the reaction mixture was stirred at 25° C. for 4 h. After completion of the reaction as indicated by TLC, the reaction mixture was concentrated under reduced pressure, quenched with aq. NaHCO₃ and extracted with EtOAc (3×30 mL). The combined organic layer was dried over Na₂SO₄, concentrated under reduced pressure. This crude residue (INT-I-5) was forwarded to the next step without further purification. R_{f}(EtOAc: heptane, 4:6)=0.4; crude yield: 0.31 g, 95%. MS: m/z 242.12 [M+H]⁺.

Step (h): Treatment of 5-(3-(difluoromethyl)-1,2,4-thiadiazol-5-yl)-2-methylaniline (INT-I-5, 0.30 g, 1.24 mmol) with ethyl 2-bromoacetate (0.3 mL, 2.49 mmol) and DIPEA (0.65 mL, 3.73 mmol) in CH₃CN (8 mL) at 100° C. for 16 h followed by work-up and purification afforded ethyl (5-(3-(difluoromethyl)-1,2,4-thiadiazol-5-yl)-2-methylphenyl)glycinate (INT-II-5b). R_{f}(EtOAc: heptane, 3:7)=0.5; yield: 0.29 g, 72%. MS: m/z 328.01 [M+H]⁺.

Step (i): Treatment of ethyl (5-(3-(difluoromethyl)-1,2,4-thiadiazol-5-yl)-2-methylphenyl)glycinate (INT-II-5, 0.14 g, 0.43 mmol) with LiOH. H₂O (53 mg, 1.28 mmol) in THF: H₂O (5:1, 6 mL) at 25° C. for 2 h followed by acidic work-up afforded (5-(3-(difluoromethyl)-1,2,4-thiadiazol-5-yl)-2-methylphenyl)glycine (INT-III-5) as a white solid. R_{f}(EtOAc: heptane, 1:1)=0.1; yield: 110 mg, 86%. MS: m/z 300.2 [M+H]⁺.

### Synthesis of (5-(3-cyclopropyl-1,2,4-thiadiazol-5-yl)-2-methylphenyl)glycine (INT-III-6)

To the solution of amine (INT-I-6, 0.4 g, 1.7 mmol) and ethyl bromoacetate (0.310 mL, 2.5 mmol, 1.5 equiv.) in DMF (10 mL), DIPEA (0.819 mL, 4.2 mmol, 2.5 equiv.) was added. The mixture was heated to 60° C. for overnight. After the reaction was completed, the mixture was poured into water and extracted with EtOAc (2×50 mL). The combined organic layer was washed with brine, dried over Na₂SO₄, filtered and evaporated under reduced pressure to dryness. The resulting crude mixture was then dissolved in ethanol, in which 2 mL of 2 N NaOH was added. The reaction was heated to 100° C. for 1 h and cooled down to rt. Ethanol was removed under reduced pressure and the residue was re-dissolved in water. To the emulsion, 2 N HCl was added the resulting solid was filtered off and dried at 40° C. in oven to afford INT-III-6 (yield: 43%, 200 mg, over two steps). LC-MS (m/z): 289.9 [M+H]⁺. Purity by HPLC-UV (254 nm)-ESI-MS: 98.5%.

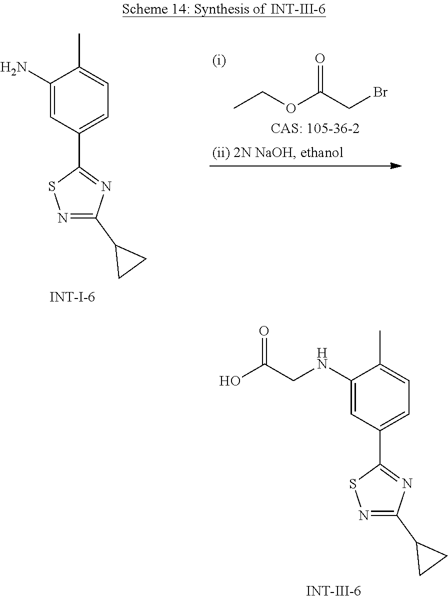

### Synthesis of (5-(3-bromo-1,2,4-thiadiazol-5-yl)-2-methylphenyl)glycine (INT-III-7)

Step (a): To a stirred solution of bromonitrobenzene derivatives (1 equiv.) in toluene or DMSO was added 4,4,4′,4′,5,5,5′,5′-octamethyl-2,2′-bi(1,3,2-dioxaborolane) in portionwise (1.1 equiv.) and potassium acetate (2.5 equiv.) and degassed the reaction mixture with N₂ atmosphere. After 10 min, [1,1′-bis(diphenylphosphino)ferrocene]dichloropalladium(II)dichloromethane complex (1.1 equv.) was added and the resulting reaction mixture was kept for stirring at 90° C. for 5 h. After completion of the reaction as indicated by TLC, the reaction mixture was filtered and the filtrate was concentrated under reduced pressure. This crude residue was purified by column chromatography using EtOAc/heptane (0 -6%) to afford nitrophenyldioxaborolane derivatives.

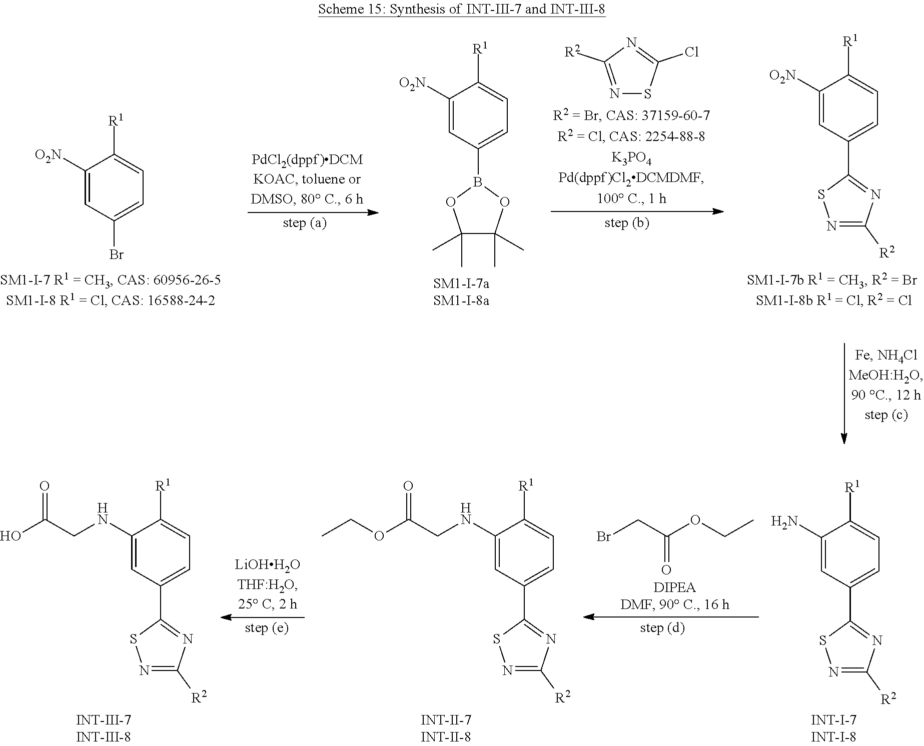

4,4,5,5-Tetramethyl-2-(4-methyl-3-nitrophenyl)-1,3,2-dioxaborolane (SM1-I-7a): Treatment of 4-bromo-1-methyl-2-nitrobenzene (SM1-I-7, 20 g, 93 mmol) with 4,4,4′,4′,5,5,5′,5′-octamethyl-2,2′-bi(1,3,2-dioxaborolane) (47.2 g, 186 mmol), potassium acetate (22.7 g, 232 mmol) and [1,1′-bis(diphenylphosphino)ferrocene]dichloropalladium(II)dichloromethane complex (7.58 g, 9.23 mmol) in toluene (220 mL) for 5 h at 90° C. followed column chromatography using 0-6% EtOAc/heptane to afford 4,4,5,5-tetramethyl-2-(4-methyl-3-nitrophenyl)-1,3,2-dioxaborolane (SM1-I-7a) as a solid. R_{f} (EtOAc: heptane, 1:9)=0.4; yield: 20.5 g, 84%.

2-(4-Chloro-3-nitrophenyl)-4,4,5,5-tetramethyl-1,3,2-dioxaborolane (SM1-I-8a): The product was obtained via treatment of 4-bromo-1-chloro-2-nitrobenzene (SM1-I-8, 10 g, 42 mmol) with 4,4,4′,4′,5,5,5′,5′-octamethyl-2,2′-bi(1,3,2-dioxaborolane; 11.8 g, 46.6 mmol), [1,1′-bis(diphenylphosphino)ferrocene]dichloropalladium(II)dichloromethane complex (0.34 g, 4.2 mmol) and potassium acetate (10.3 g, 105 mmol) in DMSO (50 mL) for 3 h at 90° C. Afterward, column chromatography purification using 0-6% EtOAc/heptane afforded 2-(4-chloro-3-nitrophenyl)-4,4,5,5-tetramethyl-1,3,2-dioxaborolane (SM1-I-8a) as a white solid, yielding: 9 g, 75% that was used directly into the next step.

Step (b): To a stirred solution of nitrophenyldioxaborolane derivatives (1 equiv.) in DMF/H₂O mixture were added the corresponding thiadiazole (1.1 equiv.), potassium phosphate (2.5 equiv.) and the reaction mixture was degassed for 5 min with N₂ atmosphere. After that, [1,1′-bis(diphenylphosphino)ferrocene]dichlopalladium dichloromethane complex (0.1 equiv.) was then added and the reaction mixture was stirred for 100° C. After completion of the reaction as indicated by TLC, the reaction mixture was diluted with ice-cold H₂O and extracted with EtOAc (3×25 mL). The combined organic layers were washed with brine, dried over Na₂SO₄ and concentrated under reduced pressure. This crude residue was purified by column chromatography using 0-12% EtOAc/heptane as an eluent to afford the corresponding nitrophenylthiadiazole derivatives.

3-Bromo-5-(4-methyl-3-nitrophenyl)-1,2,4-thiadiazole (SM1-I-7b): Treatment of 4,4,5,5-tetramethyl-2-(4-methyl-3-nitrophenyl)-1,3,2-dioxaborolane (SM1-I-7a, 10 g, 38 mmol) with 3-bromo-5-chloro-1,2,4-thiadiazole (7.52 g, 38 mmol), potassium phosphate (20.1 g, 95 mmol) and [1,1′-bis(diphenylphosphino)ferrocene]dichlopalladium dichloromethane complex (3.10 g, 3.8 mmol) in DMF (110 mL) water (5 mL) mixture for 30 min at 100° C. followed column chromatography using 0-12% EtOAc/heptane as an eluent to afford 3-bromo-5-(4-methyl-3-nitrophenyl)-1,2,4-thiadiazole (SM1-I-7b) as a white solid. R_{f}(EtOAc: heptane, 10:90)=0.3; yield: 6.2 g, 55%. MS: m/z 297.98 [M −H]⁺. ¹H-NMR (400 MHz; DMSO-d₆): δ 8.58 (s, 1H), 8.28 (d, 1H), 7.74 (d, 1H), 2.61 (s, 3H).

3-Chloro-5-(4-chloro-3-nitrophenyl)-1,2,4-thiadiazole (SM1-I-8b): Treatment of 2-(4-chloro-3-nitrophenyl)-4,4,5,5-tetramethyl-1,3,2-dioxaborolane (SM1-I-8a, 3 g, 10.6 mmol) with 3,5-dichloro-1,2,4-thiadiazole (1.89 g, 11.66 mmol), potassium phosphate (5.69 g, 26.5 mmol) and [1,1′-bis(diphenylphosphino)ferrocene]dichlopalladium dichloromethane complex (0.89 g, 1.06 mmol) in DMF (30 mL) and water (5 mL) mixture for 1 h at 90° C. followed column chromatography using 0-12% EtOAc/heptane as an eluent to afford 3-chloro-5-(4-chloro-3-nitrophenyl)-1,2,4-thiadiazole (SM1-I-8b) as a white solid (1.89 g, 62%) that was used directly for the next step.

Step (c): To a stirred solution of (3-nitrophenyl)-1,2,4-thiadiazole derivatives (1 equiv.) in EtOH and H₂O (3:1) was added iron powder (7 equiv.) followed by NH₄Cl (7 equiv.) at 25° C. under N₂atmosphere and the reaction mixture was stirred at 80° C. for 5 h. After completion of the reaction as indicated by TLC, the reaction mixture was concentrated, diluted with EtOAc and filtered through celite bed. Filtrate was extracted with EtOAc (3 times). The combined organic layers were washed with brine, dried over anhy. Na₂SO₄, filtered and concentrated under reduced pressure. This crude residue was triturated with DCM and heptane to afford the corresponding anilines.

5-(3-Bromo-1,2,4-thiadiazol-5-yl)-2-methylaniline (INT-I-7): Treatment of 3-bromo-5-(4-methyl-3-nitrophenyl)-1,2,4-thiadiazole (SM1-I-7b, 3 g, 10 mmol) with iron powder (2.8 g, 50.2 mmol) in the presence of NH₄Cl (2.7 g, 50.2 mmol) in MeOH: H₂O (8:1, 45 mL) at 90° C. for 4 h followed by work-up afforded 5-(3-bromo-1,2,4-thiadiazol-5-yl)-2-methylaniline (INT-I-7) as a yellow colour solid. R_{f} (EtOAc: heptane, 20:80)=0.2; yield: 2.5 g, 92%. MS: m/z 270.02 [M+H]⁺.

2-Chloro-5-(3-chloro-1,2,4-thiadiazol-5-yl)aniline (INT-I-8): Treatment of 3-chloro-5-(4-chloro-3-nitrophenyl)-1,2,4-thiadiazole (SM1-I-8b, 1.8 g, 6.54 mmol) with iron powder (1.8 g, 32.72 mmol) in the presence of NH₄Cl (1.79 g, 32.72 mmol) in ethanol: H₂O (3:1, 20 mL) at 90° C. for 3 h followed by work-up afforded 2-chloro-5-(3-chloro-1,2,4-thiadiazol-5-yl)aniline (INT-I-8) as a off white solid, yield: 1.4 g, 87% that used in the next step.

Step (d): To a stirred solution of (1,2,4-thiadiazol-5-yl)aniline derivative (1.0 equiv.) in DMF were added DIPEA (2.0 equiv.) and ethyl 2-bromoacetate (1.1 equiv.) under N₂ atmosphere and the reaction mixture was stirred at 100° C. for 16 h. After completion of the reaction as indicated by TLC, the reaction mixture was diluted with ice cold H₂O and extracted with EtOAc (3 times). The combined organic layers were washed with ice cold brine and dried over anhy. Na₂SO₄, filtered and concentrated under reduced pressure. This crude residue was purified by column chromatography to afford the corresponding ethyl carboxylates.

Ethyl (5-(3-bromo-1,2,4-thiadiazol-5-yl)-2-methylphenyl)glycinate (INT-II-7): Treatment of 5-(3-bromo-1,2,4-thiadiazol-5-yl)-2-methylaniline (INT-I-7, 2.5 g, 9.29 mmol) with ethyl 2-bromoacetate (1.2 mL, 11.2 mmol) in the presence of DIPEA (4.9 mL, 27.8 mmol) in DMF (20 mL) at 90 C for 16 h followed by work-up and purification by column chromatography using 0-15% EtOAc/heptane afforded ethyl (5-(3-bromo-1,2,4-thiadiazol-5-yl)-2-methylphenyl)glycinate (INT-II-7) as an off white solid. R_{f}(EtOAc: heptane, 30:70)=0.5; yield: 2.2 g, 68%. MS: m/z 356.12 [M+H]⁺.

Ethyl (2-chloro-5-(3-chloro-1,2,4-thiadiazol-5-yl)phenyl)glycinate (INT-II-8): Treatment of 2-chloro-5-(3-chloro-1,2,4-thiadiazol-5-yl)aniline (INT-I-8, 1.29 g, 4.89 mmol) with ethyl 2-bromoacetate (0.8 mL, 7.34 mmol) in the presence of DIPEA (2.6 mL, 14.67 mmol) in DMF (10 mL) at 100° C. for 16 h followed by work-up and purification by column chromatography using 0-15% EtOAc/heptane afforded ethyl (2-chloro-5-(3-chloro-1,2,4-thiadiazol-5-yl)phenyl)glycinate (INT-II-8) as an off white solid (1.1 g, 68%) that was used directly for the next step.

Step (e): To a stirred solution of (1,2,4-thiadiazol-5-yl)phenyl)glycinate derivative (1.0 equiv.) in MeOH: THF: H₂O (1:2:1), was added lithium hydroxide monohydrate (LiOH. H₂O, 3.0 equiv.) at 0° C.. The resulting reaction mixture stirred at 25° C. for 2 h. After completion of the reaction as indicated by TLC, the reaction mixture was concentrated under reduced pressure and diluted with H₂O. The aqueous layer was acidified with sat. citric acid soln. (pH=5-6) at 0° C. resulting precipitation of the product. The solid product was dried under high reduced pressure to afford the corresponding acid as a white solid.

(5-(3-Bromo-1,2,4-thiadiazol-5-yl)-2-methylphenyl)glycine (INT-III-7): Treatment of ethyl (5-(3-bromo-1,2,4-thiadiazol-5-yl)-2-methylphenyl)glycinate (INT-II-7, 1.7 g, 4.78 mmol) with LiOH. H₂O (0.6 g, 14.4 mmol) in MeOH: THF: H₂O (4:3:2, 9 mL) at 25° C. for 2 h followed by acidic work-up and precipitation afforded (5-(3-bromo-1,2,4-thiadiazol-5-yl)-2-methylphenyl)glycine (INT-III-7) as a yellow solid. R_{f}(MeOH: EtOAc, 5:95)=0.1; yield: 1.5 g, 95%. MS: m/z 328.08 [M+H

(2-Chloro-5-(3-chloro-1,2,4-thiadiazol-5-yl)phenyl)glycine (INT-III-8): Treatment of ethyl (2-chloro-5-(3-chloro-1,2,4-thiadiazol-5-yl)phenyl)glycinate (INT-II-8, 1.1 g, 3.32 mmol) with LiOH. H₂O (0.41 g, 9.96 mmol) in EtOH: THF: H₂O (4:5:2, 11 mL) at 25° C. for overnight followed by acidic work-up and precipitation afforded (2-chloro-5-(3-chloro-1,2,4-thiadiazol-5-yl)phenyl)glycine (INT-III-8) as a yellow solid, yield: 1.0 g, 65%.

### Synthesis of compounds INT-III-13, INT-III-13 (Formulae part 4)

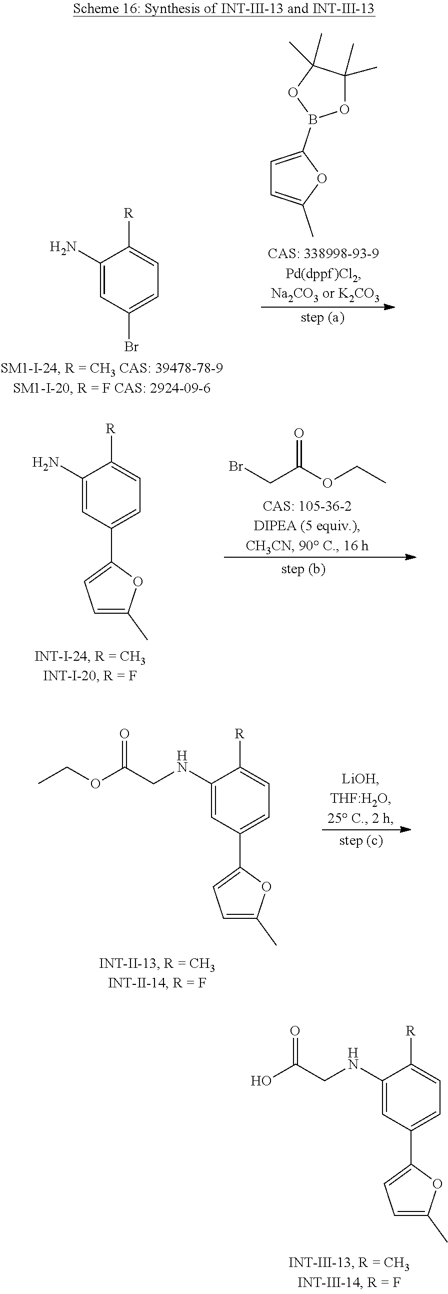

Step (a): A mixture of bromolaniline derivatives (1.0 equiv.), 5-methylfuran-2-boronic acid-pinacol ester (2 equiv.), and K₂CO₃ (1.5 equiv.) or Na₂CO₃ (2 equiv.) was loaded successively in a screw cap reaction vial. The solvent was added and the reaction mixture was degassed by ultrasonication for 20 min with purged with argon. Then, 1,1′-bis(diphenylphosphino)ferrocene]-dichloropalladium(II) (0.1 equiv.) was added under argon. The vial was heated at 100° C. for overnight. Solvent was removed in vacuo and the residue was partitioned between H₂O and DCM. The organic layer was washed with H₂O (2×20 mL), brine (20 mL), dried over MgSO₄, and solvent was removed in vacuo and the crude product was purified by using method II to give the products.

2-Methyl-5-(5-methylfuran-2-yl)aniline (INT-I-24): The compound was prepared according to step (a) using a mixture of 5-bromo-2-methylaniline (SM1-I-24, 535 mg, 2.88 mmol), 5-methylfuran-2-boronic acid-pinacol ester (1.197 g, 5.75 mmol, 2 equiv.), K₂CO₃ (596.3 mg, 4.315 mmol, 1.5 equiv.) and 1,1′-bis(diphenylphosphino)ferrocene]-dichloropalladium(II) (210.5 mg, 0.288 mmol, 0.1 equiv.) in dioxane (8 mL) and H₂O (5 mL). The crude product was purified by using method II giving INT-I-24 (406 mg, 75.3%). HPLC-UV (220-400 nm) ESI-MS Purity: 98.3%. LC-MS (m/z): 187.8 [M+H]⁺.

2-Fluoro-5-(5-methylfuran-2-yl)aniline (INT-I-20): The compound was prepared according to step (a) using a mixture of 5-bromo-2-fluoroaniline (SM1-I-20, 3.5 g, 18 mmol), 5-methylfuran-2-boronic acid-pinacol ester (3.8 g, 18 mmol, 1 equiv.), Na₂CO₃ (4.7 g, 45 mmol, 2.5 equiv.) and 1,1′-bis(diphenylphosphino)ferrocene]-dichloropalladium(II) (1.03 g, 0.9 mmol, 0.1 equiv.) in toluene (30 mL), EtOH (8 mL), and H₂O (3 mL) mixture. The crude product was purified by using method II giving INT-I-20 (2.4 g, 68%) as a yellow solid that used in the next step.

Ethyl (2-methyl-5-(5-methylfuran-2-yl)phenyl)glycinate (INT-II-13): It was prepared according to the general procedure (Step (f), Scheme 10), starting with INT-I-24 to afford INT-II-13 that used in the next step without further purification, HPLC-UV (220-400 nm) ESI-MS Purity: 86.9%. LC-MS (m/z): 274.0 [M+H]⁺.

Ethyl (2-fluoro-5-(5-methylfuran-2-yl)phenyl)glycinate (INT-II-14): It was prepared according to the general procedure (Step (g), Scheme 10), starting with INT-I-20 (2.3 g, 12 mmol) and ethyl bromoacetate (2.68 g, 24 mmol) and DIPEA (16.7 mL, 96 mmol) to afford INT-II-14 (1.7 g, 51% yield) that used in the next step without further purification.

(2-Methyl-5-(5-methylfuran-2-yl)phenyl)glycine (INT-III-13): It was prepared according to the general procedure (Step (g), Scheme 10), starting with INT-II-13 to afford INT-III-13 that used in the next step without further purification (317.5 mg, 45.3%). HPLC-UV (220-400 nm) ESI-MS Purity: 79.5%. LC-MS (m/z): 246.0 [M+H]⁺.

(2-Fluoro-5-(5-methylfuran-2-yl)phenyl)glycine (INT-III-14): It was prepared according to the general procedure (Step (g), Scheme 10), starting with INT-II-14 to afford INT-III-14 that used in the next step without further purification (1.18 g, 78% yield).

### Synthesis of (1,2,4-oxadiazol-5-yl)phenyl)glycine derivatives (INT-III-12, INT-III-15, and INT-III-16)

### Step (a): Synthesis of (3-nitrophenyl)-1,2,4-oxadiazole derivatives (SM2-I-12a, SM2-I-15a, and SM2-I-16a)

To a stirred solution of the acid SM2-I-12 and SM2-I-16 (1 equiv.) in dioxane were added the corresponding imidamide derivatives (1.0 equiv.), Et₃N (3 equiv.) and T3P (50% w/w in EtOAc, 1.5 equiv.) under N₂ atmosphere at 0° C. The resulting reaction mixture was stirred at 25° C. for 2-16 h. After completion of the reaction as indicated by TLC, the reaction mixture was quenched with ice cold H₂O and extracted with EtOAc (3 times). The combined organic layer was dried over anhy. Na₂SO₄ and concentrated under reduced pressure. The resulting crude was purified either by silica gel column chromatography or by reverse-phase chromatography to afford the corresponding product.

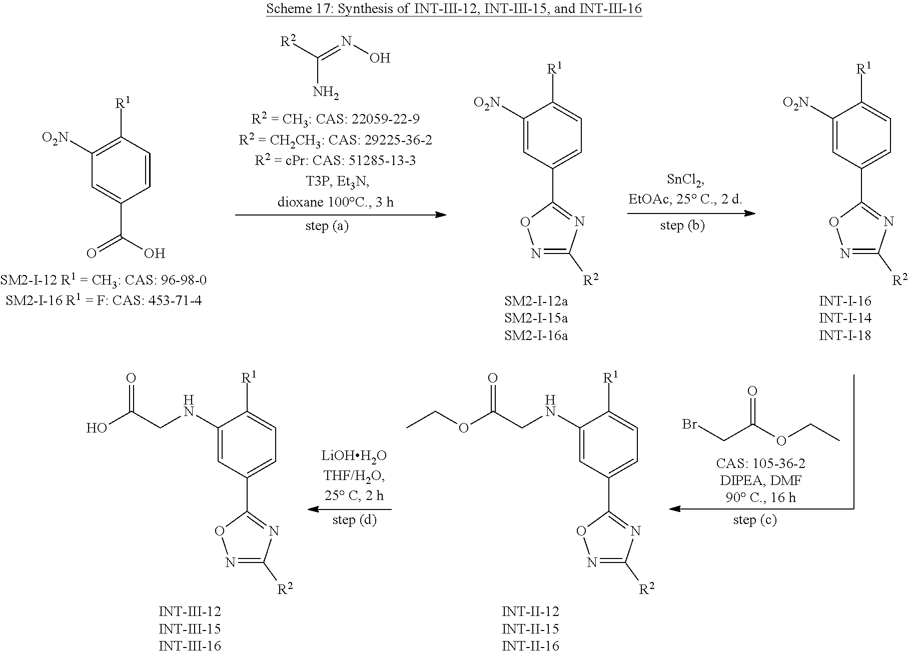

3-Methyl-5-(4-methyl-3-nitrophenyl)-1,2,4-oxadiazole (SM2-I-15a): Treatment of 4-methyl-3-nitrobenzoic acid (SM2-I-12, 7.0 g, 38.7 mmol) with N′-hydroxyacetimidamide (11.4 g, 155 mmol) in presence of T3P (68.3 mL, 50% w/w in EtOAc, 96.7 mmol) and DIPEA (17 mL, 96.7 mmol) in THF (100 mL) at 90° C. for 16 h followed by work-up and purification afforded 3-methyl-5-(4-methyl-3-nitrophenyl)-1,2,4-oxadiazoleas (SM2-I-15a) as a white solid. R_{f}(EtOAc: heptane, 50:50)=0.5; yield: 5 g 59%. MS: m/z 220.26 [M+H⁺.

5-(4-Fluoro-3-nitrophenyl)-3-methyl-1,2,4-oxadiazole (SM2-I-16a): Treatment of 4-fluoro-3-nitrobenzoic acid (SM2-I-16, 14.0 g, 75.6 mmol) with N-hydroxyacetimidamide (11.2 g, 151 mmol) in presence of T3P (267 mL, 50% w/w in EtOAc, 378 mmol) and Et₃N (52 mL, 378 mmol) in dioxane (500 mL) at 80° C. for 2 h followed by work-up and precipitation of the product with ice cold water afforded 5-(4-fluoro-3-nitrophenyl)-3-methyl-1,2,4-oxadiazole as a yellow solid. R_{f}(EtOAc: heptane, 20:80)=0.5; yield: 7.2 g, 43%.

3-Cyclopropyl-5-(4-methyl-3-nitrophenyl)-1,2,4-oxadiazole (SM2-I-12a): Treatment of 4-methyl-3-nitrobenzoic acid (14.3 g, 80 mmol) with N′-hydroxycyclopropanecarboximidamide (10 g, 100 mmol) in presence of T3P (176 mL, 50% w/w in EtOAc, 250 mmol) and Et₃N (41.5 mL, 300 mmol) in dioxane (80 mL) at 100° C. for 36 h followed by work-up and purification afforded 3-cyclopropyl-5-(4-methyl-3-nitrophenyl)-1,2,4-oxadiazole (SM2-I-12a) as an off white solid. R_{f}(EtOAc: heptane, 50:50)=0.5; yield: 8.5 g, 35%. MS: m/z 246.07 [M+H]⁺.

### Step (b): General Procedure for the Synthesis of (1,2,4-Oxadiazol-5-Yl) Aniline Derivatives

To a stirred solution (3-nitrophenyl)-1,2,4-oxadiazole (1 equiv.) in EtOAc was added SnCl₂ (5 equiv.) at 25° C. under N₂ atmosphere and the reaction mixture was stirred at 25° C. for 2 d. After completion of the reaction as indicated by TLC, the reaction mixture was filtered through celite. Water was added to the filtrate and extracted with EtOAc (3 times). The combined organic layer was washed with brine, dried over anhy. Na₂SO₄, filtered and concentrated under reduced pressure. Crude residue was purified by combi-flash chromatography using EtOAc: heptane as an eluent to afford corresponding anilines.

2-Methyl-5-(3-methyl-1,2,4-oxadiazol-5-yl)aniline (INT-I-14): Treatment of 3-methyl-5-(4-methyl-3-nitrophenyl)-1,2,4-oxadiazole (SM2-I-15a, 3 g, 13.6 mmol) with SnCl₂ (12.9 g, 68.4 mmol) in EtOAc (100 mL) at 25° C. for 2 d followed by work-up and purification afforded 2-methyl-5-(3-methyl-1,2,4-oxadiazol-5-yl)aniline (INT-I-14) as a yellow solid. R_{f}(EtOAc: heptane, 40. 60)=0.2; yield: 1.9 g, 74%. MS: m/z 188.20 [M−H]⁺.

2-Fluoro-5-(3-methyl-1,2,4-oxadiazol-5-yl)aniline (INT-I-18): Treatment of 3,5-(4-fluoro-3-nitrophenyl)-3-methyl-1,2,4-oxadiazole (SM2-I-16a, 9 g, 40 mmol) with SnCl₂ (26.7 g, 141.2 mol) in EtOAc (1 L) at 25° C. for 16 h followed by work-up and purification afforded 5-(3-ethyl-1,2,4-oxadiazol-5-yl)-2-fluoroaniline (INT-I-18) as a white solid. R_{f}(EtOAc: heptane, 20:80)=0.4; yield: 5.65 g, 73%. MS: m/z 194.06 [M+H]⁺.

5-(3-Cyclopropyl-1,2,4-oxadiazol-5-yl)-2-methylaniline (INT-I-16): Treatment of 3-cyclopropyl-5-(4-methyl-3-nitrophenyl)-1,2,4-oxadiazole (SM2-I-12a, 4 g, 16.3 mmol) with SnCl₂ (15.4 g, 81.6 mmol) in EtOAc (40 mL) at 25° C. for 3 d followed by work-up and precipitation afforded 5-(3-cyclopropyl-1,2,4-oxadiazol-5-yl)-2-methylaniline (INT-I-16) as an off white solid. R_{f} (EtOAc heptane, 30:70)=0.2; yield: 2.9 g, 83%. MS: m/z 216.14 [M+H]⁺.

### Step (c): Synthesis of (1,2,4-Oxadiazol-5-yl)Phenyl)Glycinate Derivatives

To a stirred solution of (1,2,4-thiadiazol-5-yl)aniline derivative (1.0 equiv.) in DMF were added DIPEA (2.0 equiv.) and ethyl 2-bromoacetate (1.1 equiv.) under N₂ atmosphere. This reaction mixture was stirred at 90-100° C. for 16 h. After completion of the reaction as indicated by TLC, the reaction mixture was diluted with ice cold water and extracted with EtOAc (3 times). The combined organic layers were washed with ice cold brine and dried over anhyd. Na₂SO₄, filtered and concentrated under reduced pressure. This crude residue was purified by column chromatography to afford corresponding ethyl carboxylates.

Ethyl (2-methyl-5-(3-methyl-1,2,4-oxadiazol-5-yl)phenyl)glycinate (INT-II-15): Treatment of 2-methyl-5-(3-methyl-1,2,4-oxadiazol-5-yl)aniline (INT-I-14, 1.8 g, 9.52 mmol) with ethyl 2-bromoacetate (1.2 mL, 11.4 mmol) and DIPEA (8.3 mL, 47.6 mmol) in DMF (15 mL) at 100 C for 16 h followed by work-up and purification afforded ethyl (2-methyl-5-(3-methyl-1,2,4-oxadiazol-5-yl)phenyl)glycinate (INT-II-15) as a white solid. R_{f}(EtOAc: heptane, 40:60)=0.2; yield: 1.8 g, 69%. MS: m/z 276.12 [M+H]⁺.

Ethyl (2-fluoro-5-(3-methyl-1,2,4-oxadiazol-5-yl)phenyl)glycinate (INT-II-16): Treatment of 2-fluoro-5-(3-methyl-1,2,4-oxadiazol-5-yl)aniline (INT-I-18, 5.6 g, 29 mmol) with ethyl 2-bromoacetate (3.5 mL, 31.9 mmol) and DIPEA (15 mL, 87 mmol) in DMF (50 mL) at 90° C. for 16 h followed by work-up and precipitation afforded ethyl (2-methyl-5-(3-methyl-1,2,4-oxadiazol-5-yl)phenyl)glycinate (INT-II-16) as a yellow solid. R_{f}(EtOAc: heptane, 20:80)=0.5; yield: 6.21 g, 76%. MS: m/z 280.17 (M+H)+.

Ethyl (2-methyl-5-(3-methyl-1,2,4-oxadiazol-5-yl)phenyl)glycinate (INT-II-12): Treatment of 5-(3-cyclopropyl-1,2,4-oxadiazol-5-yl)-2-methylaniline (INT-I-16, 2.8 g, 13 mmol) with ethyl 2-bromoacetate (1.7 mL, 15.6 mmol) and DIPEA (6.8 mL, 039 mmol) in DMF (30 mL) at 100 C for 16 h followed by work-up and purification afforded ethyl (5-(3-cyclopropyl-1,2,4-oxadiazol-5-yl)-2-methylphenyl)glycinate (INT-II-12) as an off white solid. R_{f}(EtOAc: heptane, 30:70)=0.4; yield: 2.6 g, 66%. MS: m/z 302.17 [M+H]⁺.

### Step (d): Synthesis of (1,2,4-Oxadiazol-5-yl)Phenyl)Glycine Derivatives

To a stirred solution of (1,2,4-thiadiazol-5-yl)phenyl)glycinate derivative (1.0 equiv.) in EtOH: THF H₂O (2:3:1), was added LiOH. H₂O (3.0 equiv.) at 0° C. The resulting reaction mixture stirred at 25° C. for 2 h. After completion of the reaction as indicated by TLC, the reaction mixture was concentrated under reduced pressure and diluted with H₂O. The aqueous layer was acidified with saturated solution of citric acid (pH=5-6) at 0° C. resulting precipitation of the product. The solid product was dried under high reduced pressure to afford corresponding acid as a white solid.

(2-Methyl-5-(3-methyl-1,2,4-oxadiazol-5-yl)phenyl)glycine (INT-III-15): Treatment of ethyl (2-methyl-5-(3-methyl-1,2,4-oxadiazol-5-yl)phenyl)glycinate (INT-TI-15, 1.8 g, 6.54 mmol) with LiOH. H₂O (0.8 g, 19.6 mmol) in EtOH: THF: H₂O (2:3:1, 14 mL) at 25° C. for 2 h followed by acidic work-up afforded (2-methyl-5-(3-methyl-1,2,4-oxadiazol-5-yl)phenyl) glycine (INT-ITT-15) as a white solid. R_{f}(MeOH: EtOAc, 5:95)=0.1; yield: 1.0 g, 62%. MS: m/z 246.03 [M−H]⁺.

(2-Fluoro-5-(3-methyl-1,2,4-oxadiazol-5-yl)phenyl)glycine (INT-III-16): Treatment of ethyl (2-fluoro-5-(3-methyl-1,2,4-oxadiazol-5-yl)phenyl)glycinate (INT-II-16, 6.2 g, 22.2 mmol) with LiOH. H₂O (5.3 g, 222 mmol) in EtOH: THF: H₂O (2:2:1, 100 mL) at 25° C. for 2 h followed by acidic work-up afforded (2-fluoro-5-(3-methyl-1,2,4-oxadiazol-5-yl)phenyl)glycine (INT-III-16) as a white solid. R_{f}(EtOAc: heptane, 40:60)=0.1; yield: 2.85 g, 52%. MS: m/z 252.13 [M+H

(5-(3-Cyclopropyl-1,2,4-oxadiazol-5-yl)-2-methylphenyl)glycine (INT-III-12): Treatment of ethyl (5-(3-cyclopropyl-1,2,4-oxadiazol-5-yl)-2-methylphenyl)glycinate (INT-II-12, 2.5 g, 8.25 mmol) with LiOH. H₂O (0.87 g, 20.6 mmol) in EtOH: THF: H₂O (2:3:1, 14 mL) at 25° C. for 2 h followed by acidic work-up afforded (5-(3-cyclopropyl-1,2,4-oxadiazol-5-yl)-2-methylphenyl)glycine (INT-III-12) as an off white solid. R_{f}(MeOH: EtOAc, 5:95)=0.1; yield: 1.8 g, 79%. MS: m/z 274.16 [M+H]⁺.

### Synthesis of Compounds INT-IV-(1-32), Scheme 1, Formulae Parts 5, 6

### Preparation of 1-(Indolin-4-yl)Ethan-1-ol (INT-IV-1)

Step (a): To a solution of 4-formyl indole (SM-IV-1a, 264 mg. 1.82 mmol, 1 equiv.) in dry THF (15 mL) under argon at 0° C. was added MeMgBr (3 M soln. in Et₂O, 1.5 mL, 4.55 mmol, 2.5 equiv.) dropwise over a time period of 30 min. The reaction mixture was stirred for 30 min at 0° C. and quenched afterwards with sat. soln. of NH₄Cl. The aq. layer was extracted three times with Et₂O. The organic layer was washed with brine, dried over MgSO₄ and concentrated under reduced pressure. The isolated crude product was chromatographed on silica gel with (cyclohexane:EtOAc, 50:50) to yield (195 mg, 1.21 mmol, 67%) of SM-IV-1b as a light yellow solid. HPLC-UV (220-400 nm) ESI-MS Purity: 86.7%. LC-MS (m/z): 143.7 [M−H₂O+H].

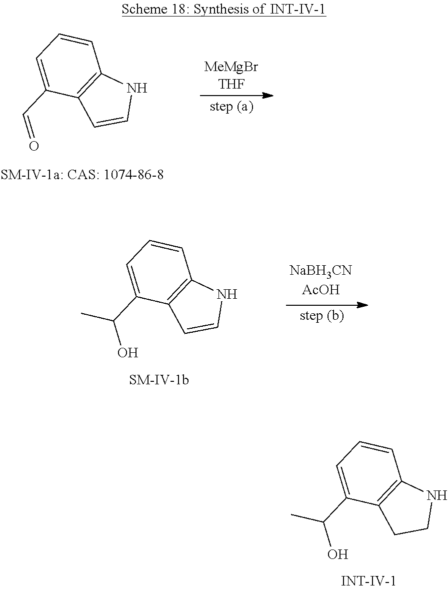

Step (b): To a solution of 1-(1H-indol-4-yl)ethan-1-ol (SM-IV-1b, 180 mg, 1.12 mmol, 1 equiv.) in AcOH (10 mL) was added NaBH₃CN (210 mg, 3.35 mmol, 3 equiv.) of at 0° C. in portions. The resulting mixture was stirred for 2 h at rt. After the reaction was completed, water (2 mL) was added and the solvent was removed under reduced pressure. The residue was taken up in EtOAc and neutralized with sat. soln. of NaHCO₃. The organic layer was separated, dried over MgSO₄ and concentrated under reduced pressure. The isolated crude product was chromatographed on silica gel with (cyclohexane:EtOAc , 50:50) to yield (65 mg, 0.40 mmol, 59%) of INT-IV-1 as a white solid. HPLC-UV (220-400 nm) ESI-MS Purity: 92%. LC-MS (m/z): 163.8 [M+H]⁺.

### Preparation of 2-(indolin-4-yl)propan-2-ol (INT-IV-2)

Step (a): In a 50 mL round flask, 4-methoxycarbonylindole (SM-IV-2a, 1 equiv.) was dissolved in DMF (generally, 50 mL for every Ig). Sodium hydride (1.2 equiv.) was added portionwise to the reaction mixture at 0° C. and the reaction mixture was stirred for 10 min at 0° C. p-Toluenesulfonyl chloride (1.1 equiv.) was added portionwise to the reaction mixture at 0° C. and the reaction mixture was stirred at rt for 1 h. Water was then carefully added, and the product was extracted with EtOAc (3×50 mL). The organic layers were combined, dried over MgSO₄, filtered, and concentrated under reduced pressure followed by purification with flash column chromatography (PE: EtOAc, 70:30, in 30 min gradient) to afford SM-IV-2b (yield: 57%).

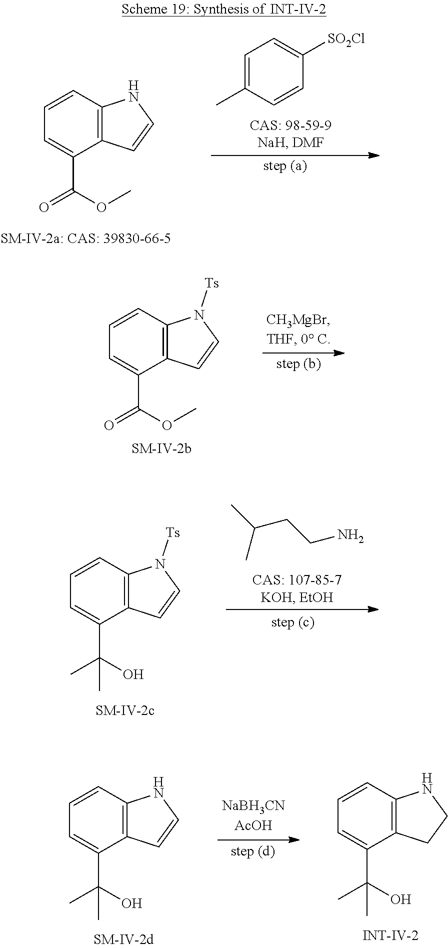

Step (b): Under dry condition, 2-(1-tosyl-1H-indol-4-yl)propan-2-ol (SM-IV-2b, 1 equiv.) in dry THF (50 mL) was put in a dry two-necked flask connected from one side with a condenser. MeMgBr (3 M soln. in Et₂O, 10 equiv.) was added dropwise using a dropping funnel at 0° C. The reaction mixture was further stirred for 3 h at 0° C. The reaction was then quenched with sat. NH₄Cl soln. and extracted with EtOAc (3×50 mL). The combined organic extracts were washed with brine and dried over anhydrous MgSO₄. The solvent was evaporated under reduced pressure followed by purification with flash column chromatography (PE: EtOAc, 70:30, in 30 min gradient) to afford SM-IV-2c (yield: 88%).

Step (c): In a round flask, 2-(1-tosyl-1H-indol-4-yl)propan-2-ol (SM-IV-2c, 1 equiv.), 3-methylbutan-1-amine (3 equiv.), and KOH (1.5 equiv.) were mixed together in EtOH (0.5 mL for every 100 mg) and heated at 100° C. in microwave for 1 h. The mixture was concentrated under reduced pressure. DCM (0.5 mL for every 100 mg starting material) was added and the combined organic layers were washed with brine and dried over anhyd. MgSO₄. The solvent was evaporated under reduced pressure followed by purification with flash column chromatography (PE: EtOAc, 70:30, in 30 min gradient) to afford SM-IV-2c (yield: 42%).

Step (d): The obtained indole SM-IV-2d (1 equiv.) was dissolved in 5 mL of acetic acid (AcOH) and cooled to 0° C. Then, NaBH₃CN (2 equiv.) was added, and the reaction mixture was stirred at rt for overnight. Afterward, H₂O (3 mL) was added and the solvents were concentrated in vacuo. The sat. soln.of NaHCO₃ was then carefully added, and the product was extracted with EtOAc (3×50 mL). Evaporation of the solvents afforded INT-IV-2 as a crude product, which was used for the next reaction without further purification (crude yield: 42%).

### Preparation of 5-methoxy-2,3-dihydro-1H-pyrrolo[3,2-b]pyridine (INT-IV-4)

Step (a): In a round bottom flask, 5-methoxy-4-azaindole (SM-IV-4a, 103 mg, 0.695 mmol) and 4-dimethylaminopyridine (DMAP, 8.49 mg, 0.070 mmol) were dissolved in CH₃CN (2 mL) and boc-anhydride (182 mg, 0.834 mmol) was added. The reaction was stirred for overnight at rt and evaporated to celite. CombiFlash purification (normal silica phase) gave 110 mg of the SM-IV-4b.

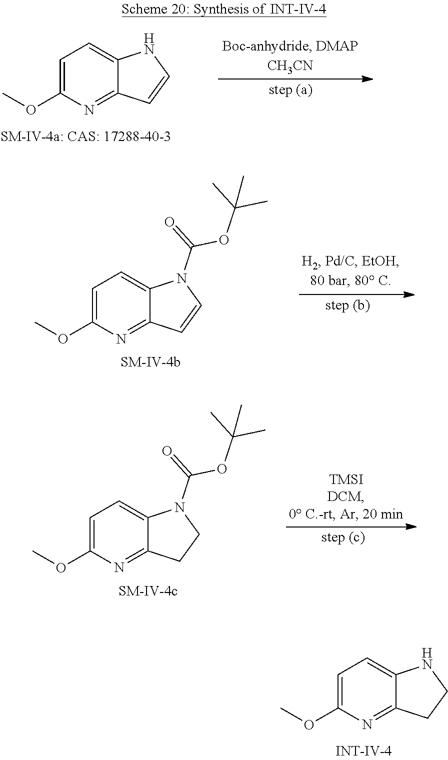 [SMILES: COc1ccc2[nH]ccc2n1 ; COc1ccc2c(ccn2C(=O)OC(C)(C)C)n1 ; COc1ccc2c(n1)CCN2C(=O)OC(C)(C)C ; COc1ccc2c(n1)CCN2]

Step (b): In the hydrogenation flask, tert-butyl 5-methoxy-1H-pyrrolo[3,2-b]pyridine-1-carboxylate (SM-IV-4b, 110 mg, 0.443 mmol) was dissolved in EtOH (11 mL) and hydrogenated by H-Cube flow instrument (10% Pd/C cartridge; flow 1 mL/min, 80 bar, 80° C.) and evaporated to dryness to obtain 98 mg of the crude product with 20% of the reduced product. The crude product was dissolved in EtOAc and hydrogenation repeated (flow 1 mL/min; 100 C, 80 bar; new 10% Pd/C cartidge) and evaporated to dryness to obtain 80 mg of the SM-IV-4c. ¹H-NMR (400 MHz, DMSO-d₆): δ 7.99 (br d, 1H), 7.58 (br s, 1H), 6.51 (br d, 1H), 4.05-3.92 (m, 2H), 3.89 (s, 3H), 3.14 (t, 2H), 1.54 (br s, 9H).

Step (c): In a 10 mL pressure tube fitted with a stirrer was charged with tert-butyl 5-methoxy-2,3-dihydro-1H-pyrrolo[3,2-b]pyridine-1-carboxylate (SM-IV-4a, 50 mg, 0.2 mmol) in anhyd. DCM (2 mL) under argon. The tube was cooled to 0° C. before the dropwise addition of a solution of trimethylsilyl iodide (120 mg, 85 μL, 0.60 mmol, 3 equiv.) in 0.5 mL anhyd. DCM under argon. The reaction was completed within 20 min. Then, MeOH (2 mL) was then added to the reaction suspension and the formed solution was stirred for 5 min. Solvent was removed in vacuo and H₂O was added to the residue and the pH was adjusted to ca. pH 2 with 1 N HCl. The solution was extracted once with EtOAc and the organic layer was washed many times with water and 1 N HCl until complete transfer of the product from the organic layer to the aqueous layer. The combined aqueous layers were basified to ca. pH 9 with 1 N NaOH. The formed precipitate was filtered off, washed with water and dried to give INT-IV-4 (yield: 22 mg, 73%) that used for the next step without any further purification.

### Preparation of 2,2,2-trifluoro-1-(indolin-4-yl)ethan-1-ol (INT-IV-5)

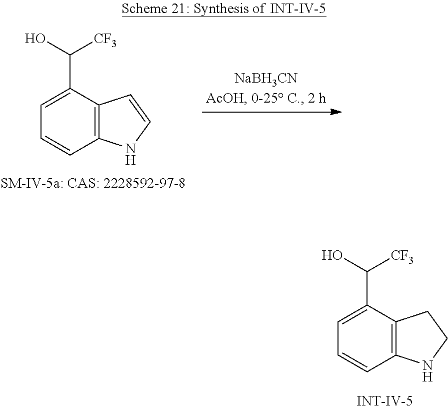 [SMILES: OC(c1cccc2[nH]ccc12)C(F)(F)F ; OC(c1cccc2c1CCN2)C(F)(F)F]

To a solution of 2,2,2-trifluoro-1-(1H-indol-4-yl)ethan-1-ol (SM-IV-5a, 500 mg, 2.32 mmol) in AcOH (10 mL) was added NaBH₃CN (219 mg, 3.49 mmol) at 0° C. The resulting reaction mixture was stirred at 25° C. for 2 h. After completion of the reaction as indicated by TLC, the reaction mixture was quenched with aq. NaHCO₃ (200 mL) and extracted with EtOAc (3×150 mL). The combined organic layer was dried over Na₂SO₄ and concentrated under reduced pressure. This crude residue was purified by combi-flash chromatography using (EtOAc: heptane, 50:50) as an eluent to afford 2,2,2-trifluoro-1-(indolin-4-yl)ethan-1-ol (INT-IV-5). R_{f}(EtOAc: heptane, 50:50)=0.25; yield: 130 mg, 26%. MS: m/z 218.04 [M+H]⁺.

### Preparation of 4-(Pyridin-3-yl)Indoline (INT-IV-6)

Step (a): Treatment of tert-butyl 4-bromoindoline-1-carboxylate (SM-IV-6a, 1.6 g, 5.38 mmol) with pyridin-3-ylboronic acid (0.80 g, 6.46 mmol) in the presence of Na₂CO₃ (1.71 g, 16.14 mmol), Pd(PPh₃)₄ (0.62 mg, 0.54 mmol) in EtOH: H₂O (3:1, 40 mL) at 90° C. for 16 h followed by work-up and purification to afford tert-butyl 4-(pyridin-3-yl)indoline-1-carboxylate (SM-IV-6b) as colourless liquid. R_{f}(EtOAc: heptane, 20:80)=0.1; yield: 1.5 g, 94%. MS: m/z 297.24 [M+H]⁺.

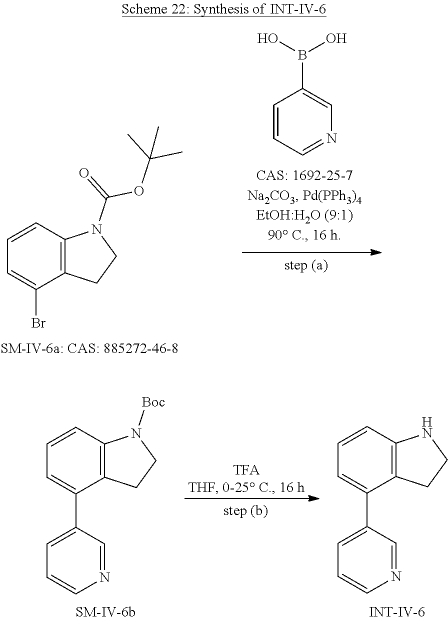 [SMILES: CC(C)(C)OC(=O)N1CCc2c(Br)cccc21 ; OB(O)c1cccnc1 ; CC(C)(C)OC(=O)N1CCc2c(-c3cccnc3)cccc21 ; c1cncc(-c2cccc3c2CCN3)c1]

Step (b): Treatment of tert-butyl 4-(pyridin-3-yl)indoline-1-carboxylate (SM-IV-6b), 1 g, 3.37 mmol) with TFA (3.84 g, 3.37 mL, 4.0 M solution, 33.7 mmol) in THF (10 mL) at 25° C. for 12 h followed by basic extraction to afford crude product. This crude residue was purified by trituration in Et₂O to afford 4-(pyridin-3-yl)indoline (INT-IV-6) as a white solid. R_{f}(EtOAc: heptane, 80:20)=0.1; yield: 0.6 g, 91%. MS: m/z 197.10 [M+H]⁺.

### Preparation of 1-(indolin-4-yl)ethan-1-one hydrogen chloride (INT-IV-7)

Step (a): Treatment of 1-(tert-butoxycarbonyl)indoline-4-carboxylic acid (SM-IV-7a, 15.5 g, 58.9 mmol) with N,O-dimethylhydroxylamine hydrochloride (5.39 g, 88.3 mmol) in the presence of HATU (33.6 g, 88.3 mmol) and DIPEA (31 mL, 177 mmol) in DMF (90 mL) at 25° C. for 16 h followed by work-up and purification by column chromatography afforded tert-butyl 4-(methoxy(methyl)-carbamoyl)indoline-1-carboxylate (SM-IV-7b) as a white solid. R_{f}(EtOAc: heptane, 30:70)=0.4; yield: 16.1 g, 89%. MS: m/z 307.26 [M+H]⁺.

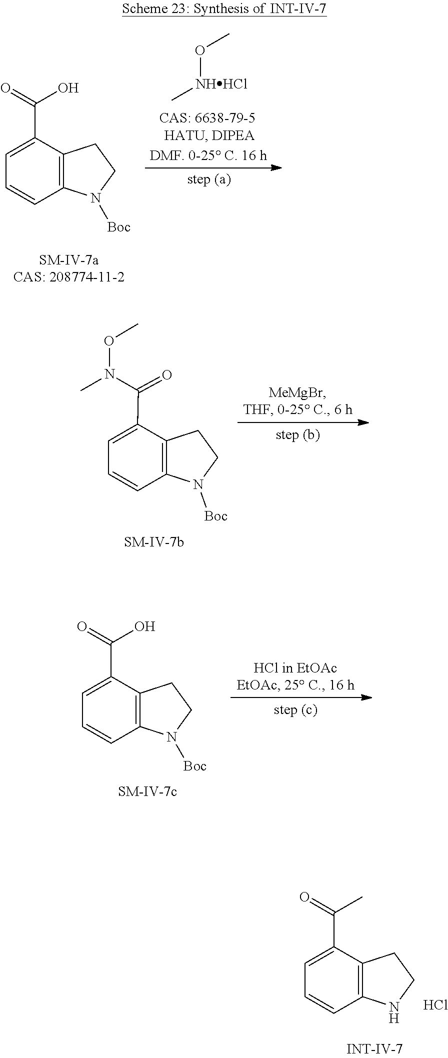

Step (b): Treatment of tert-butyl 4-(methoxy(methyl)carbamoyl)indoline-1-carboxylate (SM-IV-7b, 10 g, 32.68 mmol) with added MeMgBr (0.1 L, 3M, 326.8 mmol) in THF (150 mL) at 25° C. for 2 h followed by work-up and recrystallization with EtOH afforded tert-butyl 4-acetylindoline-1-carboxylate (SM-IV-7c) as an off white solid. R_{f}(EtOAc: heptane, 30:70)=0.7; yield: 6.2 g, 73%. MS: m/z 162.14 [M-100+H]⁺. ¹H-NMR (400 MHz; DMSO-d₆): δ 7.92 (s, 1H), 7.56 (d, 1H), 7.33 (t, 1H), 3.91 (t, 2H), 3.32 (t, 2H), 2.55 (s, 3H), 1.51 (s, 9H). Step (c): Treatment of tert-butyl 4-acetylindoline-1-carboxylate (SM-IV-7c, 4.0 g, 15.3 mmol) with HCl in EtOAc (19.1 mL, 4.0 M, 76.65 mmol) in EtOAc (30 mL) for 12 h followed by filtration of the product INT-IV-7 as an off white solid. R_{f}(EtOAc: heptane, 30:70)=0.2; crude yield: 2.2 g, 73%. MS: m/z 162.15 [M+H]⁺.

### Preparation of 4-(N-methylpiperazin-1-ylcarbonyl)indoline (INT-IV-9)

Step (a): Indole-4-carboxylic acid (SM-IV-9a, 1.2 mmol, 0.2 g), N-methylpiperazine (0.14 g, 0.15 mL, 1.1 equiv.), HATU (0.59 g, 1.25 equiv.), BtOH·H₂O (0.21 g, 1.25 equiv.) and DIPEA (0.24 mL, 0.18 g, 1.1 equiv.) were added to 15 mL of DMF, and the mixture was stirred at 60° C. for overnight. The solvent was then evaporated to dryness, and the residue was extracted with EtOAc from the sat. soln. of NaHCO₃. The product was purified by column chromatography to afford SM-IV-9b (yield: 0.21 g, 70%). LC-MS (m/z): 244.0 [M+H]⁺; purity: 96.2%.

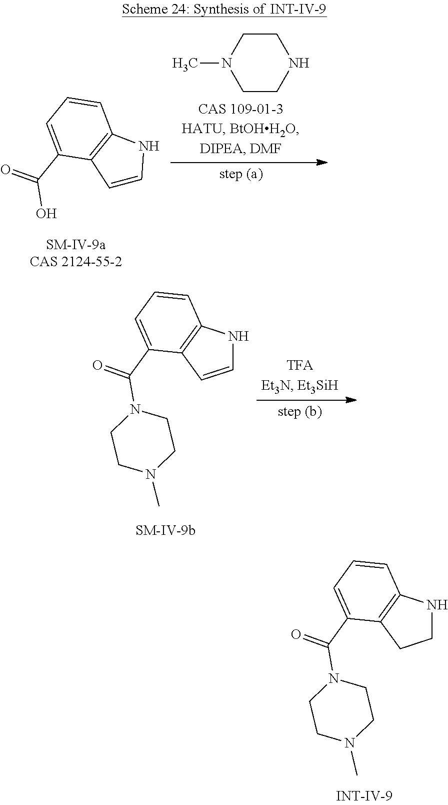

Step (b): The indole SM-IV-9b (0.3 g, 1.2 mmol) was dissolved in 5 mL of TFA and was cooled to 0° C. Then, Et₃SiH (2 equiv.) was added, and the reaction mixture was stirred at rt for overnight. Then, sat. soln. of NaHCO₃ was carefully added, and the product was extracted with DCM (3×50 mL). Evaporation of the solvent afforded the crude product INT-IV-9 (yield: 0.27 g, 90%) which was used for the next reaction without further purification. LC-MS (m/z): 245.9 [M+H]⁺; purity: 83.0%.

### Synthesis of INT-IV-12, INT-IV-13, INT-IV-15, and INT-IV-16

Step (a): In a round flask, 1-(4-hydroxyindolin-1-yl)ethan-1-one (SM-IV-12a, 1 equiv.), K₂CO₃ (1.6 equiv.) and the corresponding alkyl chloride (R—Cl, Scheme 25, 1.4 equiv.) were mixed together in anhyd. DMF (2-10 mL) and heated at 100° C. for 18 h. After cooling down to rt, water was added to the reaction mixture and the mixture was extracted with EtOAc (3×50 mL). The combined organic layers were dried over MgSO₄, concentrated under reduced pressure to afford the corresponding N-acetylated 4-O-substituted indolinederivatives SM-IV-12b, SM-IV-13b, SM-IV-15b, and SM-IV-16b as crude products that were used for next step without further purification.

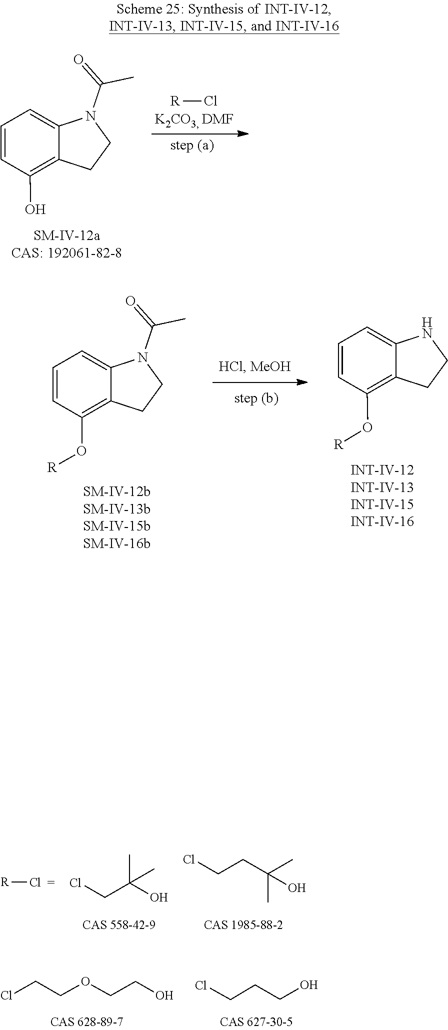

Step (b): N-Acetylated 4-O-substituted indoline derivatives SM-IV-12b, SM-IV-13b, SM-IV-15b, and SM-IV-16b were dissolved in MeOH and conc. HCl (1.25 mL for every 1 mmol) was then added. The reaction mixture was heated under reflux for 6 h. The solvent was removed under reduced pressure. Water was then added and the aq. layer was neutralized with sat. soln. of NaHCO₃. The aqueous layer was further extracted with EtOAc (3×50 mL). The combined organic layers were dried over MgSO₄, filtered off, and the solvent was removed in vacuo to afford the desired 4-O-substituted indoline derivatives INT-IV-12, INT-IV-13, INT-IV-15, and INT-IV-16.

### Preparation of 2-(indolin-4-yl)isothiazolidin-3-one 1,1-dioxide (INT-IV-18)

Step (a): A mixture of tert-butyl 4-aminoindoline-1-carboxylate (SM-IV-18a, 180 mg, 0.77 mmol) and methyl 3-(chlorosulfonyl)propanoate (144 mg, 0.77 mmol) in pyridine (1 mL) was heated at 80° C. for 4 h. The reaction mixture was quenched with water and extracted with DCM. The organic layer was dried anhyd. MgSO₄, filtered and concentrated under reduced pressure to give tert-butyl 4-((3-methoxy-3-oxopropyl)sulfonamido)indoline-1-carboxylate (SM-IV-18b) as a beige solid (238 mg crude). This compound was used in the next step without further purification.

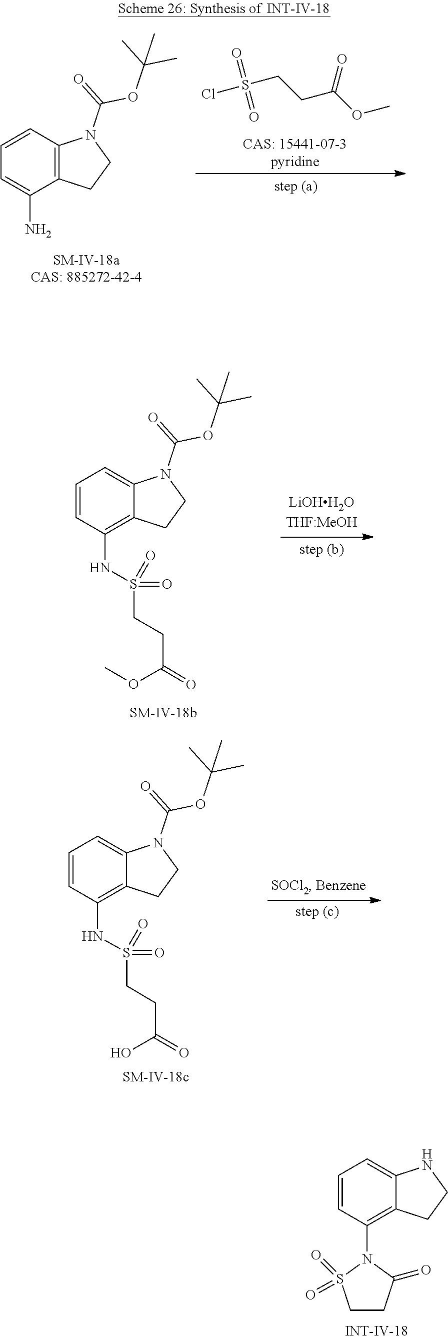

Step (b): A mixture of tert-butyl 4-((3-methoxy-3-oxopropyl)sulfonamido)indoline-1-carboxylate (SM-IV-18b, 238 mg, 0.62 mmol) and LiOH. H₂O (150 mg, 3.6 mmol) in MeOH (5 mL) and THF (5 mL) was stirred at rt for 16 h. The reaction mixture was quenched with water. The aq. phase was washed with Et₂O, acidified with diluted HCl and subsequently extracted with Et₂O. The organic layer was dried anhyd. MgSO₄, filtered and concentrated under reduced pressure. The residue was crystallized from (Et₂O: PE) to give 3-(N-(1-(tert-butoxycarbonyl)indolin-4-yl)sulfamoyl)propanoic acid (SM-IV-18c, 110 mg of crude). This compound was used in the next step without further purification.

Step (c): To a mixture of crude 3-(N-(1-(tert-butoxycarbonyl)indolin-4-yl)sulfamoyl)propanoic acid (SM-IV-18c, 250 mg, 0.675 mmol) in benzene (10 mL) was added SOCl₂ (1 mL, 13.8 mmol). The mixture was heated at 80° C. for 3 h. Subsequently, the mixture was evaporated under reduced pressure. The residue was diluted with DCM (75 mL) and washed with diluted NaOH solution. The organic layer was dried anhyd. MgSO₄, filtered and concentrated under reduced pressure to give 2-(indolin-4-yl)isothiazolidin-3-one 1,1-dioxide (INT-IV-18) as a colourless solid (80 mg crude). This compound was characterized only by TLC/MS: m/z 252 [M+H]⁺, and was used in the next step without further purification.

### Preparation of 4-(indolin-4-yl)-2,6-dimethylmorpholine hydrochloride (INT-IV-19)

Step (a): To a solution of tert-butyl 4-bromoindoline-1-carboxylate (SM-IV-6a, 0.5 g, 1.67 mmol) and 2,6-dimethylmorpholine (0.4 mL, 3.35 mmol) in anhyd. DMSO (10 mL) was added Pd(OAc)₂ (0.02 g, 0.08 mmol), BINAP (0.104 g, 0.16 mmol) and Cs₂CO₃ (1.64 g, 5.03 mmol) and the reaction mixture was heated at 100° C. for 4 h. Upon completion of the reaction, it was poured into ice H₂O to get solids. The precipitated solids were filtered and purified by flash column chromatography using to get 0.27 g of tert-butyl 4-(2,6-dimethylmorpholino)indoline-1-carboxylate (SM-IV-19a) as a gummy solid. MS: m/z 333.4 [M+H]⁺.

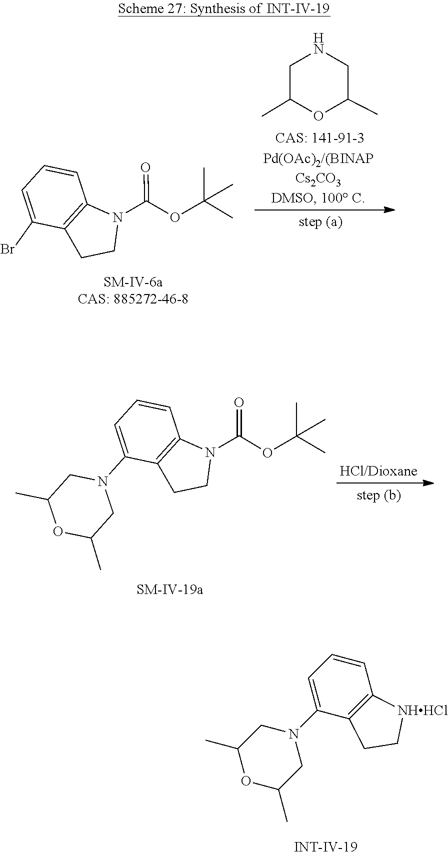

Step (b): To a solution of tert-butyl 4-(2,6-dimethylmorpholino)indoline-1-carboxylate (SM-IV-19a, 0.27 g, 8.12 mmol) in anhyd. 1,4 Dioxane (2 mL) was added HCl in dioxane (5 mL, 4.0 M) and stirred for 4 h. Upon completion of the reaction, it was concentrated under reduced pressure. The crude material was washed with EtOAc to get 0.2 g of 4-(indolin-4-yl)-2,6-dimethylmorpholine hydrochloride (INT-IV-19) as a yellow solid.

### Preparation of 8-(indolin-4-yl)-8-azabicyclo[3.2.1]octan-3-ol (INT-IV-20)

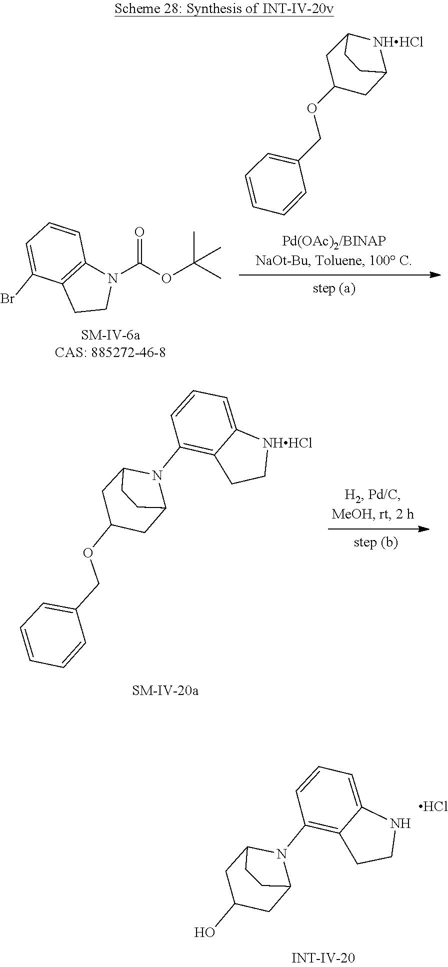

Step (a): To a solution of tert-butyl 4-bromoindoline-1-carboxylate (SM-IV-6a, 500 mg, 1.67 mmol) and 3-(benzyloxy)-8-azabicyclo[3.2.1]octane (402 mg, 1.85 mmol) in anhyd. toulene (7 mL) was added Pd(OAc)₂ (37 mg, 0.17 mmol), BINAP (105 mg, 0.17 mmol) and sodium tert-butoxide (1.1 g, 11.7 mmol) and the reaction mixture was heated at 100° C. for 5 h. Upon completion of the reaction, the reaction mixture was quenched with H₂O and extracted with EtOAc (3×25 mL). The combined organic layer was washed with brine, dried over anhyd. Na₂SO₄ and concentrated under reduced pressure. This crude residue was purified by flash column chromatography using to get 0.49 g of SM-IV-20a that was used directly for the next step.

Step (b): In a 10 mL hydrogenation flask, a mixture of 4-(3-(benzyloxy)-8-azabicyclo[3.2.1]octan-8-yl)indoline hydrochloride (INT-IV-20a, 50 mg, 0.135 mmol) and palladium catalyst (10% Pd/C, 14.3 mg, 0.0135 mmol, 0.1 equiv.) in MeOH (2 mL). The reaction flask was connected to a hydrogen gas generator and was stirred at rt under a pressure of 50 psi for 2 h. The reaction mixture was filtered through a pad of celite, washed with MeOH, and the filtrate was dried in vacuo giving INT-IV-20 (yield: 45.4 mg, 77.8%).

### Preparation of 4-(1H-Imidazol-1-yl)indoline (INT-IV-21)

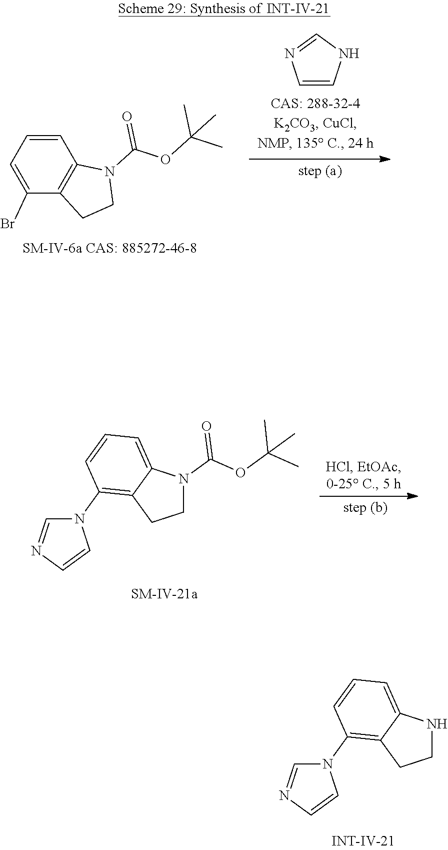 [SMILES: c1c[nH]cn1 ; CC(C)(C)OC(=O)N1CCc2c(Br)cccc21 ; CC(C)(C)OC(=O)N1CCc2c1cccc2-n1ccnc1 ; c1cc2c(c(-n3ccnc3)c1)CCN2]

Step (a): To a solution of tert-butyl 4-bromoindoline-1-carboxylate (SM-IV-6a, 100 mg, 0.34 mmol) in NMP (5 mL), were added 1H-imidazole (35 mg, 0.51 mmol), K₂CO₃ (235 mg, 1.7 mmol) at 25° C. This reaction mixture was degassed with argon for 15 min. Subsequently CuCl (7 mg, 0.051 mmol) was added to the reaction mixture and the reaction mixture was again degassed with argon for 5 min. The resulting reaction mixture was heated at 135° C. for 24 h. After completion of the reaction as indicated by TLC, the reaction mixture was quenched with water and extracted with EtOAc (3×25 mL). The combined organic layer was washed with brine, dried over anhyd. Na₂SO₄ and concentrated under reduced pressure. This crude residue was purified by combi-flash chromatography using 0-20% EtOAc in heptane as an eluent to afford tert-butyl 4-(1H-imidazol-1-yl)indoline-1-carboxylate (SM-IV-21a) as a yellow semi solid. R_{f} (EtOAc: heptane, 20:80)=0.1; yield: 90 mg, 93%. MS: m/z 286.20 [M+H]⁺.

Step(b): Treatment of tert-butyl 4-(1H-imidazol-1-yl)indoline-1-carboxylate (SM-IV-21a, 200 mg, 0.70 mmol) with HCl in EtOAc (3.5 mL, 4.0 M, 14 mmol) in EtOAc (3 mL) at 25° C. for 5 h followed by basic extraction afforded 4-(1H-Imidazol-1-yl)indoline (INT-IV-21). This crude residue was forwarded to the next step without further purification. R_{f}(EtOAc: heptane, 95:05)=0.1; crude yield: 110 mg, 85%. MS: m/z 186.08 [M+H]⁺.

### Preparation of 1-(indolin-4-yl)pyrrolidine-2,5-dione (INT-IV-22)

Step (a): To a solution af 4-amino-2,3-dihydro-indole-1-carboxylic acid tert-butyl ester (SM-IV-18a, 100 mg, 0.43 mmol) in DCM (5 mL), Et₃N (118 μL, 0.85 mmol) was added, followed by 4-DMAP (6.25 mg, 0.05 mmol). Succinyl chloride (70.5 μL, 0.64 mmol) was then added at 0° C. After 1 h at 0° C., the reaction mixture was stirred for 18 h at rt. Water was then added and the aqueous layer was extracted with EtOAc (3×50 mL). The combined organic layers were dried over MgSO₄, filtered off, and the solvent was removed under vacuum to afford indoline SM-IV-22a (yield: 100 mg, 74%) that used for the next step without further purifications.

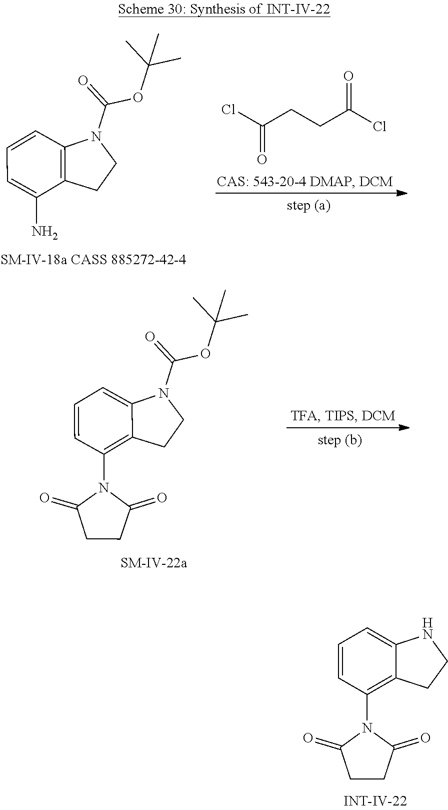 [SMILES: CC(C)(C)OC(=O)N1CCc2c(N)cccc21 ; O=C(Cl)CCC(=O)Cl ; CC(C)(C)OC(=O)N1CCc2c1cccc2N1C(=O)CCC1=O ; O=C1CCC(=O)N1c1cccc2c1CCN2]

Step (b): To a solution of 4-(2,5-dioxopyrrolidin-1-yl)indoline-1-carboxylate (SM-IV-22a, 100 mg, 0.16 mmol, 1 equiv.) in DCM (2 mL) were added subsequently 0.10 mL of TIPS (2.5%) and 2 mL of TFA (20%). After that, the reaction mixture was stirred at rt for 2 h, then the volatiles were removed under reduced pressure then water was added. The water phase was then neutralized with sat. soln. of NaHCO₃, and the product was extracted with EtOAc (3×50 mL). The combined organic layers were dried over MgSO₄, filtered off, and the solvent was removed under vacuum to afford 1-(indolin-4-yl)pyrrolidine-2,5-dione INT-IV-22 (yield: 47 mg, 69%) that was used for the next step without further purifications.

### Preparation of 1-(Indolin-4-yl)Pyrrolidin-3-Ol (INT-IV-23)

Step (a): Treatment of tert-butyl 4-bromoindoline-1-carboxylate (SM-IV-6a, 1.0 g, 3.36 mmol) with pyrrolidin-3-ol (1.3 g, 4.37 mmol) in the presence of NaOtBu (1.6 g, 16.8 mmol), palladium(II) acetate (75 mg, 0.34 mmol) and BINAP (208 mg, 0.34 mmol) in toluene (10 mL) at 100° C. for 2 h followed by work-up and purification afforded tert-butyl 4-(3-hydroxypyrrolidin-1-yl)indoline-1-carboxylate (SM-IV-23a) as an off white solid. R_{f}(EtOAc: heptane: 40:60)=0.3; yield: 500 mg, 49%. MS: m/z 305.20 [M+H]⁺.

Step (b): Treatment of tert-butyl 4-(3-hydroxypyrrolidin-1-yl)indoline-1-carboxylate (SM-IV-23a, 0.4 g, 1.32 mmol) with TFA (1.0 mL, 13.2 mmol) in DCM (5 mL) at 25° C. for 4 h followed by basic work-up afforded 1-(indolin-4-yl)pyrrolidin-3-ol (INT-IV-23) as an off white solid. R_{f}(EtOAc: heptane, 60 :40)=0.2; yield: 150 mg, 56%. MS: m/z 205.09 [M+H]⁺.

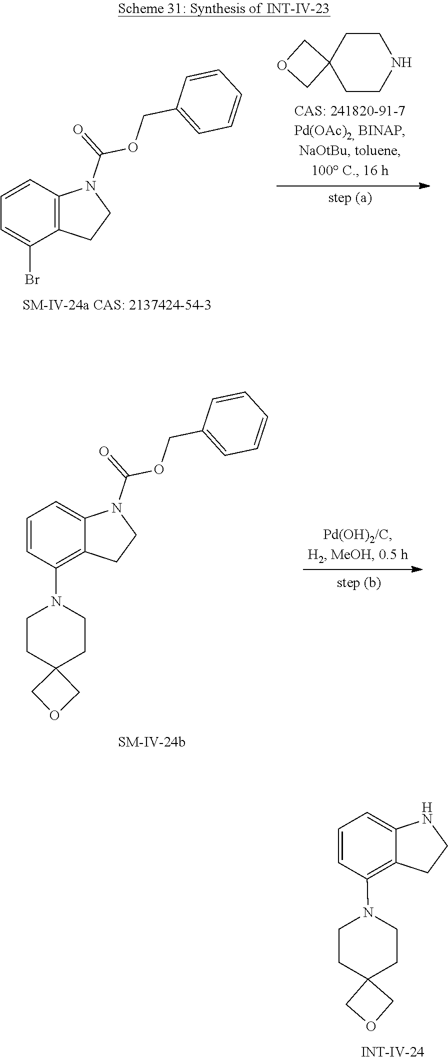 [SMILES: O=C(OCc1ccccc1)N1CCc2c(Br)cccc21 ; C1CC2(CCN1)COC2 ; O=C(OCc1ccccc1)N1CCc2c(N3CCC4(CC3)COC4)cccc21 ; c1cc2c(c(N3CCC4(CC3)COC4)c1)CCN2]

### Preparation of 7-(indolin-4-yl)-2-oxa-7-azaspiro[3.5]nonane (INT-IV-24)

Step (a): Treatment of benzyl 4-(4-(tert-butoxycarbonyl)piperazin-1-yl)indoline-1-carboxylate (SM-IV-24a, 2.0 g, 6.02 mmol) with 2-oxa-7-azaspiro[3.5]nonane (0.76 g, 6.02 mmol) in the presence of NaOtBu (1.44 g, 15.1 mmol), palladium(II) acetate (134 mg, 0.60 mmol) and BINAP (373 mg, 0.60 mmol) in toluene (40 mL) at 100° C. for 16 h followed by work-up and purification afforded benzyl 4-(2-oxa-7-azaspiro[3.5]nonan-7-yl)indoline-1-carboxylate (SM-IV-24b) as white solid. R_{f} (EtOAc heptane, 30:70)=0.1; Yield: 500 mg, 23%. MS: m/z 379.01. [M+H]⁺.

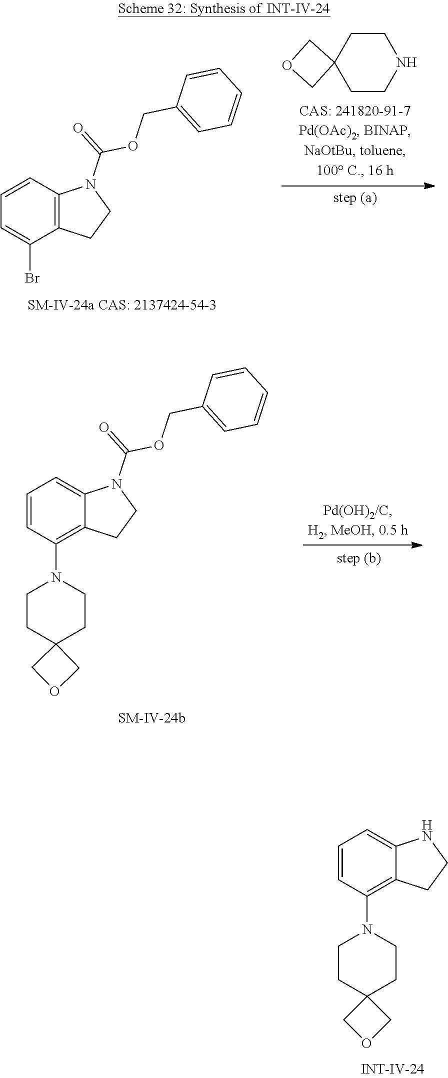 [SMILES: O=C(OCc1ccccc1)N1CCc2c(Br)cccc21 ; C1CC2(CCN1)COC2 ; O=C(OCc1ccccc1)N1CCc2c(N3CCC4(CC3)COC4)cccc21 ; c1cc2c(c(N3CCC4(CC3)COC4)c1)CCN2]

Step (b): To a solution of benzyl 4-(2-oxa-7-azaspiro[3.5]nonan-7-yl)indoline-1-carboxylate (SM-IV-24b, 0.5 g, 1.37 mmol) in MeOH (20 mL), was added 10% Pd(OH)₂/C (0.57 g, 0.20 mmol) at 25° C. in N₂atmosphere. The resulting reaction mixture was stirred at 25° C. under 60 psi H₂ gas pressure at parr-shaker for 30 min. After completion of the reaction as indicated by TLC, the reaction mixture was filtered through celite and washed with MeOH. Filtrate was concentrated under reduced pressure to get crude. This crude residue INT-IV-24 was forwarded to the next step without further purification. R_{f} (EtOAc: heptane, 50:50)=0.4; crude yield: 0.24 g, 72%. MS: m/z 245.12 [M+H]⁺.

### Preparation of 1-(Indolin-4-yl)Imidazolidine-2,4-Dione (INT-IV-25)

Step (a): To 2-chloroacetyl isocyanate (280 mg, 2.35 mmol) was added a solution of tert-butyl 4-aminoindoline-1-carboxylate (SM-IV-18a, 400 mg, 1.7 mmol) in DCM (7 mL) at rt under stirring. After 30 min of stirring at rt, no further starting material could be detected by TLC (EtOAc: DCM, 5:95). The mixture was diluted with additional DCM (50 mL) and washed with sat. NaHCO₃ soln. (25 mL). The organic layer was dried anhydr. MgSO₄, filtered and concentrated under reduced pressure. Cystallization from ether gave 580 mg tert-butyl 4-(3-(2-chloroacetyl)ureido)indoline-1-carboxylate (SM-IV-25a) as yellowish crystals. This compound was used in the next step without further purification.

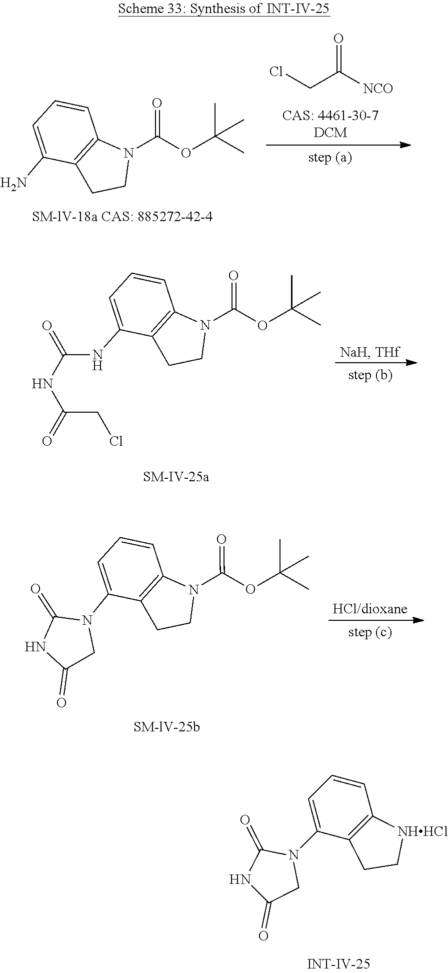

Step (b): To a solution of tert-butyl 4-(3-(2-chloroacetyl)ureido)indoline-1-carboxylate (SM-IV-25a, 211 mg, 0.60 mmol) in THF (40 mL) was added NaH (250 mg, 6.3 mmol, 60% in paraffin). After stirring at rt for 0.5 h, no further starting material could be detected by TCL. The mixture was concentrated under reduced pressure. The resulting residue was dissolved in water (150 mL) and washed with DCM (30 mL). The aqueous phase was acidified with diluted HCl until a pH of 6 was reached. Subsequently the aqueous phase was extracted with DCM. The organic layer was dried anhyd. MgSO₄, filtered and concentrated under reduces pressure. Crystallization of the residue thus abtained from petroleum ether, led to 160 mg of tert-butyl 4-(2,4-dioxoimidazolidin-1-yl)indoline-1-carboxylate (SM-IV-25b) as beige crystals. This was used in the next step without further purification.

Step (c): To a solution of HCl indioxane (4 N, 10 mL) was added tert-butyl 4-(2,4-dioxoimidazolidin-1-yl)indoline-1-carboxylate (SM-IV-25b). The reaction was controlled by TLC (MeOH: DCM, 1:99). The mixture was stirred at rt for 2 h and subsequently concentrated under reduced pressure. The resulting residue was treated with PE and the unsoluble material was filtered under reduced pressure leading to 65 mg of 1-(indolin-4-yl)imidazolidine-2,4-dione hydrochloride (INT-IV-25) as a beige solid. This compound was used in the next step without further purification.

### Preparation of 1-(indolin-4-ylamino)-2-methylpropan-2-ol hydrogen chloride (INT-IV-26)

Step (a): Treatment of tert-butyl 4-bromoindoline-1-carboxylate (SM-IV-6a, 200 mg, 0.67 mmol) with 1-amino-2-methylpropan-2-ol (60 mg, 0.67 mmol) in the presence of NaOtBu (198 mg, 2.02 mmol), palladium(II) acetate (15 mg, 0.067 mmol) and BINAP (42 mg, 0.07 mmol) in toluene (8 mL) at 100° C. for 16 h followed by extraction and purification afforded tert-butyl 4-((2-hydroxy-2-methylpropyl)amino)indoline-1-carboxylate (SM-IV-26a) as a yellow solid. R_{f}(EtOAc: heptane, 30:70)=0.3; yield: 200 mg, 98%. MS: m/z 207.06 [M−100+H]⁺.

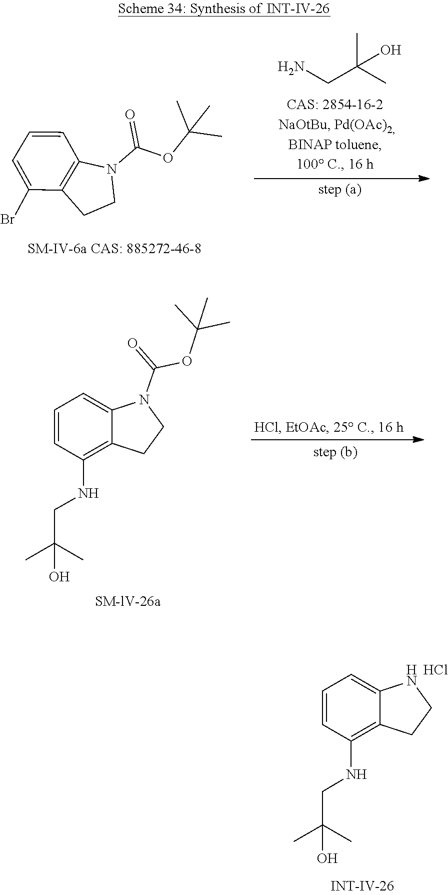

Step (b): Treatment of tert-butyl 4-((2-hydroxy-2-methylpropyl)amino)indoline-1-carboxylate (SM-IV-26a, 200 mg, 0.65 mmol) with HCl in EtOAc (5.0 mL, 4.0 M, 19.5 mmol) in EtOAc (5 mL) at 25° C. for 12 h followed by trituration with EtOAc afforded tert-butyl 4-((2-hydroxy-2-methylpropyl)amino)indoline-1-carboxylate (INT-IV-26) as a yellow solid. R_{f}(EtOAc: heptane: 95 05)=0.1; yield: 130 mg; 83%. MS: m/z 207.06 [M+H]⁺.

### Preparation of 4-aminosulfonylindoline (INT-IV-27)

Step (a): In a round flask, 4-bromoindole (SM-IV-27a, 10 mmol, 2 g, 1 equiv.) was dissolved in 20 mL of anhyd. THF and 20 mL anhyd. Et₂O. The solution was cooled to 0° C., and NaH (0.43 g of 60% dispersion in mineral oil, 1.06 equiv.) was carefully added. The mixture was stirred at 0° C. for 15 min, then cooled to −80° C., and t-BuLi (2 equiv., 10.7 mL of 1.9 M soln. in pentane) was slowly added, so that the temperature of the reaction mixture was below −75° C. After 30 min, SO₂ gas was bubbled through the formed suspension over 1 h. The reaction mixture was then left to warm up to rt for overnight, then cooled to 0° C., and AcOH (1.1 equiv., 0.58 g) was added. After stirring for 30 min., 20 mL of Et₂O was added, and the precipitate was filtered off and washed with Et₂O. The solid residue was suspended in 20 mL of Et₂O, cooled to 0° C., and N-chlorosuccinimide (1 equiv., 1.4 g) was carefully added. The mixture was stirred 1.5 h at 0° C., then the solid was filtered off, washed with Et₂O, evaporated to dryness to obtain (yield: 1 g, 45%) of SM-IV-27b. LC-MS (m/z): 211.9 [M+H]⁺; purity: 96.4%.

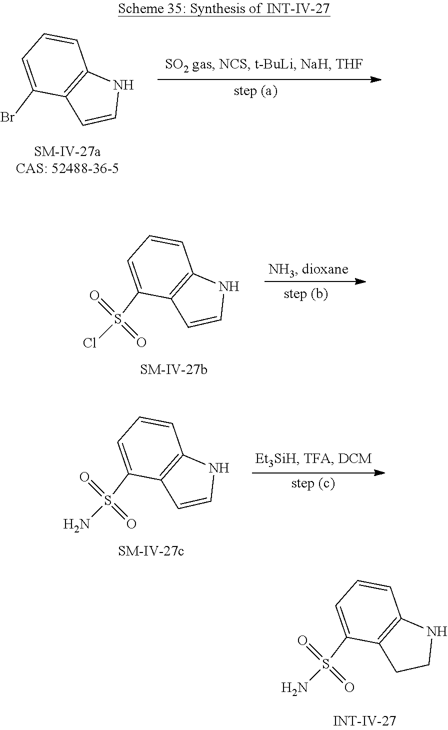 [SMILES: Brc1cccc2[nH]ccc12 ; O=S(=O)(Cl)c1cccc2[nH]ccc12 ; NS(=O)(=O)c1cccc2[nH]ccc12 ; NS(=O)(=O)c1cccc2c1CCN2]

Step (b): Indole-4-sulfonyl chloride (SM-IV-27b, 0.52 mmol, 0.11 g) in 5 mL of dry dioxane was slowly added to the 0.5 M solution of ammonia in dioxane (5.2 mL, 5 equiv.) at 0° C. The mixture was stirred for 1 h at rt, and the solvent was evaporated to dryness to provide the product SM-IV-27c that was used for the next reaction without further purification (90 mg, 90% yield). ¹H NMR (500 MHz, DMSO-d₆): δ 11.49 (s, 1H), 7.62 (dt, 1H), 7.54-7.47 (m, 2H), 7.25-7.14 (m, 3H), 6.65-7.05 (m, 1H). LC-MS (m/z): 197.0 [M+H]⁺; purity: 91.1%.

Step (c): The obtained 4-aminosulfonylindole (SM-IV-27c, 150 mg, 0.76 mmol) was added to 10 mL of DCM and cooled to 0° C. Then TFA (1 mL) and Et₃SiH (2 equiv.) were added, and the reaction mixture was stirred at rt for overnight. The reaction mixture was basified by NaOH 50% solution to pH-14, impurities washed with DCM, the water phase was then neutralized, and the product was extracted with EtOAc (3×50 mL) to afford INT-IV-27 (yield: 76 mg, 50%).

### Preparation of 4-methylaminosulfonylindoline (INT-IV-28)

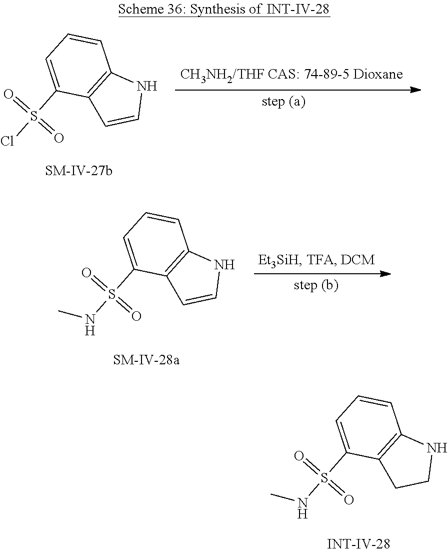

Step (a): Indole-4-sulfonyl chloride (SM-IV-27b, 70 mg, 0.32 mmol, 1 equiv.) in 5 mL of dry dioxane was slowly added to the 2 M solution of methylamine in THF (1.6 mL, 10 equiv.) at 0° C. The mixture was stirred for 1 h at rt, and the solvent was evaporated under reduced pressure to provide the product SM-IV-28a that was used for the next reaction without further purification (yield: 64 mg, 94%). LC-MS (m/z): 211.0 [M+H]⁺; purity: 95.2%.

Step (b): This step was described before (see step (c) for INT-IV-27) from 4-methylaminosulfonylindoline (SM-IV-28a, 68 mg, 0.32 mmol) and the obtained product INT-IV-28 (yield: 64 mg, 93%). LC-MS (m/z): 212.8 [M+H]⁺; purity: 62.3%.

### Preparation of 4-Methylsulfonylaminoindole (INT-IV-29)

Step (a): In a round bottom flask, 4-aminoindole (SM-IV-29a, 150 mg, 1.1 mmol) and Et₃N (0.19 mL, 140 mg, 1.2 equiv.) were dissolved in 10 mL of DCM and cooled to 0° C. Methanesulfonyl chloride (0.09 mL, 140 mg, 1.05 equiv.) was slowly added, and the mixture was stirred for an additional 1 h. The solvent was evaporated to dryness, the residue was dissolved in 1 N NaOH solution, washed with EtOAc, acidified to pH-5 with 1 N HCl soln., and the product was extracted with EtOAc (3×50 mL) to obtain SM-IV-29b (yield: 0.24 g, 99%). LC-MS (m/z): 211.0 [M+H]⁺.

Step (b): The step was previously described (see step (c) for INT-IV-27) from 4-methylsulfonylaminoindole (SM-IV-29b, 140 mg, 0.66 mmol) to afford INT-IV-29 (yield: 0.13 g, 93%). LC-MS (m/z): 212.9 [M+H]⁺; purity: 65.5%.

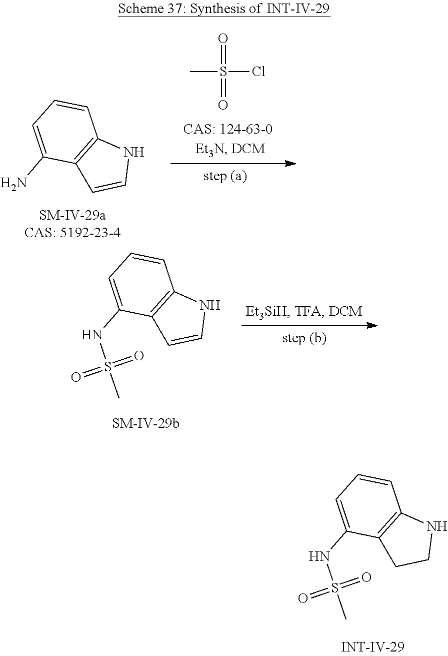

### Preparation of 2,2,2-trifluoro-1-(4-(3-hydroxyoxetan-3-yl)-114-indolin-1-yl)ethan-1-one (INT-IV-30)

Step (a): To a stirred solution of tert-butyl 4-bromoindoline-1-carboxylate (SM-IV-6a, 500 mg, 1.68 mmol) in dry THF (25 mL) was added n-BuLi (1.4 mL, 1.4 M, 2.01 mmol) at −78° C. dropwise over the period of 5 min then the reaction mixture was stirred for 30 min. at same temperature. After that, oxetan-3-one (362 mg, 5.03 mmol) was added to the reaction mixture at −78° C. This reaction mixture was stirred for 0.5 h at 25° C. After completion of the reaction as indicated by TLC, the reaction mixture was quenched with saturated NH₄Cl soln. (10 mL) at 0° C. and extracted with EtOAc (3×20 mL). The combined organic layer was dried over anhyd. Na₂SO₄ and concentrated under reduced pressure. This crude residue was purified by combiflash column chromatography using 0-70% EtOAc/heptane as an eluent and to afford tert-butyl 4-(3-hydroxyoxetan-3-yl)indoline-1-carboxylate (SM-IV-30a) as a yellow semi solid. R_{f}(EtOAc: heptane, 50:50)=0.3; yield: 180 mg, 37%. MS: m/z 192.12 [M−100+H]⁺.

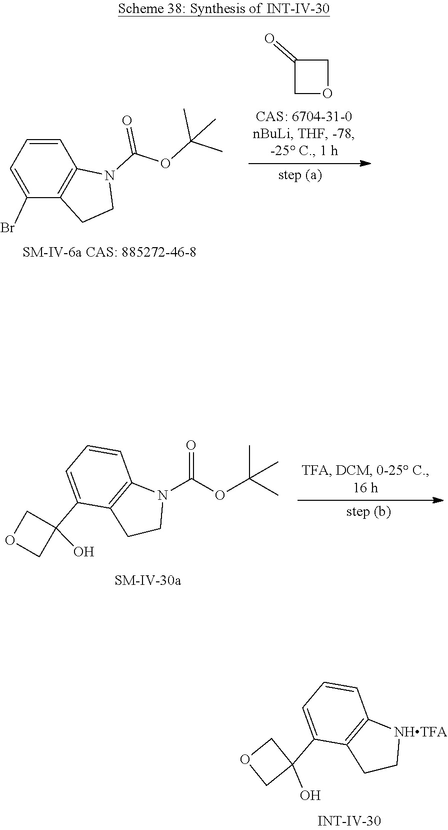

Step (b): Treatment of tert-butyl 4-(3-hydroxyoxetan-3-yl)indoline-1-carboxylate (SM-IV-30a, 160 mg, 0.55 mmol) in DCM (4 mL) with TFA (0.21 mL, 2.75 mmol) at 25° C. for 16 h followed by washing of the crude product with heptane afforded (4-(3-hydroxyoxetan-3-yl)-114-indolin-1-yl)ethan-1-one trifluoroacetate (INT-IV-30). R_{f}(EtOAc: heptane, 80:20)=0.1; crude yield: 120 mg, 76%. MS: m z 192.11 [M+H]⁺.

### Preparation of 1-(indolin-4-yl)azetidin-3-ol (INT-IV-32)

Step (a): Treatment of tert-butyl 4-bromoindoline-1-carboxylate (SM-IV-6a, 1.0 g, 3.35 mmol) with azetidin-3-ol hydrogen chloride (1.83 g, 16.8 mmol) in the presence of NaOtBu (3.2 g, 33.5 mmol), palladium(II) acetate (0.04 g, 0.17 mmol) and BINAP (0.21 g, 0.34 mmol) in toluene (10 mL) at 100° C. for 1 h followed by work-up and purification afforded tert-butyl 4-(3-hydroxyazetidin-1-yl)indoline-1-carboxylate (SM-IV-32a) as a white solid. R_{f} (EtOAc: heptane, 70:30)=0.2; yield: 410 mg, 42%. MS: m/z 291.17 [M+H]⁺.

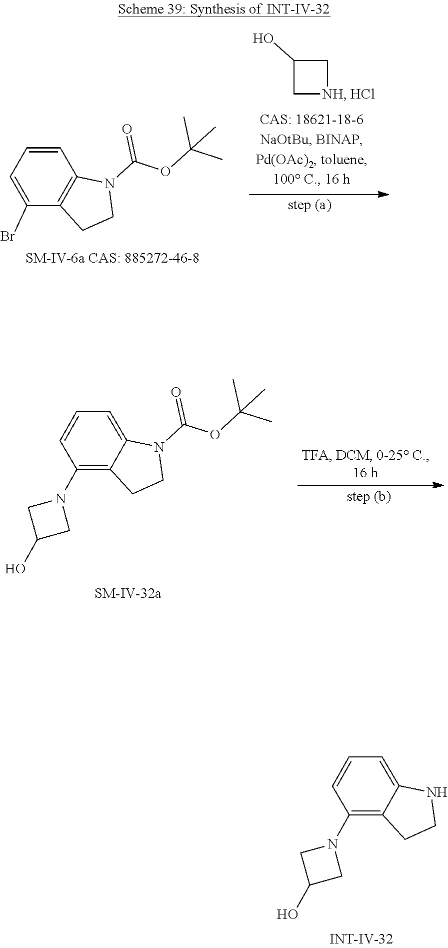

Step (b): Treatment of tert-butyl 4-(3-hydroxyazetidin-1-yl)indoline-1-carboxylate (SM-IV-32a, 0.4 g, 1.37 mmol) with TFA (2.0 mL, 26.03 mmol) in DCE (2 mL) at 25° C. for 3 h followed by work-up (DCE) and precipitation of the product using Et₂O afforded 1-(indolin-4-yl)azetidin-3-ol (SM-IV-32) as a white solid. R_{f}(EtOAc: heptane, 95:5)=0.2; yield: 110 mg, 42%. MS: m/z 191.19 [M+H]⁺.

### Synthesis of the of the Final Examples (12-57) from Pathway A-2

### General Procedure E: Synthesis Method of Formula a, Step 8, Scheme 1

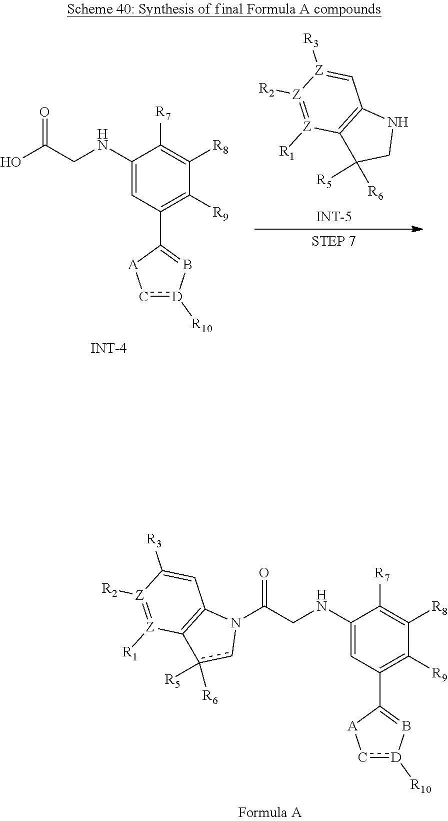

Indoline derivatives, INT-IV, Formulae parts 5 and 6 (1 equiv.) and Et₃N (2 equiv.) were first dissolved in 5 mL of DCM. Then, a mixture of the corresponding acid, INT-III, Formulae parts 3 and 4 (1.1 equiv.) and T3P (1.1 equiv., 50% solution in DCM/EtOAc) in 5 mL of DCM were then added to the solution. The resulted mixture was stirred at rt for overnight. After the completion of the reaction, the solvent was evaporated to dryness and a sat. soln. of NaHCO₃ was added and the products were extracted with EtOAc (3×50 mL). The collected organic layers were combined, dried over MgSO₄, filtered, and concentrated under reduced pressure. The products were subjected to purification with the suitable chromatographic methods to afford the pure products.

TABLE 2
Reaction and purification conditions of the final examples 1-12 syntheized via STEP 6,
1 (Pathway A)

| | | | | Synthesis | |
|---|---|---|---|---|---|
| | | | | method & | |
| | | | | Difference | Purification |
| | | | INT-III | in | Method & |
| Ex- | | INT-IV | (Formulae | Conditions | Difference in |
| ample | | (Formulae | parts 3 & | if relevant | conditions if |
| Nr. | Structure | parts 5 & 6) | 4) | (Yield %) | relevant |
| 12 | 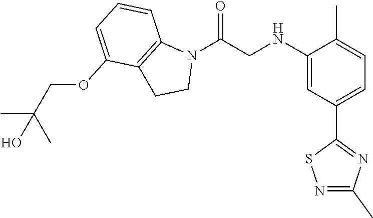 [SMILES: Cc1nsc(-c2ccc(C)c(NCC(=O)N3CCc4c(OCC(C)(C)O)cccc43)c2)n1] | INT-IV-12 | INT-III-1 | E (40) | II |
| 13 | 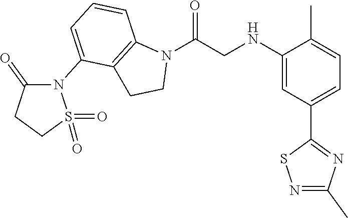 [SMILES: Cc1nsc(-c2ccc(C)c(NCC(=O)N3CCc4c3cccc4N3C(=O)CCS3(=O)=O)c2)n1] | INT-IV-18 | INT-III-1 | E (65) | I 0.5% MeOH in DCM |
| 14 | 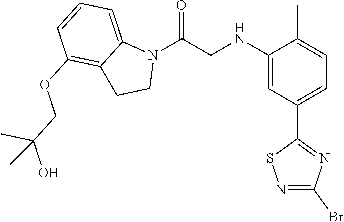 [SMILES: Cc1ccc(-c2nc(Br)ns2)cc1NCC(=O)N1CCc2c(OCC(C)(C)O)cccc21] | INT-IV-12 | INT-III-7 | E (58) | II |
| 15 | 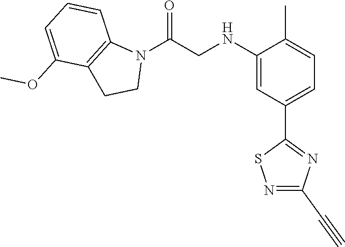 [SMILES: C#Cc1nsc(-c2ccc(C)c(NCC(=O)N3CCc4c(OC)cccc43)c2)n1] | INT-IV-10 | INT-III-4 | E HATU instead of T3P DIPEA instead of Et₃N, DMF instead of DCM (27) | Triturated with MeOH |
| 16 | 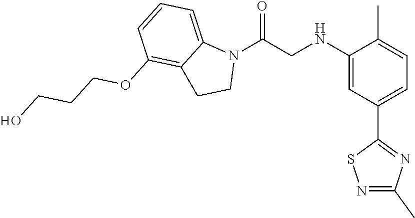 [SMILES: Cc1nsc(-c2ccc(C)c(NCC(=O)N3CCc4c(OCCCO)cccc43)c2)n1] | INT-IV-16 | INT-III-1 | E (42) | II |
| 17 | 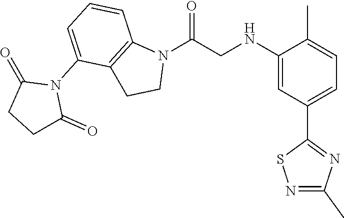 [SMILES: Cc1nsc(-c2ccc(C)c(NCC(=O)N3CCc4c3cccc4N3C(=O)CCC3=O)c2)n1] | INT-IV-22 | INT-III-1 | E (12) | II |
| 18 | 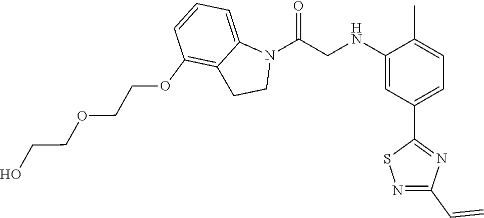 [SMILES: C=Cc1nsc(-c2ccc(C)c(NCC(=O)N3CCc4c(OCCOCCO)cccc43)c2)n1] | INT-IV-15 | INT-III-3 | E (41) | II |
| 19 | 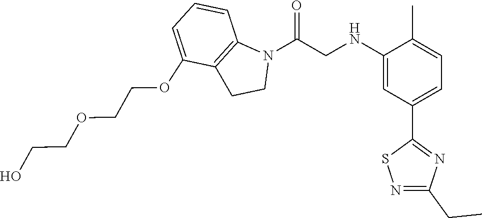 [SMILES: CCc1nsc(-c2ccc(C)c(NCC(=O)N3CCc4c(OCCOCCO)cccc43)c2)n1] | INT-IV-15 | INT-III-2 | B (20) | II |
| 20 | 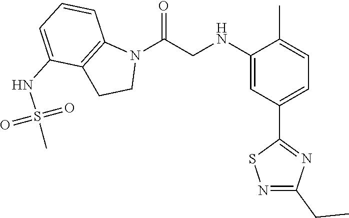 [SMILES: CCc1nsc(-c2ccc(C)c(NCC(=O)N3CCc4c(NS(C)(=O)=O)cccc43)c2)n1] | INT-IV-29 | INT-III-2 | E (51) | I & washed with EtOH |
| 21 | 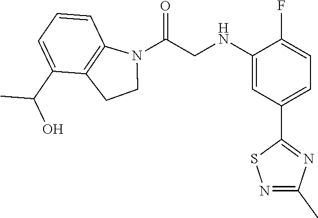 [SMILES: Cc1nsc(-c2ccc(F)c(NCC(=O)N3CCc4c(C(C)O)cccc43)c2)n1] | INT-III-10 | INT-III-10 | E (45) | II |
| 22 | 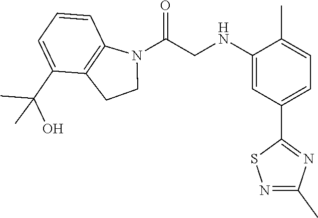 [SMILES: Cc1nsc(-c2ccc(C)c(NCC(=O)N3CCc4c3cccc4C(C)(C)O)c2)n1] | INT-IV-2 | INT-III-1 | E DMF instead of DCM (75) | Recrystallizati on with EtOH |
| 23 | 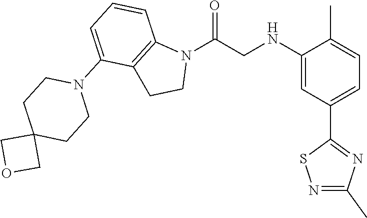 [SMILES: Cc1nsc(-c2ccc(C)c(NCC(=O)N3CCc4c(N5CCC6(CC5)COC6)cccc43)c2)n1] | INT-IV-24 | INT-III-1 | E HATU instead of T3P, DIPEA instead of Et₃N, DMF instead of DCM (22) | I EtOAc:heptane |
| 24 | 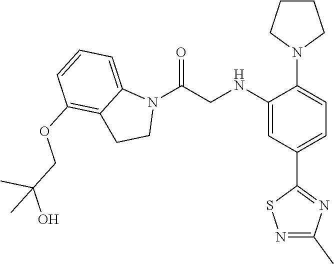 [SMILES: Cc1nsc(-c2ccc(N3CCCC3)c(NCC(=O)N3CCc4c(OCC(C)(C)O)cccc43)c2)n1] | INT-IV-12 | INT-III-11 | E (27) | II |
| 25 | 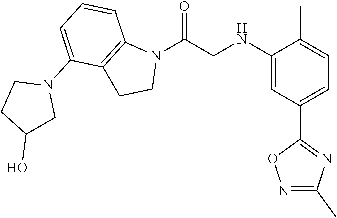 [SMILES: Cc1noc(-c2ccc(C)c(NCC(=O)N3CCc4c(N5CCC(O)C5)cccc43)c2)n1] | INT-IV-23 | INT-III-15 | E, DMF instead of DCM (62) | I EtOAc:heptane |
| 26 | 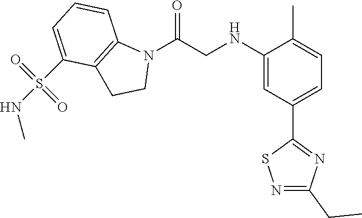 [SMILES: CCc1nsc(-c2ccc(C)c(NCC(=O)N3CCc4c3cccc4S(=O)(=O)NC)c2)n1] | INT-IV-28 | INT-III-2 | E (38) | I |
| 27 | 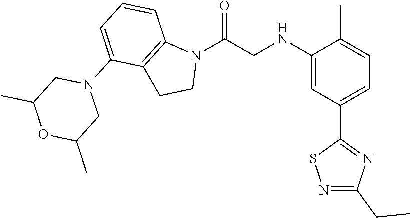 [SMILES: CCc1nsc(-c2ccc(C)c(NCC(=O)N3CCc4c(N5CC(C)OC(C)C5)cccc43)c2)n1] | INT-IV-19 | INT-III-2 | E HATU instead of T3P, DIPEA instead of Et₃N, DMF instead of DCM (15) | II |
| 28 | 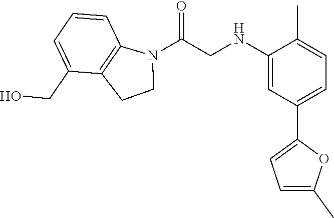 [SMILES: Cc1ccc(-c2ccc(C)c(NCC(=O)N3CCc4c(CO)cccc43)c2)o1] | INT-IV-3 | INT-III-13 | E (61) | II |
| 29 | 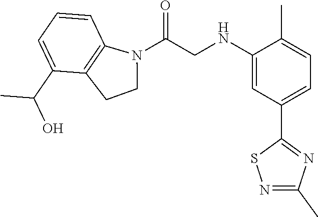 [SMILES: Cc1nsc(-c2ccc(C)c(NCC(=O)N3CCc4c(C(C)O)cccc43)c2)n1] | INT-IV-1 | INT-III-1 | E (60) | I DCM:MeOH |
| 30 | 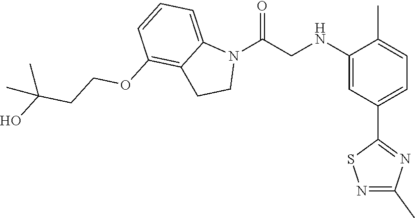 [SMILES: Cc1nsc(-c2ccc(C)c(NCC(=O)N3CCc4c(OCCC(C)(C)O)cccc43)c2)n1] | INT-IV-13 | INT-III-1 | E (39) | II |
| 31 | 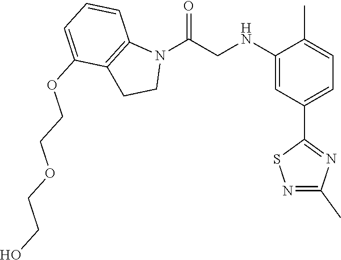 [SMILES: Cc1nsc(-c2ccc(C)c(NCC(=O)N3CCc4c(OCCOCCO)cccc43)c2)n1] | INT-IV-15 | INT-III-1 | E (47) | II |
| 32 | 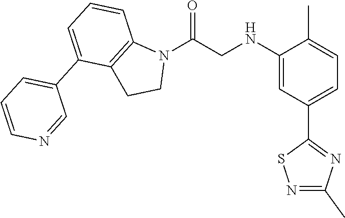 [SMILES: Cc1nsc(-c2ccc(C)c(NCC(=O)N3CCc4c(-c5cccnc5)cccc43)c2)n1] | INT-IV-6 | INT-III-1 | E HATU instead of T3P, DIPEA instead of Et₃N (11) | Precipitation with ice-cold H₂O, trituration with MeOH |
| 33 | 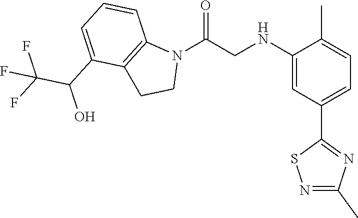 [SMILES: Cc1nsc(-c2ccc(C)c(NCC(=O)N3CCc4c(C(O)C(F)(F)F)cccc43)c2)n1] | INT-IV-5 | INT-III-1 | E (33) | I EtOAc:heptane |
| 34 | 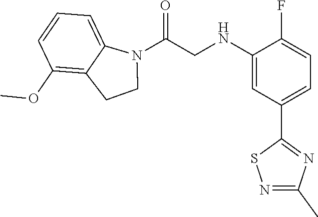 | INT-IV-10 | INT-III-9 | E (45) | The product was washed with DCM |
| 35 | 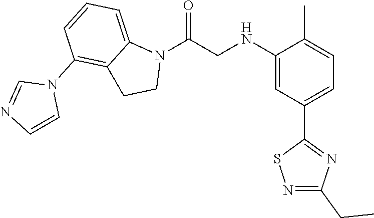 [SMILES: CCc1nsc(-c2ccc(C)c(NCC(=O)N3CCc4c3cccc4-n3ccnc3)c2)n1] | INT-IV-21 | INT-III-2 | E HATU instead of T3P (45) | Precipitation with ice-cold H₂O, recrystallizetion with DCM/Et₂O II |
| 36 | 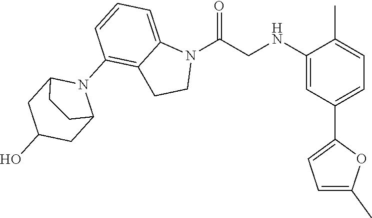 [SMILES: Cc1ccc(-c2ccc(C)c(NCC(=O)N3CCc4c3cccc4N3C4CCC3CC(O)C4)c2)o1] | INT-IV-20 | INT-III-13 | E (27) | II |
| 37 | 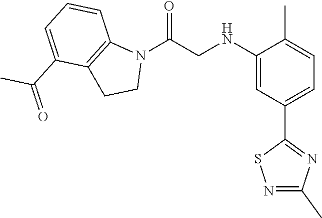 [SMILES: CC(=O)c1cccc2c1CCN2C(=O)CNc1cc(-c2nc(C)ns2)ccc1C] | INT-IV-7 | INT-III-1 | E DMF instead of DCM (72) | Recrystallization with EtOH |
| 38 | 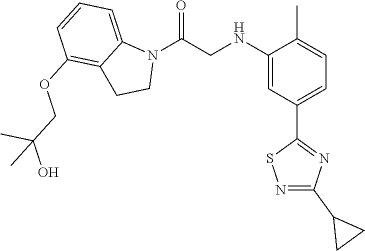 [SMILES: Cc1ccc(-c2nc(C3CC3)ns2)cc1NCC(=O)N1CCc2c(OCC(C)(C)O)cccc21] | INT-IV-12 | INT-III-6 | E (49) | I |
| 39 | 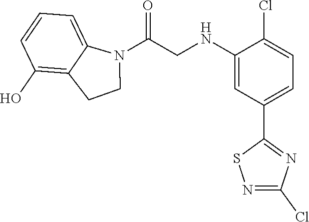 [SMILES: O=C(CNc1cc(-c2nc(Cl)ns2)ccc1Cl)N1CCc2c(O)cccc21] | INT-IV-11 | INT-III-8 | E (65) | II |
| 40 | 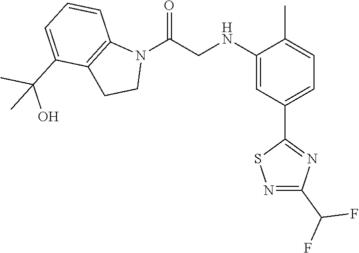 [SMILES: Cc1ccc(-c2nc(C(F)F)ns2)cc1NCC(=O)N1CCc2c1cccc2C(C)(C)O] | INT-IV-2 | INT-III-5 | E DMF instead of DCM DIPEA instead of Et₃N (46) | Precipitation of the product in ice cold H₂O_{?} |
| 41 | 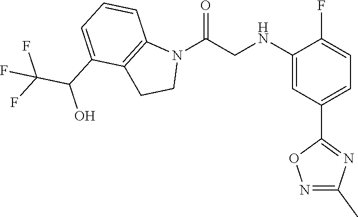 [SMILES: Cc1noc(-c2ccc(F)c(NCC(=O)N3CCc4c(C(O)C(F)(F)F)cccc43)c2)n1] | INT-IV-5 | INT-III-16 | E (61) | II |
| 42 | 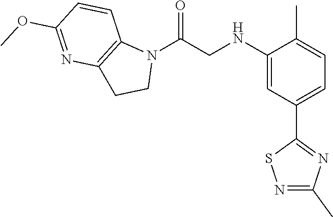 | INT-IV-4 | INT-III-1 | E (7) | II |
| 43 | 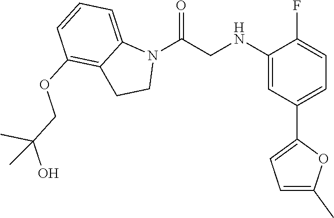 [SMILES: Cc1ccc(-c2ccc(F)c(NCC(=O)N3CCc4c(OCC(C)(C)O)cccc43)c2)o1] | INT-IV-12 | INT-III-14 | E (25) | II |
| 44 | 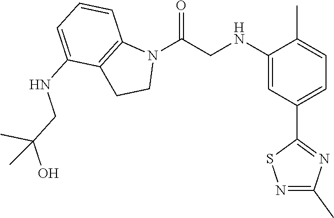 [SMILES: Cc1nsc(-c2ccc(C)c(NCC(=O)N3CCc4c(NCC(C)(C)O)cccc43)c2)n1] | INT-IV-26 | INT-III-1 | E DMF instead of DCM (38) | Precipitation with ice-cold H₂O, recrystallization with DMSO/H₂O |
| 45 | 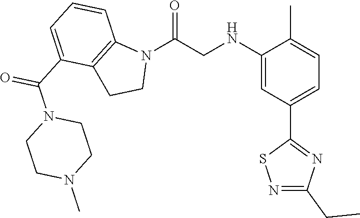 [SMILES: CCc1nsc(-c2ccc(C)c(NCC(=O)N3CCc4c(C(=O)N5CCN(C)CC5)cccc43)c2)n1] | INT-IV-9 | INT-III-2 | E (26) | I DCM:MeOH |
| 46 | 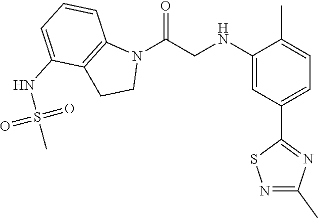 [SMILES: Cc1nsc(-c2ccc(C)c(NCC(=O)N3CCc4c(NS(C)(=O)=O)cccc43)c2)n1] | INT-IV-29 | INT-III-1 | E (35) | I Washed little EtOH |
| 47 | 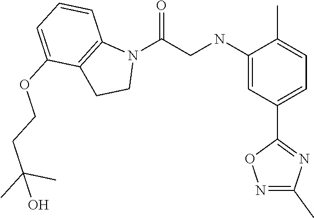 [SMILES: Cc1noc(-c2ccc(C)c(NCC(=O)N3CCc4c(OCCC(C)(C)O)cccc43)c2)n1] | INT-IV-13 | INT-III-15 | E DMF instead of DCM (65) | I EtOAc:Heptane |
| 48 | 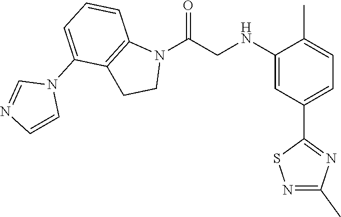 [SMILES: Cc1nsc(-c2ccc(C)c(NCC(=O)N3CCc4c3cccc4-n3ccnc3)c2)n1] | INT-IV-21 | INT-III-1 | E HATU instead of T3P DIPEA instead of Et₃N (37) | Precipitation ice cold-water and trituration with Et₂O |
| 49 | 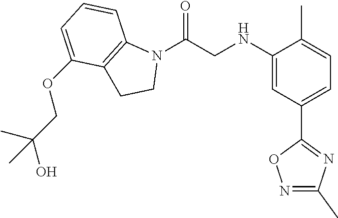 [SMILES: Cc1noc(-c2ccc(C)c(NCC(=O)N3CCc4c(OCC(C)(C)O)cccc43)c2)n1] | INT-IV-12 | INT-III-15 | E, DMF instead of DCM (64) | I EtOAc:Heptane |
| 50 | 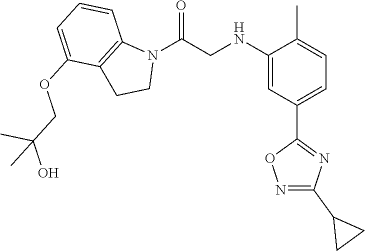 [SMILES: Cc1ccc(-c2nc(C3CC3)no2)cc1NCC(=O)N1CCc2c(OCC(C)(C)O)cccc21] | INT-IV-12 | INT-III-12 | E DMF instead of DCM (52) | Purification by RP-HPLC |
| 51 | 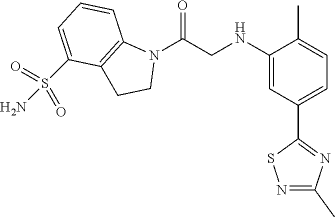 [SMILES: Cc1nsc(-c2ccc(C)c(NCC(=O)N3CCc4c3cccc4S(N)(=O)=O)c2)n1] | INT-IV-27 | INT-III-1 | E (15) | I DCM:MeOH |
| 52 | 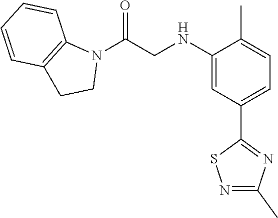 [SMILES: Cc1nsc(-c2ccc(C)c(NCC(=O)N3CCc4ccccc43)c2)n1] | INT-IV-17 | INT-III-1 | E (13) | II |
| 53 | 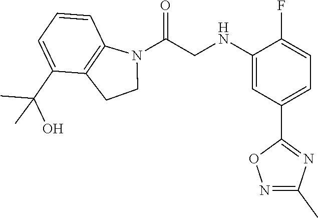 [SMILES: Cc1noc(-c2ccc(F)c(NCC(=O)N3CCc4c3cccc4C(C)(C)O)c2)n1] | INT-IV-2 | INT-III-16 | E (54) | II |
| 54 | 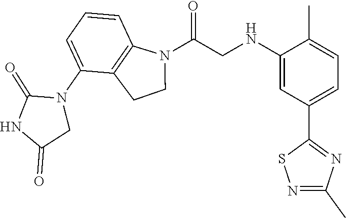 [SMILES: Cc1nsc(-c2ccc(C)c(NCC(=O)N3CCc4c3cccc4N3CC(=O)NC3=O)c2)n1] | INT-IV-25 | INT-III-1 | E (46) | I MeOH:DCM |
| 55 | 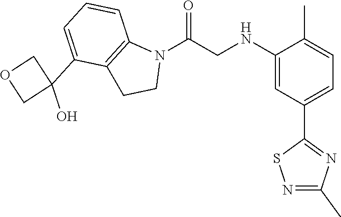 [SMILES: Cc1nsc(-c2ccc(C)c(NCC(=O)N3CCc4c3cccc4C3(O)COC3)c2)n1] | INT-IV-30 | INT-III-1 | E, DIPEA instead of Et₃N, DMF instead of DCM (22) | I |
| 56 | 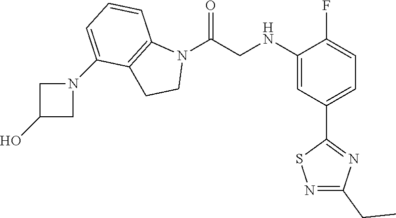 [SMILES: CCc1nsc(-c2ccc(F)c(NCC(=O)N3CCc4c(N5CC(O)C5)cccc43)c2)n1] | INT-IV-32 | INT-III-10 | E, HATU instead of T3P, DIPEA instead of Et₃N, DMF instead of DCM (61) | Precipitation with cold H₂O, recryst. with EtOH |
| 57 | 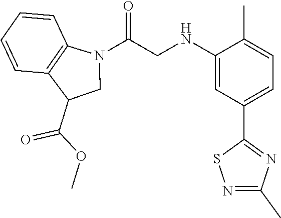 [SMILES: COC(=O)C1CN(C(=O)CNc2cc(-c3nc(C)ns3)ccc2C)c2ccccc21] | INT-IV-31 | INT-III-1 | E (78) | Precipitate washed with DCM, sat.soln. of NaHCO₃, and H₂O |

### Preparation of Example 58

Step (a): In a 100 mL flask fitted with a stirrer was loaded with tryptophol (CAS: 526-55-6, SM-58a, 2 g, 12.4 mmol) and anhyd. pyridine (20 mL) under argon. The solution was cooled to 0° C. in an ice bath. Acetic ahydride (1.77 g, 1.64 mL, 17.4 mmol, 1.4 equiv.) was added dropwise and the solution was stirred under argon at rt for overnight and then H₂O (80 mL) was added, and the reaction mixture was stirred for 20 min. It was transferred into a separatory funnel, and extracted with DCM (3×25 mL). The combined organic layers were dried on MgSO₄, and solvent removed in vacuo giving 2-(1H-indol-3-yl)ethyl acetate SM-58b (yield: 86.5%). HPLC-UV (220-400 nm) ESI-MS Purity: 97%. LC-MS (m/z): 204.1 [M+H]⁺. ¹H NMR (500 MHz, DMSO-d₆): δ 10.83 (s, 1H), 7.53 (ddt, 1H), 7.33 (dt, 1H), 7.18-7.16 (m, 1H), 7.06 (ddd, 1H), 6.97 (ddd, 1H), 4.23 (t, 2H), 2.99 (td, 2H), 1.99 (s, 3H).

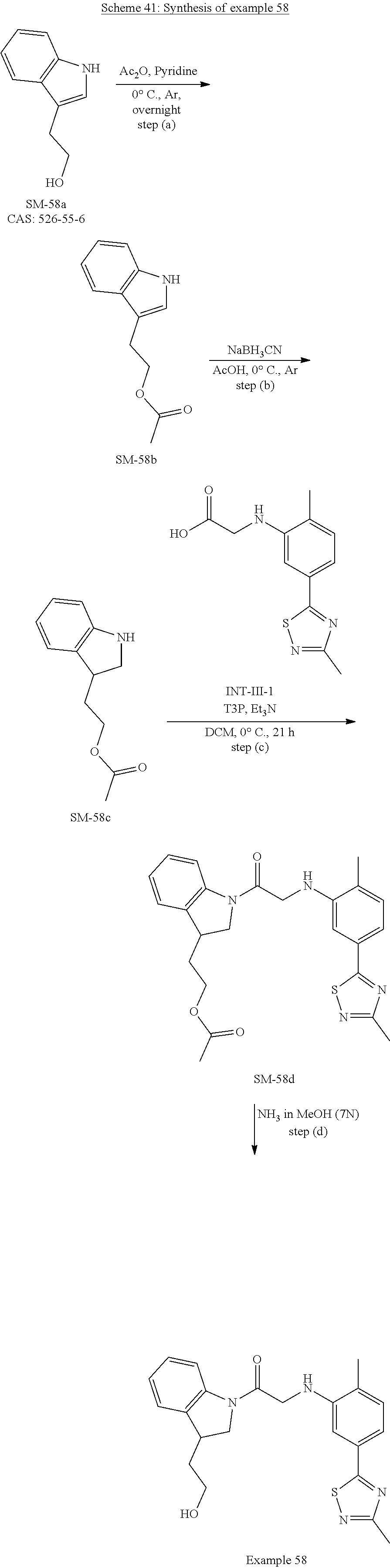

Step (b): Sodium cyanoborohydride (NaBH₃CN, 361.2 mg, 5.75 mmol, 1.5 equiv.) was added portionwise to a solution of 2-(1H-indol-3-yl)ethyl acetate (SM-58b, 777.8 mg, 3.83 mmol) in AcOH (10 mL) at 5° C. under argon. It was left stirring at rt until complete reaction. The reaction mixture was cooled on ice then slowly basified by addition of 40% aq. NaOH soln. until pH 10-11. It was then extracted with DCM (3×25 mL). The combined organic layers were dried over MgSO₄, and solvent removed in vacuo giving 2-(indolin-3-yl)ethylacetate SM-58c (yield: 63.8%) as white powder. HPLC-UV (220-400 nm) ESI-MS Purity: 62.4%. LC-MS (m/z): 205.8 [M+H]⁺. It was used for the next step without further purification.

Step (c): A solution of T3P (50% in DCM, 2.348 mg, 3.69 mmol, 1.5 equiv.) in 10 mL DCM was added dropwise to a mixture of 2-(indolin-3-yl)ethyl acetate (SM-58c, 808 mg, 2.46 mmol), 2-methyl-5-(3-methyl-1,2,4-thiadiazol-5-yl)phenyl)glycine (INT-III-1, 711.4 mg, 2.7 mmol, 1.1 equiv.), and Et₃N (746.8 mg, 1.0 mL, 7.38 mmol, 3 equiv.) in 15 mL DCM at 0° C. The resulting solution was stirred at rt for 21 h. It was washed with H₂O (2×25 mL), sat. NaHCO₃ soln. (25 mL), brine (25 mL), dried over MgSO₄, filtered off, and solvent removed in vacuo. The crude product was purified by column chromatography on silica gel using (EtOAc: cyclohexane, 20:80) giving 2-(1-((2-methyl-5-(3-methyl-1,2,4-thiadiazol-5-yl)phenyl)glycyl)indolin-3-yl)ethyl acetate SM-58d (yield: 61%) as a light beige powder. HPLC-UV (220-400 nm) ESI-MS purity: 97%. LC-MS (m/z): 451.4 [M+H]⁺.

Step (d): In a screw cap vial, 7.5 mL of ammonia solution in MeOH (7 N) was added to (SM-58d, 300 mg, 0.667 mmol) and the mixture was stirred at rt for 7 d. The reaction mixture was filtered through a büchner funnel, washed with MeOH then H₂O, and dried giving example 58 (yield: 95%) as a white powder. ¹H NMR (600 MHz, DMSO-d₆) δ 8.07 (d, 1H), 7.28 (d, 1H), 7.21-7.16 (m, 3H), 7.08 (s, 1H), 7.04 (t, 1H), 5.37 (t, 1H), 4.63 (t, 1H), 4.38 (t, 1H), 4.20 (dd, 2H), 3.96 (dd, 1H), 3.61-3.52 (m, 3H), 2.60 (s, 3H), 2.23 (s, 3H), 2.02-1.94 (m, 1H), 1.73-1.64 (m, 1H). HPLC-UV (220-400 nm) ESI-MS purity: 96%. LC-MS (m/z): 409.3 [M+H]+.

### Preparation of Example 59

Step (a): In a 10 mL round flask fitted with a stirrer was charged with example 57 (200 mg, 0.49 mmol) and LiOH. H₂O (25.8 mg, 0.615 mmol, 1.3 equiv.).CH₃CN (2 mL) and H₂O (0.5 mL) were then added and the mixture was stirred at rt for 1.5 h. The reaction mixture was transferred to a beaker, and ca. 15 mL water was added. It was acidified with conc. HCl to ca. pH 2. The formed precipitate was filtered off, washed with H₂O then dried over MgSO₄ giving 1-((2-methyl-5-(3-methyl-1,2,4-thiadiazol-5-yl)phenyl)glycyl)indoline-3-carboxylic acid, (SM-59a, 192 mg, 100%) as a light yellow powder. HPLC-UV (220-400 nm) ESI-MS Purity: 98.6%. LC-MS (m/z): 409.1[M+H]⁺.

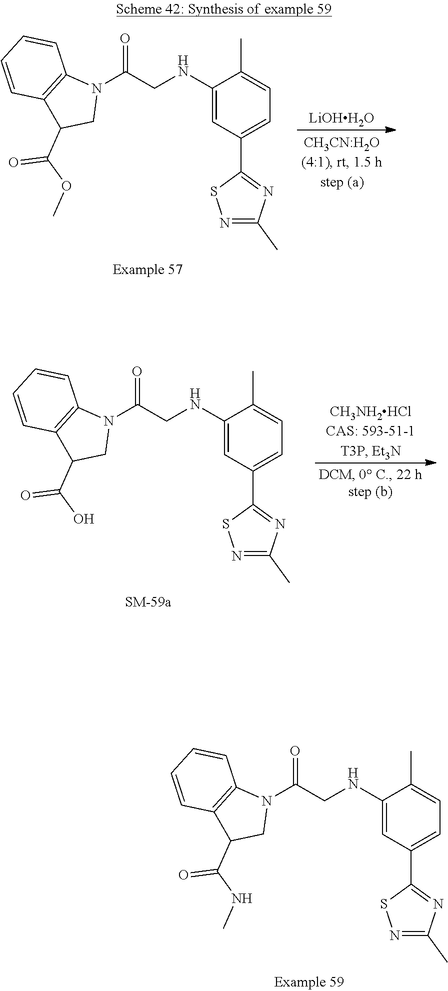

Step (b): A solution of T3P (50% in DCM, 117.1 mg, 0.184 mmol, 1.5 equiv.) in 1 mL DCM was added dropwise to a mixture of (SM-59a, 50 mg, 0.122 mmol), methylamine hydrochloride (12.4 mg, 0.184 mmol, 1.5 equiv.), and Et₃N (37.16 mg, 51.2 μL, 0.367 mmol, 3 equiv.) in 5 mL DCM at 0° C. The resulting solution was stirred at rt for 22 h. It was washed with H₂O (2×25 mL), sat. NaHCO₃ soln. (25 mL), brine (25 mL), dried over MgSO₄, filtered off, and solvent removed in vacuo giving example 59 (yield: 19.4%) as a white powder.

### Preparation of Example 60

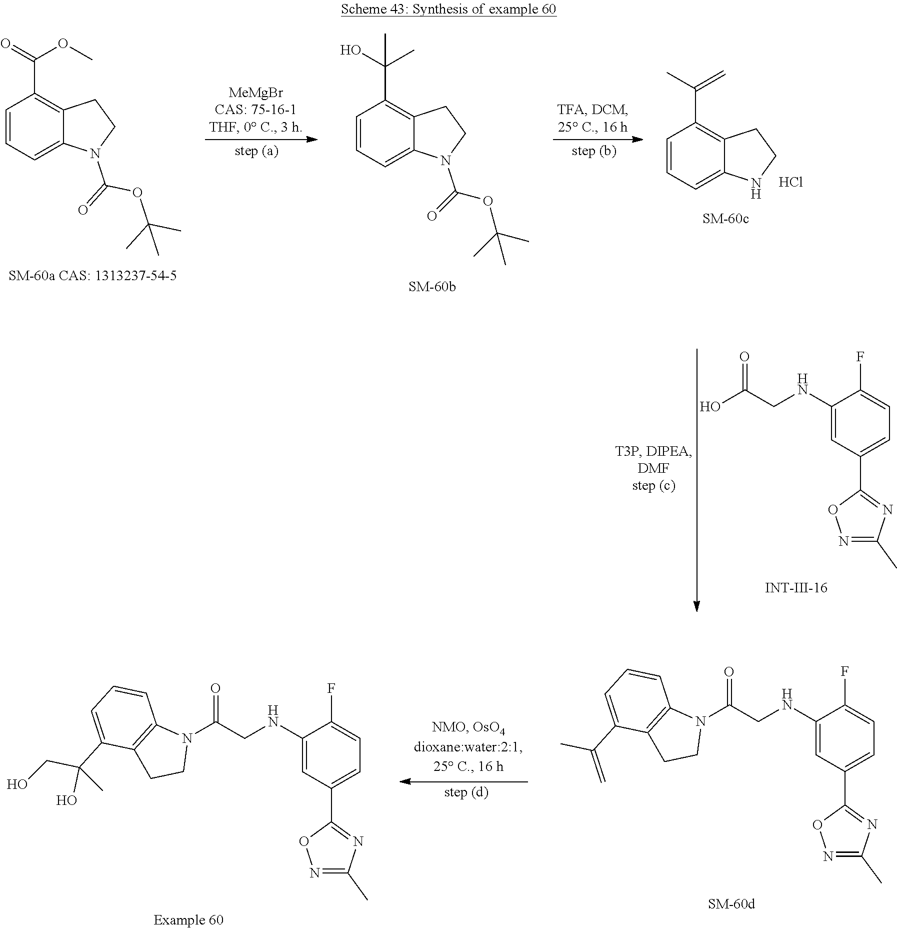

Step (a): To a stirred solution of 1-(tert-butyl) 4-methyl indoline-1,4-dicarboxylate (SM-60a, 10 g, 36.10 mmol) in THF (250 mL) was added MeMgBr (42.1 mL, 3 M in Et₂O, 126 mmol) at 0° C. under N₂ atmosphere and the reaction mixture was stirred for 3 h at 25° C. After completion of the reaction as indicated by TLC, the reaction mixture was quenched with aq. NH₄Cl and extracted with EtOAc (3×25 mL). The combined organic layer was washed with brine, dried with anhyd. Na₂SO₄ and concentrated under reduced pressure. This crude residue was purified by column chromatography using (EtOAc: heptane, 30:70) as an eluent to afford tert-butyl 4-(2-hydroxypropan-2-yl)indoline-1-carboxylate (SM-60b) as a white solid. R_{f}(EtOAc: heptane, 30:70)=0.3; yield: 80%. LC-MS (m/z): 278.54 [M+H]⁺.

Step (b): Treatment of tert-butyl 4-(2-hydroxypropan-2-yl)indoline-1-carboxylate (SM-60b, 5.0 g, 18.1 mmol) with TFA (27.8 mL, 361 mmol) in DCM (30 mL) at 25° C. for 16 h followed by basic work-up (NH₄OH) and purification by combiflash column chromatography using (EtOAc: heptane, 20:80) as an eluent afforded 4-(prop-1-en-2-yl)indoline hydrogen chloride (SM-60c) as a white solid. R_{f} (EtOAc: heptane, 30:70)=0.4; yield: 71%. LC-MS (m/z): 160.02 [M+H]⁺.

Step (c): Treatment of (2-fluoro-5-(3-methyl-1,2,4-oxadiazol-5-yl)phenyl)glycine (INT-III-16, 100 mg, 0.40 mmol) with 4-(prop-1-en-2-yl)indoline (SM-60c, 57.0 mg, 0.36 mmol) in the presence of T3P (0.42 mL, 50% w/w in EtOAc, 0.6 mmol) and DIPEA (0.21 mL, 1.2 mmol) in DMF (10 mL) at 25° C. for 2 h followed by purification by column chromatography to afford 2-((2-fluoro-5-(3-methyl-1,2,4-oxadiazol-5-yl)phenyl)amino)-1-(4-(prop-1-en-2-yl)indolin-1-yl)ethan-1-one (SM-60d) as a white solid. R_{f}(EtOAc: heptane, 50:50)=0.5; yield: 80%. LC-MS (m/z): 393.07 [M+H]⁺.

Step (d): Treatment of 2-((2-fluoro-5-(3-methyl-1,2,4-oxadiazol-5-yl)phenyl)amino)-1-(4-(prop-1-en-2-yl)indolin-1-yl)ethan-1-one (SM-60d, 100 mg, 0.26 mmol) with osmium tetroxide (OsO₄, 0.03 mL, 4% w/w in H₂O, 0.03 mmol) in the presence of NMO (177 mg, 50% w/w in H₂O, 0.76 mmol) in dioxane: H₂O (2:1, 30 mL) at 25° C. for 16 h followed by extraction and purification to afford 1-(4-(1,2-dihydroxypropan-2-yl)indolin-1-yl)-2-((2-fluoro-5-(3-methyl-1,2,4-oxadiazol-5-yl)phenyl)amino)ethan-1-one (example 60) as a white solid. R_{f}(EtOAc: heptane, 50:50)=0.3; yield: 45%.

### Preparation of Example 61

Step (a): Treatment of (5-(3-(difluoromethyl)-1,2,4-thiadiazol-5-yl)-2-methylphenyl)glycine (INT-III-5, 50 mg, 0.17 mmol) with 4-(prop-1-en-2-yl)indoline (SM-60c, 30 mg, 0.20 mmol) in the presence of T3P (0.24 mL, 50% w/w in EtOAc, 0.34 mmol) and DIPEA (0.15 mL, 0.85 mmol) in DMF (4 mL) at 25° C. for 16 h followed by precipitation of the product with ice cold water to afford 2-((5-(3-(difluoromethyl)-1,2,4-thiadiazol-5-yl)-2-methylphenyl)amino)-1-(4-(prop-1-en-2-yl)indolin-1-yl)ethan-1-one (SM-61a) as a white solid. R_{f}(EtOAc: heptane, 50:50)=0.5; yield: 74%.

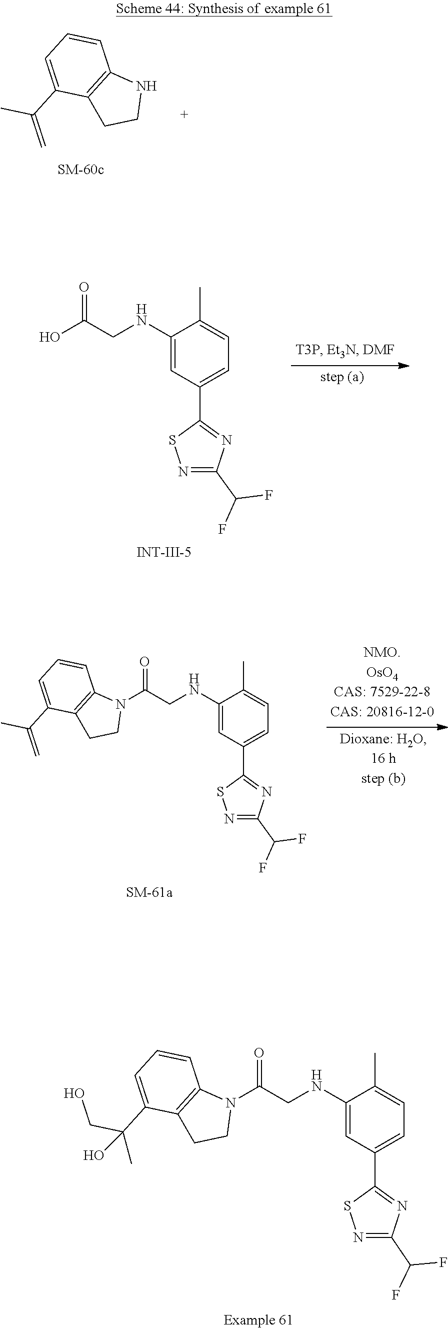 [SMILES: C=C(C)c1cccc2c1CCN2 ; Cc1ccc(-c2nc(C(F)F)ns2)cc1NCC(=O)O ; C=C(C)c1cccc2c1CCN2C(=O)CNc1cc(-c2nc(C(F)F)ns2)ccc1C ; Cc1ccc(-c2nc(C(F)F)ns2)cc1NCC(=O)N1CCc2c1cccc2C(C)(O)CO]

Step (b): Treatment of 2-((5-(3-(difluoromethyl)-1,2,4-thiadiazol-5-yl)-2-methylphenyl)amino)-1-(4-(prop-1-en-2-yl)indolin-1-yl)ethan-1-one (SM-61a, 50 mg, 0.11 mmol) with osmium tetroxide (0.01 mL, 4% w/w in H₂O, 0.007 mmol) in the presence of NMO (77 mg, 50% w/w in H₂O, 0.34 mmol) in dioxane: H₂O (5:1, 6 mL) at 25° C. for 16 h followed by extraction and purification to afford 2-((5-(3-(difluoromethyl)-1,2,4-thiadiazol-5-yl)-2-methylphenyl)amino)-1-(4-(1,2-dihydroxypropan-2-yl)indolin-1-yl)ethan-1-one (example 61) as a yellow solid. R_{f}(EtOAc: heptane, 50:50)=0.2; yield: 42%.

### Preparation of Examples 62, 63, 64, and 65

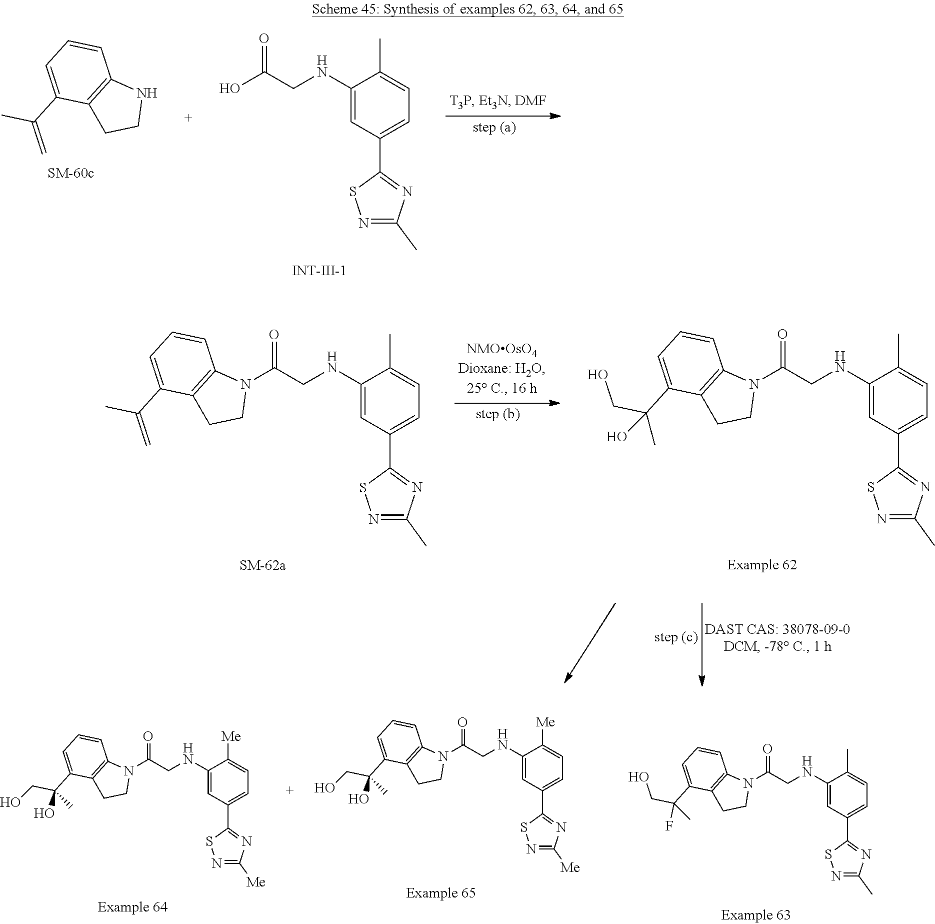

Step (a): Treatment of (2-methyl-5-(3-methyl-1,2,4-thiadiazol-5-yl)phenyl)glycine (SM-60c, 1.16 g, 4.41 mmol) with 4-(prop-1-en-2-yl)indoline (700 mg, 4.41 mmol) in the presence of T3P (6.2 mL, 50% w/w in EtOAc, 8.82 mmol) and DIPEA (2.27 g, 17.6 mmol) in DMF (12 mL) at 25° C. for 2 h followed by precipitation of the product with cold H₂O afforded 2-((2-methyl-5-(3-methyl-1,2,4-thiadiazol-5-yl)phenyl)amino)-1-(4-(prop-1-en-2-yl)indolin-1-yl)ethan-1-one (SM-62a) as a white solid. LC-MS (m/z): 405.19 [M+H]⁺. R_{f}(EtOAc: heptane, 50:50)=0.5; yield: 84%.

Step (b): Treatment of 2-((2-methyl-5-(3-methyl-1,2,4-thiadiazol-5-yl)phenyl)amino)-1-(4-(prop-1-en-2-yl)indolin-1-yl)ethan-1-one (SM-62a, 1.0 g, 2.5 mmol) with OsO₄ (0.6 mL, 4% w/w in H₂O, 0.49 mmol) in the presence of NMO (1.7 g, 50% w/w in H₂O, 7.4 mmol) in dioxane: H₂O (10 mL) at 25° C. for 16 h followed by extraction and purification to afford the corresponding example 62 as racemic mixture. This racemic mixture example 62 was purified by chiral HPLC purification using ChiralPak IG (4.6×250 mm) 5 m column and 50/50: (0.1% Et₃N in n-Hexane)/EtOH as an eluent affording the enantiomer 1, example 64 at (RT: 14.854 min) and enantiomer 2, example 65 at (RT: 19.317 min) as an off white solid. R_{f}(EtOAc: heptane, 5:5)=0.3; yield: Enantiomer 1 (example 64): 170 mg, 16%, Enantiomer 2 (example 65): 200 mg, 18%.

Step (c): To a stirred solution of 1-(4-(1,2-dihydroxypropan-2-yl)indolin-1-yl)-2-((2-methyl-5-(3-methyl-1,2,4-thiadiazol-5-yl)phenyl)amino)ethan-1-one (example 62, 200 mg, 0.46 mmol) in DCM (10 mL) was added DAST (0.12 mL, 0.92 mmol) at −78° C. under N₂atmosphere dropwise and the reaction mixture was stirred at −78° C. for 1 h. After that, the reaction mixture was allowed to warm to 25° C. and kept for stirring for 16 h. After completion of the reaction as indicated by TLC, the reaction mixture was concentrated under reduced pressure, diluted with H₂O and extracted with EtOAc (3×25 mL). The combined organic layer was dried over anhyd. Na₂SO₄ and concentrated under reduced pressure. This crude residue was purified by HPLC reverse phase purification to afford 1-(4-(2-fluoro-1-hydroxypropan-2-yl)indolin-1-yl)-2-((2-methyl-5-(3-methyl-1,2,4-thiadiazol-5-yl)phenyl)amino)ethan-1-one (example 63) as an off white solid. R_{f}(EtOAc: heptane, 80:20)=0.4; yield: 13%.

### Preparation of Example 66

Step (a): Treatment of 4-bromoindoline (SM-66a, 500 mg, 1.80 mmol) with (5-(3-ethyl-1,2,4-thiadiazol-5-yl)-2-methylphenyl)glycine (INT-III-2, 391 mg, 1.98 mmol) in the presence of HATU (1.36 g, 3.60 mmol), DIPEA (0.94 mL, 5.4 mmol) in DMF (10 mL) at 25° C. for 16 h followed by precipitation of the product with ice cold H₂O to afford 1-(4-bromoindolin-1-yl)-2-((5-(3-ethyl-1,2,4-thiadiazol-5-yl)-2-methylphenyl) amino) ethan-1-one (SM-66b) as an off white solid. R_{f} (EtOAc heptane, 30:70)=0.6; yield: 73LC-MS (m/z): 457.05 [M+H]⁺.

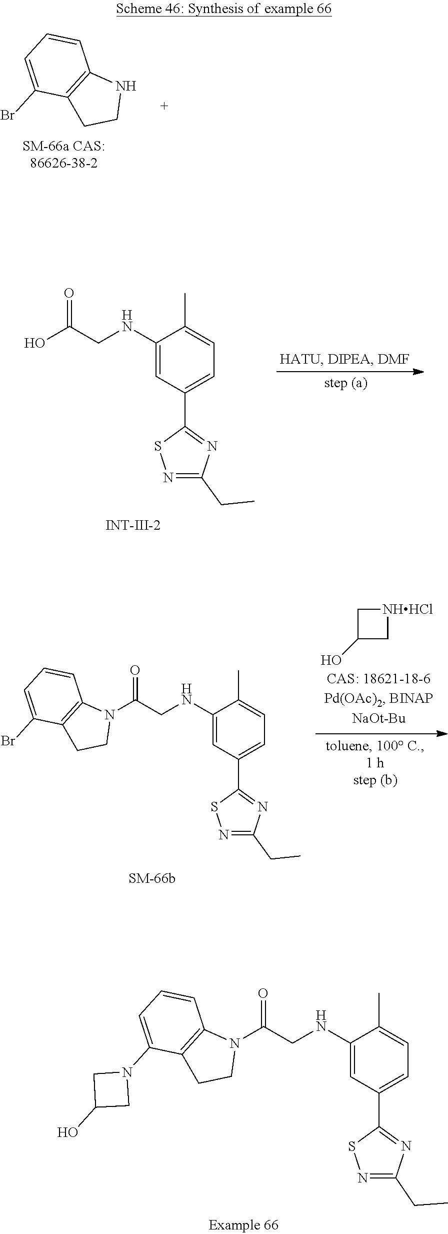

Step (b): To a solution of 1-(4-bromoindolin-1-yl)-2-((5-(3-ethyl-1,2,4-thiadiazol-5-yl)-2-methylphenyl)amino)ethan-1-one (SM-66b, 200 mg, 0.43 mmol) in toluene (10 mL), were added azetidin-3-ol hydrogenchloride (143 mg, 1.31 mmol) and NaOtBu (206 mg, 2.15 mmol) at 25° C. This reaction mixture was degassed with argon for 15 min and subsequently palladium(II) acetate (5 mg, 0.02 mmol) and BINAP (26 mg, 0.04 mmol) were added to the reaction mixture and was again degassed with argon for 5 min. The resulting reaction mixture was heated at 100° C. for 1 h. After completion of the reaction as indicated by TLC, the reaction mixture was quenched with H₂O and extracted with EtOAc (3×25 mL). The combined organic layer was washed with brine, dried over anhyd. Na₂SO₄and concentrated under reduced pressure. This crude residue was purified by combiflash chromatography using (EtOAc: heptane, 40:60) as an eluent to afford 2-((5-(3-ethyl-1,2,4-thiadiazol-5-yl)-2-methylphenyl)amino)-1-(4-(3-hydroxyazetidin-1-yl)indolin-1-yl)ethan-1-one (example 66) as an off white solid. R_{f} (EtOAc: heptane, 95:5)=0.1; yield: 20%.

### Preparation of Example 67

Step (a): To a stirred solution of 1-(4-(2-hydroxypropan-2-yl)indolin-1-yl)-2-((2-methyl-5-(3-methyl-1,2,4-thiadiazol-5-yl)phenyl)amino)ethan-1-one (example 22, 400 mg, 0.95 mmol) in DCM (30 mL) was added Et₃N (3.9 mL, 28.5 mmol) at 0° C. under N₂ atmosphere and after 10 min, the acryloyl chloride (1.29 g, 14.25 mmol) was added to the reaction mixture dropwise. The resulting mixture was stirred at 25° C. for 2 h. After completion of the reaction as indicated by TLC, the reaction mixture was diluted with ice cold H₂O (100 mL) and extracted with EtOAc (3×50 mL). The combined organic layer was washed with brine solution (30 mL), dried over anhyd. Na₂SO₄ and evaporated under reduced pressure. This crude residue was purified by combi flash column chromatography using 0-5% MeOH/EtOAc system to afford 2-(1-((2-methyl-5-(3-methyl-1,2,4-thiadiazol-5-yl)phenyl)glycyl)indolin-4-yl) propan-2-yl acrylate (SM-67a) as an off white solid. R_{f}(EtOAc: heptane, 80:20)=0.5; yield: 24%.

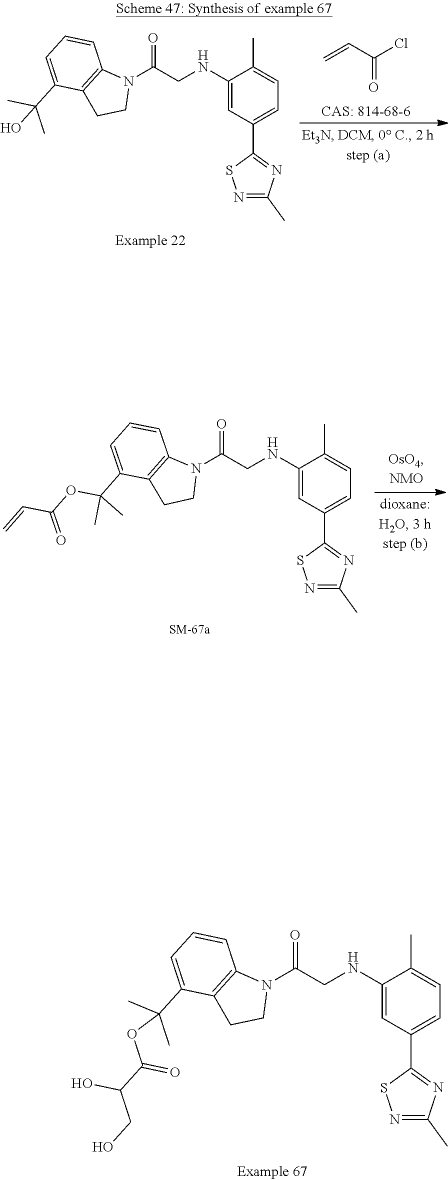

Step (b): To a stirred solution of 2-(1-((2-methyl-5-(3-methyl-1,2,4-thiadiazol-5-yl)phenyl)-glycyl)indolin-4-yl)propan-2-yl acrylate (SM-67a, 110 mg, 0.23 mmol) and NMO (64 mg, 50% w/w in H₂O, 0.28 mmol) in dioxane: H₂O (10:1, 11 mL) was added OsO₄ (0.02 mL, 4% w/w in H₂O, 0.01 mmol) at 0° C. Resulting reaction mixture was kept for stirring at 25° C. for 16 h. After completion of the reaction as indicated by TLC, the reaction mixture was concentrated under reduced pressure and the residue was diluted with H₂O and extracted with EtOAc (3×25 mL). The combined organic layer was dried over anhyd. Na₂SO₄, filtered and concentrated under reduced pressure. This crude residue was purified by combi flash chromatography using (EtOAc: heptane, 20:80) as an eluent to afford 2-(1-((2-methyl-5-(3-methyl-1,2,4-thiadiazol-5-yl)phenyl)glycyl)indolin-4-yl)propan-2-yl 2,3-dihydroxy-propanoate (example 67) as a white solid. R_{f}(EtOAc: heptane, 95:5)=0.2; yield: 60%.

### Preparation of Example 68

Step (a): Treatment of tert-butyl 4-bromoindoline-1-carboxylate (SM-IV-6a, 167 mg, 0.56 mmol) with 3-(benzyloxy)pyrrolidinehydrogen chloride (100 mg, 0.56 mmol) in the presence of NaOtBu (161 mg, 1.69 mmol), palladium(II) acetate (12 mg, 0.06 mmol) and BINAP (34 mg, 0.06 mmol) in toluene (5 mL) at 100° C. for 1 h followed by work-up and purification afforded tert-butyl 4-(3-(benzyloxy)pyrrolidin-1-yl)indoline-1-carboxylate (SM-68a) as a brown solid. R_{f}(EtOAc: heptane, 50 :50)=0.3; yield: 77%. LC-MS (m/z): 395.30 [M+H]⁺.

Step (b): Treatment of tert-butyl 4-(3-(benzyloxy)pyrrolidin-1-yl)indoline-1-carboxylate (SM-68a, 170 mg, 0.43 mmol) with HCl in EtOAc (0.6 mL, 4.0 M, 2.58 mmol) in EtOAc (3 mL) at 25° C. for 5 h followed by basic work-up afforded 4-(3-(benzyloxy)pyrrolidin-1-yl)indoline (SM-68b) as a brown semisolid. R_{f}(EtOAc: heptane, 95:5)=0.1; yield: 79%. LC-MS (m/z): 295.13 [M+H]⁺.

Step (c): Treatment of 4-(3-(benzyloxy)pyrrolidin-1-yl)indoline (SM-68b, 100 mg, 0.34 mmol) with (2-methyl-5-(3-methyl-1,2,4-thiadiazol-5-yl)phenyl)glycine (INT-III-1, 98 mg, 0.37 mmol) in the presence of HATU (258 mg, 0.68 mmol) and DIPEA (0.17 mL, 1.02 mmol) in DMF (3 mL) at 25° C. for 5 h followed by precipitation with cold water and recrystallization of the product with EtOH to afford 1-(4-(3-(benzyloxy)pyrrolidin-1-yl)indolin-1-yl)-2-((2-methyl-5-(3-methyl-1,2,4-thiadiazol-5-yl)phenyl)amino)ethan-1-one (SM-68c) as an off white solid. R_{f}(EtOAc: heptane, 95:5)=0.3; yield: 99%. LC-MS (m/z): 540.28 [M+H]⁺.

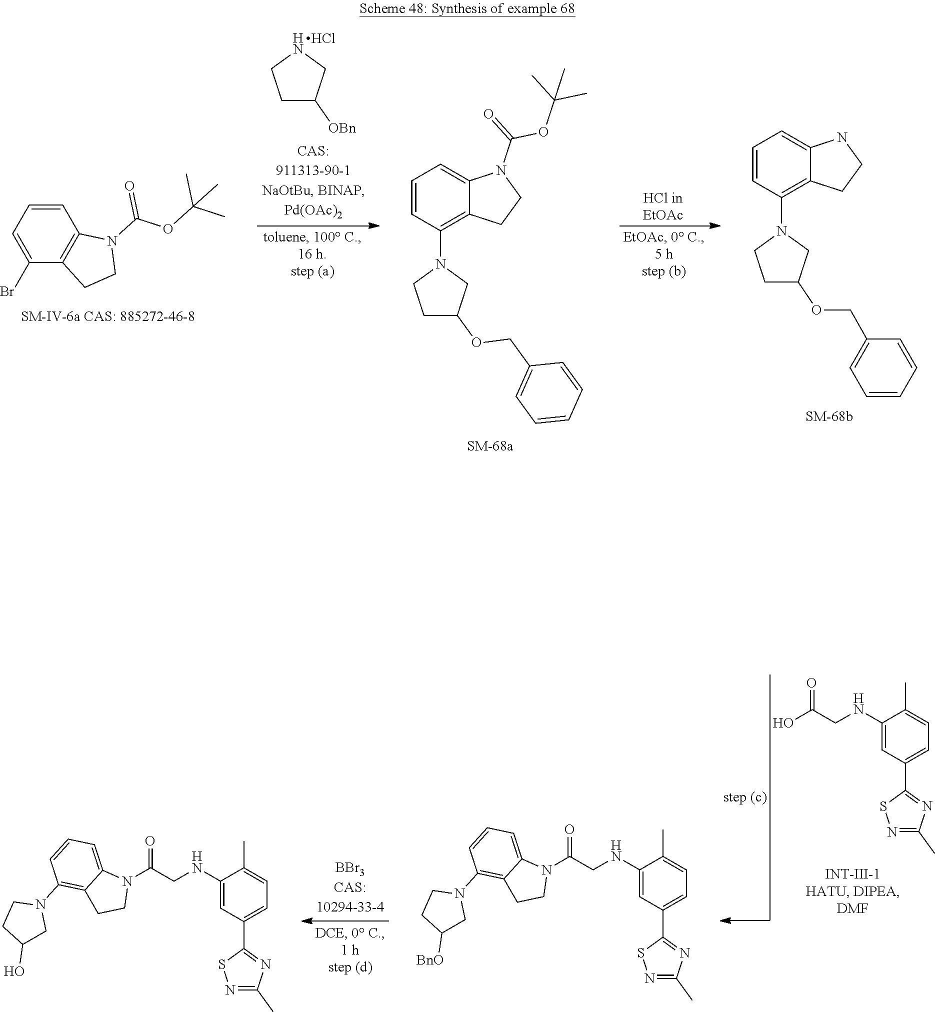

Step (d): To a solution of 1-(4-(3-(benzyloxy)pyrrolidin-1-yl)indolin-1-yl)-2-((2-methyl-5-(3-methyl-1,2,4-thiadiazol-5-yl)phenyl)amino)ethan-1-one (SM-68c, 120 mg, 0.22 mmol) in DCE (4 mL) was added BBr₃ (1.1 mL, 1.0 M in DCM, 1.11 mmol) at 0° C. The reaction mixture was allowed to warm to 25° C. and stirred for 2 h. After completion of the reaction as indicated by TLC, the reaction mixture was diluted with MeOH (10 mL) at 0° C. and stirred for 10 min at 25° C. After this, the reaction mixture was quenched with sat. NaHCO₃ soln. and extracted with EtOAc (3×20 mL). The combined organic layer was washed with brine, dried over anhyd. Na₂SO₄ and concentrated under reduced pressure. This crude residue was purified by combi flash chromatography using 0-10% MeOH/EtOAc as an eluent to afford 1-(4-(3-hydroxypyrrolidin-1-yl)indolin-1-yl)-2-((2-methyl-5-(3-methyl-1,2,4-thiadiazol-5-yl)phenyl)amino)ethan-1-one (example 68). R_{f}(EtOAc: heptane, 95:5)=0.1; yield: 30%.

### Preparation of Example 69

Step (a): To a stirred soln. of 1-(tert-butyl) 4-methyl indoline-1,4-dicarboxylate (SM-60a, 1 g, 4 mmol) in THF (25 mL) was added 7 mL of 1 M solution of DIBAL-H in THF (7 mmol) at −70° C. dropwise over 1 h. the reaction mixture was stirred at rt for 16 h. The reaction mixture was cooled at 0° C. and Rochelle's salt solution (11 mL) was carefully added (strong exothermic). The resulting mixture was stirred for 2 h at rt and then was filtered and concentrated under reduced pressure to remove the volatile solvent. The resulting solution was diluted with EtOAc (150 mL) and organic layer was washed with Rochelle's salt solution then with brine. The combined organic layers were dried over Na₂SO₄ the evaporated under reduced pressure to get crude SM-69a.

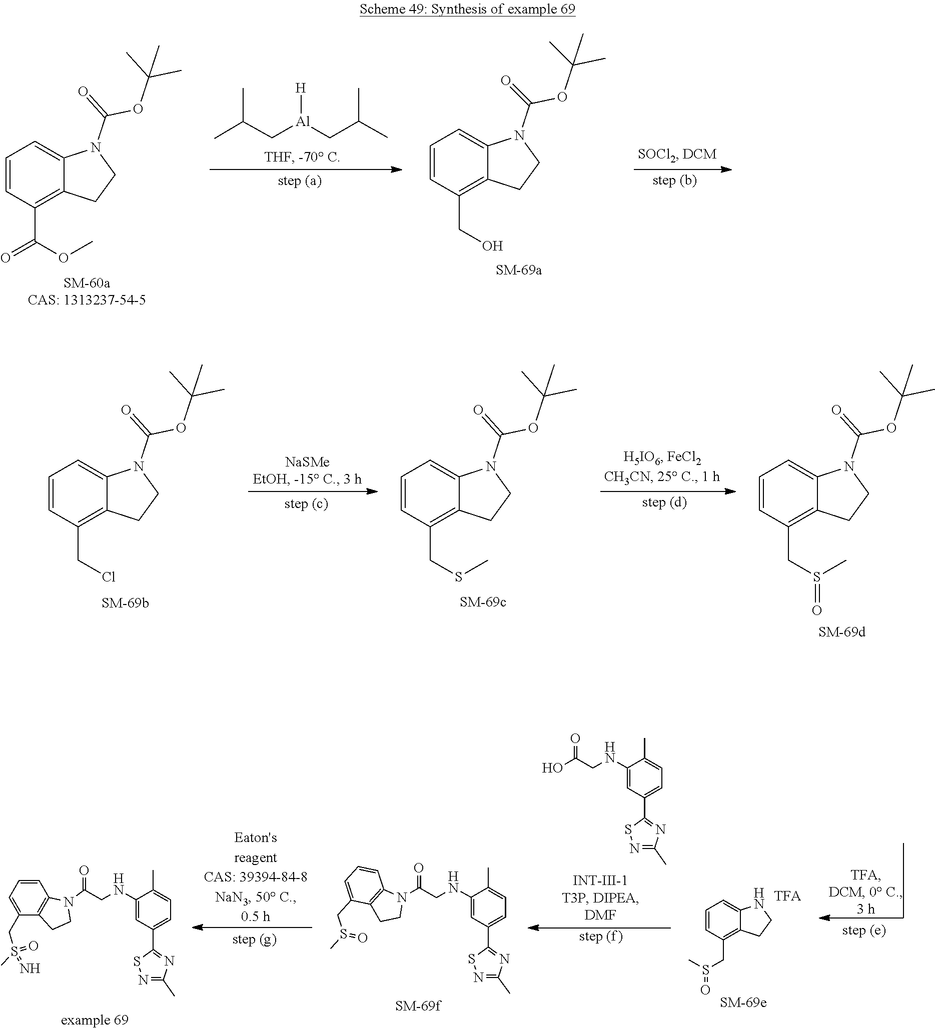

Step (b): Thionyl chloride (0.64 mL, 8.8 mmol) was added to a stirred solution of tert-butyl 4-(hydroxymethyl)indoline-1-carboxylate (SM-69a, 2.0 g, 8.0 mmol) in DCM (30 mL) at 0° C. the reaction mixture was stirred at 0° C. for 1 h then stirred at 25° C. for 4 h. The reaction mixture was concentrated under reduced pressure then dried under high vacuum to get SM-69b that was used directly in the next step.

Step (c): To a stirred solution of tert-butyl 4-(chloromethyl)indoline-1-carboxylate (SM-69b, 1.9 g, 7.11 mmol) in EtOH (50 mL) was added sodium methanethiolate (0.99 g, 14.23 mmol) at −20° C. The resulting reaction mixture was stirred at 25° C. for 3 h. After completion of the reaction as indicated by TLC, the reaction mixture was concentrated under reduced pressure, diluted with H₂O (40 mL) and extracted with EtOAc (3×80 mL), washed with brine solution. The combined organic layer was dried over anhyd. Na₂SO₄ and concentrated under reduced pressure and dried under high reduced pressure. This crude residue SM-69c was forwarded to next step without further purification. R_{f}(EtOAc: heptane, 2:8)=0.3; crude yield: 1.9 g, 96%. LC-MS (m/z): 180.08 [M−100+H]⁺.

Step (d): To a stirred sol. of tert-butyl 4-((methylthio)methyl)indoline-1-carboxylate (SM-69c, 1.7 g, 6.1 mmol) in CH₃CN (30 mL) was added orthoperiodic acid (1.5 g, 6.6 mmol) followed by iron (III) chloride (20 mg, 0.12 mmol) at 25° C. The resulting reaction mixture was kept for stirring for 2 h at 25° C. After completion of the reaction as indicated by TLC, the reaction was diluted with H₂O and extracted with EtOAc (3×90 mL). The combined organic layers were washed with brine solution, dried over anhyd. Na₂SO₄ and evaporated under reduced pressure. This crude residue was purified by flash column chromatography using (MeOH: DCM, 5:95) as an eluent to afford tert-butyl 4-((methylsulfinyl)methyl)indoline-1-carboxylate (SM-69d). R_{f}(MeOH: DCM, 5:95)=0.3; yield: 26%. LC-MS (m/z): 241.12 [M−56+H]⁺.

Step (e): Treatment of tert-butyl 4-((methylsulfinyl)methyl)indoline-1-carboxylate (SM-69d, 450 mg, 1.1 mmol) with TFA (0.01 mL, 0.17 mmol) in DCM (2 mL) for 3 h followed by work-up afforded crude 4-((methylsulfinyl)methyl)indoline trifluoroacetate (SM-69e). This crude residue was forwarded to the next step without further purification. R_{f}(EtOAc: heptane, 90:10)=0.25; crude yield: 0.28 g. LC-MS (m/z): 196.03 [M+H]⁺. ¹H-NMR (400 MHz; DMSO-d₆): δ 10.02 (br s, 1H), 7.23-7.43 (m, 2H), 7.25 (d, 1H), 4.22 (d, 1H), 4.02 (d, 1H), 3.69 (t, 2H), 3.25 (t, 2H), 2.56 (s, 3H).

Step (f): Treatment of (2-methyl-5-(3-methyl-1,2,4-thiadiazol-5-yl)phenyl)glycine (INT-III-1, 0.24 g, 0.91 mmol) with 4-((methylsulfinyl)methyl)indoline (SM-69e, 266 mg, 0.96 mmol) in the presence of T3P (0.99 mL, 50% w/w in EtOAc, 1.36 mmol) and DIPEA (0.48 mL, 2.73 mmol) in DMF (8 mL) at 25° C. for 3 h followed by extraction and trituration with MeOH, EtOAc, and heptane to afford 2-((2-methyl-5-(3-methyl-1,2,4-thiadiazol-5-yl)phenyl)amino)-1-(4-((methylsulfinyl) methyl)indolin-1-yl)ethan-1-one (SM-69f) as a white solid. R_{f}(MeOH: DCM, 5:95)=0.5; yield: 50%. LC-MS (m/z): 441.28 [M+H]⁺.

Step (g): To a stirred solution of 2-((2-methyl-5-(3-methyl-1,2,4-thiadiazol-5-yl)phenyl)amino)-1-(4-((methylsulfinyl)methyl)indolin-1-yl)ethan-1-one (SM-69f, 150 mg, 0.34 mmol) in Eaton's reagent (2 mL) was added NaN₃ (44 mg, 0.68 mmol). The reaction mixture was kept for stirring at 50° C. for 0.5 h. After completion of the reaction as indicated by TLC, the reaction mixture was quenched with sat. NaHCO₃ soln. and extracted with EtOAc (3×20 mL). The combined organic layer was washed with brine solution, dried over anhyd. Na₂SO₄ and evaporated under reduced pressure. This crude residue was purified by HPLC reverse phase purification to afford imino(methyl)((1-((2-methyl-5-(3-methyl-1,2,4-thiadiazol-5-yl) phenyl)glycyl)indolin-4-yl)methyl)sulfanone (example 69) as a white solid. R_{f} (EtOAc: heptane, 90:10)=0.4; yield: 12%. LC-MS (m/z): 456.15 [M+H]⁺.

### Preparation of Example 70

Step (a): To a solution of benzyl 4-bromoindoline-1-carboxylate (SM-IV-24a, 2.0 g, 6.04 mmol) in EtOH: H₂O (10:1, 22 mL), were added 1-(tetrahydro-2H-pyran-2-yl)-3-(4,4,5,5-tetramethyl-1,3,2-dioxaborolan-2-yl)-1H-pyrazole (2.51 g, 9.06 mmol), Na₂CO₃ (1.92 g, 18.1 mmol) at 25° C. This reaction mixture was degassed with argon for 15 min and Pd(PPh₃)₄(0.70 g, 0.60 mmol) was added to the mixture and it was again degassed with argon for 5 min. The resulting reaction mixture was heated at 100° C. for 6 h. After completion of the reaction as indicated by TLC, the reaction mixture was filtered through celite. Filtrate was concentrated under reduced pressure and the residue was purified by combiflash chromatography using (EtOAc: heptane, 30:70) as an eluent to afford benzyl 4-(1-(tetrahydro-2H-pyran-2-yl)-1H-pyrazol-3-yl)indoline-1-carboxylate (SM-70a) as a sticky liquid. R_{f} (EtOAc: heptane, 30:70)=0.2; yield: 1.9 g, 78%. LC-MS (m/z): 404.15 [M+H]⁺.

Step (b): Treatment of benzyl 4-(1-(tetrahydro-2H-pyran-2-yl)-1H-pyrazol-3-yl)indoline-1-carboxylate (SM-70a, 1.5 g, 3.72 mmol) with 10% Pd/C (50% wet, 10% w/w, 1.5 g) in the presence of H₂ gas (60 PSi) in THF: MeOH (10:1, 44 mL) at 25° C. for 12 h followed by filteration and HPLC reverse phase purification to afford 4-(1-(tetrahydro-2H-pyran-2-yl)-1H-pyrazol-3-yl)indoline (SM-70b) as a white solid. R_{f} (EtOAc: heptane, 80:20)=0.2; yield: 48%. LC-MS (m/z): 270.15 [M+H]⁺.

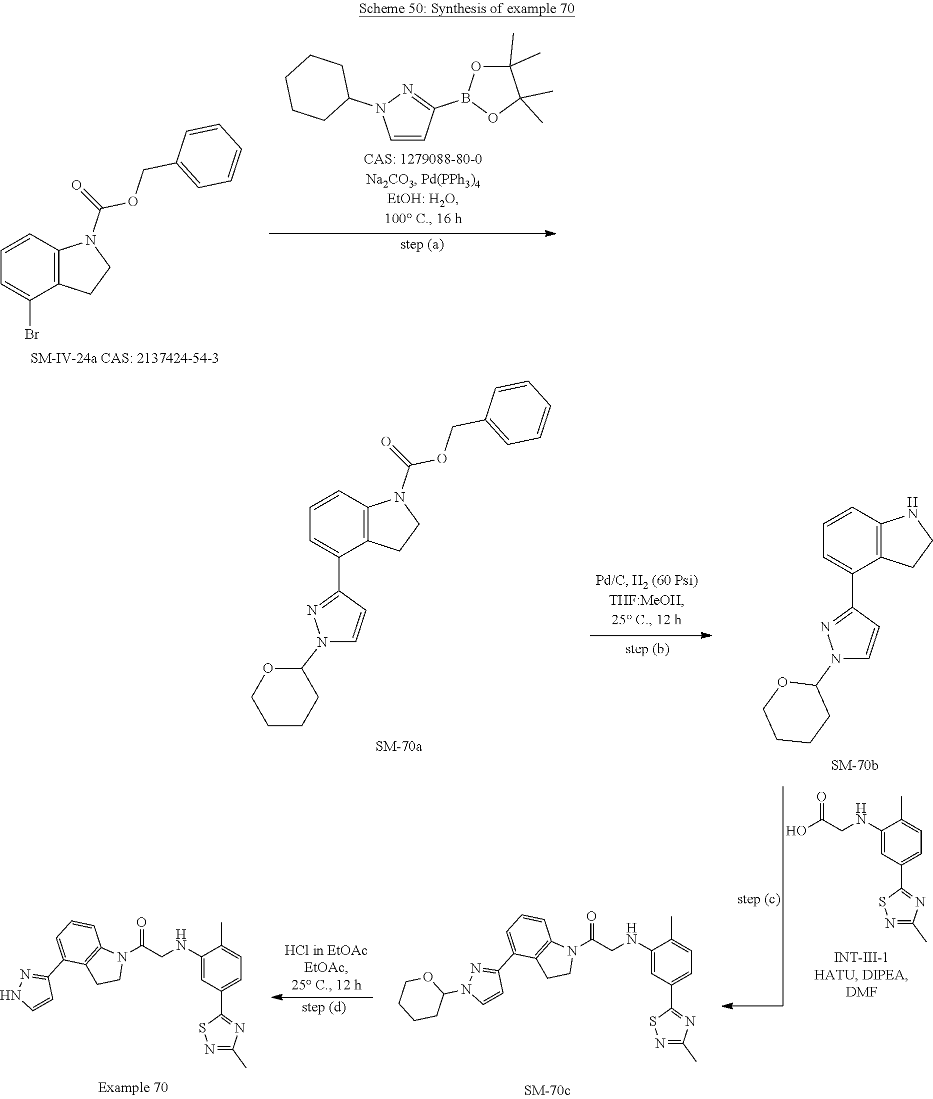

Step (c): Treatment of 4-(1-(tetrahydro-2H-pyran-2-yl)-1H-pyrazol-3-yl)indoline (SM-70b, 250 mg, 0.93 mmol) with (2-methyl-5-(3-methyl-1,2,4-thiadiazol-5-yl)phenyl)glycine (INT-III-1, 0.29 g, 1.11 mmol) in the presence of HATU (0.71 g, 1.86 mmol) and DIPEA (0.49 mL, 2.79 mmol) in DMF (3 mL) at 25° C. for 16 h followed by purification by column chromatography to afford2-((2-methyl-5-(3-methyl-1,2,4-thiadiazol-5-yl)phenyl)amino)-1-(4-(1-(tetrahydro-2H-pyran-2-yl)-1H-pyrazol-3-yl)indolin-1-yl)ethan-1-one (SM-70c) as a yellow solid. R_{f}(EtOAc: heptane, 80:20)=0.6; yield: 31%. LC-MS (m/z): 515.17 [M+H]⁺.

Step (d): To a solution of 2-((2-methyl-5-(3-methyl-1,2,4-thiadiazol-5-yl)phenyl)amino)-1-(4-(1-(tetrahydro-2H-pyran-2-yl)-1H-pyrazol-3-yl)indolin-1-yl)ethan-1-one (SM-70c, 25 mg, 0.048 mmol) in EtOAc (2 mL) was added HCl in EtOAc (1.0 mL, 1.0 M, 21.0 mmol) at 0° C. The resulting reaction mixture was kept stirring at 25° C. for 16 h. The resulting reaction mixture was concentrated under reduced pressure followed by precipitation of the product by MTBE to afford 1-(4-(1H-pyrazol-3-yl)indolin-1-yl)-2-((2-methyl-5-(3-methyl-1,2,4-thiadiazol-5-yl)phenyl)amino)ethan-1-one (example 70) as an off white solid. R_{f}(EtOAc: heptane, 50:50)=0.3; yield: 97%.

### Preparation of Example 71

Step (a): Treatment of (5-(3-ethyl-1,2,4-thiadiazol-5-yl)-2-fluorophenyl)glycine (INT-III-10, 800 mg, 2.84 mmol) with 4-bromoindoline (SM-66a, 564 mg, 2.84 mmol) in the presence of HATU (1.6 g, 4.26 mmol) and DIPEA (1.5 mL, 8.52 mmol) in DMF (10 mL) at 25° C. for 16 h followed by extraction and purification by column chromatography to afford 1-(4-bromoindolin-1-yl)-2-((5-(3-ethyl-1,2,4-thiadiazol-5-yl)-2-fluorophenyl) amino) ethan-1-one (SM-71a) as a white solid. R_{f}(EtOAc: heptane, 20:80)=0.4; yield: 62%. LC-MS (m/z): 461.03 [M+H]⁺.

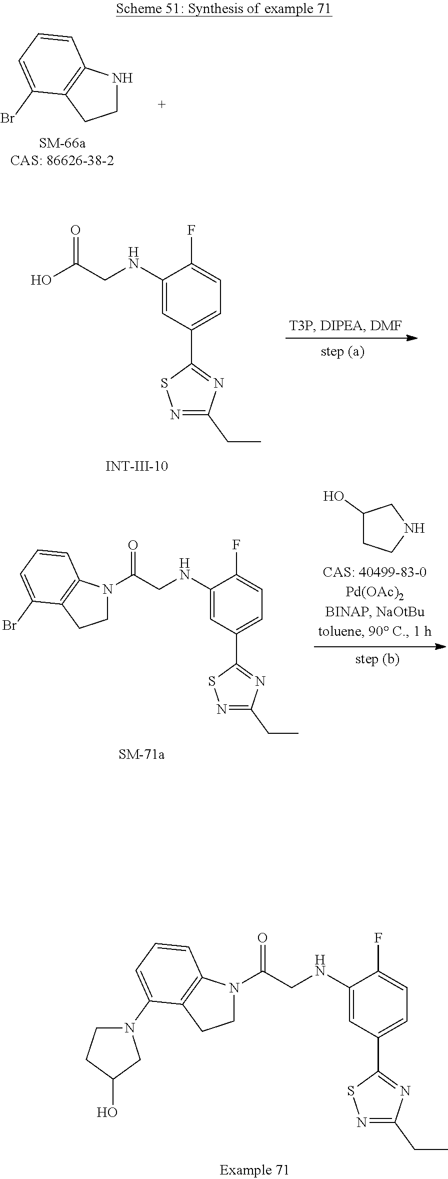 [SMILES: Brc1cccc2c1CCN2 ; CCc1nsc(-c2ccc(F)c(NCC(=O)O)c2)n1 ; CCc1nsc(-c2ccc(F)c(NCC(=O)N3CCc4c(Br)cccc43)c2)n1 ; OC1CCNC1 ; CCc1nsc(-c2ccc(F)c(NCC(=O)N3CCc4c(N5CCC(O)C5)cccc43)c2)n1]

Step (b): Treatment of 1-(4-bromoindolin-1-yl)-2-((5-(3-ethyl-1,2,4-thiadiazol-5-yl)-2-fluorophenyl)amino)ethan-1-one (SM-71a, 300 mg, 0.65 mmol) with pyrrolidin-3-ol (85 mg, 0.98 mmol) in the presence of NaOtBu (187 mg, 1.96 mmol), palladium(II) acetate (14 mg, 0.07 mmol) and BINAP (40 mg, 0.07 mmol) in toluene (10 mL) at 100° C. for 4 h followed by extraction and purification to afford 2-((5-(3-ethyl-1,2,4-thiadiazol-5-yl)-2-fluorophenyl)amino)-1-(4-(3-hydroxypyrrolidin-1-yl)indolin-1-yl)ethan-1-one (example 71) as a yellow solid. R_{f}(EtOAc: heptane, 60:40)=0.1; yield: 15%. LC-MS (m/z): 468.15 [M+H]⁺.

### Preparation of Example 72

Step (a): Treatment of benzyl 4-bromoindoline-1-carboxylate (SM-IV-24a, 1.0 g, 3.02 mmol) with tert-butyl piperazine-1-carboxylate (0.67 g, 3.62 mmol) in the presence of NaOtBu (0.87 g, 9.06 mmol), palladium (II) acetate (67 mg, 0.30 mmol) and BINAP (187 mg, 0.30 mmol) in toluene (10 mL) at 100° C. for 2 h followed by work-up and purification afforded benzyl 4-(4-(tert-butoxycarbonyl)piperazin-1-yl)indoline-1-carboxylate (SM-72a) as an off white solid. R_{f}(EtOAc: heptane, 20:80)=0.3, yield: 61%. LC-MS (m/z): 438.19 [M+H]⁺.

Step (b): To a solution of benzyl 4-(4-(tert-butoxycarbonyl)piperazin-1-yl)indoline-1-carboxylate (SM-72a, 0.8 g, 1.83 mmol) in THF (10 mL), was added 10% Pd/C (50% wet, 10% w/w, 1.14 g) at 25° C. in N₂ atmosphere. The resulting reaction mixture was stirred at 25° C. under 60 psi H₂ gas pressure at parr-shaker for 2 h. After completion of the reaction as indicated by TLC, the reaction mixture was filtered through celite and washed with MeOH. Filtrate was concentrated under reduced pressure, followed by precipitation of the product using heptane to afford 4-(1-(tetrahydro-2H-pyran-2-yl)-1H-pyrazol-3-yl)indoline (SM-72b) as an off white solid. R_{f}(EtOAc: heptane, 40:60)=0.3; yield: 63%. LC-MS (m/z): 204.23 [M−100+H]⁺.

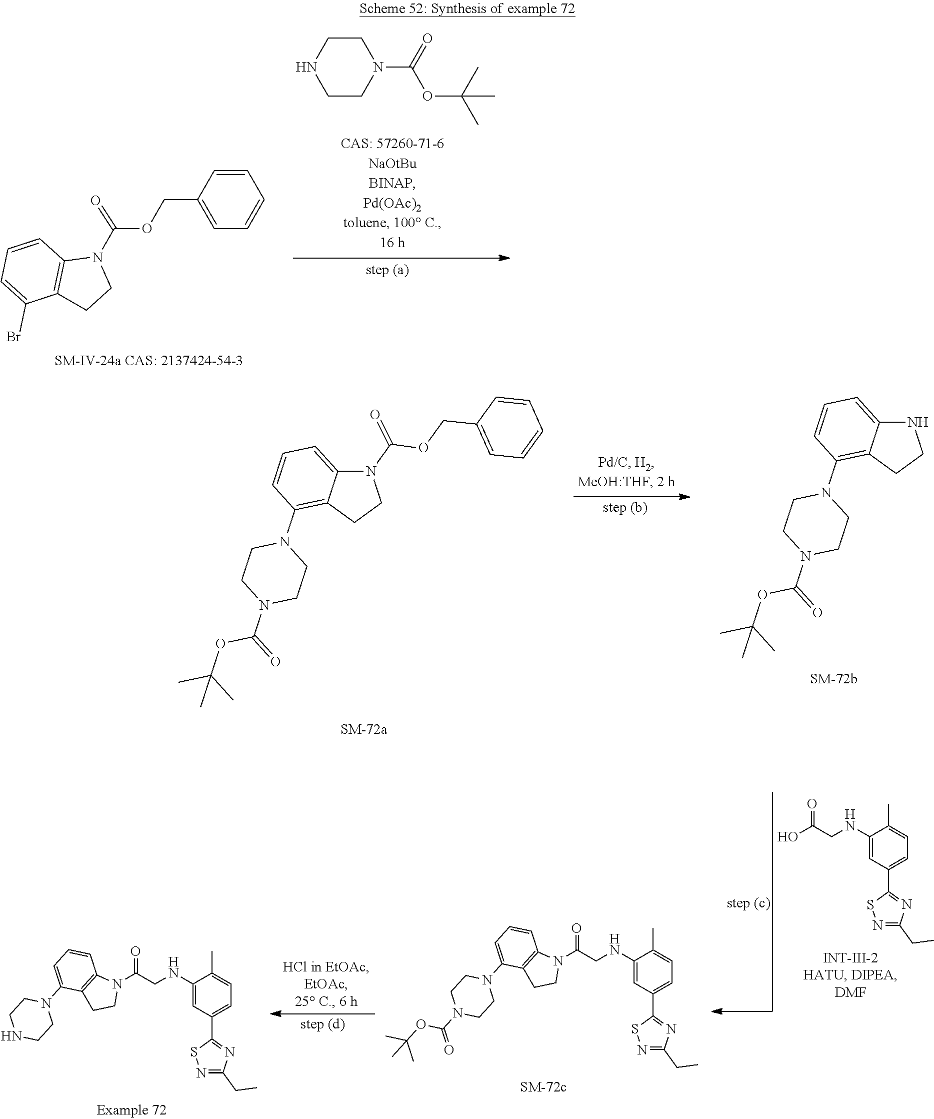

Step (c): Treatment of (5-(3-ethyl-1,2,4-thiadiazol-5-yl)-2-methylphenyl)glycine (INT-III-2, 228 mg, 0.82 mmol) with tert-butyl 4-(indolin-4-yl)piperazine-1-carboxylate (SM-72b, 250 mg, 0.82 mmol) in the presence of HATU (623 mg, 1.64 mmol) and DIPEA (0.71 mL, 4.10 mmol) in DMF (10 mL) at 25° C. for 16 h followed by precipitation of the product with cold H₂O to afford tert-butyl 4-(1-((5-(3-ethyl-1,2,4-thiadiazol-5-yl)-2-methylphenyl)glycyl)indolin-4-yl)piperazine-1-carboxylate (SM-72c) as a pink solid. R_{f}(EtOAc: heptane, 95:5)=0.3; yield: 298 mg, 65%. LC-MS (m/z): 563.27 [M+H]⁺.

Step (d): Treatment of tert-butyl 4-(1-((5-(3-ethyl-1,2,4-thiadiazol-5-yl)-2-methylphenyl)glycyl)indolin-4-yl)piperazine-1-carboxylate (SM-72c, 140 mg, 0.25 mmol) with HCl in EtOAc (2.0 mL, 1.0 M; 2.0 mmol) in EtOAc (1 mL) at 25° C. for 16 h followed by basic extraction to afford 2-((5-(3-ethyl-1,2,4-thiadiazol-5-yl)-2-methylphenyl)amino)-1-(4-(piperazin-1-yl)indolin-1-yl)ethan-1-one (example 72) as an off white solid. R_{f}(EtOAc: heptane, 8:2)=0.1; yield: 52%. LC-MS (m/z): 463.22 [M+H]⁺.

### Preparation of Example 73

Step (a): Treatment of benzyl 4-(4,4,5,5-tetramethyl-1,3,2-dioxaborolan-2-yl)indoline-1-carboxylate (SM-73a, 5.0 g, 13.2 mmol) with 4-bromo-1-((2-(trimethylsilyl) ethoxy) methyl)-1H-imidazole (5.4 g, 19.8 mmol) in the presence of Na₂CO₃ (4.19 g, 39.6 mmol) and Pd(PPh₃)₄ (1.52 g, 1.32 mmol) in EtOH (100 mL) at 100° C. for 16 h followed by extraction and purification by column chromatography on silica gel using (EtOAc: cyclohexane, 40:60) to afford benzyl 4-(1-((2-(trimethylsilyl)ethoxy)methyl)-1H-imidazol-4-yl)indoline-1-carboxylate (SM-73b) as a colorless oil. R_{f}(EtOAc: heptane, 50:50)=0.2; yield: 20%. LC-MS (m/z): 450.28 [M+H]⁺.

Step (b): Treatment of benzyl 4-(1-((2-(trimethylsilyl)ethoxy)methyl)-1H-imidazol-4-yl)indoline-1-carboxylate (SM-73b, 0.3 g, 0.67 mmol) with 10% Pd/C (50% wet, 10% w/w, 0.2 g) in the presence of H₂ gas (60 PSi) in MeOH (10 mL) at 25° C. for 3 h followed by filtration to afford 4-(1-((2-(trimethylsilyl)ethoxy)methyl)-1H-imidazol-4-yl)indoline (SM-73c) as a colorless oil which was proceeded for the next step without further purification. R_{f}(EtOAc: heptane, 90:10)=0.1; crude yield: 94%. LC-MS (m/z): 316.04 [M+H]⁺.

Step (c): Treatment of (2-methyl-5-(5-methylfuran-2-yl)phenyl)glycine (INT-III-13, 0.07 g, 0.31 mmol) with 4-(1-((2-(trimethylsilyl)ethoxy)methyl)-1H-imidazol-4-yl)indoline (SM-73c, 0.1 g, 0.34 mmol) in the presence of T3P (0.38 mL, 50% w/w in EtOAc; 0.61 mmol) and Et₃N (0.12 mL; 0.92 mmol) in DMF (5 mL) at 25° C. for 16 h followed by extraction to afford 2-((2-methyl-5-(5-methylfuran-2-yl)phenyl)amino)-1-(4-(1-((2-(trimethylsilyl)ethoxy)methyl)-1H-imidazol-4-yl)indolin-1-yl)ethan-1-one (SM-73d) which was proceeded for the next step without further purification. R_{f}(EtOAc: heptane, 50:50)=0.5; yield: 0.16 g, 98%. LC-MS (m/z): 543.37 [M+H]⁺.

Step (d): To a stirred solution of 2-((2-methyl-5-(5-methylfuran-2-yl)phenyl)amino)-1-(4-(1-((2-(trimethylsilyl)ethoxy)methyl)-1H-imidazol-4-yl)indolin-1-yl)ethan-1-one (SM-73d, 0.16 g, 0.30 mmol) in EtOAc (5 mL) at 0° C. was added 1 M HCl in EtOAc (5 mL). After stirring for 2 h the reaction mixture was concentrated under reduced pressure and the residue was washed with Et₂O (2×20 mL). The solid obtained was dried anhyd. Na₂SO₄ under reduced pressure to give 1-(4-(1H-imidazol-4-yl)indolin-1-yl)-2-((2-methyl-5-(5-methylfuran-2-yl)phenyl)amino)ethan-1-one (example 73) as an off a white solid. R_{f}(EtOAc. heptane, 80:20)=0.2; yield: 0.08 g, 65%. LC-MS (m/z): 413.17 [M+H]⁺.

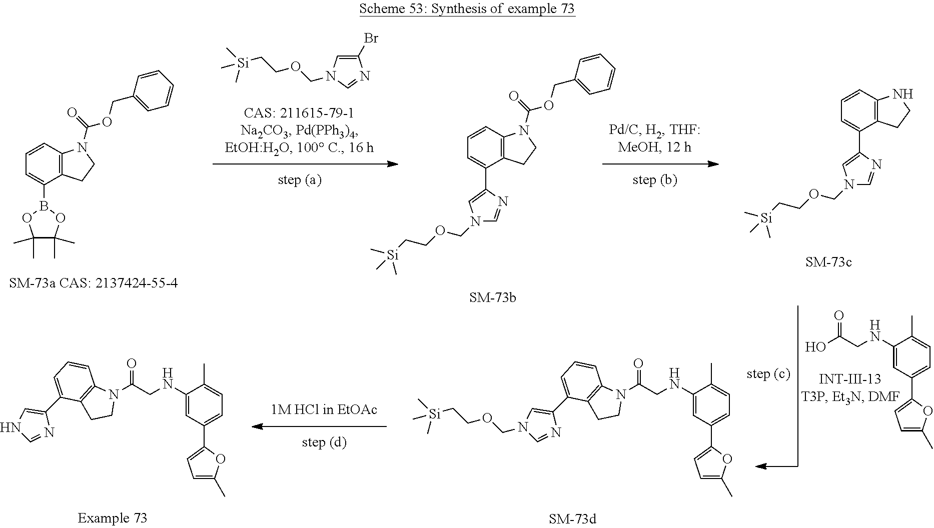

### Preparation of 4-Aminoindoline Derivatives Examples 74 and 75

Step (a): In a round flask, 4-nitroindole (SM-74a, 1 equiv.) was dissolved in TFA (9 mL for lg) then Et₃SiH (2.6 equiv.) was added. The reaction mixture was stirred at 50° C. for 3 h. The mixture was concentrated under reduced pressure. The residue was diluted with H₂O and neutralized by the addition of sat. NaHCO₃ soln. (20 mL). Extraction with EtOAc was done and the mixture was stirred for 10 min. The organic phase was separated and the aq. phase was extracted with EtOAc (2×50 mL). The organic layers were combined, dried over MgSO₄, filtered, and concentrated under reduced pressure. The residue was then triturated with PE (25 mL) to afford the nitroindoline SM-74b that was used for the next reaction without further purification (yield: 79%).

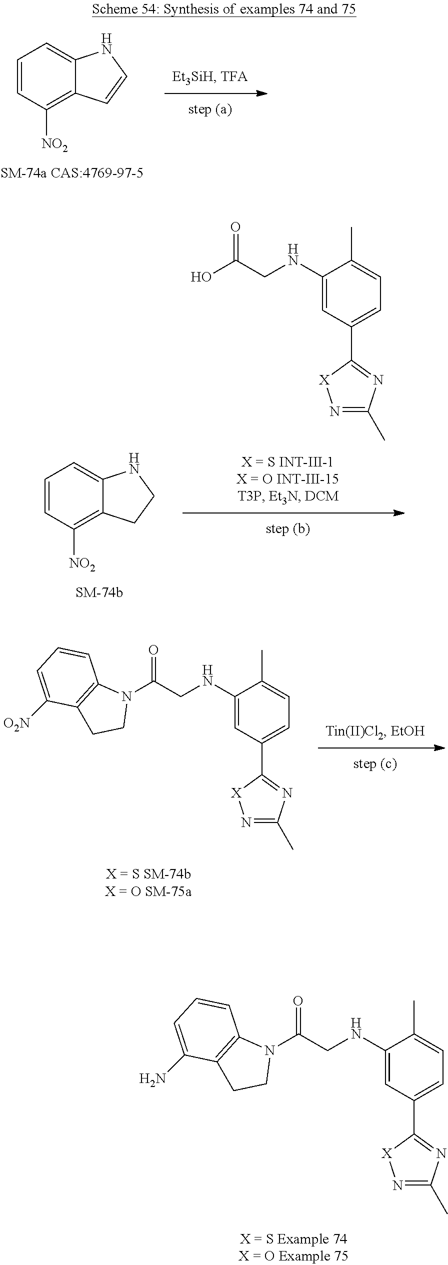

Step (b): The obtained 4-nitroindoline (SM-74b, 1 equiv.) and Et₃N (2 equiv.) were dissolved in 10 mL of DCM. A mixture of the corresponding acid (1.1 equiv.) and T3P (1.1 equiv., 50% solution in DCM) in 10 mL of DCM was added to the solution. The resulting mixture was stirred at rt for 18 h. The solvent was evaporated to dryness. Saturated NaHCO₃ soln. was added and the aq. phase was extracted with EtOAc (3×50 mL). The organic layers were combined, dried over MgSO₄, filtered, and concentrated under reduced pressure, followed by purification with method II to afford the products SM-74b and SM75a as a yellow solid.

Step (c): The obtained nitro compounds (SM-74b and SM75a, 1 equiv.) were suspended in EtOH (10 mL for every 100 mg). SnCl₂ (8 equiv.) was added and the resulting mixture was stirred at 70° C. for 18 h. The mixture was concentrated under reduced pressure. The residue was then diluted with H₂O and neutralized by the addition of sat. NaHCO₃ soln (50 mL). The aq. phase was extracted with EtOAc (4×50 mL). The organic layers were combined, dried over MgSO₄, filtered, and concentrated under reduced pressure followed by purification with method II to afford the products (examples 74 and 75) as a white solid.

### Preparation of Example 76

In a round bottom flask, 1-(4-aminoindolin-1-yl)-2-((2-methyl-5-(3-methyl-1,2,4-thiadiazol-5-yl)phenyl)amino)ethan-1-one (example 74, 130 mg, 0.34 mmol) was dissolved in acetic anhydride (2 mL). The reaction mixture was stirred at rt for 18 h. Water was then added and the aq. layer was extracted with EtOAc (3×50 mL). The combined organic layers were washed with sat. NaHCO₃ soln. one time, H₂O two times, dried over MgSO₄, filtered off, and solvent removed in vacuo to afford the crude product that was purified with method II to give the pure product example 76 (yield: 35%).

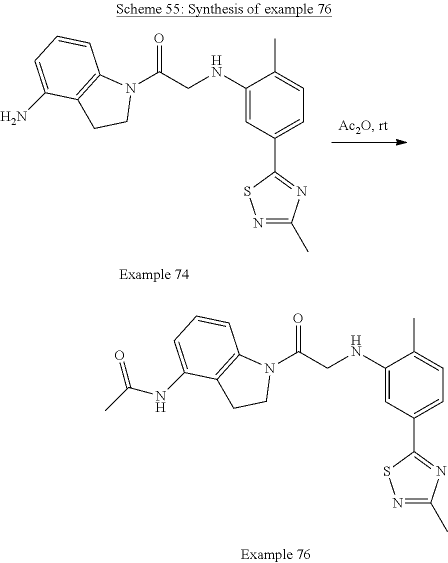

### Preparation of Example 77

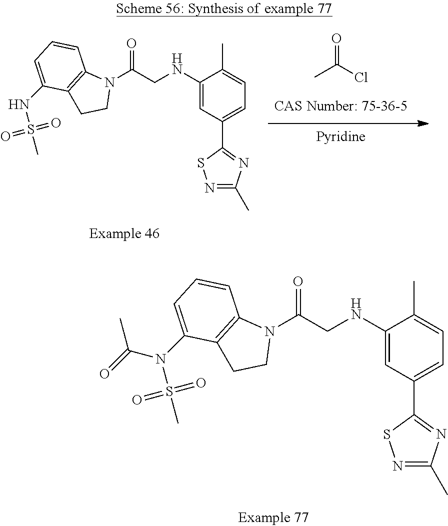

To a stirred soln. of N-(1-((2-methyl-5-(3-methyl-1,2,4-thiadiazol-5-yl)phenyl)glycyl)indolin-4-yl)methanesulfonamide (example 46, 50 mg, 0.11 mmol) in pyridine (1 mL). Acetyl chloride (15 μL, 0.21 mmol) was added to the reaction mixture and stirred at rt for 18 h. Water was then added and the aq. layer was extracted with EtOAc (3×50 mL). The combined organic layers were washed with sat. NaHCO₃ soln. one time, H₂O two times, dried over MgSO₄, filtered off, and the solvent was removed in vacuo to afford the crude product that was purified with method II to give the pure product example 77 (yield: 28%).

### Preparation of Example 78

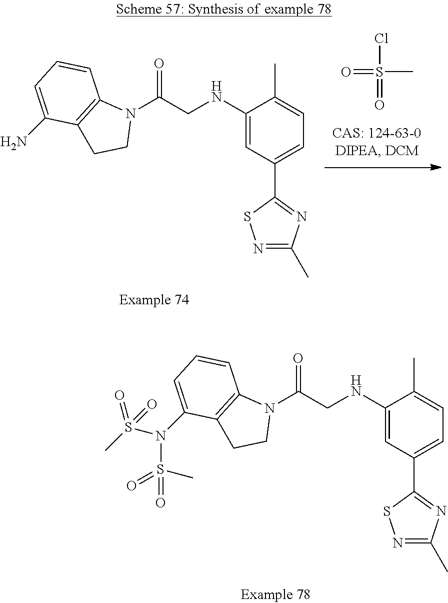

At 0° C., to a solution of 1-(4-aminoindolin-1-yl)-2-((2-methyl-5-(3-methyl-1,2,4-thiadiazol-5-yl)phenyl)amino)ethan-1-one (example 74, 1 equiv.) and DIPEA (3 equiv.) in DCM (30 mL) was added methanesulfonyl chloride (2 equiv.) dropwise. The reaction mixture was stirred at 0° C. for 1 h, and then allowed to warm to rt for 16 h. The resulting mixture was diluted with sat. NaHCO₃ soln. (30 mL) and extracted with DCM (3×30 mL). The combined organic layers were dried and concentrated followed by flash column chromatography (DCM:EtOAc, 50:50, in 30 min gradient) to afford the product example 78 as a yellow solid (yield: 70%).

### Preparation of Example 79

To a stirred soln. of example 74 (50 mg, 0.13 mmol) in AcOH (1 mL), furan-2,5-dione (17 mg, 0.17 mmol) was added to the reaction mixture and was refluxed for 1 h. After cooling to rt, the reaction mixture was poured over ice affording a precipitate, which was filtered off and washed with sat. NaHCO₃ soln. then H₂O. The crude product was dried then purified with method II (DCM:MeOH gradient from 0 to 10%) to give the pure product example 79 (yield: 17%).

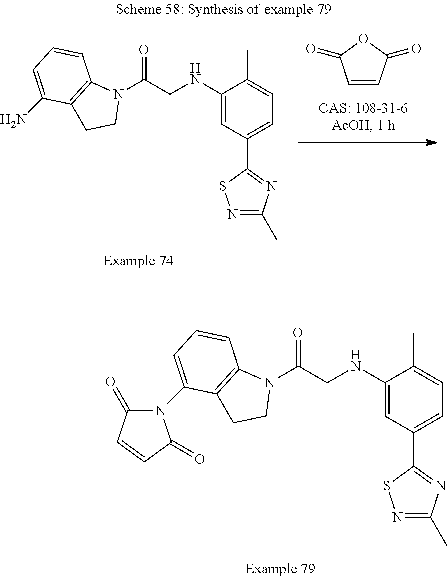 [SMILES: Cc1nsc(-c2ccc(C)c(NCC(=O)N3CCc4c(N)cccc43)c2)n1 ; O=C1C=CC(=O)O1 ; Cc1nsc(-c2ccc(C)c(NCC(=O)N3CCc4c3cccc4N3C(=O)C=CC3=O)c2)n1]

### Preparation of Example 80

Step (a): The SM-80a was prepared according to method E using example 74 (100 mg, 0.13 mmol) and (tert-butoxycarbonyl)aspartic acid (34 mg, 0.14 mmol) and DMF. The crude product was purified with method II (DCM:MeOH gradient from 0 to 10%) to give the pure product SM-80a (yield: 59%) as a white solid. HPLC-UV (220-400 nm) ESI-MS Purity: 94%. LC-MS (m/z): 577.4 [M+H]⁺.

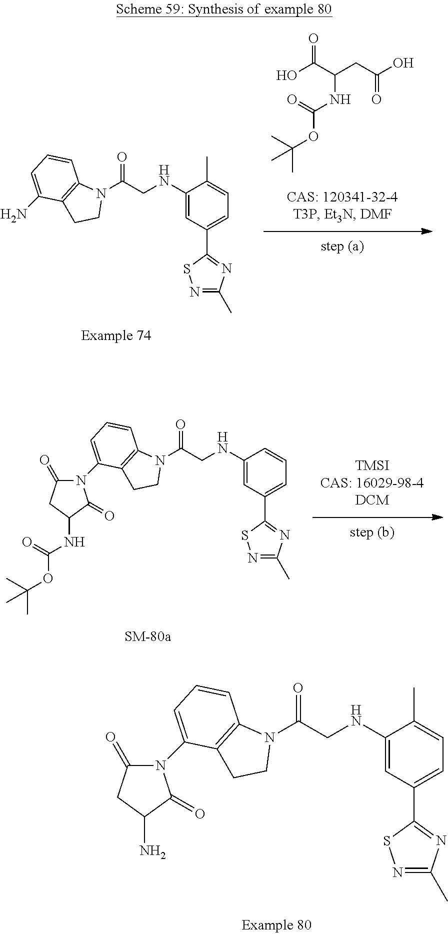 [SMILES: Cc1nsc(-c2ccc(C)c(NCC(=O)N3CCc4c(N)cccc43)c2)n1 ; CC(C)(C)OC(=O)NC(CC(=O)O)C(=O)O ; Cc1nsc(-c2cccc(NCC(=O)N3CCc4c3cccc4N3C(=O)CC(NC(=O)OC(C)(C)C)C3=O)c2)n1 ; Cc1nsc(-c2ccc(C)c(NCC(=O)N3CCc4c3cccc4N3C(=O)CC(N)C3=O)c2)n1]

Step (b): In a 10 mL pressure tube fitted with a stirrer was charged with SM-80a (50 mg, 0.09 mmol) in anhyd. DCM (4 mL) under Ar atmosphere. The tube was cooled to 0° C. before the dropwise addition of a solution of trimethylsilyl iodide (TMSI, 26 mg, 0.13 mmol, 1.5 equiv.) in 0.5 mL anhyd. DCM under argon. The reaction was complete within 45 min. MeOH (5 mL) was then added to the reaction suspension and the formed solution was stirred for 5 min. Solvent was removed in vacuo. Water was added to the residue and the pH was adjusted to ca. pH 2 with 1 N HCl. The solution was extracted once with EtOAc. The organic layer was washed many times with water and 1 N HCl until complete transfer of the product from the organic layer to the aq. layer. The combined aq. layers were basified to ca. pH 9 with saturated NaHCO₃. The formed precipitate was filtered off, washed with water and dried then purified with method II (DCM:MeOH gradient from 0 to 10%) giving example 80 (yield: 51%) as a yellow powder.

### Preparation of Example 81

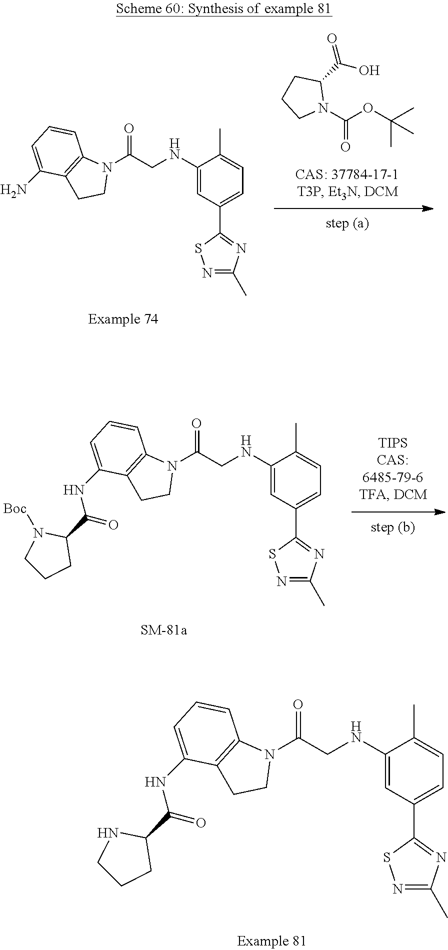 [SMILES: Cc1nsc(-c2ccc(C)c(NCC(=O)N3CCc4c(N)cccc43)c2)n1 ; CC(C)(C)OC(=O)N1CCC[C@@H]1C(=O)O ; Cc1nsc(-c2ccc(C)c(NCC(=O)N3CCc4c(NC(=O)[C@H]5CCCN5C(=O)OC(C)(C)C)cccc43)c2)n1 ; Cc1nsc(-c2ccc(C)c(NCC(=O)N3CCc4c(NC(=O)[C@H]5CCCN5)cccc43)c2)n1]

Step (a): To a solution of example 74 (40 mg, 0.11 mmol, 1 equiv.), N-Boc-D-proline (28 mg, 0.13 mmol, 1.2 equiv.) and Et₃N (0.04 mL, 0.32 mmol, 3 equiv.) in DCM, a solution of T3P (50% in DCM, 100 mg, 0.16 mmol, 1.5 equiv.) was added dropwise at 0° C. The resulting reaction mixture was stirred for overnight at rt. Afterwards, the mixture was washed with sat. NaHCO₃ soln., H₂O, brine and dried over MgSO₄. After evaporation of the solvent under reduced pressure. The crude product was purified by column chromatography on silica gel (DCM:MeOH, 90:10) to afford SM-81a as a light yellow solid; (yield: 79%).

Step (b): To a solution of SM-81a (50 mg, 0.09 mmol, 1 equiv.) in 4 mL DCM were added subsequently triisopropylsilane (0.13 mL) and TFA (1 mL). After this, the reaction mixture was stirred at rt for 1 h and then the volatiles were removed under reduced pressure. The crude product was purified by column chromatography on silica gel (DCM:MeOH, 90:10) giving 14 mg (33%) of example 81 as a light yellow solid.

### Preparation of Example 82

Step (a): In a reaction tube, 0,0′-diacetyl-L-tartaric anhydride (114 mg, 0.53 mmol, 5 equiv.) were dissolved in DCM and the solution mixture was cooled to 0° C. Then, example 74 (40 mg, 0.11 mol, 1 equiv.) was added to the solution in portions. The reaction mixture was stirred for 3 h. Afterwards the precipitated solid was filtered off and washed with DCM, yielding 42 mg (65%) of SM-82a as a yellow solid. HPLC-UV (220-400 nm) ESI-MS Purity: 98.4%. LC-MS (m/z): 596.3 [M+H]⁺.

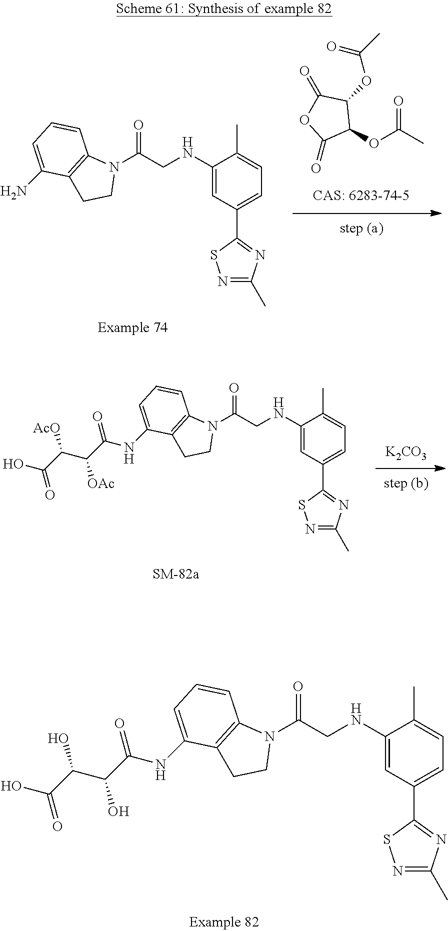

Step (b): The SM-82a (32 mg, 0.054 mmol, 1 equiv.) was suspended in water. Afterwards (25 mg, 0.16 mmol, 3 equiv.) of K₂CO₃ was added to this suspension. The resulting mixture was stirred for 3 h. Then, 2 M HCl solution was added to the reaction mixture until the pH value was turned to weakly acidic. The precipitated solid was filtered off and washed with H₂O to yield 15 mg (54%) of example 82 as a yellow solid.

### Preparation of Example 83

Step (a): To a solution of 1-(4-aminoindolin-1-yl)-2-((2-methyl-5-(3-methyl-1,2,4-oxadiazol-5-yl)phenyl)amino)ethan-1-one (example 75, 44 mg, 0.089 mmol) was added ethyl isocyanato acetate (0.3 mL, 2.67 mmol). The mixture was stirred for 1 h at rt. Subsequently volatile materials were removed under reduced pressure. The resulting intermediate SM-83a was only characterized by TLC/MS (MS: m/z 493 [M+H]⁺) and directly used for the next step.

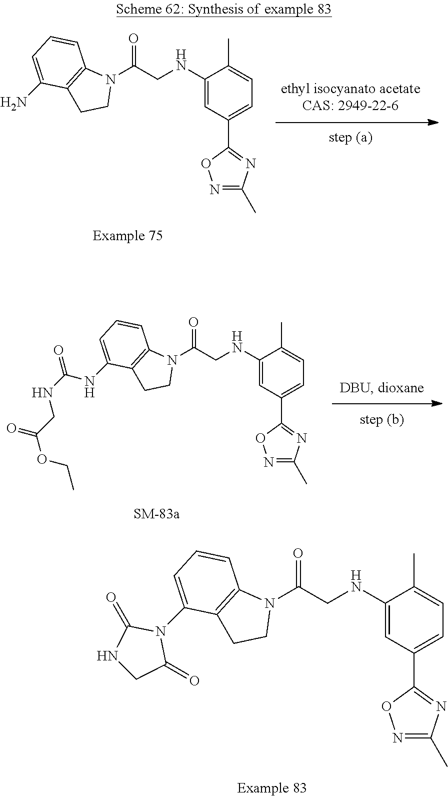 [SMILES: Cc1noc(-c2ccc(C)c(NCC(=O)N3CCc4c(N)cccc43)c2)n1 ; CCOC(=O)CNC(=O)Nc1cccc2c1CCN2C(=O)CNc1cc(-c2nc(C)no2)ccc1C ; "DBU, dioxane" ; Cc1noc(-c2ccc(C)c(NCC(=O)N3CCc4c3cccc4N3C(=O)CNC3=O)c2)n1]

Step (b): To a solution of crude SM-83a (37 mg, 0.075 mmol) in dioxane (10 mL) was added DBU (0.3 mL). The mixture was heated at 110° C. for 1 h. The reaction mixture was concentrated under reduced pressure and the residue was treated with diluted HCl. The resulting precipitate was filtered under reduced pressure, washed with water and dried at 50° C. in the oven. The crude material was purified by column chromatography over silica gel, using (DCM:MeOH, 99.5:0.5) as an eluent to give 4.5 mg of 3-(1-((2-methyl-5-(3-methyl-1,2,4-oxadiazol-5-yl)phenyl)glycyl)indolin-4-yl)imidazolidine-2,4-dione (example 83) as a colorless solid.

### Preparation of Examples 84, 85 and 86

Step (a): The compound SM-84a and the examples 1 and 2 were prepared as described in general procedure D, STEP 8 from the chloroacetylindololine and the corresponding amine derivatives.

Step (b): In a round flask, BBr₃ (1 M solution in DCM, 2.5 equiv.) was added dropwise at 0° C. to a solution of SM-84a, example 1 or example 2 (1 equiv.) in DCM. The reaction mixture was stirred atrt for 18 h. Solution of NaHCO₃ (1 N) was added to the mixture and was extracted with DCM (2×50 mL). The combined organic layers were dried over MgSO₄ and concentrated under vacuum followed purification with chromatographic method I to afford the products 84, 85, and 86 as white solids.

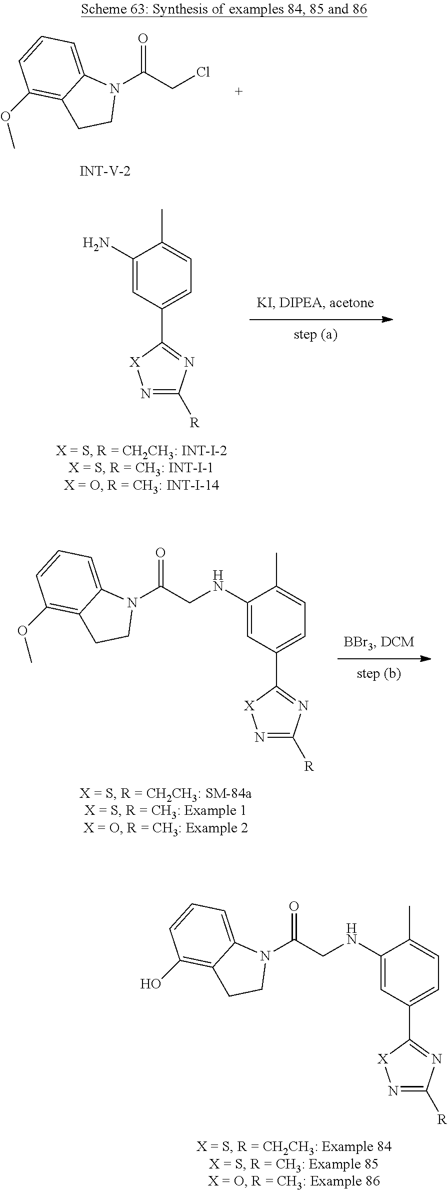

### Preparation of Example 87

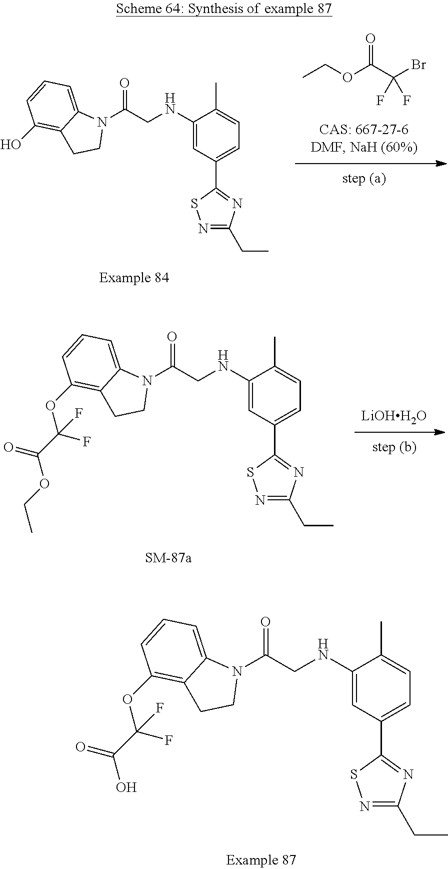

Step (a): To a solution of example 84 (100 mg, 0.25 mmol) in DMF, NaH (30 mg, 0.75 mmol, 3 equiv.) was added slowly for 10 min. at 0° C. Ethyl 2-bromo-2,2-difluoroacetate (0.045 mL, 0.35 mmol) was added slowly and the mixture was stirred at rt for 14 h. After the reaction was completed, the mixture was poured into water and stirred for 10 min. The resulting solid was filtered and dried. The product SM-87a was used for the next step without isolation.

Step (b): To the solution of SM-87a (80 mg, 0.15 mmol) in THF (2 mL), the solution of LiOH H₂O (91 mg, 0. 45 mmol) in THF: H₂O (1:1) was added slowly and stirred for 2 h. The mixture was evaporated and the resulting residue was dissolved in water. To that residue, 2 N HCl was added to precipitate the product example 87, which was filtered and dried to give the desired product as yellow solid and the yield was 8% over two steps.

### Synthesis of Examples 88 and 89

Step (a): A solution of example 33 or SM-89a (1 equiv.) and Et₃N (1.5 equiv.) in 10 mL of DCM was cooled to 0° C., and methanesulfonyl chloride in 0.5 mL of DCM (1.2 equiv.) was added dropwise. The mixture was warmed to rt and then transferred to a separating funnel and separated using EtOAc and H₂O. The organic extract was washed with 1 N HCl and saturated brine solution and dried over MgSO₄. The resulting intermediates SM-88a and SM-89b were used directly to the next step without characterization.

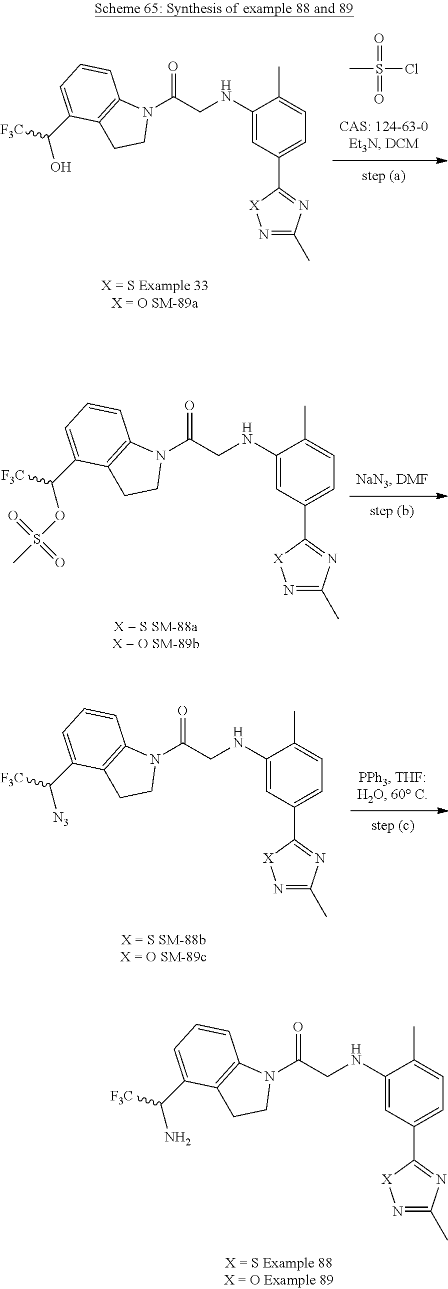

Step (b): The obtained intermediates SM-88a or SM-89b (1 equiv.) and NaN₃ (2 equiv.) were dissolved in 10 mL of DMF and the resulting mixture was heated up 80° C. for 18 h. The resulting mixture was allowed to reach to rt and then was poured onto ice water. The preciptates SM-88b or SM-89c was filtered off and used directly to the next step without characterization.

Step (c): A mixture of azido derivatives SM-88b or SM-89c (1 equiv.) and triphenylphosphine (PPh₃, 1.5 equiv.) in THF (5.0 mL) and H₂O (0.10 mL) was stirred at 60° C. for 18 h. The reaction mixture was then concentrated under vacuum and the obtained example 88 and example 89 was purified using method II.

### Synthesis of Examples 90 and 91

Step (a): A solution of tert-butyl 4-chloro-1H-pyrrolo[3,2-c]pyridine-1-carboxylate (SM-90a, 3.0 g, 12 mmol, 1 equiv.) and potassium acetate (3.5 g, 36 mmol, 3 equiv.) in EtOH (25 mL) was stirred and degassed under N₂ atmosphere for 10 min. in a steel pressure vessel. PdCl₂(dppf)-DCM adduct (1.5 g, 1.8 mmol, 0.15 equiv.) was added to the stirred solution at 25° C. Then, the reaction mixture was filled with CO gas at 55 psi and the reaction mixture was then stirred at 80° C. for 16 h. After completion of the reaction which was monitored by checking the TLC with (EtOAc: Heptane, 50:50), the reaction mixture was concentrated under reduced pressure obtained as light yellow oil. The crude product was purified by flash column chromatography using by (EtOAc: Heptane, 50:50) to afford SM-90b. LC-MS: (m/z): 289 [M−H]⁺. (Purity: 91%). ¹H NMR (400 MHz, DMSO-d₆): δ 8.57 (d, 1H), 8.19 (d, 1H), 7.93 (d, 1H), 7.19 (d, 1H), 4.39-4.44 (q, 2H), 1.65 (s, 9H), 1.33-1.40 (t, 3H).

Step (b): To a stirred solution of 1-(tert-butyl) 4-ethyl 1H-pyrrolo[3,2-c]pyridine-1,4-dicarboxylate (SM-90b, 2.3 g, 7.9 mmol 1 equiv.) in MeOH (100 mL) was added 10% of Pd(OH)₂/C (2.8 g, 20% Wt, 4.0 mmol, 0.5 equiv.) at 25° C. under N₂ atmosphere. Then, the reaction mixture was stirred at 25° C. under H₂ atmosphere for 16 h. After the reaction was completed, the mixture was filtered through celite pad, and the filtrate was concentrated under reduced pressure obtained 1-(tert-butyl) 4-ethyl 2,3-dihydro-1H-pyrrolo[3,2-c]pyridine-1,4-dicarboxylate (SM-90c) as an off white solid. LC-MS: (m/z): 293.19 [M+H]⁺. (Purity: 98%). ¹H NMR (400 MHz, DMSO-d₆): δ 8.39 (d, 1H), 7.67 (br s, 1H), 4.28-4.34 (m, 2H), 4.13-−4.14 (m, 2H), 3.33-3.39 (m, 2H), 1.52 (s, 9H), 1.29-1.33 (t, 3H).

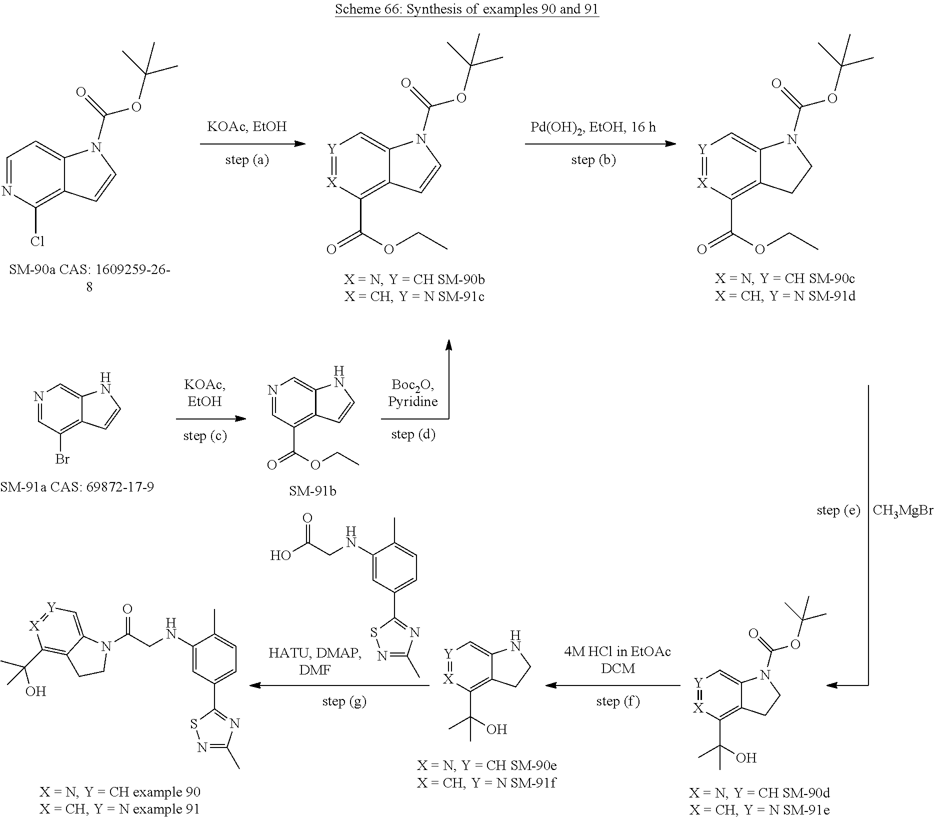

1-(tert-Butyl) 4-ethyl 2,3-dihydro-1H-pyrrolo[2,3-c]pyridine-1,4-dicarboxylate (SM-91d): The compound was prepared according to step (b) from 1-(tert-butyl) 4-ethyl 1H-pyrrolo[2,3-c]pyridine-1,4-dicarboxylate (SM-91c, 200 mg, 689 mol, 1 equiv.) and 10% of Pd(OH)₂/C (200 mg, 10% Wt, 0.14 mmol, 0.21 equiv.) at 25° C. in MeOH (20 mL) under N₂ atmosphere to afford 1-(tert-butyl) 4-ethyl 2,3-dihydro-1H-pyrrolo[2,3-c]pyridine-1,4-dicarboxylate (SM-91d) as an off white solid. LC-MS: (m/z): 293.19 [M+H]⁺. (Purity: 97%).

Step (c): A solution of 4-bromo-1H-pyrrolo[2,3-c]pyridine (SM-91a, 500 mg, 2.53 mmol, 1 equiv.) and potassium acetate (1.2 g, 12.69 mmol, 5 equiv.) in EtOH (20 mL) was stirred and degassed under N₂ atmosphere for 10 min. in a steel pressure vessel. PdCl₂(dppf)-DCM adduct (412 mg, 0.5 mmol, 0.2 equiv.) was added to the stirred solution at 25° C. Then, the reaction mixture was filled with CO gas at 55 psi and the reaction mixture was then stirred at 85° C. for 16 h. After completion of the reaction which was monitored by checking the TLC, (EtOAc: MeOH, 95:5). The crude was purified by silica gel combiflash column chromatography using 12g column, the desired product was eluted in 100% EtOAc, was then dried under high vacuum afforded ethyl 1H-pyrrolo[2,3-c]pyridine-4-carboxylate SM-91b (350 mg, 72%) as yellowish sticky solid; (Purity: 91%).

Step (d): To a solution of ethyl 1H-pyrrolo[2,3-c]pyridine-4-carboxylate (SM-91b, 300 mg, 1.58 mmol, 1 equiv.) in THF (10 mL), was added pyridine (0.64 mL, 624 mg, 7.89 mmol, 5 equiv.) followed by boc₂O (516 mg, 0.55 mL, 2.37 mmol, 1.5 equiv.) at rt and the reaction mixture was then stirred at 25° C. for 16 h. The reaction mixture was concentrated completely and quenched with H₂O (20 mL) and extracted with EtOAc (3×20 mL), the organic layers were combined, dried over anhyd. Na₂SO₄ and concentrated under vacuum. The crude was purified by silica gel combiflash column chromatography using 12g column, the desired product was eluted in 30% EtOAc/Heptane, was then dried under high vacuum afforded ethyl 1H-pyrrolo[2,3-c]pyridine-4-carboxylate (SM-91c); LC-MS: (m/z): 291.18 [M +H]⁺. (Purity: 94.5%).

Step (e): To a stirred solution of 2,3-dihydro-1H-pyrrolo[2,3-b]pyridine derivatives (1 equiv.) in THF (30 mL), MeMgBr (3 M in Et₂O, 5 equiv.) was added dropwise at 0° C. under N₂ atmosphere. The reaction mixture was then stirred at 25° C. under H₂ atmosphere for 1 h and the progress of the reaction was monitored by TLC using (EtOAc: Heptane; 50:50) as an eluent system. The reaction mixture was quenched with sat. NH₄Cl soln. (~5 mL) and the crude was extracted with EtOAc (3×20 mL). The organic layers were combined, dried over anhyd. Na₂SO₄ and concentrated under vacuum. The crude was purified by combiflash column chromatography using 60% EtOAc/Heptane as an eluent system to afford the pure product.

tert-Butyl 4-(2-hydroxypropan-2-yl)-2,3-dihydro-1H-pyrrolo[3,2-c]pyridine-1-carboxylate (SM-90d): Treatment of 1-(tert-butyl) 4-ethyl 2,3-dihydro-1H-pyrrolo[3,2-c]pyridine-1,4-dicarboxylate (SM-90c, 1 g, 3.4 mmol) with MeMgBr (2.0 g, 5.7 mL, 3.0 M in Et₂O, 17 mmol in THF (30 mL)) for 1 h at 25° C., followed by purification to afford tert-butyl 4-(2-hydroxypropan-2-yl)-2,3-dihydro-1H-pyrrolo[3,2-c]pyridine-1-carboxylate (SM-90d) as a white solid, yield 54%. LC-MS: (m/z): 279.16 [M +H]⁺. (Purity: 93%).

tert-Butyl 4-(2-hydroxypropan-2-yl)-2,3-dihydro-1H-pyrrolo[2,3-c]pyridine-1-carboxylate (SM-91e): Treatment of 1-(tert-butyl) 4-ethyl 2,3-dihydro-1H-pyrrolo[2,3-c]pyridine-1,4-dicarboxylate (SM-91d, 180 mg, 6.16 mmol, 1 equiv.) with MeMgBr (367 mg, 1 mL, 3 M in Et₂O, 3.08 mmol, 5 equiv.) in THF (10 mL) for 1 h at 25° C. followed purification to afford tert-butyl 4-(2-hydroxypropan-2-yl)-2,3-dihydro-1H-pyrrolo[2,3-c]pyridine-1-carboxylate (SM-91e) as a white solid, yielding: 77%. LC-MS: (m/z): 277.21 [M−H]⁺. (Purity: 83%).

Step (f): To a stirred solution of tert-butyl 2,3-dihydro-1H-pyrrolo[2,3-b]pyridine carboxylate derivatives (1 equiv.) in DCM (5 mL), 4 M HCl in EtOAc or TFA (10 equiv.) was added and then stirred the reaction mixture at 25° C. for 16 h. The reaction was monitored by checking TLC (EtOAc: Heptane; 50:50) using as an eluent system. The solvent was removed under reduced pressure and EtOAc was added to the resulting solid and stirred for 10 min and was then filtered through sintered glass to give the desired solid product.

2-(2,3-Dihydro-1H-pyrrolo[3,2-c]pyridin-4-yl)propan-2-ol (SM-90e): The compound was synthesized from tert-butyl 4-(2-hydroxypropan-2-yl)-2,3-dihydro-1H-pyrrolo[3,2-c]pyridine-1-carboxylate (SM-90d, 550 mg, 1.98 mmol) in DCM (5 mL) and (720 mg, 5 mL, 4 M, 19.8 mmol) of 4 M HCl in EtOAc to afford 2-(2,3-dihydro-1H-pyrrolo[3,2-c]pyridin-4-yl)propan-2-ol (SM-90e) as solid product, yield: 75%. LC-MS: (m/z): 179.2 [M+H]⁺. (Purity: 95%).

2-(2,3-Dihydro-1H-pyrrolo[2,3-c]pyridin-4-yl)propan-2-ol (SM-91f): The compound was synthesized from tert-butyl 4-(2-hydroxypropan-2-yl)-2,3-dihydro-1H-pyrrolo[2,3-c]pyridine-1-carboxylate (SM-91e, 140 mg, 0.503 mmol, 1 equiv.) in DCM (5 mL) and TFA (573 mg, 387 L, 5.03 mmol) was added and the reaction mixture was stirred for 16 h at 25° C. to afford 2-(2,3-dihydro-1H-pyrrolo[2,3-c]pyridin-4-yl)propan-2-ol (SM-91f) as solid product, yield: 93%. LC-MS: (m/z): 179.04 [M+H]⁺. (Purity: 94%).

Step (g): To a stirred soln. of (2-methyl-5-(3-methyl-1,2,4-thiadiazol-5-yl)phenyl)glycine (INT-III-1, 1 equiv.) in DMF (2 mL), were added the corresponding azaindoline (1 equiv.) and DMAP (5 equiv.) under N₂ atmosphere. Then, the reaction mixture was cooled to 0° C. and HATU (2 equiv.) was added and the mixture was stirred at 25° C. for 2 h. The reaction was monitored by checking TLC using (EtOAc: Heptane; 80:20) as an eluent system. The reaction mixture was diluted with H₂O and extracted with EtOAc (3×20 mL). The organic layer was washed with brine soln., dried over anhyd. Na₂SO₄ and was then evaporated under reduced pressure. The purification was done through flash column chromatography (12g cartridge) and the compound was eluted in (EtOAc: Heptane, 55:45) to give the desired products.

1-(4-(2-Hydroxypropan-2-yl)-2,3-dihydro-1H-pyrrolo[3,2-c]pyridin-1-yl)-2-((2-methyl-5-(3-methyl-1,2,4-thiadiazol-5-yl)phenyl)amino)ethan-1-one (Example 90): Treatment of 2-(2,3-dihydro-1H-pyrrolo[3,2-c]pyridin-4-yl)propan-2-ol (SM-90e, 100 mg, 5.61 mol) with (2-methyl-5-(3-methyl-1,2,4-thiadiazol-5-yl)phenyl)glycine (INT-III-1, 148 mg, 5.61 mmol) in the presence of HATU (427 mg, 1.12 mmol) and DMAP (343 mg, 2.81 mmol) in DMF (2 mL) at 25° C. for 2 h to afford 1-(4-(2-hydroxypropan-2-yl)-2,3-dihydro-1H-pyrrolo[3,2-c]pyridin-1-yl)-2-((2-methyl-5-(3-methyl-1,2,4-thiadiazol-5-yl)phenyl)amino)ethan-1-one (example 90) as a white solid. Yield: 9.9%. LC-MS (m/z): 424.13 [M+H]⁺ (Purity: 98%).

1-(4-(2-Hydroxypropan-2-yl)-2,3-dihydro-1H-pyrrolo[2,3-c]pyridin-1-yl)-2-((2-methyl-5-(3-methyl-1,2,4-thiadiazol-5-yl)phenyl)amino)ethan-1-one (Example 91): Treatment of 2-(2,3-dihydro-1H-pyrrolo[2,3-c]pyridin-4-yl)propan-2-ol (SM-91f, 90.00 mg, 5.05 mmol) with (2-methyl-5-(3-methyl-1,2,4-thiadiazol-5-yl)phenyl)glycine (INT-III-1, 133.0 mg, 5.045 mmol) in the presence of T3P (321.3 mg, 1.01 mmol) and DIPEA (195.8 mg, 0.26 mL, 1.52 mmol) in DMF (5 mL) at 25° C. for 16 h to afford 1-(4-(2-hydroxypropan-2-yl)-2,3-dihydro-1H-pyrrolo[2,3-c]pyridin-1-yl)-2-((2-methyl-5-(3-methyl-1,2,4-thiadiazol-5-yl)phenyl)amino)ethan-1-one (example 91) as a white solid. Yield: 4.6%. LC-MS (m/z): 424.13 [M+H]⁺ (Purity: 99%).

### Synthesis of Example 92

Step (a): To a stirred solution of 1-(tert-butyl) 4-ethyl 2,3-dihydro-1H-pyrrolo[3,2-c]pyridine-1,4-dicarboxylate (SM-90a, 3.5 g, 12 mmol, 1 equiv.) in THF (50 mL) was added LiAlH₄ (0.91 g, 9.6 mL, 2.5 M, 24 mmol, 2 equiv.) solution in THF at 0° C. dropwise under N₂ atmosphere and the reaction mixture was stirred at 0° C. for 1 h. The reaction was monitored by checking TLC (EtOAc: Heptane; 80 20) using as an eluent system. The reaction mixture was quenched with sat. Na₂SO₄ soln. (3 mL) at 0° C. and the resulting solution was stirred for 20 min. at 0° C. Then, K₂CO₃ (5 g) was added to the reaction mixture and was stirred again at 0° C. to 25° C. for 20 minutes. The reaction mixture was then diluted with 10% MeOH/EtOAc soln. (200 mL) and was stirred for additional 10 min. and the crude was then filtered off through celite pad and was washed with EtOAc (20 mL). Filtrate was concentrated completely under vacuum afforded SM-92a as oily yellowish semisolid LC-MS (m/z): 251.15 [M+H]+(Purity: 66%).

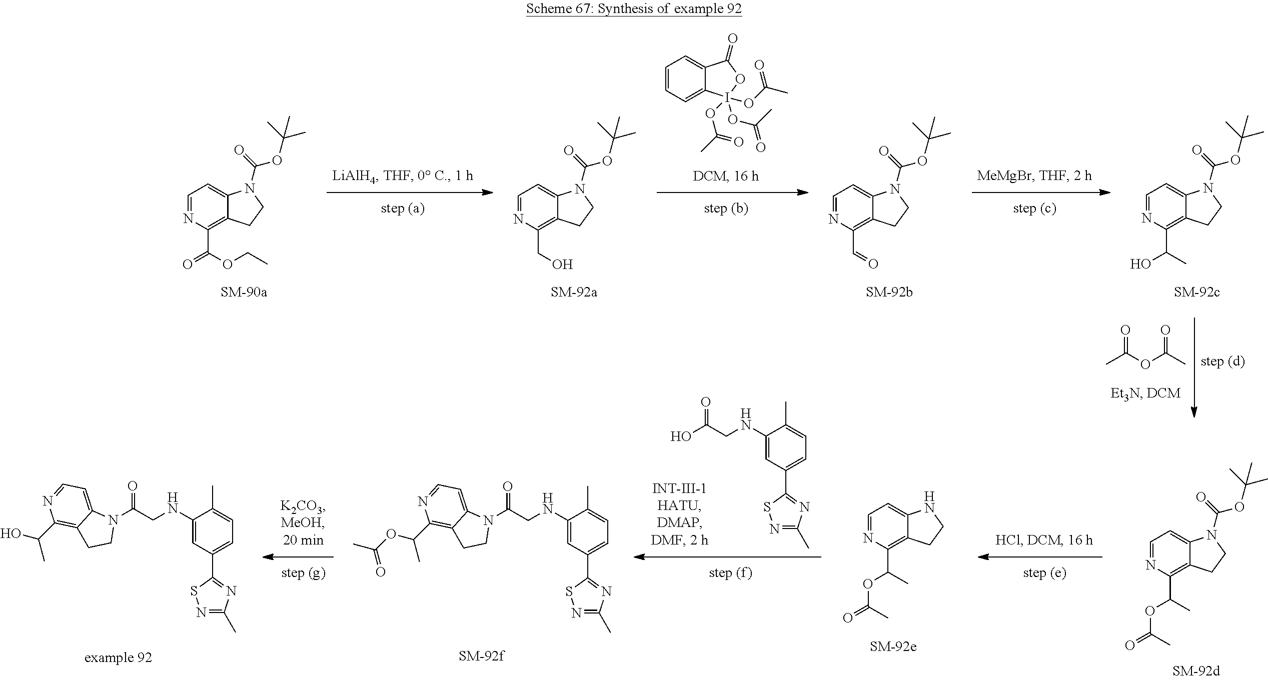

Step (b): To a stirred solution of tert-butyl 4-(hydroxymethyl)-2,3-dihydro-1H-pyrrolo[3,2-c]pyridine-1-carboxylate (SM-92a, 3.0 g, 12 mmol, 1 equiv.) in DCM (60 mL) was added 3-oxo-115-benzo[d][1,2]iodaoxole-1,1,1(3H)-triyl triacetate (7.6 g, 18 mmol, 1.5 equiv.) under N₂ atmosphere at 25° C. and the reaction mixture was then stirred at the same temp. for 16 h. The reaction was monitored by checking TLC (EtOAc: Heptane, 50:50) using as an eluent system. The reaction mixture was filtered through celite pad, and the filtrate was concentrated and directly purified by combi-flash column chromatography using 40 g Combi Column cartridge and compound was eluted by (EtOAc: Heptane 30:70) to afford SM-92b. LC-MS (m/z): 247.86 [M−H]f (Purity: 27%).and the product was proceeded to the next step.

Step (c): To a stirred solution of tert-butyl 4-formyl-2,3-dihydro-1H-pyrrolo[3,2-c]pyridine-1-carboxylate (SM-92b, 400 mg, 1.61 mmol, 1 equiv.) in THF (10 mL) was added MeMgBr (576 mg, 2 mL, 3 M in Et₂O, 4.83 mmol, 3 equiv.) at 0° C. under N₂ atmosphere and the reaction mixture was stirred at 0° C. to 25° C. for 2 h. The reaction was monitored by checking TLC (EtOAc: Heptane, 50:50) using as an eluent system. The reaction mixture was quenched with NH₄Cl sat. soln. (10 mL) at 0° C. and the crude was extracted with EtOAc (3×20 mL). The organic layers were combined, dried over anhyd. Na₂SO₄ and concentrated under vacuum. The crude was purified by combiflash column chromatography and the desired product SM-92c was eluted in 70% EtOAc/Heptane system. LC-MS (m/z): 265.2 [M+H]⁺ (Purity: 84%).

Step (d): To a stirred solution of tert-butyl 4-(1-hydroxyethyl)-2,3-dihydro-1H-pyrrolo[3,2-c]pyridine-1-carboxylate, HCl (SM-92c, 600 mg, 1.99 mmol, 1 equiv.) in DCM (5 mL) was added Et₃N (1.01 g, 1.4 mL, 9.97 mmol, 5 equiv.) followed by acetic anhydride (407 mg, 377 μL, 3.99 mmol, 2 equiv.) at 0° C. under N₂ atmosphere and the reaction mixture was stirred at 0° C. to 25° C. for 1 h. The reaction was monitored by checking TLC (EtOAc: Heptane, 50:50) using as an eluent system. The reaction mixture was concentrated completely under vacuum and the crude was directly purified by combi flash column chromatography. Pure product was eluted in 80% EtOAc/Heptane and analyzed by LCMS and ¹H NMR were consistent with desired product SM-92d. LC-MS (m/z): 307.19 [M+H]⁺ (Purity: 70%).

Step (e): To a stirred solution of tert-butyl 4-(1-acetoxyethyl)-2,3-dihydro-1H-pyrrolo[3,2-c]pyridine-1-carboxylate (SM-92d, 450 mg, 1.47 mmol, 1 equiv.) in DCM (5 mL) was added HCl (536 mg, 4 mL, 4 M, 14.7 mmol, 10 equiv.) at 0° C. under N₂ atmosphere and the reaction mixture was stirred at 0° C. to 25° C. for 16 h. The reaction was monitored by checking TLC (EtOAc: Heptane; 50:50) using as an eluent system. The reaction mixture was concentrated under vacuum and washed with heptane. Solid product was dried well under vacuum to afford SM-92e and used for the next step. LC-MS (m/z): 207.09 [M+H]⁺ (Purity: 88%).

Step (f): To a stirred solution of 1-(2,3-dihydro-1H-pyrrolo[3,2-c]pyridin-4-yl)ethyl acetate (SM-92e, 150 mg, 727 μmol, 1 equiv.) and (2-methyl-5-(3-methyl-1,2,4-thiadiazol-5-yl)phenyl)glycine (192 mg, 727 μmol, 1 equiv.) in DMF (15 mL) was added DMAP (444 mg, 3.64 mmol, 5 equiv.) followed by HATU (553 mg, 1.45 mmol, 2 equiv.) at 0° C. under N₂ atmosphere and the reaction mixture was stirred at 0° C. to 25° C. for 2 h. The reaction was monitored by checking TLC (EtOAc: Heptane; 50:50) using as an eluent system. The reaction mixture was quenched with ice cold H₂O and the crude was crude was extracted with EtOAc (3×20 mL). The organic layers were combined, dried over anhyd. Na₂SO₄ and concentrated under vacuum. The crude was purified by combiflash column chromatography and the desired product SM-92f was eluted in 70% EtOAc/Heptane system. The product was isolated and used for the next step. LC-MS (m/z): 452.11 [M+H]⁺ (Purity: 50%).

Step (g): To a stirred solution of 1-(1-((2-methyl-5-(3-methyl-1,2,4-thiadiazol-5-yl)phenyl)glycyl)-2,3-dihydro-1H-pyrrolo[3,2-c]pyridin-4-yl)ethyl acetate (SM-92f, 100 mg, 221 μmol, 1 equiv.) in MeOH (5 mL) was added K₂CO₃ (153 mg, 1.11 mmol, 5 equiv.) at 0° C. under N₂ atmosphere and the reaction mixture was stirred at 0° C. for 20 min. The reaction was monitored by checking TLC (EtOAc: MeOH, 90:10) using as an eluent system. The reaction mixture was diluted with EtOAc (30 mL) and crude was filtered through celite pad and the filtrate was concentrated and purified by reverse phase column chromatography to afford example 92. Yield: 9%. LC-MS (m/z): 408.01 [M−H]⁺ (Purity: 99%).

### Synthesis Pathway B

In synthesis pathway B, SM4 indole can be modified or build in before STEP9. In STEP9 indole position 3 acylation can be done via Friedel Crafts using e.g. chloroaceyl chloride and AlCl₃ as reagents (X=halogen). In STEP10, INT-I like anilines are alkylated with INT-VI using inorganic bases (like Na₂CO₃ or NaH) or organic bases (like DIPEA or Et₃N) to give Formula B. In STEP11, SM-5 can be directly converted to Formula B using various alkylated agents (Scheme 68). Formula B can be further modified which will be described in the following examples separately if done.

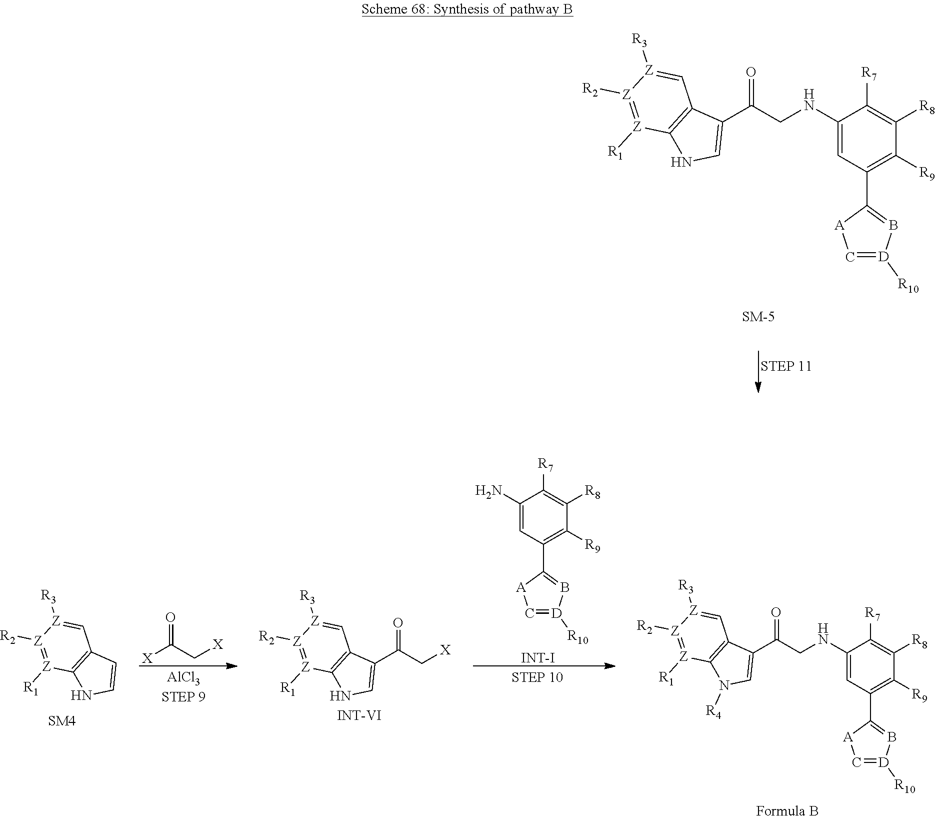

### General Procedure F: Synthesis of INT-VI-(1-7), Formulae Part 8

Chloroacetyl chloride (1 equiv.) was added very slowly over 30 min to a stirred solution of the corresponding indoles (CAS numbers on the scheme 66, 1 equiv.) in dry toluene (15 mL) and pyridine (1 equiv.) at 60° C. under argon atmosphere. After the addition was completed, the reaction mixture was stirred at 60° C. for further 1-4 h. The solution was then cooled to rt and then 20 mL of water and 3 mL of MeOH were added to quench the reaction. The resulted suspension was stirred at rt for 1-4 h. After that time, the suspension was filtered and the obtained solid was washed with water and recrystallized from EtOH to obtain the desired intermediates VI 1-7, Formulae part 8.

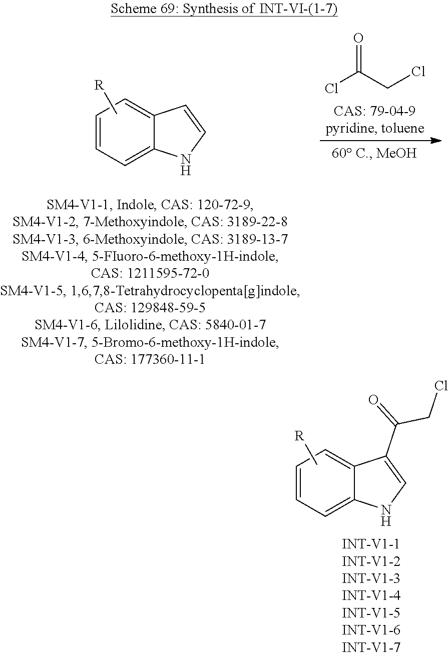

### Preparation of (2-chloroacetyl)-3,4-dihydro-[1,4]diazepino[6,7,1-hi]indol-1(2H)-one (INT-VI-8)

Step (a): A solution of 1H-indole-7-carboxylic acid SM4-VI-8 (2.0 g, 12.4 mmol), 2-chloroethan-1-amine hydrochloride (1.4 g, 12.4 mmol), DIPEA (11.1 mL; 62.5 mmol) and HATU (9.4 g, 24.8 mmol) in DMF (30 mL) was stirred at 25° C. for 16 h then heated at 80° C. for 5 h. Reaction mixture was poured into ice water and extracted with EtOAc (3×30 mL). The combined organic layer was concentrated under reduced pressure and residue was purified by column chromatography on silica gel using 30% EtOAc in hexane to give INT-VI-8a; yield: 70%. LC-MS: m/z 286.12 [M+H]⁺.

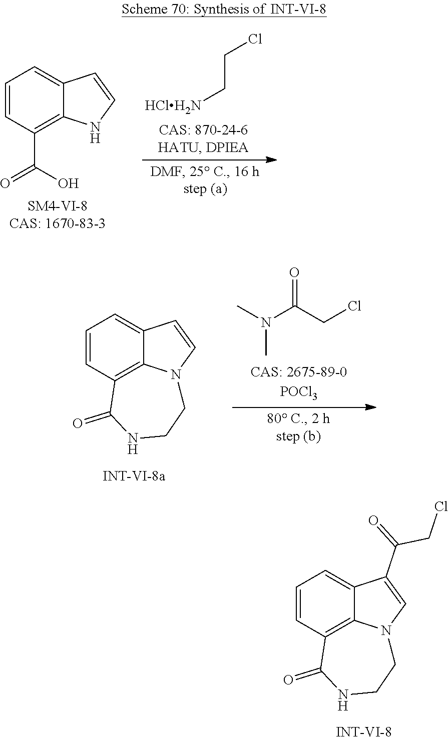

Step (b): A solution of POCl₃ (1 mL, 10.75 mmol) and 2-chloro-N,N-dimethylacetamide (1.2 mL, 8.06 mmol) was stirred under N₂ atmosphere at rt for 10 min. INT-VI-8a (1.0 g, 5.37 mmol) was added to the above mixture and heated at 80° C. for 2 h. The dark brown thick liquid was poured slowly in ice H₂O mixture and pH was adjusted to 8-9 using 1 M aq. NaOH solution and extracted with EtOAc (3×20 mL). Organic layers were dried over anhyd. Na₂SO₄, filtered and concentrated under reduced pressure and the residue was purified by column chromatography on silica gel using 50-60% EtOAc in hexane to give INT-VI-8.

### Preparation of 2-chloro-1-(3-hydroxy-3-methyl-3,4-dihydro-2H-[1,4]oxazepino[2,3,4-hi]indol-7-yl)ethan-1-one (INT-VI-9)

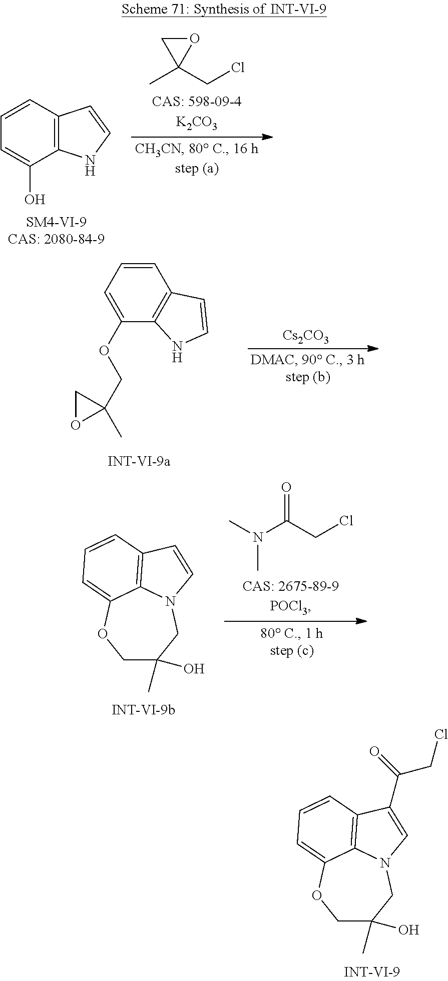

Step (a): A solution of 1H-indol-7-ol SM4-VI-9 (0.5 g, 3.75 mmol), K₂CO₃ (1.4 g, 11.3 mmol) and 2-(chloromethyl)-2-methyloxirane (0.60 g, 5.63 mmol) in CH₃CN (30 mL) was stirred at 80° C. for 16 h. The reaction mixture was diluted with H₂O (50 mL) and extracted with EtOAc (2×50 mL). The organic layer was dried over anhyd. Na₂SO₄, filtered and concentrated under reduced pressure. The residue was purified by column chromatography on silica gel using (EtOAc: hexane, 30:70) to give INT-VI-9a as an off white solid; yield: 0.4 g, 53%. LC-MS: m/z 202.11 [M−H]⁺.

Step (b): A solution of INT-VI-9a (0.3 g, 1.47 mmol) and Cs₂CO₃ (0.96 g, 2.95 mmol) in N,N-dimethylacetamide (2.5 mL) was heated at 80° C. for 2 h. The reaction mixture was diluted with H₂O (30 mL) and extracted with EtOAc (2×30 mL). The organic layer was dried over anhyd. Na₂SO₄, filtered and concentrated under reduced pressure to give INT-VI-9b as an off white solid; yield: 0.25 g, 83%. LC-MS: m/z 204.06 [M+H]⁺.

Step (c): A solution of INT-VI-9b (0.15 g, 0.73 mmol), 2-chloro-N,N-dimethylacetamide (0.162 g, 1.1 mmol) and POCl₃ (0.15 g, 1.1 mmol) was stirred at 80° C. in a 30 mL sealed tube for 1 h. Reaction mixture was poured in ice water mixture and pH was made basic using sat. NaHCO₃ solution and extracted with EtOAc (3×25 mL). The organic layer was dried over anhyd Na₂SO₄, filtered and concentrated under reduced pressure. The residue was triturated with (DCM: hexane, 10:90) and dried to give INT-VI-9 as a pale brown solid.

### General Procedure G: Synthesis of Intermediates INT-VI-(10-14), Formulae Part 8

A suspension of the corresponding indole/azaindole (CAS numbers on scheme 69, 1 equiv.) and AlCl₃ (2.5-5 equiv.) in DCM (50 mL) was stirred at rt for 1 h. The reaction mixture was cooled to 0° C., a solution of chloroacetyl chloride in 20 mL of DCM was added dropwise, and the reaction mixture was stirred for 2-18 h. Then, MeOH (15-50 mL) was carefully added to quench the reaction and then the solvents were removed under reduced pressure. The residue was dissolved in 100 mL of a sat. NaHCO₃ soln. and extracted with EtOAc (3×50 mL) and dried under vacuum. The resulted residue was crystallized from (hexane: EtOAc, 60:40) to obtain intermediates INT-VI-(10-14), Formulae part 8.

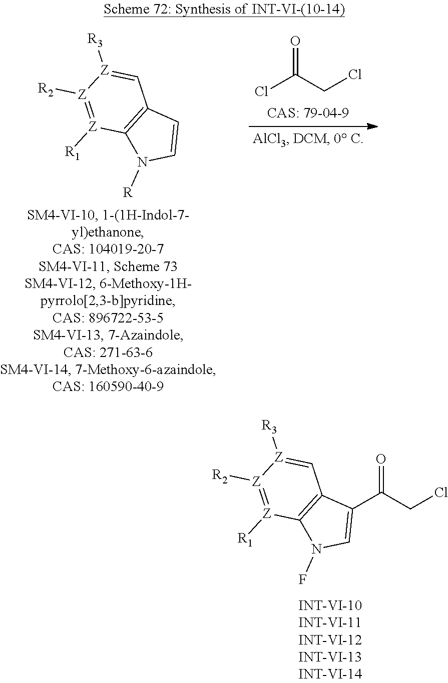

### Synthesis of 5-fluoro-6-methoxy-1-methyl-1H-indole (precursor of SM4-VI-11)

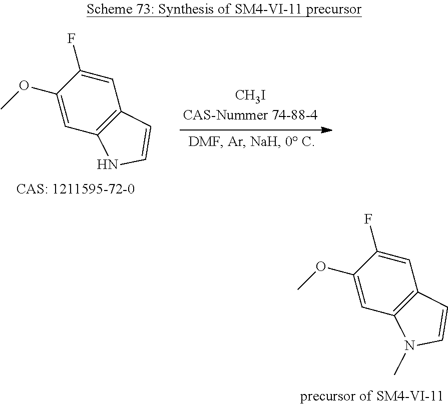

In a 10 mL round bottom flask fitted with a stirrer was loaded with 5-fluoro-6-methoxy-1H-indole (247.5 mg, 1.5 mmol) and 3 mL anhydrous DMF under argon. The solution was cooled to 0° C. in an ice bath. Iodomethane (255.5 mg, 112.1 μL, 1.8 mmol, 1.2 equiv.) was then added followed by the portionwise addition of NaH (60% suspension in mineral oil, 54 mg, 2.25 mmol, 1.5 equiv.) over a period of 15 min. Reaction was quenched after 60 h by addition to an ice bath then extracted with diethyl ether. The combined organic layers were washed with brine (1×20), dried over MgSO₄, filtered off, and the solvent was removed in vacuo giving the desired compound. LC-MS (m/z): 208.1 [M+H]⁺. Yield: 100%.

### Preparation of 2-methyl-5-(5-methyl-1,2,4-oxadiazol-3-yl)aniline (INT-I-22, Formulae part 2)

Step (a): In a 50 mL round flask, 3-amino-4-methylbenzonitrile (SM3-I-22, 1 g, 7.6 mmol) was dissolved in EtOH (50 mL) and treated with hydroxylamine hydrochloride (1.05 g, 15.1 mmol) and Na₂CO₃ (802 mg, 7.6 mmol). The mixture was stirred at rt for 10 min. and then H₂O (3.2 mL) was added. The reaction was stirred at rt for overnight. The solvent was removed under reduced pressure. Water (100 mL) was added and the formed precipitate was filtered, washed with H₂O and PE to afford (E,Z)-3-amino-N-hydroxy-4-methylbenzimidamide SM3-I-22a.

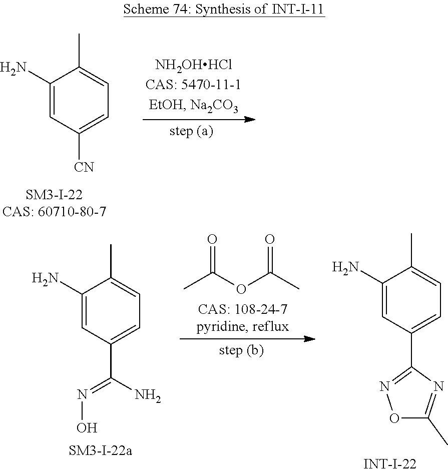

Step (b): SM3-I-22a (200 mg, 1.2 mmol) was dissolved in dry pyridine (2 mL) and treated with a solution of acetic anhydride (114 μL, 1.2 mmol) in pyridine (2 mL). The reaction was heated to reflux and stirred for overnight. After that time, the reaction was allowed to reach rt and the solvent was removed under reduced pressure. The final compound was isolated after flash chromatography in silica gel and (cyclohexane:EtOAc, 70:30) as an eluent (150 mg, 66% yield). LC-MS (m/z): 190.20 [M+H]⁺. Purity by HPLC-UV (254 nm)-ESI-MS: 95.8%.

TABLE 3
Characterization of building blocks INT-I-(22-23), Scheme 68, Formulae part 2

| | | | Appearance in |
|---|---|---|---|
| INT | | ¹H-NMR and/or MS data | the final |
| number | Structure | (Purity and/or Yield) | examples |
| I-22 | 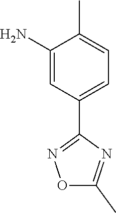 | ¹H-NMR (500 MHz, DMSO-d₆): δ 9.35 (s, 1H), 6.94 (d, 1H), 6.92 (d, 1H), 6.78 (dd, 1H), 5.52 (s, 1H), 4.82 (s, 1H), 2.07 (s, 2H). LC-MS: (m/z): 166.15 [M + H]⁺. (Purity: 96.8%. Yield: 88%) | 106 |
| I-23 | 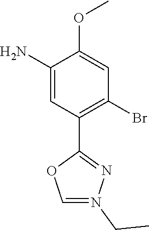 | ¹H-NMR (500 MHz; DMSO-d₆): δ 7.31 (s, 1H), 7.15 (s, 1H), 5.28 (s, 2H), 3.87 (s, 3H), 2.77 (q, 2H), 1.27 (t, 3H). LC-MS: (m/z): 299 [M + H]⁺. (Purity: 98.4%. Yield: 20%). | 108, 109 |

### Synthesis of 4-bromo-5-(3-ethyl-1,2,4-oxadiazol-5-yl)-2-methoxyaniline (INT-I-23, Formulae part 2)

In 50 mL round flask, NBS (356 mg, 2 mmol) was added portionwise to a stirred solution of 5-(3-ethyl-1,2,4-oxadiazol-5-yl)-2-methoxyaniline (INT-I-17, 439 mg, 2 mmol) in 10 mL dry DMF at 0° C. under argon away from light. The reaction mixture was allowed to stir at rt for 18 h. The reaction mixture was poured into cold water then extracted with EtOAc (3×50 mL). The combined organic layers were washed with brine (20 mL) and then was dried over MgSO₄, filtered, and concentrated in vacuum. Purification was done using column chromatography on silica gel using DCM: EtoAc, 90:10.

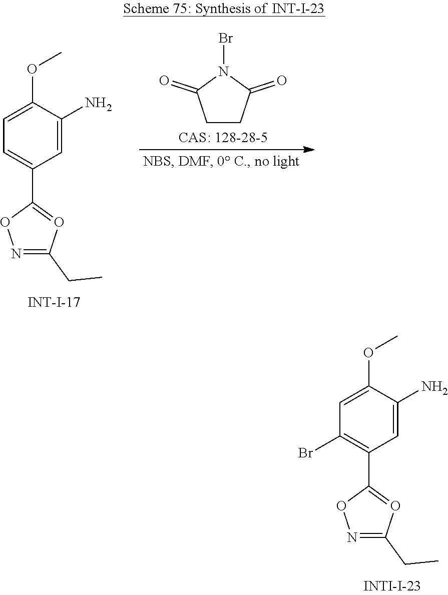

### Synthesis Pathway B, General Method F

A solution of the appropriate aniline (2 equiv.) in acetone (30 mL) was mixed with the appropriate acyl(aza)indole/indoline (1 equiv.), potassium iodide (2 equiv.) and DIPEA (3 equiv.). The reaction mixture was heated to reflux while stirring for 5-18 h. After the reaction was completed, the mixture was allowed to reach rt and the solvent was removed under reduced pressure. Water (50 mL) was then added and the product was extracted with EtOAc (3×50 mL). The combined organic extracts were washed with 1 M HCl (3×50 mL), then with brine (2×50 mL) and dried over MgSO₄ or Na₂SO₄, filtered, and concentrated under vacuum. The reaction mixture was subjected to chromatography purifications on silica gel to obtain the pure products.

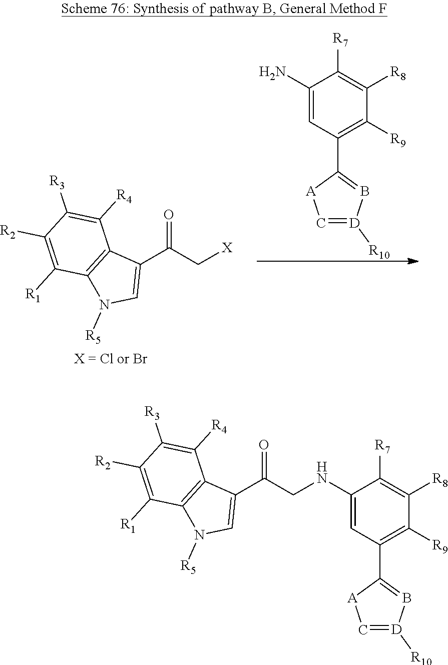

The following examples have been synthesized following Pathway B:

TABLE 4
Reaction and purification conditions of the final examples 90-107 synthesized via STEP
10, scheme 68 (Pathway B)

| | | INT-VI | INT-I | Synthesis | |
|---|---|---|---|---|---|
| | | (Formulae | (Formulae | method | Purification |
| Example | Structure | part 7) | part 2) | (Yield %) | Method |
| 93 | 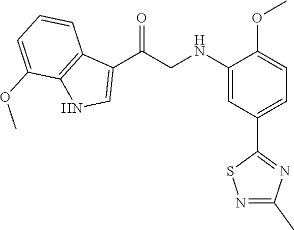 [SMILES: COc1ccc(-c2nc(C)ns2)cc1NCC(=O)c1c[nH]c2c(OC)cccc12] | INT-VI-2 | INT-I-12 | F (25) | I |
| 94 | 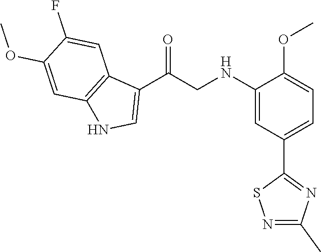 | INT-VI-4 | INT-I-12 | F (43) | I recrystallization (MeOH:H₂O, 1:1) |
| 95 | 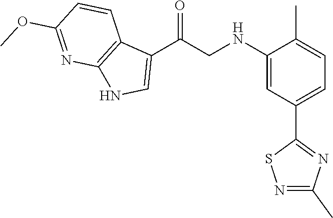 | INT-VI-13 | INT-I-1 | F (20) | I |
| 96 | 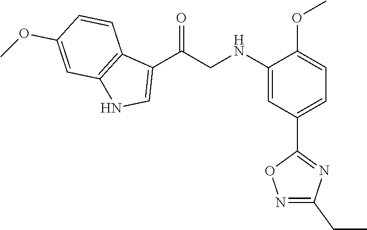 [SMILES: CCc1noc(-c2ccc(OC)c(NCC(=O)c3c[nH]c4cc(OC)ccc34)c2)n1] | INT-VI-3 | INT-I-17 | F (42) | I |
| 97 | 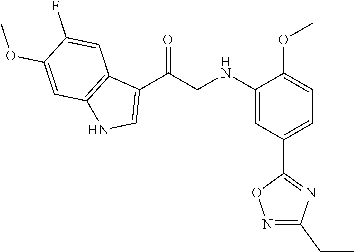 [SMILES: CCc1noc(-c2ccc(OC)c(NCC(=O)c3c[nH]c4cc(OC)c(F)cc34)c2)n1] | INT-VI-4 | INT-I-17 | F (35) | I; recrystallization (cyclohexane: EtOAc 8:2) |
| 98 | 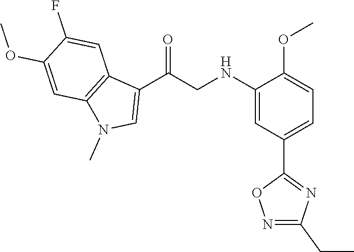 [SMILES: CCc1noc(-c2ccc(OC)c(NCC(=O)c3cn(C)c4cc(OC)c(F)cc34)c2)n1] | INT-VI-11 | INT-I-17 | F (6.8) | I |
| 99 | 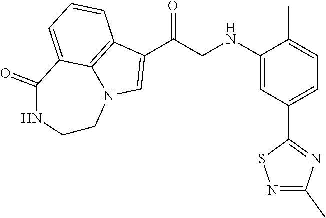 [SMILES: Cc1nsc(-c2ccc(C)c(NCC(=O)c3cn4c5c(cccc35)C(=O)NCC4)c2)n1] | INT-VI-8 | INT-I-1 | F (18) | purified by reverse phase prep HPLC; R_{f} (EtOAc:heptane, 9:1) = 0.3 |
| 100 | 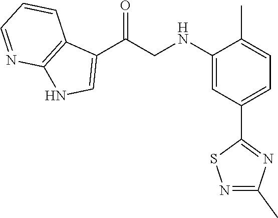 [SMILES: Cc1nsc(-c2ccc(C)c(NCC(=O)c3c[nH]c4ncccc34)c2)n1] | INT-VI-12 | INT-I-1 | F (16) | II |
| 101 | 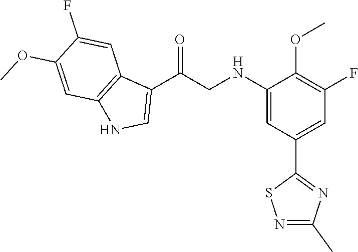 [SMILES: COc1cc2[nH]cc(C(=O)CNc3cc(-c4nc(C)ns4)cc(F)c3OC)c2cc1F] | INT-VI-4 | INT-I-13 | F (15) | I; recrystallization (MeOH:H₂O, 1:1) |
| 102 | 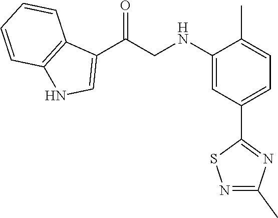 [SMILES: Cc1nsc(-c2ccc(C)c(NCC(=O)c3c[nH]c4ccccc34)c2)n1] | INT-VI-1 | INT-I-1 | F (23) | I crystallization (cyclohexane: EtOAc, 3:1) |
| 103 | 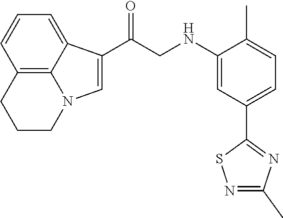 [SMILES: Cc1nsc(-c2ccc(C)c(NCC(=O)c3cn4c5c(cccc35)CCC4)c2)n1] | INT-VI-6 | INT-I-1 | F (26) | I; recrystallized from EtOAc then washed with DEE |
| 104 | 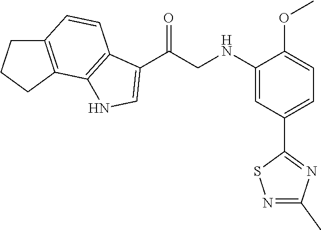 [SMILES: COc1ccc(-c2nc(C)ns2)cc1NCC(=O)c1c[nH]c2c3c(ccc12)CCC3] | INT-VI-5 | INT-I-12 | F (43) | I; recrystallization (MeOH:H₂O, 1:1) |
| 105 | 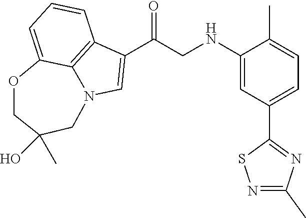 [SMILES: Cc1nsc(-c2ccc(C)c(NCC(=O)c3cn4c5c(cccc35)OCC(C)(O)C4)c2)n1] | INT-VI-8 | INT-I-1 | F; NaI, not KI; NaHCO₃, not DIPEA; CH₃CN not acetone (15) | I; R_{f} (EtOAc: heptane, 5:5) = 0.2; fFollowed by trituration with Et₂O and drying |
| 106 | 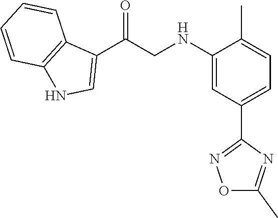 [SMILES: Cc1nc(-c2ccc(C)c(NCC(=O)c3c[nH]c4ccccc34)c2)no1] | INT-VI-1 | INT-I-22 | F (24) | I |
| 107 | 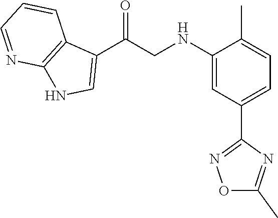 [SMILES: Cc1nc(-c2ccc(C)c(NCC(=O)c3c[nH]c4ncccc34)c2)no1] | INT-VI-12 | INT-I-14 | F (26) | I; (DCM:EtOH, 9:1) and crystallization with (EtOAc) |
| 108 | 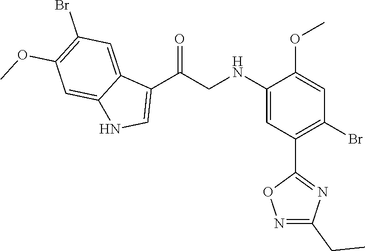 [SMILES: CCc1noc(-c2cc(NCC(=O)c3c[nH]c4cc(OC)c(Br)cc34)c(OC)cc2Br)n1] | INT-VI-7 | INT-I-23 | F (20) | (DCM:EtOAc, 90:10), then crystallization (cyclohexane: EtOAc, 3:1) |
| 109 | 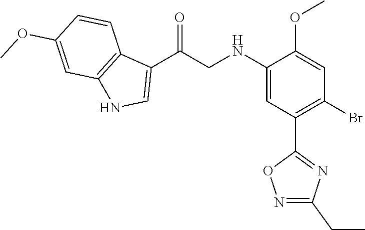 [SMILES: CCc1noc(-c2cc(NCC(=O)c3c[nH]c4cc(OC)ccc34)c(OC)cc2Br)n1] | INT-VI-3 | INT-I-23 | F (58) | (EtOAc:DCM 10:90), recrystallization (cyclohexane:EtOAc, 3:1) |
| 110 | 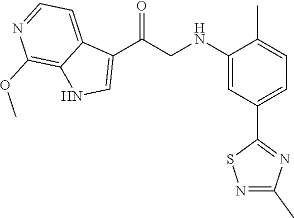 [SMILES: COc1nccc2c(C(=O)CNc3cc(-c4nc(C)ns4)ccc3C)c[nH]c12] | INT-VI-14 | INT-I-1 | F | II |

### Synthesis of Example III

Step (a): DIPEA (2.25 mmol, 400 L) and KI (0.75 mmol, 125 mg) were added to a solution of 2-chloro-1-(1H-indol-3-yl)ethan-1-one (INT-VI-1, 0.75 mmol, 145 mg) and 5-(3-ethyl-1,2,4-oxadiazol-5-yl)-2-methyl-aniline (INT-I-15, 1.5 mmol, 305 mg) in acetone (25 mL) and the resulting solution was stirred at reflux for 16 h. After that time 30 mL of H₂O were added and the resulting solution was extracted with EtOAc (3×25 mL). The combined organic layers were washed with brine (30 mL) and dried over MgSO₄. After flash chromatography (PE: EtOAc, 70:30 to 50:50) and crystallization (cyclohexane: ethyl acetate, 3:1).The product 2-((5-(3-Ethyl-1,2,4-oxadiazol-5-yl)-2-methylphenyl)amino)-1-(1H-indol-3-yl)ethan-1-one was obtained (SM-111a) as pale yellow solid; (yield: 31%). LC-MS (m/z): 361 [M+H]⁺; Purity by HPLC-UV (254 nm)-ESI-MS: 98.6%.

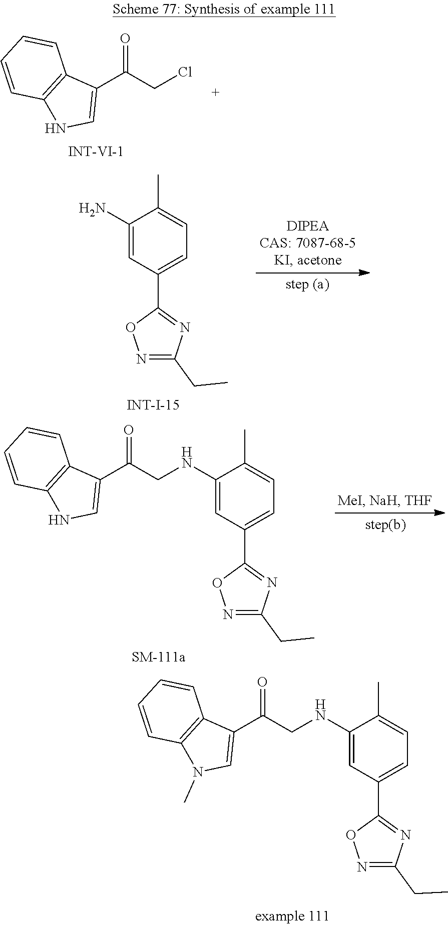

Step (b): In a round flask, NaH 60% in mineral oil (0.18 mmol, 7.2 mg) was added portion wise to a solution of SM-111a (0.18 mmol, 65 mg) in THF (2 mL) at 0° C. under N₂ atmosphere. After stirring for 15 min at rt, Mel (0.22 mmol, 14 L) was added and the resulting mixture was stirred at rt for 2 h. After the reaction was completed, water (30 mL) were added to the reaction mixture and the resulting solution was extracted with EtOAc (3×20 mL). The combined organic layers were washed with brine, dried over MgSO₄ and evaporated. After flash chromatography (PE: EtOAc, 60:40 to 40:60) followed by crystallization with (cyclohexane:EtOAc, 3:1) to afford example III as pale yellow solid; (yield: 40%). LC-MS (m/z): 375 [M+H]⁺; Purity by HPLC-UV (254 nm)-ESI-MS: 98.7%.

### Preparation of Example 112

Step (a): DIPEA (2.25 mmol, 400 L) and KI (0.75 mmol, 125 mg) which were added to a solution 1-(7-acetyl-1H-indol-3-yl)-2-chloroethan-1-one (INT-VI-10, 0.75 mmol, 176 mg) and 2-methyl-5-(3-methyl-1,2,4-thiadiazol-5-yl)aniline (INT-I-1, 1.5 mmol, 307.5 mg) in acetone (25 mL) and the resulting solution was stirred at reflux for 16 h. After that time 30 mL of water were added and the resulting solution was extracted with EtOAc (3×25 mL). The combined organic layers were washed with brine (30 mL) and dried over MgSO₄. After flash chromatography (DCM:EtOAc, 95:5) and crystallization (cyclohexane:EtOAc, 3:1) to afford SM-112a as pale yellow solid (yield: 55%). LC-MS (m/z): 405.1 [M+H]⁺; Purity by HPLC-UV (254 nm)-ESI-MS: 99%.

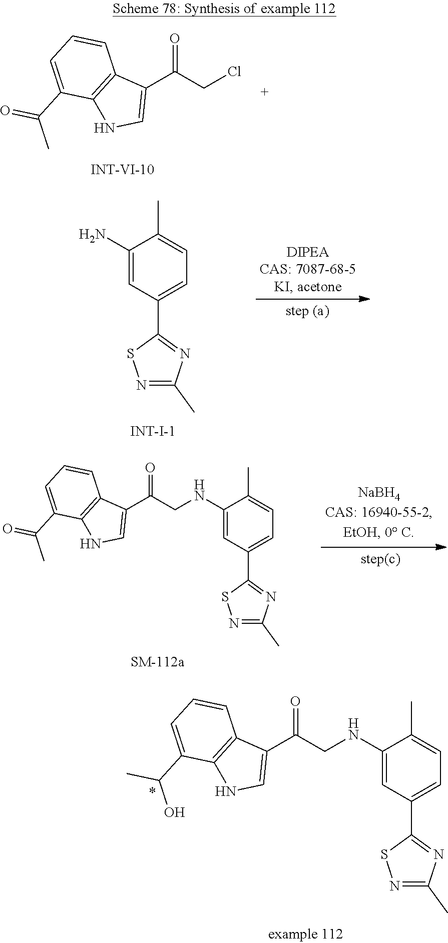

Step (b): Addition of 1-(7-acetyl-1H-indol-3-yl)-2-((2-methyl-5-(3-methyl-1,2,4-thiadiazol-5-yl)phenyl)amino)ethan-1-one (SM-112a, 1 equiv.) at 0° C. to a stirred suspension of NaBH₄ (1.5 equiv.) in 6 mL of EtOH and the resulting mixture was allowed to warmed up and stirred at rt for 4 h. After the reaction was completed and the solvent was concentrated in vacuo, a mixture of (EtOH: H₂O, 1:1, 10 mL) was added. After the reaction was completed, H₂O (30 mL) was added to the reaction mixture and the resulting solution was extracted with EtOAc (3×20 mL). The combined organic layers were washed with brine, dried over MgSO₄ and evaporated. After flash chromatography (PE: EtOAc, 60:40 to 40:60) and crystallization (cyclohexane:EtOAc, 3:1) example 112 was obtained as a white solid; (yield: 35%). LC-MS (m/z): 407 [M+H]⁺; Purity by HPLC-UV (254 nm)-ESI-MS: 98.9%.

### Synthesis Pathway C

In the synthetic pathway C, SM6 or SM7 can be modified before STEP12 and STEP14. In STEP12 and STEP14, C—C coupling can be done via Suzuki or Negishi coupling of boronic acid or boronic acid ester (SM6 or SM8) with halogenated 6-membered heterocycle SM8. Alternatively, the chemistry can be inverted vice versa and convert SM6 or SM7 to boronic acid or boronic acid ester and utilize 2-halogen in pyridyl/used heterocycle. In STEP13 nitro reduction to aniline is following Pathway A (Scheme 1), conditions specified below in given examples. The alkylation of STEP17 aniline can be done with typical alkylating agentes like alpha halogenated alkyl esters. The hydrolysis of STEP18 ester is performed typically with inorganic bases like LiOH or NaOH. In STEP19 amidation, several amidation methods can be used like EDCI/DIPEA, HOBT/DIPEA but mostly done using mild T3P solution in EtOAc or DMF to afford Formula C (Scheme 79). SM4 indole can be modified or build in before STEP15. In STEP15 indole position 3 acylation can be done via Friedel Crafts using e.g. chloroaceyl chloride and AlCl₃ as reagents (X=halogen). In STEP16, INT-I like anilines are alkylated with INT-VI using inorganic bases (like Na₂CO₃ or NaH) or organic bases (like DIPEA or Et₃N) to afford Formula D. Formulas C and D can be further derivatized which is decrypted in upcoming examples, if modified.

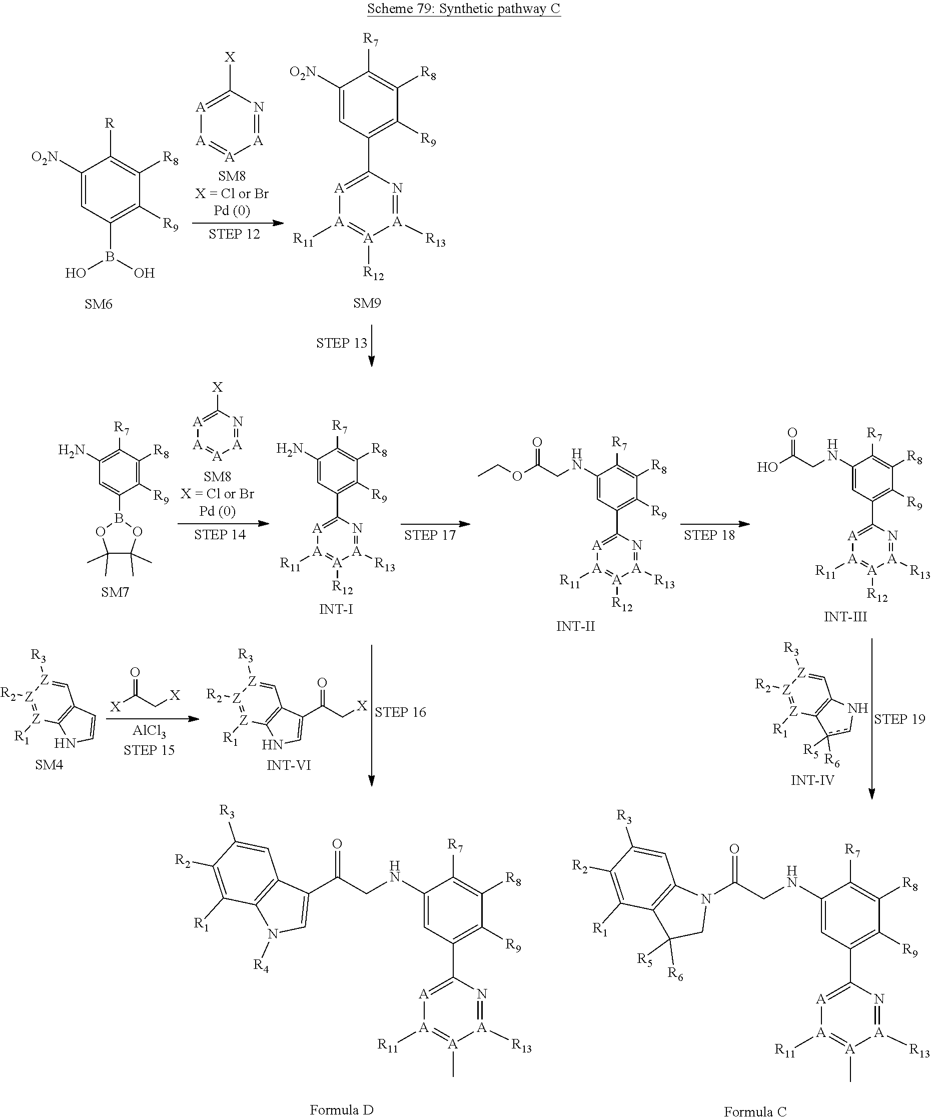

The following examples have been synthesized following pathway C:

### Synthesis of intermediates INT-III-17, INT-III-18, INT-III-19, INT-III-20, INT-III-21, and INT-III-22

### Step (a): General procedure for the synthesis of 3-(pyridin-2-yl)aniline derivatives INT-I-27, INT-I-28, INT-1I-21a, INT-I-25, INT-I-26, and INT-I-29

A stirred solution of 3-(4,4,5,5-tetramethyl-1,3,2-dioxaborolan-2-yl)aniline derivatives (1.1 equiv.) and halopyridinyl derivatives (1.0 equiv.) in the presence of Na₂CO₃ (3.0 equiv.) in EtOH: H₂O (10:1, 33 mL) are phased together in a round flask and the reaction mixture was degassed with nitrogen for 5 min. After 5 min, Pd catalyst ([1,1′-bis(diphenylphosphino)ferrocene]dichlopalladium dichloromethane complex or tetrakis(triphenylphosphine)palladium(0)) (0.01 equiv.) was added. The reaction mixture was stirred at 100° C. After completion of the reaction as indicated by TLC, the reaction mixture was diluted with ice-cold water and extracted with EtOAc (2×500 mL). The combined organic layer was washed with brine (3×200 mL), dried over Na₂SO₄ and concentrated under reduced pressure. This crude residue was purified by column chromatography to afford the corresponding amine derivatives.

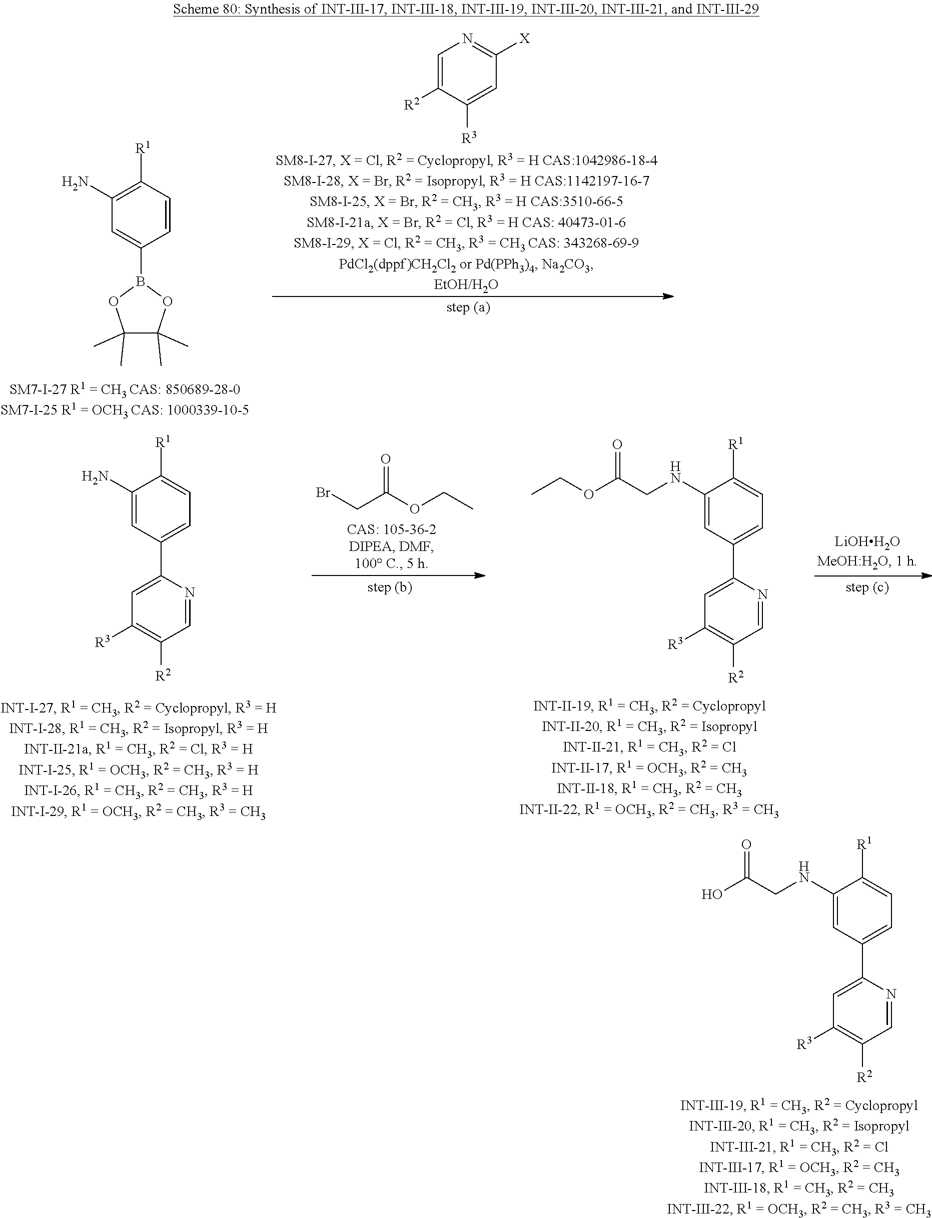

5-(5-Cyclopropylpyridin-2-yl)-2-methylaniline (INT-I-27): Treatment of 2-chloro-5-cyclopropylpyridine (SM8-I-27, 2.2 g, 9.44 mmol) with 2-methyl-5-(4,4,5,5-tetramethyl-1,3,2-dioxaborolan-2-yl)aniline (SM7-I-27, 1.5 g, 10.4 mmol) in the presence of Na₂CO₃ (3.0 g, 28.3 mmol) and Pd(PPh₃)₄(1.09 g, 0.94 mmol) in EtOH: H₂O (10:1, 33 mL) at 100° C. for 16 h followed by extraction and purification by purified by column chromatography using 0-50% EtOAc: heptane as an eluent to afford 5-(5-cyclopropylpyridin-2-yl)-2-methylaniline INT-I-27 as a yellow sticky solid. R_{f} (EtOAc: heptane, 50:50)=0.2; yield: 1.5 g, 64%. LC-MS (m/z): 225.04 [M+H]⁺.

5-(5-Isopropylpyridin-2-yl)-2-methylaniline (INT-I-28): Treatment of 2-bromo-5-isopropylpyridine (SM8-I-28, 1.89 g, 9.44 mmol) with 2-methyl-5-(4,4,5,5-tetramethyl-1,3,2-dioxaborolan-2-yl)aniline (SM7-I-27, 1.5 g, 10.4 mmol) in the presence of Na₂CO₃ (3.0 g, 28.3 mmol) and Pd(PPh₃)₄(1.09 g, 0.94 mmol) in EtOH: H₂O (10:1, 33 mL) at 100° C. for 16 h followed by extraction and purification by purified by column chromatography using 0-50% EtOAc: heptane as an eluent to afford ethyl (5-(5-isopropylpyridin-2-yl)-2-methylphenyl)glycinate (INT-I-28) as a yellow solid that used for the next step.

5-(5-Chloropyridin-2-yl)-2-methylaniline (INT-1I-21a): Treatment of 2-bromo-5-chloropyridine (SM8-I-21a, 2.9 g, 15.02 mmol) with 2-methyl-5-(4,4,5,5-tetramethyl-1,3,2-dioxaborolan-2-yl)aniline (SM7-I-27, 3.5 g, 15.02 mmol) in the presence of Na₂CO₃ (4.0 g, 37.55 mmol) and Pd(PPh₃)₄(1.7 g, 1.5 mmol) in EtOH: H₂O (20:3, 23 mL) at 90° C. for 16 h followed by extraction and purification by purified by column chromatography using 0-20% EtOAc: heptane as an eluent to afford ethyl (5-(5-chloropyridin-2-yl)-2-methylphenyl)glycinate (INT-1I-21a) as a yellow solid yield: 1.7 g, 53% that used for the next step.

2-Methoxy-5-(5-methylpyridin-2-yl)aniline (INT-I-25): Treatment of 2-bromo-5-methylpyridine (SM8-I-25, 4.33 g, 24.09 mmol) with 2-methoxy-5-(4,4,5,5-tetramethyl-1,3,2-dioxaborolan-2-yl)aniline (SM7-I-25, 5 g, 20.00 mmol) in the presence of Na₂CO₃ (6.36 g, 60 mmol) and Pd(PPh₃)₄ (2.31 g, 2.0 mmol) in EtOH: H₂O (20:3, 23 mL) at 90° C. for 6 h followed by extraction and purification by purified by column chromatography using 0-20% EtOAc: heptane as an eluent to afford ethyl (2-methoxy-5-(5-methylpyridin-2-yl)phenyl)glycinate (INT-I-25) as a yellow oil yield: 3.0 g, 70% 2-Methyl-5-(5-methylpyridin-2-yl)aniline (INT-I-26): Treatment of 2-bromo-5-methylpyridine (SM8-I-25, 7.3 g, 42.91 mmol) with 2-methyl-5-(4,4,5,5-tetramethyl-1,3,2-dioxaborolan-2-yl)aniline (SM7-I-27, 10 g, 42.91 mmol) in the presence of Na₂CO₃ (11.3 g, 107.2 mmol) and Pd(PPh₃)₄(4.9 g, 4.2 mmol) in EtOH: H₂O (40:6, 46 mL) at 90° C. for 16 h followed by extraction and purification by purified by column chromatography using 0-30% EtOAc: heptane as an eluent to afford ethyl (5-(5-chloropyridin-2-yl)-2-methylphenyl)glycinate (INT-I-26) as a yellow solid yield: 3 g, 35% that used for the next step.

5-(4,5-Dimethylpyridin-2-yl)-2-methoxyaniline (INT-I-29): Treatment of 2-bromo-4,5-dimethylpyridine (SM8-I-29, 285 mg, 2.05 mmol) with 2-methoxy-5-(4,4,5,5-tetramethyl-1,3,2-dioxaborolan-2-yl)aniline (SM7-I-25, 500 g, 2.05 mmol) in the presence of Na₂CO₃ (530 mg, 5.00 mmol) and Pd(PPh₃)₄ (2.3 g, 0.22 mmol) in dioxane: H₂O (5:0.1, 5.1 mL) at 90° C. for 6 h followed by extraction and purification by purified by column chromatography using 0-20% EtOAc: heptane as an eluent to afford ethyl (2-methoxy-5-(5-methylpyridin-2-yl)phenyl)glycinate (INT-I-29) as a yellow oil yield: 300 mg, 87% that used for the next step.

### Step (b): General procedure for the synthesis of ethyl (3-(pyridin-2-yl)phenyl)glycinate INT-1I-19, INT-1I-20, INT-1I-21, INT-1I-17, INT-1I-18 and INT-1I-22

To a stirred solution of 3-(pyridin-2-yl)aniline derivatives (1.0 equiv.) in DMF were added DIPEA (2.0 equiv.) and ethyl 2-bromoacetate (1.1 equiv.) under N₂ atmosphere. This reaction mixture was stirred at 100° C. for 16 h. After completion of the reaction as indicated by TLC, reaction mixture was diluted with ice cold water and extracted with EtOAc (3 times). The combined organic layer was washed with ice cold brine and dried over anhy. Na₂SO₄, filtered and concentrated under reduced pressure. This crude residue was purified by column chromatography to afford the corresponding ethyl carboxylates.

Ethyl (5-(5-cyclopropylpyridin-2-yl)-2-methylphenyl)glycinate (INT-II-19): Treatment of 5-(5-cyclopropylpyridin-2-yl)-2-methylaniline (INT-I-27, 1.5 g, 6.69 mmol) with ethyl 2-bromoacetate (1.1 mL, 10 mmol) in the presence of DIPEA (5.75 mL, 33.5 mmol) in DMF (40 mL) at 100° C. for 5 h followed by extraction and purification by column chromatography using 0-30% EtOAc: heptane as an eluent to afford ethyl (5-(5-cyclopropylpyridin-2-yl)-2-methylphenyl)glycinate INT-II-19 as a yellow sticky solid. R_{f}(EtOAc: heptane, 50:50)=0.5; yield: 1.2 g, 58%. LC-MS (m/z): 311.18 [M+H]⁺.

Ethyl (5-(5-isopropylpyridin-2-yl)-2-methylphenyl)glycinate (INT-II-20): Treatment of 5-(5-isopropylpyridin-2-yl)-2-methylaniline (INT-I-28, 1.3 g, 5.75 mmol) with ethyl 2-bromoacetate (1.4 g, 8.62 mmol) in the presence of DIPEA (5.1 mL, 28.75 mmol) in DMF (30 mL) at 100° C. for 5 h followed by extraction and purification by column chromatography using 20-25% EtOAc: heptane as an eluent to afford ethyl (5-(5-isopropylpyridin-2-yl)-2-methylphenyl)glycinate INT-II-20 as a yellow semisolid. yield: 700 mg, 39% that used in the next step.

Ethyl (2-methyl-5-(5-methylpyridin-2-yl)phenyl)glycinate (INT-II-21): Treatment of 5-(5-chloropyridin-2-yl)-2-methylaniline (INT-II-21a,1.3 g, 5.96 mmol) with ethyl 2-bromoacetate (1.5 g, 8.94 mmol) in the presence of DIPEA (3.8 g, 29.8 mmol) in DMF (20 mL) at 100° C. for 16 h followed by extraction and purification by column chromatography using 15-20% EtOAc: heptane as an eluent to afford ethyl (2-methyl-5-(5-methylpyridin-2-yl)phenyl)glycinate INT-II-21 as a off white solid. ; yield: 800 mg, 44% that used in the next step.

Ethyl (2-methoxy-5-(5-methylpyridin-2-yl)phenyl)glycinate (INT-II-17): Treatment of 2-methoxy-5-(5-methylpyridin-2-yl)aniline (INT-I-25, 0.98 g, 4.57 mmol) with ethyl 2-bromoacetate (0.76 mL, 6.86 mmol) in the presence of DIPEA (1.593 mL, 9.15 mmol) in DMF (5 mL) at 60° C. for 2 h under N₂ atmosphere. The reaction was then cooled down to rt, EtOAc was added and washed with H₂O (50 mL) to remove DMF. The organic phase was dried and evaporated to dryness to obtain 1.135 g of ethyl 2-((2-methoxy-5-(5-methylpyridin-2-yl)phenyl)amino)acetate (INT-II-17).

Ethyl (2-methyl-5-(5-methylpyridin-2-yl)phenyl)glycinate (INT-II-18): Treatment of 2-methyl-5-(5-methylpyridin-2-yl)aniline (INT-I-26, 3 g, 15.15 mmol) with ethyl 2-bromoacetate (3.8 g, 22.72 mmol) in the presence of DIPEA (14 mL, 75.75 mmol) in DMF (60 mL) at 60° C. for 2 h under N₂ atmosphere. The reaction was then cooled down to rt, EtOAc was added and washed with H₂O (50 mL) to remove DMF. The organic phase was dried and evaporated to dryness to obtain 2.1 g (49% yield) of ethyl (2-methyl-5-(5-methylpyridin-2-yl)phenyl)glycinate (INT-II-18) that used in the next step.

Ethyl (5-(4,5-dimethylpyridin-2-yl)-2-methoxyphenyl)glycinate (INT-II-22): Treatment of 5-(4,5-dimethylpyridin-2-yl)-2-methoxyaniline (INT-I-29, 300 mg, 1.3 mmol) with ethyl 2-bromoacetate (0.89 mL, 1.4 mmol) in the presence of DIPEA (0.69 mL, 3.9 mmol) in DMF (60 mL) at 60° C. for 2 h under N₂ atmosphere. The reaction was then cooled down to rt, EtOAc was added and washed with H₂O (5 mL) to remove DMF. The organic phase was dried and evaporated to dryness to obtain 200 mg (48% yield) of ethyl (5-(4,5-dimethylpyridin-2-yl)-2-methoxyphenyl)glycinate (INT-II-22) that used in the next step.

### Step (c): General procedure for the synthesis of (3-(pyridin-2-yl)phenyl)glycine derivatives INT-III-19, INT-III-20, INT-III-21, INT-III-17, INT-III-18 and INT-III-22

To a stirred solution of ethyl (3-(pyridin-2-yl)phenyl)glycinate derivative (1.0 equiv.) in MeOH: THF : H₂O (1:2:1), was added lithium hydroxide monohydrate (LiOH. H₂O, 3.0 equiv.) at 0° C. The resulting reaction mixture stirred at room temperature. After completion of the reaction as indicated by TLC, the reaction mixture was concentrated under reduced pressure and diluted with H₂O. The aqueous layer was acidified with sat. citric acid soln. (pH=5-6) at 0° C. resulting precipitation of the product. The solid product was dried under high reduced pressure to afford the corresponding acid as a white solid.

(5-(5-Cyclopropylpyridin-2-yl)-2-methylphenyl)glycine (INT-III-19): Treatment of ethyl (5-(5-cyclopropylpyridin-2-yl)-2-methylphenyl)glycinate (INT-II-19, 0.9 g, 2.90 mmol) with LiOH. H₂O (0.30 g, 7.25 mmol) in THF: MeOH: H₂O (2:1:1, 20 mL) at 25° C. for 1 h followed by precipitation to afford (5-(5-cyclopropylpyridin-2-yl)-2-methylphenyl)glycine INT-III-19 as an off white solid. R_{f} (EtOAc: heptane, 50:50)=0.1; yield: 0.65 g, 79%. LC-MS (m/z): 281.08 [M−H

(5-(5-Isopropylpyridin-2-yl)-2-methylphenyl)glycine (INT-III-20): Treatment of ethyl (5-(5-isopropylpyridin-2-yl)-2-methylphenyl)glycinate (INT-II-20, 700 mg, 2.24 mmol) with LiOH. H₂O (183 mg, 4.48 mmol) in THF: MeOH: H₂O (2:1:1, 20 mL) at 25° C. for 1 h followed by precipitation to afford (5-(5-isopropylpyridin-2-yl)-2-methylphenyl)glycine INT-III-20 as an white solid. R_{f}(EtOAc: heptane, 50:50)=0.1; yield: 500 mg, 70%.

Ethyl (5-(5-chloropyridin-2-yl)-2-methylphenyl)glycinate (INT-III-21): Treatment of ethyl (5-(5-chloropyridin-2-yl)-2-methylphenyl)glycinate (INT-II-21, 600 mg, 1.97 mmol) with LiOH. H₂O (160 mg, 3.94 mmol) in THF: MeOH: H₂O (2:1:1, 7 mL) at 25° C. for 1 h followed by precipitation to afford (5-(5-chloropyridin-2-yl)-2-methylphenyl)glycine INT-III-21 as an pale yellow solid. R_{f}(EtOAc: heptane, 50:50)=0.1; yield: 500 mg, 92%.

(2-Methoxy-5-(5-methylpyridin-2-yl)phenyl)glycine (INT-III-17): Treatment of ethyl (2-methoxy-5-(5-methylpyridin-2-yl)phenyl)glycinate (INT-II-17, 1.6 g, 5.3 mmol) with LiOH. H₂O (0.65 mg, 15.99 mmol) in THF: MeOH: H₂O (20:10: 10, 40 mL) at 25° C. for 1 h followed by precipitation to afford (2-methoxy-5-(5-methylpyridin-2-yl)phenyl)glycine INT-III-17 as an off white solid. R_{f} (EtOAc heptane, 50:50)=0.1; yield: 500 mg, 92%.

(2-Methyl-5-(5-methylpyridin-2-yl)phenyl)glycine (INT-III-18): Treatment of ethyl (2-methyl-5-(5-methylpyridin-2-yl)phenyl)glycinate (INT-II-18, 2.1 g, 7.39 mmol) with LiOH. H₂O (621 mg, 14.78 mmol) in THF: MeOH: H₂O (7:2:2, 11 mL) at 25° C. for 1 h followed by precipitation to afford (2-methyl-5-(5-methylpyridin-2-yl)phenyl)glycine INT-TT-18 as an off white solid. R_{f}(EtOAc: heptane, 50 50)=0.1; yield: 1.9(g, 65Y.

(5-(4,5-Dimethylpyridin-2-yl)-2-methoxyphenyl)glycine (INT-TTT-22): Treatment of ethyl (5-(4,5-dimethylpyridin-2-yl)-2-methoxyphenyl)glycinate (INT-II-22, 180 mg, 0.56 mmol) with LiOH. H₂ (3 mg, 0.16 mmol) in EtOH: H₂O (1:0. 1, 1.1 mL) at 25° C. for 1 h followed by precipitation to afford (5-(4,5-dimethylpyridin-2-yl)-2-methoxyphenyl)glycine INT-TTI-XX as an off white solid. R_{f}(EtOAc: heptane, 50:50)=0.1; yield: 105 mg, 64% that used in the next step.

TABLE 5
Reaction and purification conditions of the final examples 113-121 synthesized via STEP
19, scheme 79 (Pathway C)

| | | INT-IV | INT-III | Synthesis | |
|---|---|---|---|---|---|
| | | (Formulae | (Formulae | method | Purification |
| Example | Structure | parts 5, 6) | part 3) | (Yield %) | Method |
| 113 | 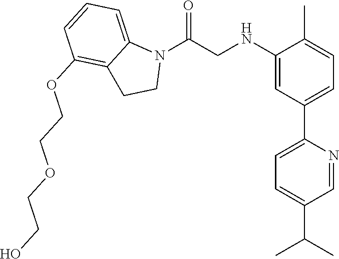 [SMILES: Cc1ccc(-c2ccc(C(C)C)cn2)cc1NCC(=O)N1CCc2c(OCCOCCO)cccc21] | INT-IV-15 | INT-III-20 | E (41) | II |
| 114 | 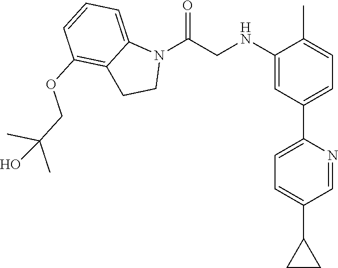 [SMILES: Cc1ccc(-c2ccc(C3CC3)cn2)cc1NCC(=O)N1CCc2c(OCC(C)(C)O)cccc21] | INT-IV-12 | INT-III-19 | E; DMF instead of DCM and DIPEA instead of Et₃N (59) | II extraction R_{f} (EtOAc:heptane, 50:50) = 0.3 |
| 115 | 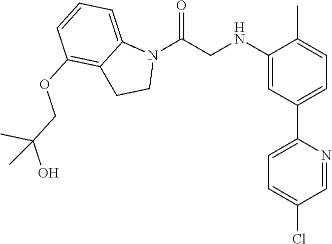 [SMILES: Cc1ccc(-c2ccc(Cl)cn2)cc1NCC(=O)N1CCc2c(OCC(C)(C)O)cccc21] | INT-IV-12 | INT-III-21 | E (20) | II |
| 116 | 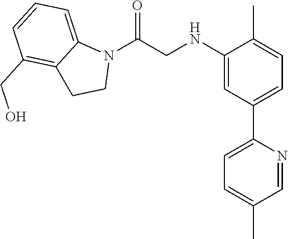 [SMILES: Cc1ccc(-c2ccc(C)c(NCC(=O)N3CCc4c(CO)cccc43)c2)nc1] | INT-IV-3 | INT-III-18 | E (64) | Precipitation, then washed with DCM, sat. NaHCO₃ soln., and H₂O |
| 117 | 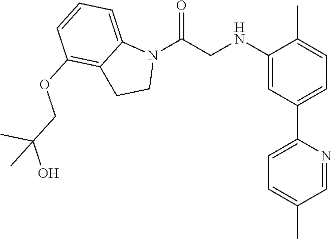 [SMILES: Cc1ccc(-c2ccc(C)c(NCC(=O)N3CCc4c(OCC(C)(C)O)cccc43)c2)nc1] | INT-IV-12 | INT-III-18 | E (56) | II |
| 118 | 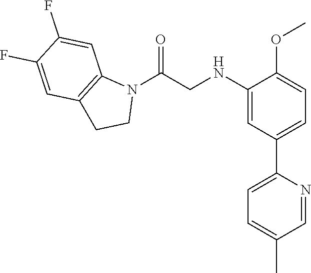 [SMILES: COc1ccc(-c2ccc(C)cn2)cc1NCC(=O)N1CCc2cc(F)c(F)cc21] | INT-IV-14 | INT-III-17 | E (27.2) | (C18 RediSep Column; H₂O:HCO₂H/ CH₃CN:HCO₂H |
| 119 | 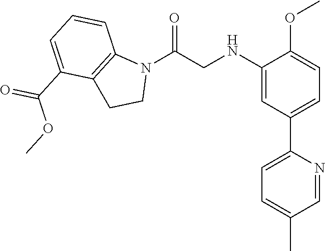 [SMILES: COC(=O)c1cccc2c1CCN2C(=O)CNc1cc(-c2ccc(C)cn2)ccc1OC] | INT-IV-8 | INT-III-17 | E (66.5) | II (Heptane- EtOAc:EtOH 3:1) & DCM- MeOH |
| 120 | 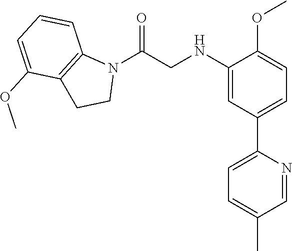 [SMILES: COc1ccc(-c2ccc(C)cn2)cc1NCC(=O)N1CCc2c(OC)cccc21] | INT-V-2 | INT-I-25 | D (11.8) | II; (Heptane- EtOAc: EtOH gradient) & triturated with CH₃CN—H₂O |
| 121 | 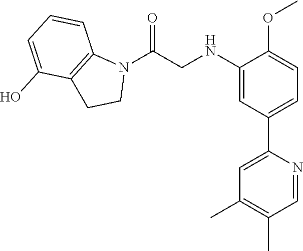 [SMILES: COc1ccc(-c2cc(C)c(C)cn2)cc1NCC(=O)N1CCc2c(O)cccc21] | INT-IV-11 | INT-III-22 | E (10) | II; (Heptane- EtOAc, 80:20) |

### Synthesis of Example 122

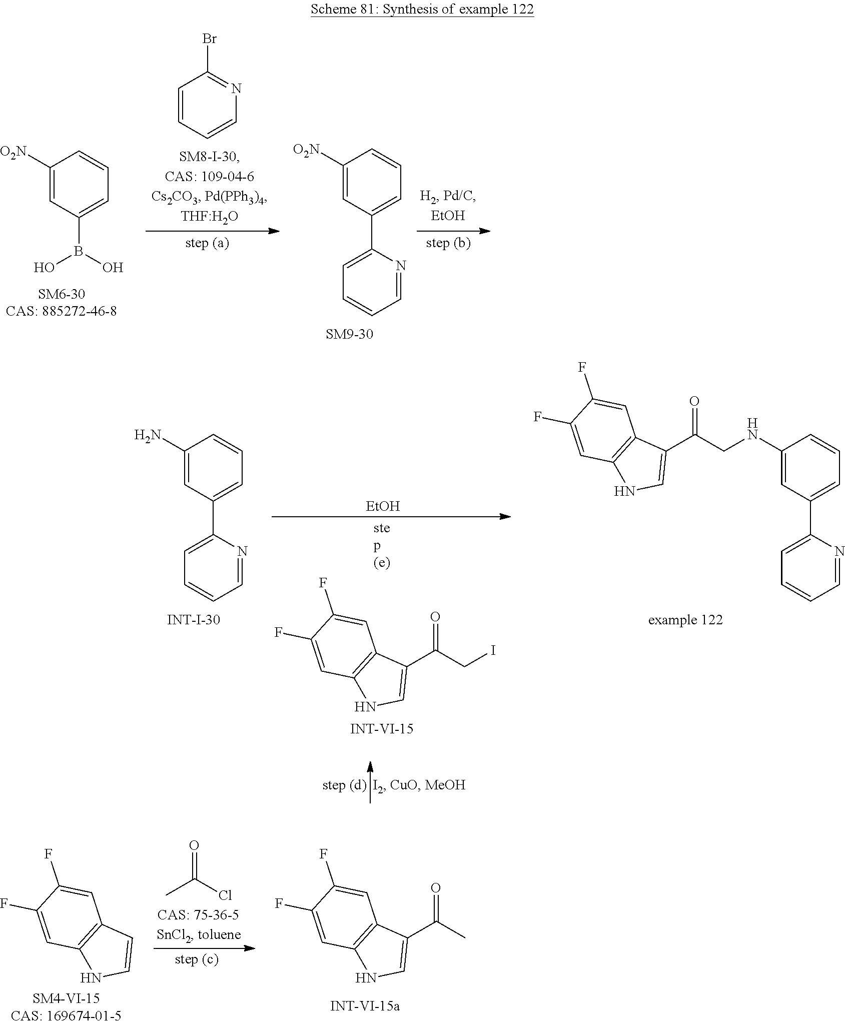

Step (a): In a microwave vial, 3-nitrophenylboronic acid (SM6-30, 250 mg, 1.50 mmol), 2-bromopyridine (SM7-T-30, 0.150 ml, 1.57 mmol), Cs₂CO₃ (1.464 g, 4.49 mmol), Pd(O)(PPh₃)₄(173 mg, 0.150 mmol), THF H₂O (8:2, 10 mL) were charged and irratiated for total 90 min. at 100° C. The reaction mixture was cooled down, diluted with DCM and washed twice with H₂O. The organic phase was dried and evaporated to dryness to obtain 450 mg of the crude material. CombiFlash purification (4g silica Gold; heptane-EtOAc gradient) gave 220 mg of SM9-30.

Step (b): The obtained 2-(3-nitrophenyl)pyridine (SM9-30, 220 mg, 1.01 mmol) was dissolved in EtOH (2.5 mL) and reduced by H-Cube flow hydrogenation instrument (cartridge 10% Pd/C, flow 1.5 mL/min, temperature 22° C. and pressure 1 bar). Collected solution was filtered to dryness to obtain 128 mg of INT-I-30.

Step (c): The commercially available 5,6-difluoroindole (SM4-VI-15, 1 g, 6.53 mmol), toluene (25 mL) and acetyl chloride (0.93 mL, 13.06 mmol) were charged into the reaction flask and cooled to 0° C. Tin(IV)chloride (1M in DCM, 13.06 mL, 13.06 mmol) was added slowly to the reaction mixture. Strong precipitation was obtained and more toluene (10 mL) was added before the complete addition of tin(IV)chloride. The reaction was stirred for 75 min. at rt, cooled to 0° C. and quenched with slow addition of H₂O. The mixture was washed with 3×EtOAc (precipitate dissolved in bigger amounts), organic phases were combined, dried and evaporated to dryness to obtain 2.1 g of the crude material. Afterward DCM, EtOAc and H₂O were added, the mixture was stirred and the precipitate was filtered off. The precipitate was dissolved in EtOAc, combined with filtrate organic phase, dried and ovaporated to dryness to obtain 1.3 g of INT-VI-15a.

Step (d): The obtained 1-(5,6-difluoro-1H-indol-3-yl)ethanone (INT-VI-15a, 500 mg; 2.56 mmol), Iodine (975 mg, 3.84 mmol), copper(II)oxide (nano powder <50 nm particle size) (306 mg, 3.84 mmol) and MeOH (15 mL) were charged in the reaction flask and refluxed for 1 h. The reaction was cooled to rt, diluted with DCM, washed with sat. soln. of Na₂S₂O₃ and then with H₂O. The organic phase was dried with phase separator and evaporated to dryness to obtain 300 mg of INT-VI-15.

Step (e): In a reaction flask, 3-(pyridin-2-yl)aniline (INT-I-30, 10.6 mg, 0.062 mmol), EtOH (2 mL) and 1-(5,6-difluoro-1H-indol-3-yl)-2-iodoethanone (INT-VI-15, 20 mg, 0.062 mmol) were charged and stirred at rt for 3 days. After that, the reaction mixture was evaporated to dryness. The residual material was dissolved in DCM, washed with H₂O, dried and evaporated to dryness. The crude product was purified with CombiFlash purification (4g silica; EtOAc-heptane gradient) gave 13 mg of example 122.

TABLE 6
Analytical data and P2X4 biological activity data of the selected final examples

| | | | | LC/ | |
|---|---|---|---|---|---|
| | | P2X4 | | ESI- | Purity |
| | | receptor | | MS | data % |
| | | inhibitory | | ([M + | & |
| Example | Structure | potency* | Chemical name | H]⁺) | apperence |
| 1 | 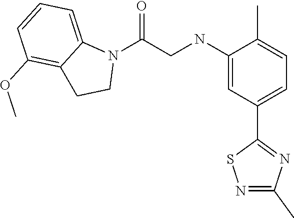 [SMILES: COc1cccc2c1CCN2C(=O)CNc1cc(-c2nc(C)ns2)ccc1C] | +++++ | 1-(4-methoxyindolin-1- yl)-2-((2-methyl-5- (3-methyl-1,2,4- thiadiazol-5-yl)phenyl)- amino)ethan-1-one. | 395.3 | 96.89 White solid |
| 2 | 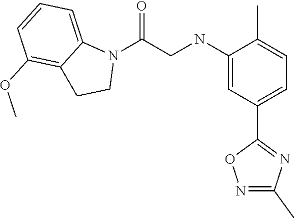 [SMILES: COc1cccc2c1CCN2C(=O)CNc1cc(-c2nc(C)no2)ccc1C] | +++++ | 1-(4-methoxyindolin- 1-yl)-2-((2-methyl- 5-(3-methyl-1,2,4- oxadiazol-5-yl)phenyl)- amino)ethan-1-one. | 379.44 | 95.5 Yellowish solid |
| 3 | 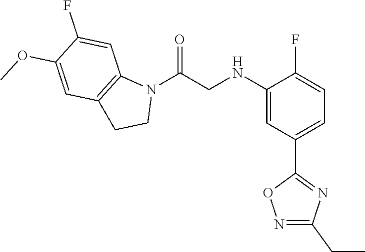 [SMILES: CCc1noc(-c2ccc(F)c(NCC(=O)N3CCc4cc(OC)c(F)cc43)c2)n1] | ++++ | 2-((5-(3-ethyl-1,2,4- oxadiazol-5-yl)-2- fluorophenyl)amino)-1- (6-fluoro-5- methoxyindolin-1- yl)ethan-1-one. | 415.1 | 95 White solid |
| 4 | 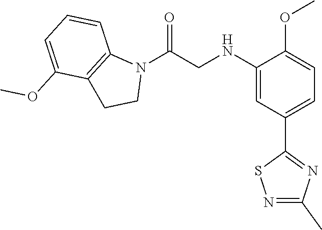 | ++++ | 2-((2-methoxy-5-(3- methyl-1,2,4- thiadiazol-5-yl)phenyl)- amino)-1-(4- methoxyindolin- 1-yl)ethan-1-one. | 411.38 | 98.5 White solid |
| 5 | 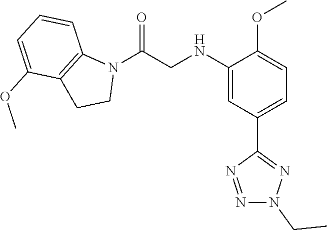 [SMILES: CCn1nnc(-c2ccc(OC)c(NCC(=O)N3CCc4c(OC)cccc43)c2)n1] | ++++ | 2-((5-(2-ethyl-2H- tetrazol-5-yl)-2- methoxyphenyl) amino)-1-(4- methoxyindolin-1- yl)ethan-1-one. | 409.1 | 98.4 White solid |
| 6 | 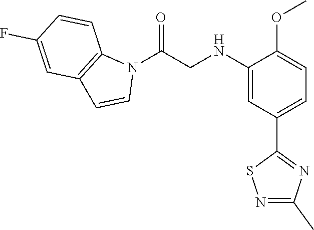 [SMILES: COc1ccc(-c2nc(C)ns2)cc1NCC(=O)n1ccc2cc(F)ccc21] | ++ | 1-(5-fluoro-1H-indol-1- yl)-2-((2-methoxy-5-(3- methyl-1,2,4-thiadiazol- 5-yl)phenyl)amino-) ethan-1-one. | 397.2 | 94.4 White solid |
| 7 | 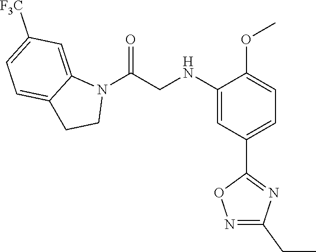 [SMILES: CCc1noc(-c2ccc(OC)c(NCC(=O)N3CCc4ccc(C(F)(F)F)cc43)c2)n1] | + | 2-(5-(3-ethyl-1,2,4- oxadiazol-5-yl)-2- methoxyphenylamino)- 1-(6-(trifluoromethyl) indolin-1-yl)ethanone. | 447.13 | 93.4 White solid |
| 8 | 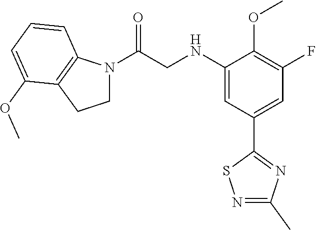 [SMILES: COc1cccc2c1CCN2C(=O)CNc1cc(-c2nc(C)ns2)cc(F)c1OC] | ++ | 2-((3-fluoro-2-methoxy- 5-(3-methyl-1,2,4- thiadiazol-5-yl)phenyl) amino)-1-(4- methoxyindolin-1-yl) ethan-1-one. | 429.1 | 96.6 Yellowish powder |
| 9 | 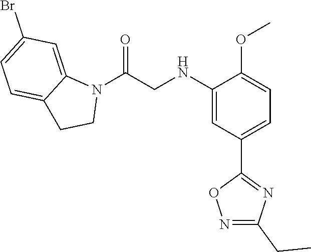 [SMILES: CCc1noc(-c2ccc(OC)c(NCC(=O)N3CCc4ccc(Br)cc43)c2)n1] | +++ | 1-(6-bromoindolin-1-yl)- 2-((5-(3-ethyl-1,2,4- oxadiazol-5-yl)-2- methoxyphenyl)amino) ethan-1-one. | 457.1 | 97.3 Yellow powder |
| 10 | 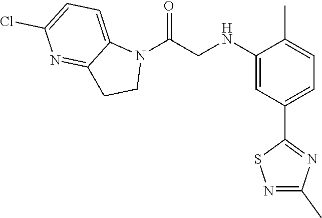 [SMILES: Cc1nsc(-c2ccc(C)c(NCC(=O)N3CCc4nc(Cl)ccc43)c2)n1] | ++ | 1-(5-chloro-2,3-dihydro- 1H-pyrrolo[3,2-b]pyridin- 1-yl)-2-((2-methyl-5- (3-methyl-1,2,4- thiadiazol-5-yl)phenyl) amino)ethan-1-one. | 400 | 98.3 White powder |
| 11 | 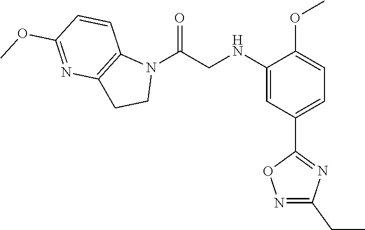 [SMILES: CCc1noc(-c2ccc(OC)c(NCC(=O)N3CCc4nc(OC)ccc43)c2)n1] | ++++ | 2-((5-(3-ethyl-1,2,4- oxadiazol-5-yl)-2- methoxyphenyl) amino)-1-(5- methoxy-2,3-dihydro-1H- pyrrolo[3,2-b]pyridin-1- yl)ethan-1-one. | 410 | 94.4 Off white powder |
| 12 | 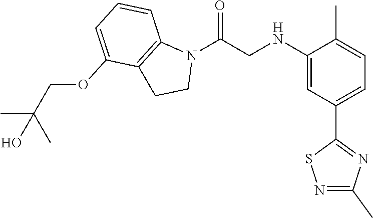 [SMILES: Cc1nsc(-c2ccc(C)c(NCC(=O)N3CCc4c(OCC(C)(C)O)cccc43)c2)n1] | +++++ | 1-(4-(2-hydroxy-2- methylpropoxy)indolin-1- yl)-2-((2-methyl-5- (3-methyl-1,2,4- thiadiazol-5-yl)phenyl) amino). | 453.2 | 95.93 Beige solid |
| 13 | 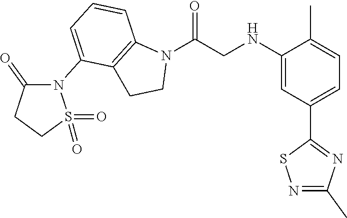 [SMILES: Cc1nsc(-c2ccc(C)c(NCC(=O)N3CCc4c3cccc4N3C(=O)CCS3(=O)=O)c2)n1] | +++++ | 2-(1-((2-methyl-5-(3- methyl-1,2,4-thiadiazol- 5-yl)phenyl)glycyl) indolin-4-yl) isothiazolidin-3-one 1,1-dioxide. | 498.4 | 98.1 Pale yellow crystals |
| 14 | 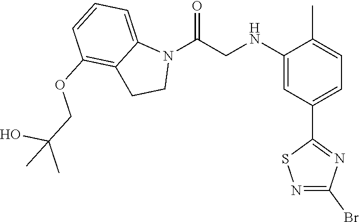 [SMILES: Cc1ccc(-c2nc(Br)ns2)cc1NCC(=O)N1CCc2c(OCC(C)(C)O)cccc21] | +++++ | 2-((5-(3-bromo-1,2,4- thiadiazol-5-yl)-2- methylphenylamino)-1- (4-(2-hydroxy-2- methylpropoxy)indolin- 1-yl)ethan-1-one. | 517.1 | 95.6 Yellow powder |
| 15 | 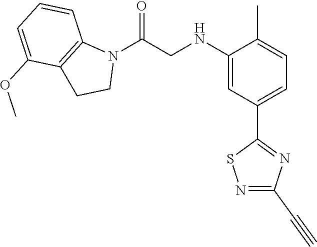 [SMILES: C#Cc1nsc(-c2ccc(C)c(NCC(=O)N3CCc4c(OC)cccc43)c2)n1] | +++++ | 2-((5-(3-ethynyl-1,2,4- thiadiazol-5-yl)-2- methylphenyl)amino)-1- (4-methoxyindolin-1- yl)ethan-1-one. | 405.1 | 97.28 Off white solid |
| 16 | 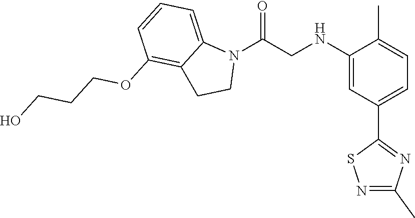 [SMILES: Cc1nsc(-c2ccc(C)c(NCC(=O)N3CCc4c(OCCCO)cccc43)c2)n1] | +++++ | 1-(4-(3- hydroxypropoxy)indolin- 1-yl)-2-((2-methyl-5-(3- methyl-1,2,4- thiadiazol-5- yl)phenyl)amino) ethan-1-one. | 439.2 | 97.06 White solid |
| 17 | 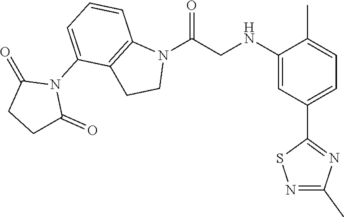 [SMILES: Cc1nsc(-c2ccc(C)c(NCC(=O)N3CCc4c3cccc4N3C(=O)CCC3=O)c2)n1] | +++++ | 1-(1-((2-methyl-5-(3- methyl-1,2,4-thiadiazol- 5-yl)phenyl)glycyl) indolin-4-yl) pyrrolidine-2,5-dione. | 462.0 | 96.22 Beige solid |
| 18 | 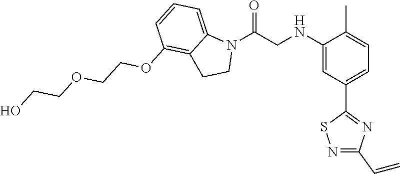 [SMILES: C=Cc1nsc(-c2ccc(C)c(NCC(=O)N3CCc4c(OCCOCCO)cccc43)c2)n1] | +++++ | 1-(4-(2-(2- hydroxyethoxy)ethoxy) indolin-1-yl)-2-((2- methyl-5-(3-vinyl- 1,2,4-thiadiazol-5- yl)phenyl)amino) ethan-1-one. | 481.2 | 98.24 Beige solid |
| 19 | 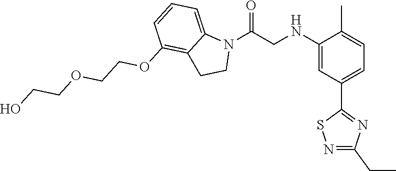 [SMILES: CCc1nsc(-c2ccc(C)c(NCC(=O)N3CCc4c(OCCOCCO)cccc43)c2)n1] | +++++ | 2-((5-(3-ethyl-1,2,4- thiadiazol-5-yl)-2- methylphenyl)amino)-1- (4-(2-(2- hydroxyethoxy)ethoxy) indolin-1-yl)ethan-1-one | 483.2 | 97.61 Beige solid |
| 20 | 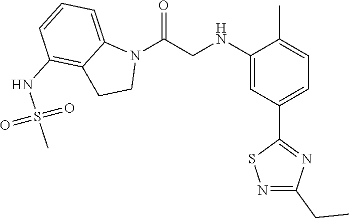 [SMILES: CCc1nsc(-c2ccc(C)c(NCC(=O)N3CCc4c(NS(C)(=O)=O)cccc43)c2)n1] | +++++ | N-(1-((5-(3-ethyl-1,2,4- thiadiazol-5-yl)-2- methylphenyl)glycyl) indolin-4-yl) methanesulfonamide. | 472.3 | 98.7 White solid |
| 21 | 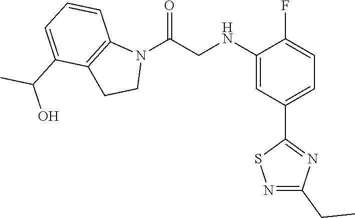 [SMILES: CCc1nsc(-c2ccc(F)c(NCC(=O)N3CCc4c(C(C)O)cccc43)c2)n1] | +++++ | 2-((5-(3-ethyl-1,2,4- thiadiazol-5-yl)-2- fluorophenyl)amino)-1- (4-(1-hydroxyethyl) indolin-1-yl)ethan-1- one. | 427.2 | 98.6 White powder |
| 22 | 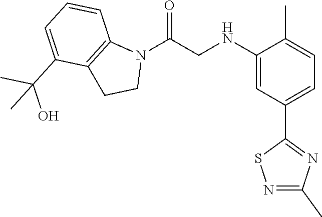 [SMILES: Cc1nsc(-c2ccc(C)c(NCC(=O)N3CCc4c3cccc4C(C)(C)O)c2)n1] | +++++ | 1-(4-(2-hydroxypropan- 2-yl)indolin-1-yl)-2- ((2-methyl-5-(3- methyl-1,2,4-thiadiazol- 5-yl)phenyl)amino) ethan-1-one. | 423.25 | (>95%) White solid |
| 23 | 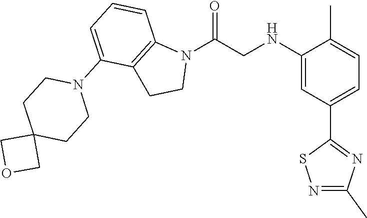 [SMILES: Cc1nsc(-c2ccc(C)c(NCC(=O)N3CCc4c(N5CCC6(CC5)COC6)cccc43)c2)n1] | +++++ | 1-(4-(2-oxa-7- azaspiro[3.5]nonan-7- yl)indolin-1-yl)-2- ((2-methyl-5-(3- methyl-1,2,4- thiadiazol-5-yl)phenyl)- amino)ethan-1-one. | 490.30 | (>95%) White solid |
| 24 | 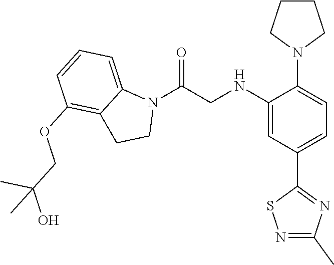 [SMILES: Cc1nsc(-c2ccc(N3CCCC3)c(NCC(=O)N3CCc4c(OCC(C)(C)O)cccc43)c2)n1] | +++++ | 1-(4-(2-hydroxy-2- methylpropoxy)indolin- 1-yl)-2-((5-(3-methyl- 1,2,4-thiadiazol-5-yl)-2- (pyrrolidin-1-yl)phenyl)- amino)ethan-1-one. | 508.3 | 95.12 Yellow solid |
| 25 | 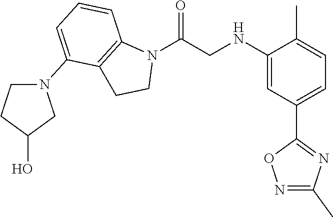 [SMILES: Cc1noc(-c2ccc(C)c(NCC(=O)N3CCc4c(N5CCC(O)C5)cccc43)c2)n1] | +++++ | 1-(4-(3-hydroxy- pyrrolidin-1-yl) indolin-1-yl)-2-((2- methyl-5-(3-methyl- 1,2,4-oxadiazol-5-yl) phenyl)amino)ethan-1- one. | 434.26 | (>95%) White solid |
| 26 | 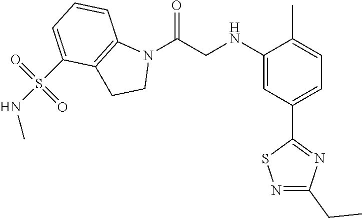 [SMILES: CCc1nsc(-c2ccc(C)c(NCC(=O)N3CCc4c3cccc4S(=O)(=O)NC)c2)n1] | +++++ | 1-((5-(3-ethyl-1,2,4- thiadiazol-5-yl)-2- methylphenyl)glycyl)-N- methylindoline-N- sulfonamide. | 472.4 | 96.2 White solid |
| 27 | 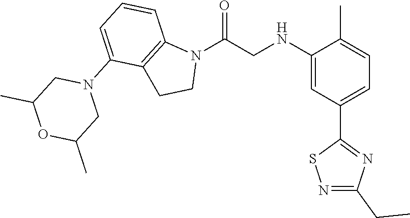 [SMILES: CCc1nsc(-c2ccc(C)c(NCC(=O)N3CCc4c(N5CC(C)OC(C)C5)cccc43)c2)n1] | +++++ | 1-(4-(2,6- dimethylmorpholino) indolin-1-yl)-2-((5-(3- ethyl-1,2,4- thiadiazol-5-yl)-2- methylphenyl)amino) ethan-1-one. | 492.0 | 99.58 White solid |
| 28 | 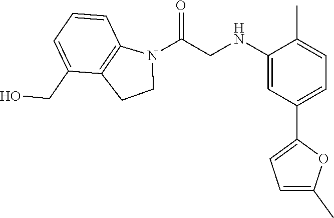 [SMILES: Cc1ccc(-c2ccc(C)c(NCC(=O)N3CCc4c(CO)cccc43)c2)o1] | +++++ | 1-(4-(hydroxymethyl) indolin-1-yl)-2-((2- methyl-5-(5- methylfuran-2-yl) phenyl)amino)ethan-1- one. | 377.2 | 99.0 Beige powder |
| 29 | 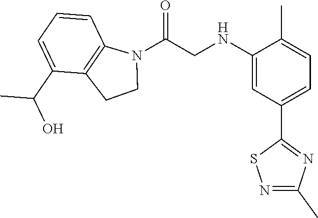 [SMILES: Cc1nsc(-c2ccc(C)c(NCC(=O)N3CCc4c(C(C)O)cccc43)c2)n1] | ++++ | 1-(4-(1-hydroxyethyl) indolin-1-yl)-2-((2- methyl-5-(3-methyl- 1,2,4-thiadiazol-5-yl) phenyl)amino)ethan-1- one. | 409.1 | 96.9 White Beige |
| 30 | 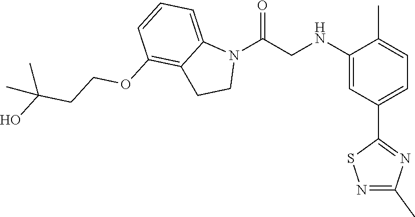 [SMILES: Cc1nsc(-c2ccc(C)c(NCC(=O)N3CCc4c(OCCC(C)(C)O)cccc43)c2)n1] | +++++ | 1-(4-(3-hydroxy-3- methylbutoxy)indolin- 1-yl)-2-((2-methyl-5- (3-methyl-1,2,4- thiadiazol-5-yl) phenyl)amino)ethan-1- one. | 467.3 | 97.32 White solid |
| 31 | 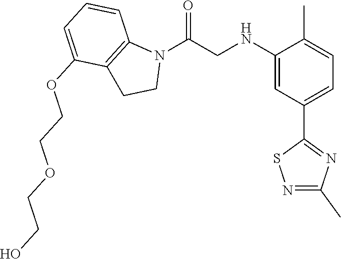 [SMILES: Cc1nsc(-c2ccc(C)c(NCC(=O)N3CCc4c(OCCOCCO)cccc43)c2)n1] | ++++ | 1-(4-(2-(2-hydroxyethoxy)- ethoxy)indolin-1-yl)-2-((2- methyl-5-(3-methyl-1,2,4- thiadiazol-5-yl)phenyl)- amino)ethan-1-one. | 469.2 | 96.36 Light brown solid |
| 32 | 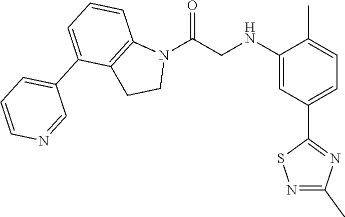 [SMILES: Cc1nsc(-c2ccc(C)c(NCC(=O)N3CCc4c(-c5cccnc5)cccc43)c2)n1] | +++++ | 2-((2-methyl-5-(3-methyl- 1,2,4-thiadiazol-5-yl) phenyl)amino)-1-(4- (pyridin-3-yl)indolin-1- yl)ethan-1-one. | 442.21 | (>95%) White solid |
| 33 | 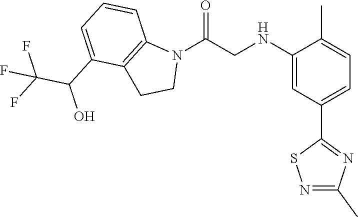 [SMILES: Cc1nsc(-c2ccc(C)c(NCC(=O)N3CCc4c(C(O)C(F)(F)F)cccc43)c2)n1] | ++++ | 2-((2-methyl-5-(3-methyl- 1,2,4-thiadiazol-5-yl) phenyl)amino)-1-(4- (2,2,2-trifluoro-1- hydroxyethyl)indolin-1- yl)ethan-1-one. | 463.14 | (>95%) White solid |
| 34 | 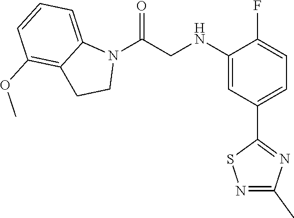 [SMILES: COc1cccc2c1CCN2C(=O)CNc1cc(-c2nc(C)ns2)ccc1F] | ++++ | 2-((2-fluoro-5-(3-methyl- 1,2,4-thiadiazol-5-yl) phenyl)amino)-1-(4- methoxyindolin-1-yl)ethan- 1-one. | 339 | 98.5 White solid |
| 35 | 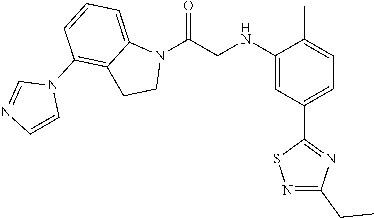 [SMILES: CCc1nsc(-c2ccc(C)c(NCC(=O)N3CCc4c3cccc4-n3ccnc3)c2)n1] | ++++ | 1-(4-(1H-imidazol-1-yl) indolin-1-yl)-2-((5-(3- ethyl-1,2,4-thiadiazol-5- yl)-2-methyl- phenyl)amino)ethan-1-one. | 445.20 | (>95%) White solid |
| 36 | 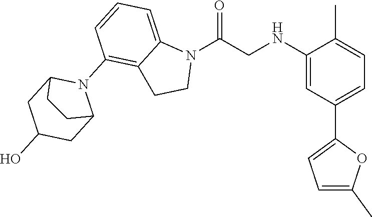 [SMILES: Cc1ccc(-c2ccc(C)c(NCC(=O)N3CCc4c3cccc4N3C4CCC3CC(O)C4)c2)o1] | ++++ | 1-(4-(3-hydroxy-8- azabicyclo[3.2.1]octan-8- yl)indolin-1-yl)-2-((2- methyl-5-(5- methylfuran-2-yl) phenyl)amino)ethan-1-one. | 472.2 | 98.0 White powder |
| 37 | 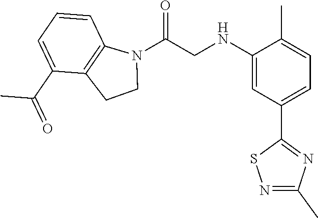 [SMILES: CC(=O)c1cccc2c1CCN2C(=O)CNc1cc(-c2nc(C)ns2)ccc1C] | ++++ | 1-(4-acetylindolin-1-yl)- 2-((2-methyl-5-(3- methyl-1,2,4-thiadiazol-5- yl)phenyl)amino)ethan- 1-one. | 407.10 | (>95%) White solid |
| 38 | 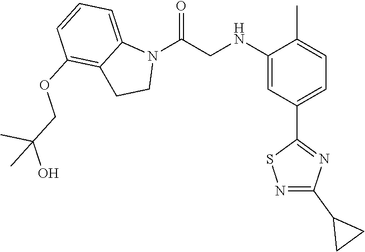 [SMILES: Cc1ccc(-c2nc(C3CC3)ns2)cc1NCC(=O)N1CCc2c(OCC(C)(C)O)cccc21] | ++++ | 2-((5-(3-cyclopropyl- 1,2,4-thiadiazol-5-yl)-2- methylphenyl)amino)-1- (4-(2-hydroxy-2- methylpropoxy)- indolin-1-yl)ethan-1-one. | 479.2 | 98.6 Light yellow solid |
| 39 | 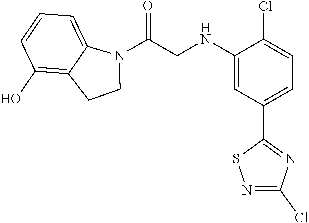 [SMILES: O=C(CNc1cc(-c2nc(Cl)ns2)ccc1Cl)N1CCc2c(O)cccc21] | ++++ | 2-((2-chloro-5-(3-chloro- 1,2,4-thiadiazol-5-yl) phenyl)amino)-1-(4- hydroxyindolin-1-yl) ethan-1-one. | 420.9 | 94.78 Yellow solid |
| 40 | 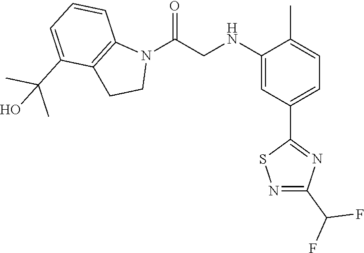 [SMILES: Cc1ccc(-c2nc(C(F)F)ns2)cc1NCC(=O)N1CCc2c1cccc2C(C)(C)O] | ++++ | 2-((5-(3-(difluoromethyl)- 1,2,4-thiadiazol-5-yl)-2- methylphenyl)amino)-1- (4-(2-hydroxypropan-2- yl)indolin-1-yl) ethan-1-one. | 459.20 | (>95%) White solid |
| 41 | 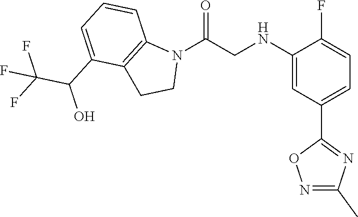 [SMILES: Cc1noc(-c2ccc(F)c(NCC(=O)N3CCc4c(C(O)C(F)(F)F)cccc43)c2)n1] | ++++ | 2-((2-fluoro-5-(3-methyl- 1,2,4-oxadiazol-5-yl) phenyl)amino)-1-(4- (2,2,2-trifluoro-1- hydroxyethyl)indolin-1- yl)ethan-1-one. | 451.2 | 97.82 White solid |
| 42 | 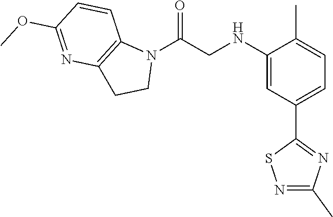 | ++++ | 1-(5-methoxy-2,3- dihydro-1H-pyrrolo[3,2-b] pyridin-1-yl)-2-((2-methyl- 5-(3-methyl-1,2,4- thiadiazol-5-yl)phenyl)- amino)ethan-1-one. | 396.0 | 99.09 Beige solid |
| 43 | 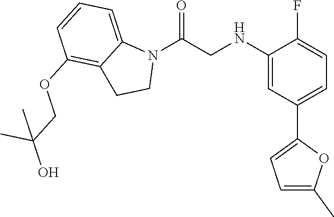 [SMILES: Cc1ccc(-c2ccc(F)c(NCC(=O)N3CCc4c(OCC(C)(C)O)cccc43)c2)o1] | ++++ | 2-((2-fluoro-5-(5- methylfuran-2-yl)phenyl) amino)-1-(4-(2-hydroxy- 2-methylpropoxy)- indolin-1-yl)ethan-1-one. | 439.1 | 98.2 White powder |
| 44 | 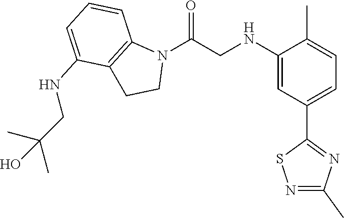 [SMILES: Cc1nsc(-c2ccc(C)c(NCC(=O)N3CCc4c(NCC(C)(C)O)cccc43)c2)n1] | ++++ | 1-(4-((2-hydroxy-2- methylpropyl)amino) indolin-1-yl)-2-((2-methyl- 5-(3-methyl-1,2,4- thiadiazol-5-yl)phenyl)- amino)ethan-1-one. | 452.30 | (>95%) White solid |
| 45 | 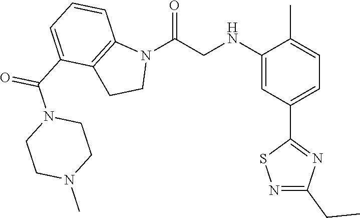 [SMILES: CCc1nsc(-c2ccc(C)c(NCC(=O)N3CCc4c(C(=O)N5CCN(C)CC5)cccc43)c2)n1] | ++++ | 2-((5-(3-ethyl-1,2,4- thiadiazol-5-yl)-2- methylphenyl)amino)-1- (4-(4-methylpiperazine-1- carbonyl)indolin-1-yl) ethan-1-one. | 505.3 | 96.2 yellow solid |
| 46 | 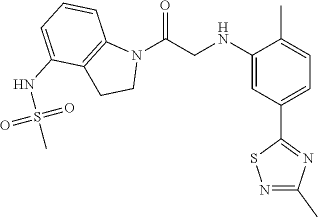 [SMILES: Cc1nsc(-c2ccc(C)c(NCC(=O)N3CCc4c(NS(C)(=O)=O)cccc43)c2)n1] | ++++ | N-(1-((2-methyl-5-(3- methyl-1,2,4-thiadiazol-5- yl)phenyl)glycyl)indolin- 4-yl)methanesulfonamide. | 458.2 | 96.3 White solid |
| 47 | 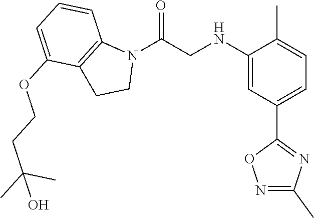 [SMILES: Cc1noc(-c2ccc(C)c(NCC(=O)N3CCc4c(OCCC(C)(C)O)cccc43)c2)n1] | ++++ | 1-(4-(3-hydroxy-3- methylbutoxy)indolin-1- yl)-2-((2-methyl-5-(3- methyl-1,2,4-oxadiazol-5- yl)phenyl)amino)ethan-1- one. | 451.32 | (>95%) Off white solid |
| 48 | 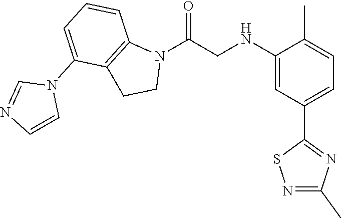 [SMILES: Cc1nsc(-c2ccc(C)c(NCC(=O)N3CCc4c3cccc4-n3ccnc3)c2)n1] | ++++ | 1-(4-(1H-imidazol-1-yl) indolin-1-yl)-2-((2- methyl-5-(3-methyl-1,2,4- thiadiazol-5-yl)phenyl) amino)ethan-1-one. | 431.15 | (>95%) Off white solid |
| 49 | 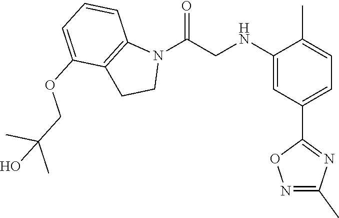 [SMILES: Cc1noc(-c2ccc(C)c(NCC(=O)N3CCc4c(OCC(C)(C)O)cccc43)c2)n1] | +++ | 1-(4-(2-hydroxy-2- methylpropoxy)indolin-1- yl)-2-((2-methyl-5-(3- methyl-1,2,4-oxadiazol-5- yl)phenyl)amino)ethan- 1-one. | 437.33 | (>95%) Off white solid |
| 50 | 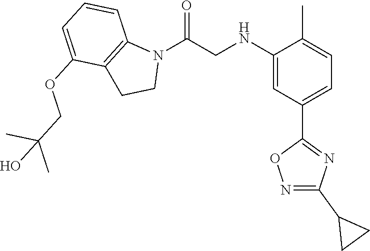 [SMILES: Cc1ccc(-c2nc(C3CC3)no2)cc1NCC(=O)N1CCc2c(OCC(C)(C)O)cccc21] | +++ | 2-((5-(3-cyclopropyl-1,2,4- oxadiazol-5-yl)-2- methylphenyl)amino)-1- (4-(2-hydroxy-2- methylpropoxy)- indolin-1-yl)ethan-1-on. | 463.36 | (>95%) Off white solid |
| 51 | 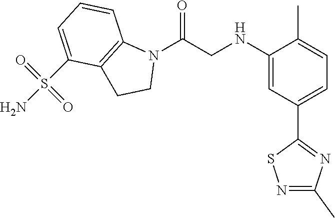 [SMILES: Cc1nsc(-c2ccc(C)c(NCC(=O)N3CCc4c3cccc4S(N)(=O)=O)c2)n1] | +++ | 1-((2-methyl-5-(3-methyl- 1,2,4-thiadiazol-5-yl) phenyl)glycyl)- indoline-4-sulfonamide. | 444.1 | 96.2 White solid |
| 52 | 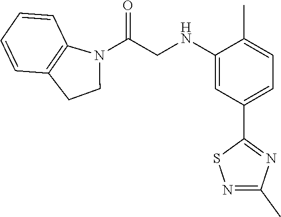 [SMILES: Cc1nsc(-c2ccc(C)c(NCC(=O)N3CCc4ccccc43)c2)n1] | +++ | 1-(indolin-1-yl)-2-((2- methyl-5-(3-methyl- 1,2,4-thiadiazol-5-yl) phenyl)amino)ethan-1- one. | 365.1 | 96.44 White solid |
| 53 | 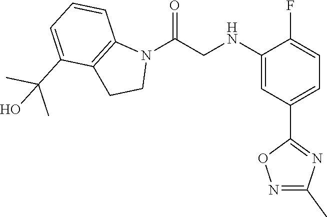 [SMILES: Cc1noc(-c2ccc(F)c(NCC(=O)N3CCc4c3cccc4C(C)(C)O)c2)n1] | ++++ | 2-((2-fluoro-5-(3-methyl- 1,2,4-oxadiazol-5-yl) phenyl)amino)-1-(4-(2- hydroxypropan-2-yl)- indolin-1-yl)ethan-1-one. | 411.3 | 96.72 White solid |
| 54 | 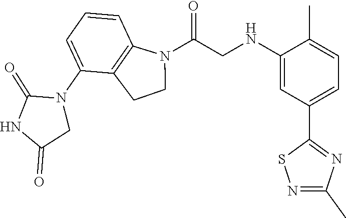 [SMILES: Cc1nsc(-c2ccc(C)c(NCC(=O)N3CCc4c3cccc4N3CC(=O)NC3=O)c2)n1] | ++ | 1-(1-((2-methyl-5-(3- methyl-1,2,4-thiadiazol- 5-yl)phenyl)glycyl) indolin-4-yl) imidazolidine-2,4-dione. | 463.2 | 96.9 Pale yellow solid |
| 55 | 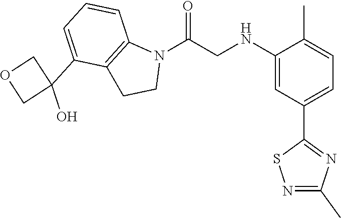 [SMILES: Cc1nsc(-c2ccc(C)c(NCC(=O)N3CCc4c3cccc4C3(O)COC3)c2)n1] | +++++ | 1-(4-(3-hydroxyoxetan-3- yl)indolin-1-yl)-2-((2- methyl-5-(3-methyl-1,2,4- thiadiazol-5-yl)phenyl) amino)ethan-1-one. | 437.15 | (>95%) Off white solid |
| 56 | 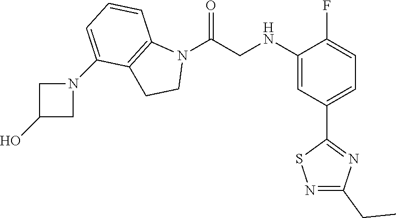 [SMILES: CCc1nsc(-c2ccc(F)c(NCC(=O)N3CCc4c(N5CC(O)C5)cccc43)c2)n1] | +++++ | 2-((5-(3-ethyl-1,2,4- thiadiazol-5-yl)-2- fluorophenyl)amino)-1- (4-(3-hydroxyazetidin-1- yl)indolin-1-yl)ethan-1- one. | 454.19 | (>95%) Off white solid |
| 57 | 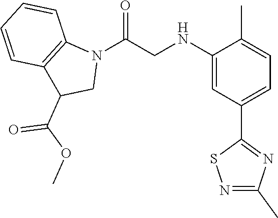 [SMILES: COC(=O)C1CN(C(=O)CNc2cc(-c3nc(C)ns3)ccc2C)c2ccccc21] | + | methyl 1-((2-methyl-5-(3- methyl-1,2,4- thiadiazol-5-yl)- phenyl)glycyl)indoline- 3-carboxylate. | 423.1 | 98.5 Light yellow powder |
| 58 | 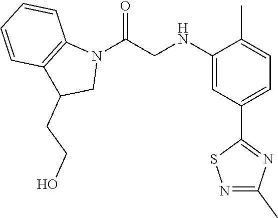 [SMILES: Cc1nsc(-c2ccc(C)c(NCC(=O)N3CC(CCO)c4ccccc43)c2)n1] | + | 1-(3-(2-hydroxyethyl) indolin-1-yl)-2-((2- methyl-5-(3-methyl- 1,2,4-thiadiazol-5- yl)phenyl)amino)ethan-1- one. | 409.3 | 96 White powder |
| 59 | 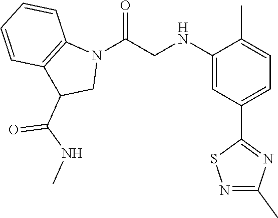 [SMILES: CNC(=O)C1CN(C(=O)CNc2cc(-c3nc(C)ns3)ccc2C)c2ccccc21] | + | N-methyl-1-((2-methyl-5- (3-methyl-1,2,4-thiadiazol- 5-yl)phenyl)glycyl) indoline-3-carboxamide. | 422.2 | 95.3 White powder |
| 60 | 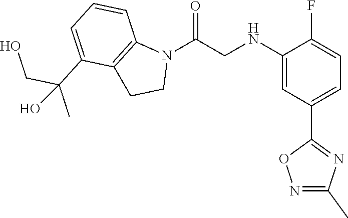 [SMILES: Cc1noc(-c2ccc(F)c(NCC(=O)N3CCc4c3cccc4C(C)(O)CO)c2)n1] | +++ | 1-(4-(1,2-dihydroxy- propan-2-yl)indolin-1- yl)-2-((2-fluoro-5- (3-methyl-1,2,4-oxadiazol- 5-yl)phenyl)amino)ethan- 1-one. | 427.37 | (>95%) White solid |
| 61 | 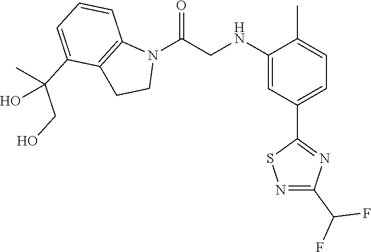 [SMILES: Cc1ccc(-c2nc(C(F)F)ns2)cc1NCC(=O)N1CCc2c1cccc2C(C)(O)CO] | +++++ | 2-((5-(3-(difluoromethyl)- 1,2,4-thiadiazol-5-yl)-2- methylphenyl)amino)-1- (4-(1,2-dihyroxypropan- 2-yl)indolin-1-yl) ethan-1-one. | 475.24 | (>95%) Yellow solid |
| 62 | 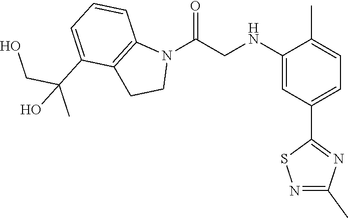 [SMILES: Cc1nsc(-c2ccc(C)c(NCC(=O)N3CCc4c3cccc4C(C)(O)CO)c2)n1] | +++++ | (R,S)-1-(4-(1,2-dihydroxy- propan-2-yl)indolin-1-yl)- 2-((2-methyl-5-(3-methyl- 1,2,4-thiadiazol-5-yl) phenyl)amino)ethan-1-one. | 439.23 | (>95%) White solid |
| 63 | 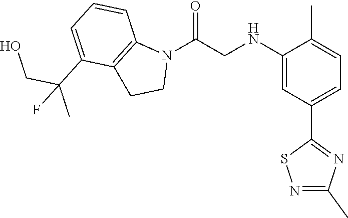 [SMILES: Cc1nsc(-c2ccc(C)c(NCC(=O)N3CCc4c3cccc4C(C)(F)CO)c2)n1] | +++++ | (R,S)-1-(4-(2- fluoro-1-hydroxy- propan-2-yl)indolin-1-yl)- 2-((2-methyl-5-(3-methyl- 1,2,4-thiadiazol-5- yl)phenyl)amino)ethan-1- one. | 441.12 | (>95%) Off white solid |
| 64 | 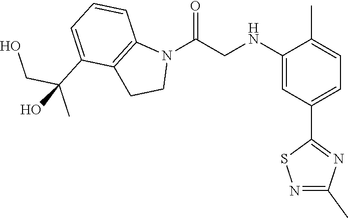 [SMILES: Cc1nsc(-c2ccc(C)c(NCC(=O)N3CCc4c3cccc4[C@@](C)(O)CO)c2)n1] | +++++ | (R)-1-(4-(1,2-dihydroxy- propan-2-yl)indolin-1-yl)- 2-((2-methyl-5-(3-methyl- 1,2,4-thiadiazol-5-yl) phenyl)amino)ethan-1-one. | 439.22 | (>95%) Off white solid |
| 65 | 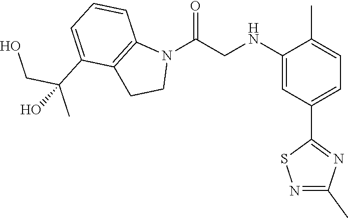 [SMILES: Cc1nsc(-c2ccc(C)c(NCC(=O)N3CCc4c3cccc4[C@](C)(O)CO)c2)n1] | +++++ | (S)-1-(4-(1,2-dihydroxy- propan-2-yl)indolin-1- yl)-2-((2-methyl-5-(3- methyl-1,2,4-thiadiazol- 5-yl)phenyl)amino) ethan-1-one. | 439.23 | (>95%) Off white solid |
| 66 | 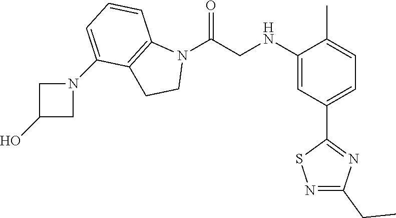 [SMILES: CCc1nsc(-c2ccc(C)c(NCC(=O)N3CCc4c(N5CC(O)C5)cccc43)c2)n1] | ++++ | 2-((5-(3-ethyl-1,2,4- thiadiazol-5-yl)-2- methylphenyl)amino)-1- (4-(3-hydroxyazetidin-1- yl)indolin-1-yl)ethan-1- one. | 450.15 | (>95%) Off white solid |
| 67 | 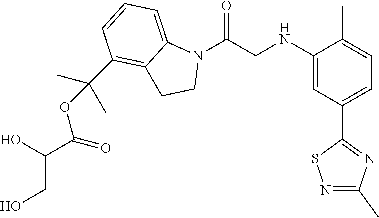 [SMILES: Cc1nsc(-c2ccc(C)c(NCC(=O)N3CCc4c3cccc4C(C)(C)OC(=O)C(O)CO)c2)n1] | ++++ | 2-(1-((2-methyl-5-(3- methyl-1,2,4-thiadiazol-5- yl)phenyl)glycyl)indolin-4- yl)propan-2-yl 2,3- dihydroxypropanoate. | 511.29 | (>95%) white solid |
| 68 | 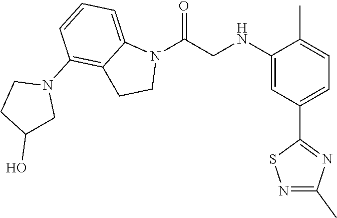 [SMILES: Cc1nsc(-c2ccc(C)c(NCC(=O)N3CCc4c(N5CCC(O)C5)cccc43)c2)n1] | ++++ | 1-(4-(3-hydroxypyrrolidin- 1-yl)indolin-1-yl)-2-((2- methyl-5-(3-methyl-1,2,4- thiadiazol-5-yl)phenyl) amino)ethan-1-one. | 450.28 | (>95%) white solid |
| 69 | 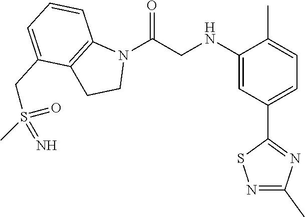 | + | imino(methyl)((1-((2- methyl-5-(3-methyl-1,2,4- thiadiazol-5-yl)phenyl) glycyl)indolin-4-yl) methyl)sulfanone. | 456.15 | (>95%) White solid |
| 70 | 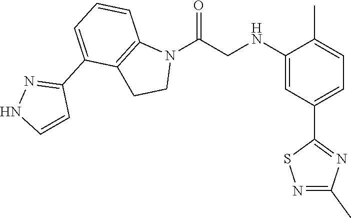 [SMILES: Cc1nsc(-c2ccc(C)c(NCC(=O)N3CCc4c(-c5cc[nH]n5)cccc43)c2)n1] | +++++ | 1-(4-(1H-pyrazol-3-yl) indolin-1-yl)-2-((2- methyl-5-(3-methyl- 1,2,4-thiadiazol-5- yl)phenyl)amino)ethan-1- one. | 431.21 | (>95%) Off white solid |
| 71 | 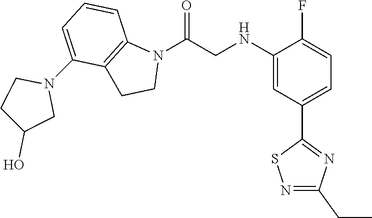 [SMILES: CCc1nsc(-c2ccc(F)c(NCC(=O)N3CCc4c(N5CCC(O)C5)cccc43)c2)n1] | +++++ | 2-((5-(3-ethyl-1,2,4- thiadiazol-5-yl)-2- fluorophenyl)amino)-1- (4-(3-hydroxypyrrolidin- 1-yl)indolin-1-yl) ethan-1-one. | 468.15 | (>95%) Yellow solid |
| 72 | 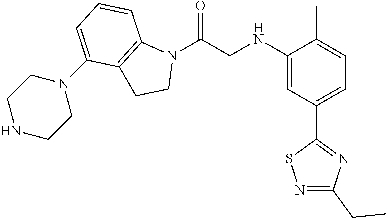 [SMILES: CCc1nsc(-c2ccc(C)c(NCC(=O)N3CCc4c(N5CCNCC5)cccc43)c2)n1] | ++++ | 2-((5-(3-ethyl-1,2,4- thiadiazol-5-yl)-2-methyl- phenyl)amino)-1-(4- (piperazin-1-yl)indolin-1- yl)ethan-1-one. | 463.22 | (>95%) Off white solid |
| 73 | 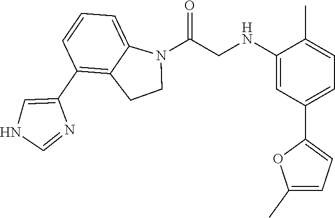 [SMILES: Cc1ccc(-c2ccc(C)c(NCC(=O)N3CCc4c(-c5c[nH]cn5)cccc43)c2)o1] | +++ | 1-(4-(1H-imidazol-4-yl) indolin-1-yl)-2-((2- methyl-5-(5-methyl- furan-2-yl)phenyl) amino)ethan-1-one. | 413.17 | (>95%) Off white solid |
| 74 | 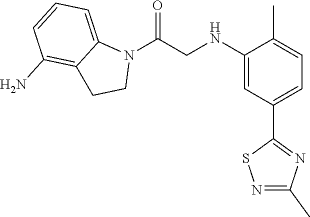 [SMILES: Cc1nsc(-c2ccc(C)c(NCC(=O)N3CCc4c(N)cccc43)c2)n1] | ++++ | 1-(4-aminoindolin-1-yl)-2- ((2-methyl-5- (3-methyl-1,2,4- thiadiazol-5-yl)phenyl)- amino)ethan-1-one. | 380.1 | 97.90 Yellow solid |
| 75 | 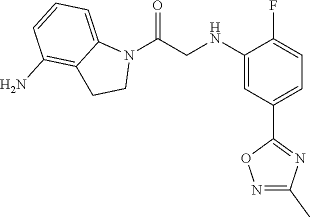 [SMILES: Cc1noc(-c2ccc(F)c(NCC(=O)N3CCc4c(N)cccc43)c2)n1] | ++ | 1-(4-aminoindolin-1-yl)- 2-((2-fluoro-5-(3- methyl-1,2,4-oxadiazol- 5-yl)phenyl)- amino)ethan-1-one. | 368.1 | 95.70 White solid |
| 76 | 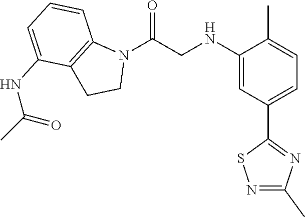 [SMILES: CC(=O)Nc1cccc2c1CCN2C(=O)CNc1cc(-c2nc(C)ns2)ccc1C] | +++++ | N-(1-((2-methyl-5-(3- methyl-1,2,4- thiadiazol-5-yl)phenyl)- glycyl)indolin-4-yl) acetamide. | 422.3 | 96.09 White solid |
| 77 | 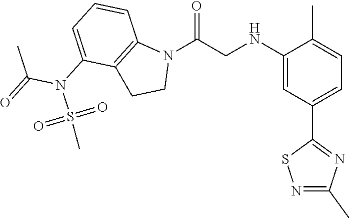 [SMILES: CC(=O)N(c1cccc2c1CCN2C(=O)CNc1cc(-c2nc(C)ns2)ccc1C)S(C)(=O)=O] | ++++ | N-(1-((2-methyl-5-(3- methyl-1,2,4-thiadiazol- 5-yl)phenyl)- glycyl)indolin-4-yl)-N- (methylsulfonyl)acetamide. | 500.3 | 98.33 White solid |
| 78 | 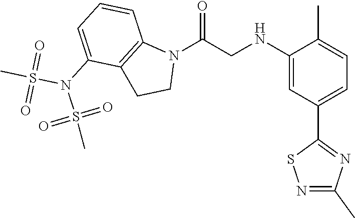 | +++++ | N-(1-((2-methyl-5-(3- methyl-1,2,4- thiadiazol-5-yl)phenyl) glycyl)indolin-4-yl)-N- (methylsulfonyl) methanesulfonamide. | 536.3 | 97.86 White solid |
| 79 | 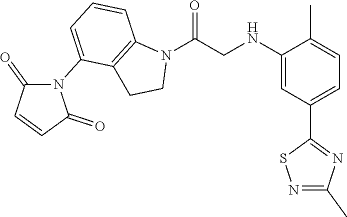 [SMILES: Cc1nsc(-c2ccc(C)c(NCC(=O)N3CCc4c3cccc4N3C(=O)C=CC3=O)c2)n1] | +++++ | 1-(1-((2-methyl-5-(3- methyl-1,2,4-thiadiazol- 5-yl)phenyl)- glycyl)indolin-4-yl)-1H- pyrrole-2,5-dione. | 460.3 | 92 White powder |
| 80 | 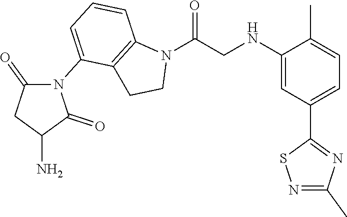 [SMILES: Cc1nsc(-c2ccc(C)c(NCC(=O)N3CCc4c3cccc4N3C(=O)CC(N)C3=O)c2)n1] | +++++ | 3-amino-1-(1-((2- methyl-5-(3-methyl-1,2,4- thiadiazol-5-yl)phenyl) glycyl)indolin-4-yl) pyrrolidine-2,5-dione. | 477.2 | 93.2 Yellow powder |
| 81 | 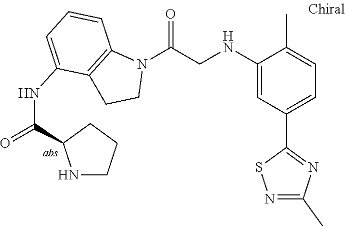 [SMILES: Cc1nsc(-c2ccc(C)c(NCC(=O)N3CCc4c(NC(=O)[C@H]5CCCN5)cccc43)c2)n1] | +++++ | (R)-N-(1-((2-methyl-5-(3- methyl-1,2,4-thiadiazol- 5-yl)phenyl)glycyl) indolin-4-yl) pyrrolidine-2- carboxamide. | 477.1 | 97.0 Light yellow solid |
| 82 | 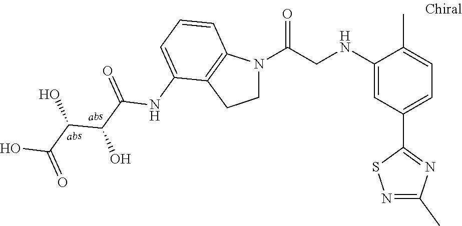 [SMILES: Cc1nsc(-c2ccc(C)c(NCC(=O)N3CCc4c(NC(=O)[C@H](O)[C@@H](O)C(=O)O)cccc43)c2)n1] | ++ | (2R,3R)-2,3-dihydroxy- 4-((1-((2-methyl-5-(3- methyl-1,2,4-thiadiazol- 5-yl)phenyl)- glycyl)indolin-4-yl) amino)-4-oxobutanoic acid. | 512.0 | 94.0 Yellow solid |
| 83 | 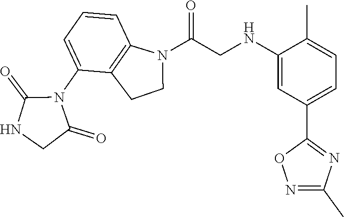 [SMILES: Cc1noc(-c2ccc(C)c(NCC(=O)N3CCc4c3cccc4N3C(=O)CNC3=O)c2)n1] | ++++ | 3-(1-((2-methyl-5-(3- methyl-1,2,4-oxadiazol- 5-yl)phenyl)glycyl) indolin-4-yl) imidazolidine-2,4-dione. | 447.2 | 98.4 Colorless solid |
| 84 | 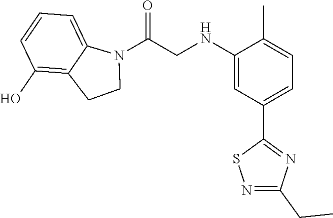 [SMILES: CCc1nsc(-c2ccc(C)c(NCC(=O)N3CCc4c(O)cccc43)c2)n1] | +++++ | 2-((5-(3-ethyl-1,2,4- thiadiazol-5-yl)-2- methylphenyl)amino)-1- (4-hydroxyindolin-1-yl) ethan-1-one. | 395.3 | 98.04 White solid |
| 85 | 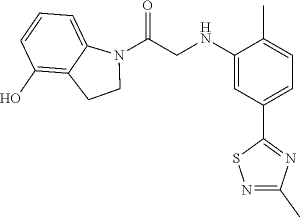 [SMILES: Cc1nsc(-c2ccc(C)c(NCC(=O)N3CCc4c(O)cccc43)c2)n1] | +++++ | 1-(4-hydroxyindolin-1- yl)-2-((2-methyl-5-(3- methyl-1,2,4- thiadiazol-5-yl)phenyl)- amino)ethan-1-one. | 381.1 | 98.15 White solid |
| 86 | 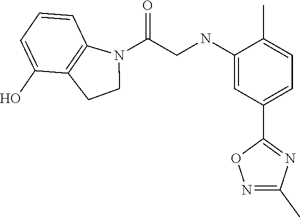 [SMILES: Cc1noc(-c2ccc(C)c(NCC(=O)N3CCc4c(O)cccc43)c2)n1] | +++ | 1-(4-hydroxyindolin-1- yl)-2-((2-methyl-5-(3- methyl-1,2,4-oxadiazol- 5-yl)phenyl)amino)- ethan-1-one. | 365.19 | 93 white solid |
| 87 | 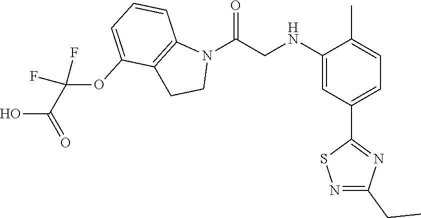 [SMILES: CCc1nsc(-c2ccc(C)c(NCC(=O)N3CCc4c(OC(F)(F)C(=O)O)cccc43)c2)n1] | +++ | 2-((1-((5-(3-ethyl-1,2,4- thiadiazol-5-yl)-2-methyl- phenyl)glycyl)indolin-4- yl)oxy)-2,2-difluoro- acetic acid. | 489.2 | 97 Yellow solid |
| 88 | 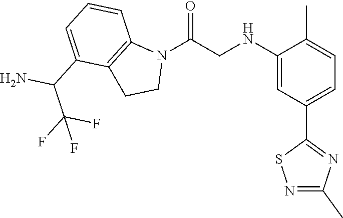 [SMILES: Cc1nsc(-c2ccc(C)c(NCC(=O)N3CCc4c(C(N)C(F)(F)F)cccc43)c2)n1] | +++ | 1-(4-(1-amino-2,2,2- trifluoroethyl)indolin- 1-yl)-2-((2-methyl-5- (3-methyl-1,2,4- thiadiazol-5-yl)phenyl)- amino)ethan-1-one. | 462.2 | 98.6 White solid |
| 89 | 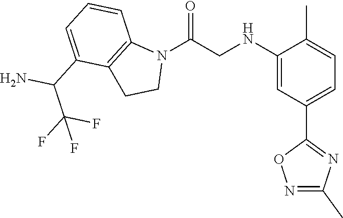 [SMILES: Cc1noc(-c2ccc(C)c(NCC(=O)N3CCc4c(C(N)C(F)(F)F)cccc43)c2)n1] | ++ | 1-(4-(1-amino-2,2,2- trifluoroethyl)indolin-1- yl)-2-((2-methyl-5-(3- methyl-1,2,4-oxadiazol- 5-yl)phenyl)- amino)ethan-1-one. | 446.1 | 96 White solid |
| 90 | 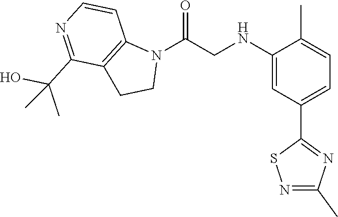 [SMILES: Cc1nsc(-c2ccc(C)c(NCC(=O)N3CCc4c3ccnc4C(C)(C)O)c2)n1] | + | 1-(4-(2-hydroxypropan-2- yl)-2,3-dihydro-1H- pyrrolo[3,2-c]pyridin-1- yl)-2-((2-methyl-5-(3- methyl-1,2,4-thiadiazol-5- yl)phenyl)amino)ethan-1- one. | 424.13 | 98 white solid |
| 91 | 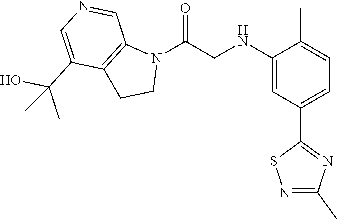 [SMILES: Cc1nsc(-c2ccc(C)c(NCC(=O)N3CCc4c3cncc4C(C)(C)O)c2)n1] | + | 1-(4-(2-hydroxypropan- 2-yl)-2,3-dihydro-1H- pyrrolo[2,3-c]pyridin-1- yl)-2-((2-methyl-5- (3-methyl-1,2,4-thiadiazol- 5-yl)phenyl)amino) ethan-1-one. | 424.13 | 99 white solid |
| 92 | 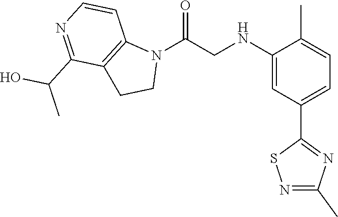 [SMILES: Cc1nsc(-c2ccc(C)c(NCC(=O)N3CCc4c3ccnc4C(C)O)c2)n1] | + | 1-(4-(1-hydroxyethyl)-2,3- dihydro-1H-pyrrolo[3,2- c]pyridin-1-yl)-2-((2- methyl-5-(3-methyl- 1,2,4-thiadiazol-5- yl)phenyl)amino)ethan- 1-one. | 408.01 | 99 White solid |
| 93 | 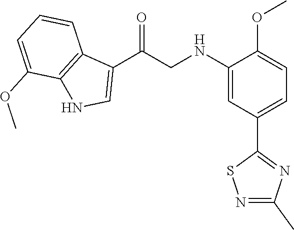 [SMILES: COc1ccc(-c2nc(C)ns2)cc1NCC(=O)c1c[nH]c2c(OC)cccc12] | +++++ | 1-(7-methoxy-1H-indol- 3-yl)-2-((2-methoxy-5- (3-methyl-1,2,4- thiadiazol-5-yl)phenyl)- amino)ethan-1-one. | 409.2 | 96.1 Beige powder |
| 94 | 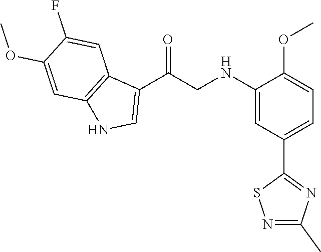 | +++++ | 1-(5-fluoro-6-methoxy-1H- indol-3-yl)-2-((2- methoxy-5-(3-methyl- 1,2,4-thiadiazol-5-yl) phenyl)amino)ethan-1- one. | 427.2 | 95.3 Beige- gray solid |
| 95 | 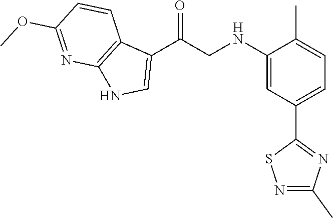 | +++++ | 1-(6-methoxy-1H- pyrrolo[2,3-b]pyridin- 3-yl)-2-((2-methyl-5- (3-methyl-1,2,4- thiadiazol-5-yl)phenyl) amino)ethan-1-one. | 394.2 | 95 Yellow solid |
| 96 | 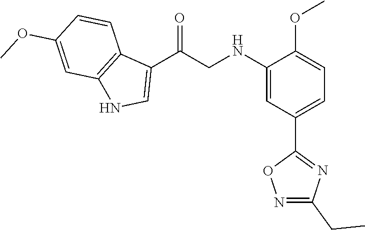 [SMILES: CCc1noc(-c2ccc(OC)c(NCC(=O)c3c[nH]c4cc(OC)ccc34)c2)n1] | +++++ | 2-((5-(3-ethyl-1,2,4- oxadiazol-5-yl)-2- methoxyphenyl)amino)- 1-(6-methoxy-1H-indol-3- yl)ethan-1-one. | 407 | 98.9 Yellow solid |
| 97 | 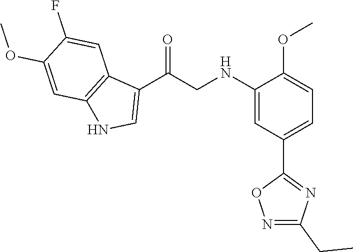 [SMILES: CCc1noc(-c2ccc(OC)c(NCC(=O)c3c[nH]c4cc(OC)c(F)cc34)c2)n1] | +++++ | 2-((5-(3-ethyl-1,2,4- oxadiazol-5-yl)-2- methoxyphenyl)amino)- 1-(5-fluoro-6- methoxy-1H-indol-3-yl) ethan-1-one. | 425.2 | 98.9 Beige powder |
| 98 | 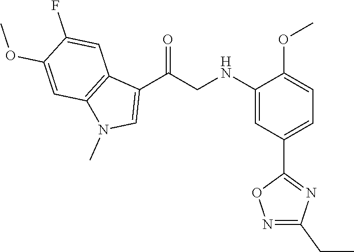 [SMILES: CCc1noc(-c2ccc(OC)c(NCC(=O)c3cn(C)c4cc(OC)c(F)cc34)c2)n1] | +++++ | 2-((5-(3-ethyl-1,2,4- oxadiazol-5-yl)-2- methoxyphenyl)amino)- 1-(5-fluoro-6-methoxy-1- methyl-1H-indol-3-yl) ethan-1-one. | 438.8 | 98.5 Light yellow powder |
| 99 | 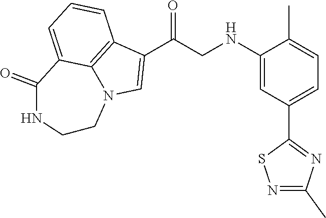 [SMILES: Cc1nsc(-c2ccc(C)c(NCC(=O)c3cn4c5c(cccc35)C(=O)NCC4)c2)n1] | ++++ | 7-((2-methyl-5-(3-methyl- 1,2,4-thiadiazol-5-yl) phenyl)glycyl)-3,4- dihydro- [1,4]diazepino[6,7,1-hi] indol-1(2H)-one. | 432.09 | (>95%) Off white solid |
| 100 | 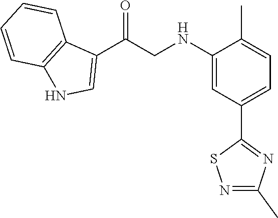 [SMILES: Cc1nsc(-c2ccc(C)c(NCC(=O)c3c[nH]c4ccccc34)c2)n1] | ++++ | 2-((2-methyl-5-(3- methyl-1,2,4- thiadiazol-5-yl)phenyl) amino)-1-(1H-pyrrolo [2,3-b]pyridin-3-yl) ethan-1-one. | 364.1 | 91.5 Beige solid |
| 101 | 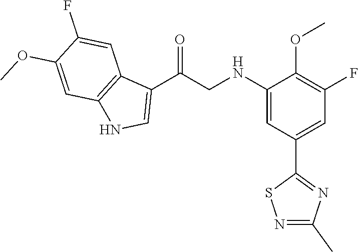 [SMILES: COc1cc2[nH]cc(C(=O)CNc3cc(-c4nc(C)ns4)cc(F)c3OC)c2cc1F] | ++++ | 2-((3-fluoro-2- methoxy-5-(3-methyl- 1,2,4-thiadiazol-5-yl) phenyl)amino)-1-(5- fluoro-6-methoxy-1H- indol-3-yl)ethan-1-one. | 445 | 95.9 Beige- gray solid |
| 102 | 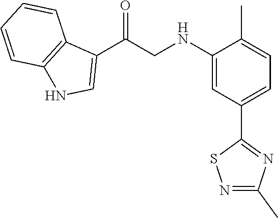 [SMILES: Cc1nsc(-c2ccc(C)c(NCC(=O)c3c[nH]c4ccccc34)c2)n1] | ++++ | 1-(1H-indol-3-yl)-2-((2- methyl-5-(3-methyl-1,2,4- thiadiazol-5-yl)phenyl) amino)ethan-1-one. | 363 | 99.4 Yellow solid |
| 103 | 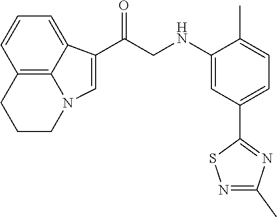 [SMILES: Cc1nsc(-c2ccc(C)c(NCC(=O)c3cn4c5c(cccc35)CCC4)c2)n1] | ++++ | 1-(5,6-dihydro-4H- pyrrolo[3,2,1-ij]quinolin- 1-yl)-2-((2-methyl-5-(3- methyl-1,2,4-thiadiazol-5- yl)phenyl)amino)ethan-1- one. | 403.4 | 96.9 Light beige powder |
| 104 | 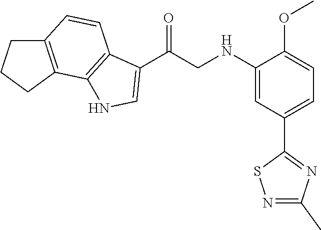 [SMILES: COc1ccc(-c2nc(C)ns2)cc1NCC(=O)c1c[nH]c2c3c(ccc12)CCC3] | ++++ | 2-((2-methoxy-5-(3- methyl-1,2,4-thiadiazol- 5-yl)phenyl)amino)-1- (1,6,7,8- tetrahydrocyclopenta[g] indol-3-yl)ethan-1-one. | 419 | 93.2 Beige- yellow solid |
| 105 | 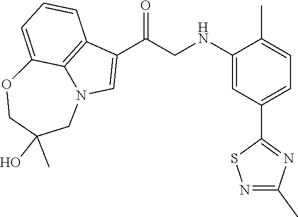 [SMILES: Cc1nsc(-c2ccc(C)c(NCC(=O)c3cn4c5c(cccc35)OCC(C)(O)C4)c2)n1] | ++++ | 1-(3-hydroxy-3-methyl- 3,4-dihydro-2H- [1,4]oxazepino[2,3,4-hi] indol-7-yl)-2-((2-methyl- 5-(3-methyl-1,2,4- thiadiazol-5-yl)phenyl)- amino)ethan-1-one. | 449.24 | (>95%) Off white solid |
| 106 | 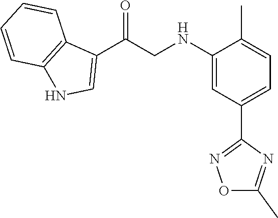 [SMILES: Cc1nc(-c2ccc(C)c(NCC(=O)c3c[nH]c4ccccc34)c2)no1] | +++ | 1-(1H-indol-3-yl)-2-((2- methyl-5-(5-methyl-1,2,4- oxadiazol-3-yl)phenyl) amino)ethan-1-one. | 347.42 | 95 Yellow solid |
| 107 | 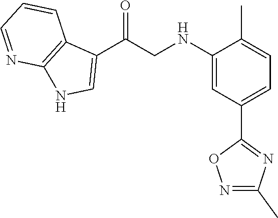 [SMILES: Cc1noc(-c2ccc(C)c(NCC(=O)c3c[nH]c4ncccc34)c2)n1] | ++ | 2-((2-methyl-5-(3-methyl- 1,2,4-oxadiazol-5-yl) phenyl)amino)-1-(1H- pyrrolo[2,3-b]pyridin-3- yl)ethan-1-one. | 348 | 96.7 White solid |
| 108 | 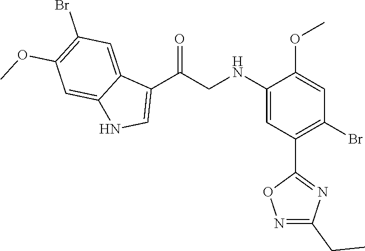 [SMILES: CCc1noc(-c2cc(NCC(=O)c3c[nH]c4cc(OC)c(Br)cc34)c(OC)cc2Br)n1] | ++ | 2-(4-bromo-5-(3-ethyl- 1,2,4-oxadiazol-5-yl)-2- methoxyphenylamino)-1- (5-bromo-6-methoxy- 1H-indol-3-yl) ethanone. | 565 | 95.3 Yellowish powder |
| 109 | 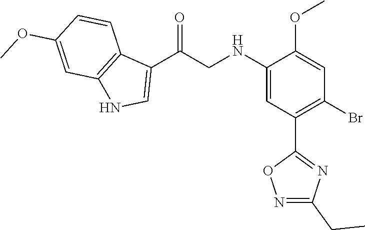 [SMILES: CCc1noc(-c2cc(NCC(=O)c3c[nH]c4cc(OC)ccc34)c(OC)cc2Br)n1] | +++ | 2-((4-bromo-5-(3-ethyl- 1,2,4-oxadiazol-5-yl)-2- methoxy- phenyl)amino)-1-(6- methoxy-1H-indol-3- yl)ethan-1-one. | 485.1 | 98.1 Beige powder |
| 110 | 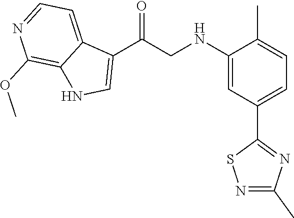 [SMILES: COc1nccc2c(C(=O)CNc3cc(-c4nc(C)ns4)ccc3C)c[nH]c12] | ++++ | 1-(7-methoxy-1H- pyrrolo[2,3-c]pyridin-3- yl)-2-((2-methyl-5-(3- methyl-1,2,4-thiadiazol- 5-yl)phenyl)amino) ethan-1-one | 394.2 | 90 yellow powder |
| 111 | 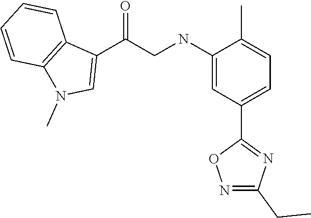 [SMILES: CCc1noc(-c2ccc(C)c(NCC(=O)c3cn(C)c4ccccc34)c2)n1] | +++ | 2-((5-(3-ethyl-1,2,4- oxadiazol-5-yl)-2- methylphenyl)amino)-1- (1-methyl-1H-indol-3- yl)ethan-1-one. | 375 | 98.7 Pale yellow solid |
| 112 | 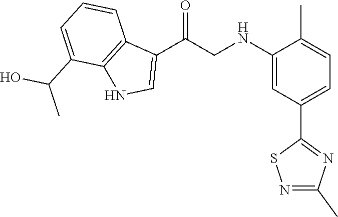 [SMILES: Cc1nsc(-c2ccc(C)c(NCC(=O)c3c[nH]c4c(C(C)O)cccc34)c2)n1] | +++++ | 1-(7-(1-hydroxyethyl)-1H- indol-3-yl)-2-((2-methyl- 5-(3-methyl-1,2,4- thiadiazol-5-yl) phenyl)amino)ethan-1-one. | 407 | 98.9 White solid |
| 113 | 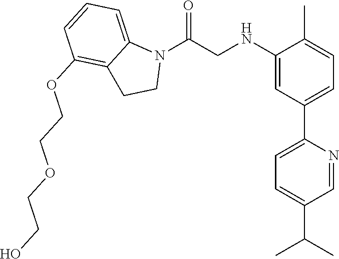 [SMILES: Cc1ccc(-c2ccc(C(C)C)cn2)cc1NCC(=O)N1CCc2c(OCCOCCO)cccc21] | +++ | 1-(4-(2-(2- hydroxyethoxy)ethoxy) indolin-1-yl)-2-((5-(5- isopropylpyridin-2-yl)-2- methylphenyl)amino) ethan-1-one. | 490.2 | 96.10 White solid |
| 114 | 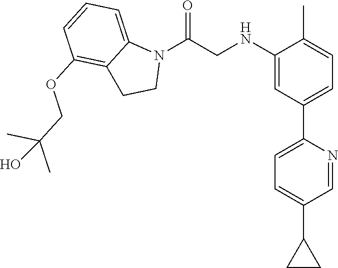 [SMILES: Cc1ccc(-c2ccc(C3CC3)cn2)cc1NCC(=O)N1CCc2c(OCC(C)(C)O)cccc21] | +++ | 2-((5-(5-cyclopropyl- pyridin-2-yl)-2-methyl- phenyl)amino)-1-(4-(2- hydroxy-2-methyl- propoxy)indolin-1-yl) ethan-1-one. | 472.29 | (>95%) White solid |
| 115 | 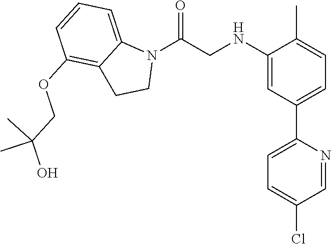 [SMILES: Cc1ccc(-c2ccc(Cl)cn2)cc1NCC(=O)N1CCc2c(OCC(C)(C)O)cccc21] | +++ | 2-((5-(5-chloropyridin-2- yl)-2-methylphenyl) amino)-1-(4-(2- hydroxy-2-methyl- propoxy)- indolin-1-yl)ethan- 1-one. | 466.2 | 95.1 White powder |
| 116 | 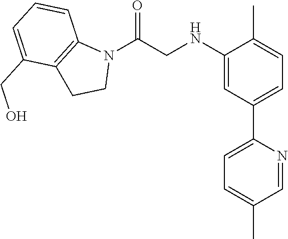 [SMILES: Cc1ccc(-c2ccc(C)c(NCC(=O)N3CCc4c(CO)cccc43)c2)nc1] | ++ | 1-(4-(hydroxymethyl) indolin-1-yl)-2-((2- methyl-5-(5- methylpyridin-2-yl) phenyl)amino)ethan-1- one. | 388.4 | 99.6 White powder |
| 117 | 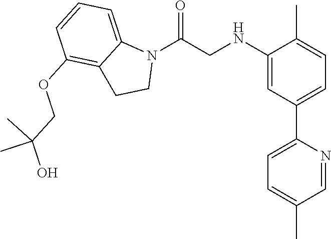 [SMILES: Cc1ccc(-c2ccc(C)c(NCC(=O)N3CCc4c(OCC(C)(C)O)cccc43)c2)nc1] | +++ | 1-(4-(2-hydroxy-2- methylpropoxy)indolin-1- yl)-2-((2-methyl-5-(5- methylpyridin-2-yl) phenyl)amino)ethan-1- one. | 446.3 | 98.58 White solid |
| 118 | 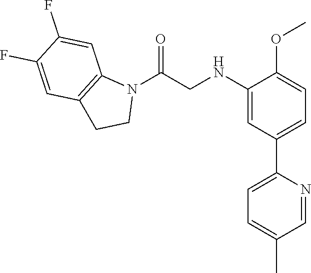 [SMILES: COc1ccc(-c2ccc(C)cn2)cc1NCC(=O)N1CCc2cc(F)c(F)cc21] | +++ | 1-(5,6-difluoroindolin-1- yl)-2-((2-methoxy-5-(5- methylpyridin-2- yl)phenyl)amino)ethan-1- one. | 410.2 | (>95%) White solid |
| 119 | 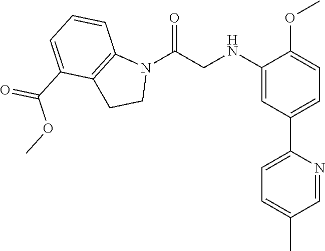 [SMILES: COC(=O)c1cccc2c1CCN2C(=O)CNc1cc(-c2ccc(C)cn2)ccc1OC] | ++ | methyl 1-((2-methoxy-5- (5-methylpyridin-2- yl)phenyl)glycyl) indoline-4-carboxylate. | 432.1 | (>95%) White solid |
| 120 | 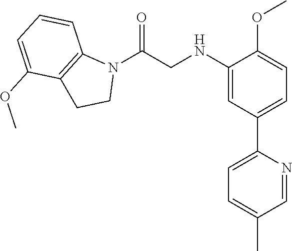 [SMILES: COc1ccc(-c2ccc(C)cn2)cc1NCC(=O)N1CCc2c(OC)cccc21] | +++ | 2-((2-methoxy- 5-(5-methyl- pyridin-2-yl) phenyl)amino)-1-(4- methoxyindolin-1-yl) ethan-1-one. | 404.2 | (>95%) White solid |
| 121 | 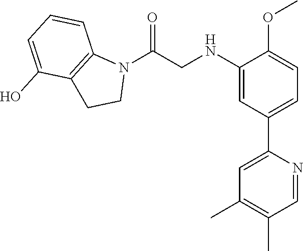 [SMILES: COc1ccc(-c2cc(C)c(C)cn2)cc1NCC(=O)N1CCc2c(O)cccc21] | + | 2-((5-(4,5-dimethyl- pyridin-2-yl)-2- methoxyphenyl)amino)-1- (4-hydroxyindolin-1-yl) ethan-1-one. | 404.2 | (>95%) White solid |
| 122 | 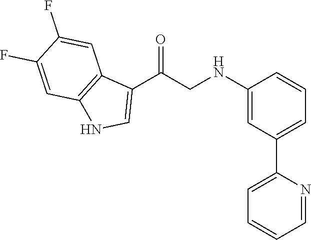 [SMILES: O=C(CNc1cccc(-c2ccccn2)c1)c1c[nH]c2cc(F)c(F)cc12] | + | 1-(5,6-difluoro-1H-indol- 3-yl)-2-((3-(pyridin-2- yl)phenyl)amino)ethan- 1-one. | 364.1 | (>95%) White solid |
| 123 | 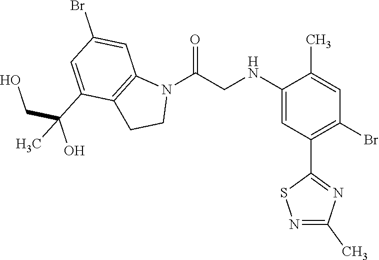 [SMILES: Cc1nsc(-c2cc(NCC(=O)N3CCc4c3cc(Br)cc4[C@@](C)(O)CO)c(C)cc2Br)n1] | + | 2-((4-bromo-2-methyl-5- (3-methyl-1,2,4- thiadiazol-5-yl)phenyl) amino)-1-(6-bromo-4- (1,2-dihydroxypropan-2- yl)indolin-1-yl)ethan- 1-one. | 593.9 | (>95%) Off white solid |

*P2X4 inhibitory potency (IC₅₀ values): + (>100 nM); ++ (70-99 nM); +++ (40-69 nM); ++++ (20-39 nM); +++++ (0.1-19 nM)

### Synthesis of P2X4 Radioligand [³H]PSB-OR-2020

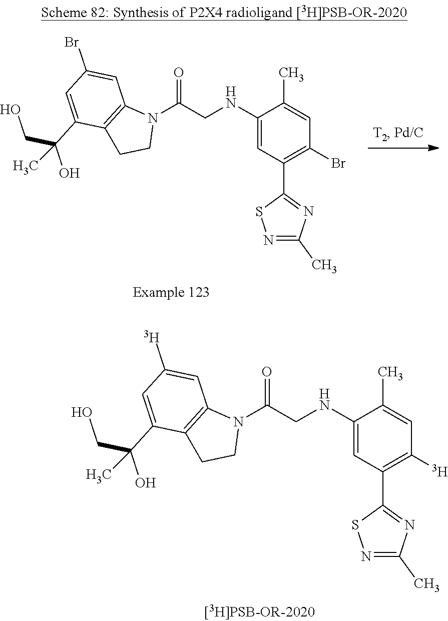 [SMILES: Cc1nsc(-c2cc(NCC(=O)N3CCc4c3cc(Br)cc4[C@@](C)(O)CO)c(C)cc2Br)n1 ; [3H]c1cc2c(c([C@@](C)(O)CO)c1)CCN2C(=O)CNc1cc(-c2nc(C)ns2)c([3H])cc1C]

The P2X4 radioligand [³H]PSB-OR-2020 was prepared via custom-labeling from a suitable bromo-substituted precurser (123) by catalytic hydrogenation using tritium gas. It was obtained with a radiochemical purity of 97.7% and a specific radioactivity of 45 Ci/mmol. The radioligand can be used for selective or specific labeling of P2X4 receptors, in general for proteins that selectively or specifically interact with the radiotracer.

### BIOLOGICAL EXPERIMENTAL DATA

### Stable Expression of the Rat, Mouse and Human P2X4 Receptors in Astrocytoma 1321N1 Cells

The rat, mouse, and human P2X4 DNA, respectively, were cloned into the retroviral vector pQCXIN. 1321N1 Astrocytoma cells were transfected with the DNA using a retroviral system as described previously. In brief, GP+envAM12 packaging cells were first transiently co-transfected with a retroviral vector to generate the retrovirus and with a vesicular stomatitis virus G protein (VSV-G) to pseudotype the virus and to increase their infection efficiency. The viruses containing the rat, mouse or human P2X4 receptor sequence were then used to create a stable astrocytoma cell line. P2X4 receptor-expressing cells were selected with G418 (800 g/mL).

### Selection of Monoclones

The selection of monoclones for rat and human P2X4 receptors was performed using the limited dilution method by plating the cells at very low cell densities (1 cell clone per well in 96-well plates). Cells were counted, and then several dilutions were made to obtain a concentration of 100 cells/10 mL. We subsequently added 100 L of the diluted cell suspension into each well expecting to obtain on average 1 cell per well. After a few days the wells that contained single clones were labeled and transferred into 24 well plates. More than 20 clones were selected for each cell line and characterized in calcium influx assay. Only the clones that showed a large calcium signal in preliminary studies were passaged to be further used for functional and binding studies. In our hands, the selected monoclonal cell lines showed stable pharmacological properties at least up to passage 30.

Measurement of Ca²⁺ influx in transfected 1321N1 astrocytoma cells

P2X receptor function was determined on the basis of agonist-mediated increases in cytosolic Ca²⁺ concentration. The fluorescent Ca²⁺chelating dye FLUO-4 was used as an indicator of the relative levels of intracellular Ca²⁺ in a 96-well format using a fluorescence imaging plate reader (Novostar, BMG, Germany). Cells were grown to confluence in 96-well black-walled tissue culture plates and loaded with FLUO-4 AM (2.4 M) in Hank's balanced salt solution (HBSS, containing 10 mM HEPES, pH 7.3, and 1% Pluronic® F127) for 1 h at 23° C. After incubation, the loaded cells were washed with the same buffer to remove extracellular FLUO-4 AM. Compound solutions were prepared in HBSS (containing 20 mM HEPES, pH 7.3) or DMSO depending on their solubility. The final DMSO concentration in the assays did not exceed 1%. This concentration of DMSO was found to be well tolerated by the cells. Fluorescence intensity was measured at 520 nm for 30 s at 0.4 s intervals. Buffer or test compounds were injected sequentially into separate wells using the automatic pipetting device. At least three independent experiments were performed in triplicate or duplicate. Antagonists were added 30 min before the addition of agonists. The assays were performed in a final volume of 200 μL. Compounds were tested at 7-8 different concentrations spanning three orders of magnitude of concentrations. Statistical significances were calculated using the unpaired two-tailed t-test.

What is claimed is:

1. A compound according to general Formula (A), general Formula (B), general Formula (C) and/or general Formula (D), 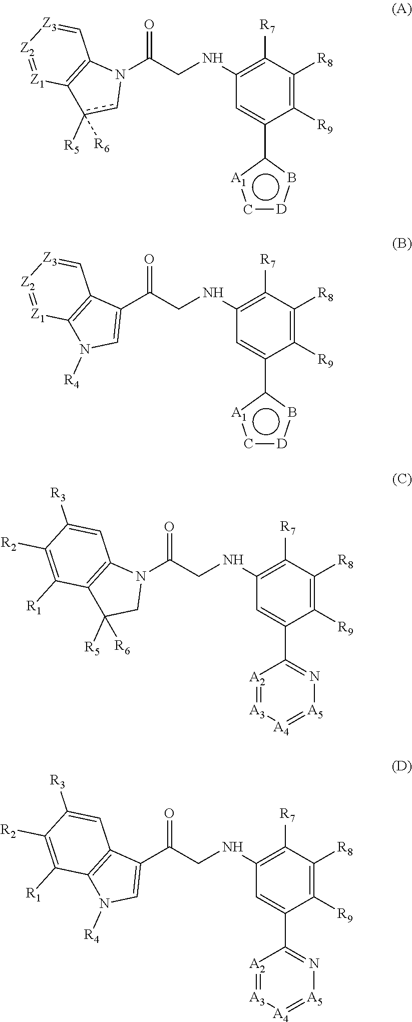
wherein, respectively independently,
Z₁ means N or C—R₁;
Z₂ means N or C—R₂;
Z₃ means N or C—R₃;
A₁ means —O—, —S—, N, —CH or C-Rₓ;
A₂ means N, —CH or C-Rₓ;
A₃ means N or C-R₁₁;
A₄ means N or C-R₁₂;
A₅ means N or C-R₁₃;
B and C, respectively independently, mean —O—, —S—, N, —CH or C-Rₓ;
D means N, N-Rₓ or C-R₁₀;
R₁, R₂, R₃, R₄, R₅, R₆, R₇, Rs, R₉, R₁₀, R₁, R₁₂ and R₁₃, respectively independently, mean —H, —F, —Cl, —Br, —I, —CN, —NO₂, —CHO, ═O, —Rₓ, —C(═O)Rₓ, —C(═O)H, —C(═O)OH, —C(═O)ORₓ, —C(═O)NH₂, —C(═O)NHRₓ, —C(═O)N(Rₓ)₂, —OH, —ORₓ, —OC(═O)H, —OC(═O)Rₓ, —OC(═O)—ORₓ, —OC(═O)NHRₓ, —OC(═O)N(Rₓ)₂, —SH, -SRₓ, —SO₃H, —S(═O)₁₋₂-Rₓ, —S(═O)₁₋₂NH₂, —S(═O)₁₋₂NHRₓ, —S(═O)₁₋₂N(Rₓ)₂, —NH₂, —NHRₓ, —N(Rₓ)₂, —N⁺(Rₓ)₃, —N⁺(Rₓ)₂ O⁻, —NH—S(═O)₁₋₂-Rₓ, —NHC(═O)Rₓ, —N(S(═O)₂—CH₃)(C(═O)Rₓ), —N(S(═O)₂—CH₃)₂, —NHC(═O)ORₓ, —NHC(═O)NH₂, —NHC(═O)NHRₓ, —NHC(═O)—N(Rₓ)₂, —Si(Rₓ)₃ or —PO(ORₓ)₂;
or R₁ and R₂ jointly form a ring, the ring atoms of which respectively independently of one another are C, S or O, wherein the ring is aromatic or non-aromatic, unsubstituted or mono- or polysubstituted by substituents selected independently of one another from the group consisting of —F, —Cl, —Br, —I, —CN, —NO₂, —CHO, ═O, —CH₃ and —OH,
or R₁ and R₄ jointly form a ring, the ring atoms of which respectively independently of one another are C, S or O, wherein the ring is aromatic or non-aromatic, unsubstituted or mono- or polysubstituted by substituents selected independently of one another from the group consisting of —F, —Cl, —Br, —I, —CN, —NO₂, —CHO, ═O, —CH₃ and —OH,
wherein in each case Rₓ, respectively independently, means —C₁₋₈-aliphatic, -C₃₋₁₂-cycloaliphatic, -aryl, heteroaryl, —C₁₋₈-aliphatic-C₃₋₁₂-cycloaliphatic, —C₁₋₈-aliphatic-aryl, —C₁₋₈-aliphatic-heteroaryl, -C₃₋₈-cycloaliphatic—C₁₋₈-aliphatic, -C₃₋₈-cycloaliphatic-aryl or -C₃-s-cycloaliphatic-heteroaryl;
wherein in each case “aliphatic”, respectively independently, means a branched or unbranched, saturated or a mono- or polyunsaturated, unsubstituted or mono- or polysubstituted, aliphatic hydrocarbon residue;
wherein in each case “cycloaliphatic”, respectively independently, means a saturated or a mono- or polyunsaturated, unsubstituted or mono- or polysubstituted, mono- or multicyclic hydrocarbon residue, either alicyclic or wherein possibly one or two or more carbon atoms are replaced independently of one another by a heteroatom S, N or O;
wherein in each case with respect to “aliphatic” and “cycloaliphatic”, “mono- or polysubstituted”, respectively independently, means the mono- or polysubstitution of one or more hydrogen atoms by —F, -Cl, —Br, —I, —CN, —NO₂, —CHO, ═O, —Rₓ, —C(═O)Rₓ, —C(═O)H, —C(═O)OH, —C(═O)ORₓ, —C(═O)NH₂, —C(═O)NHRₓ, —C(═O)N(Rₓ)₂, —OH, —ORₓ, —OC(═O)H, —OC(═O)Rₓ, —OC(═O)—ORₓ, —OC(═O)NHRₓ, —OC(═O)N(Rₓ)₂, —SH, -SRₓ, —SO₃H, —S(═O)₁₋₂-Rₓ, —S(═O)₁₋₂NH₂, —S(═O)₁₋₂NHRₓ, —NH₂, —NHRₓ, —N(Rₓ)₂, —N⁺(Rₓ)₃, —N⁺(Rₓ)₂ O⁻, —NHC(═O)Rₓ, —NHC(═O)ORₓ, —NHC(═O)NH₂, —NHC(═O)NHRₓ, —NHC(═O)—N(Rₓ)₂, —Si(Rₓ)₃ or —PO(ORₓ)₂;
wherein in each case “aryl”, respectively independently, means a carbocyclic ring system with at least one aromatic ring, but without heteroatoms in this ring, wherein, if necessary, the aryl residues can be condensed with further saturated, (partially) unsaturated or aromatic ring systems, and each aryl residue can be present in unsubstituted or mono- or polysubstituted form, wherein the aryl substituents can be the same or different and in any desired and possible position of the aryl;
wherein in each case “heteroaryl”, respectively independently, means a 5-, 6- or 7-membered cyclic aromatic residue, which contains 1, 2, 3, 4 or 5 heteroatoms, wherein the heteroatoms, the same or different, are nitrogen, oxygen or sulphur, and the heterocycle can be unsubstituted or mono- or polysubstituted; wherein in the case of the substitution on the heterocycle the substituents can be the same or different and can be in any desired and possible position of the heteroaryl; and wherein the heterocycle can also be part of a bi- or polycyclic system;
wherein in each case with respect to “aryl” and “heteroaryl”, “mono- or polysubstituted”, respectively independently, means the mono- or polysubstitution of one or more hydrogen atoms of the ring system by substituents selected from the group comprising —F, —Cl, —Br, —I, —CN, —NO₂, —CHO, ═O, —Rₓ, —C(═O)Rₓ, —C(═O)H, —C(═O)OH, —C(═O)ORₓ, —C(═O)NH₂, —C(═O)NHRₓ, —C(═O)—N(Rₓ)₂, —OH, —O(CH₂)₁₋₂O—, —ORₓ, —OC(═O)H, —OC(═O)Rₓ, —OC(═O)ORₓ, —OC(═O)NHRₓ, —OC(═O)N(Rₓ)₂, —SH, -SRₓ, —SO₃H, —S(═O)₁₋₂-Rₓ , —S(═O)₁₋₂NH₂, —NH₂, —NHRₓ, —N(Rₓ)₂, —N⁺(Rₓ)₃, —N⁺(Rₓ)₂ O⁻, —NHC(═O)Rₓ, —NHC(═O)ORₓ, —NH—C(═O)NH₂, —NHC(═O)NHRₓ, —NHC(═O)—N(Rₓ)₂, —Si(Rₓ)₃ and —PO(ORₓ)₂; wherein if necessary N-ring atoms present can be respectively oxidized;
or a physiologically acceptable salt thereof or a physiologically acceptable solvate thereof.

2. The compound according to claim 1, wherein R₇, R₈ and R₉, respectively independently, mean
—H, —F, —Cl, —Br, —I, —CN, —NO₂, —CHO, —Rₓ, —OH, —ORₓ, —SH, -SRₓ, —SO₃H, —S(═O)₁₋₂-Rₓ, —S(═O)₁₋₂NH₂, —NH₂, —NHRₓ or -N(Rₓ)₂.

3. The compound according to claim 1, wherein R₇, R₈ and R₉, respectively independently, mean —H, —F, —Cl, —Br, —I, —Rₓ, —OH, —ORₓ, —NH₂, —NHRₓ or -N(Rₓ)₂.

4. The compound of claim 1, wherein R₇, R₈ and R₉, respectively independently, mean —H, —F, —Cl, —Br, —I, —Rₓ, —OH, —ORₓ, —NH₂, —NHRₓ or -N(Rₓ)₂, wherein Rₓ, respectively independently, means —C₁₋₈-aliphatic or —C₃₋₁₂-cycloaliphatic;
wherein
—C₁₋₈-aliphatic is preferably unsubstituted —(CH₂)ₘ—CH₃, wherein m is 0, 1, 2, 3, 4, 5, 6, 7, 8 or 9; and/or
—C₃₋₁₂-cycloaliphatic is preferably piperidinyl, piperazinyl or pyrrolidinyl, in each case unsubstituted.

5. The compound of claim 1, wherein R₁₀ means —H, —F, —Cl, —Br, —I, —CN, —Rₓ, —OH, —ORₓ, —NH₂, —NHRₓ or -N(Rₓ)₂.

6. The compound of claim 1, wherein R₁₀ means —H, —F, —Cl, —Br, —I, —CN, —Rₓ, —OH, —ORₓ, —NH₂, —NHRₓ or -N(Rₓ)₂, wherein Rₓ, respectively independently, means —C₁₋₈-aliphatic or —C₃₋₁₂-cycloaliphatic;
wherein
—C₁₋₈-aliphatic is preferably —(CH₂)ₘ—CH₃, wherein m is 0, 1, 2, 3, 4, 5, 6, 7, 8 or 9, in each case either unsubstituted or mono-, di- or trisubstituted with —F, or is preferably ethenyl, ethinyl, propenyl, propinyl, butenyl or butinyl, in each case unsubstituted; and/or
—C₃₋₁₂-cycloaliphatic is preferably cyclopropyl, cyclobutyl, cyclopentyl or cyclohexyl, in each case unsubstituted.

7. The compound of claim 1, wherein
A₂ means —CH or C-Rₓ, wherein Rₓ means —C₁₋₈-aliphatic or —C₃₋₁₂-cycloaliphatic; and A₃ means C—R₁₁;
A₄ means C—R₁₂; and A₅ means C—R₁₃; wherein R₁₁, R₁₂ and R₁₃, respectively independently, mean —H, —F, —Cl, —Br, —I or —Rₓ, wherein Rₓ, respectively independently, means —C₁₋₈-aliphatic or —C₃₋₁₂-cycloaliphatic;
wherein
—C₁₋₈-aliphatic is preferably unsubstituted —(CH₂)ₘ—CH₃, wherein m is 0, 1, 2, 3, 4, 5, 6, 7, 8 or 9, or is preferably 1-methylethyl, iso-butyl or tert.-butyl, in each case unsubstituted; and/or
—C₃₋₁₂-cycloaliphatic is preferably cyclopropyl, cyclobutyl, cyclopentyl or cyclohexyl, in each case unsubstituted.

8. The compound of claim 1, wherein R₄, Rs and R₆, respectively independently, mean —H, —Rₓ, —C(═O)Rₓ, —C(═O)H, —C(═O)OH, —C(═O)ORₓ,
—C(═O)NH₂, —C(═O)NHRₓ, —C(═O)N(Rₓ)₂, —OH, —ORₓ, —OC(═O)H, —OC(═O)Rₓ, —OC(═O)—ORₓ, —OC(═O)NHRₓ or —OC(═O)N(Rₓ)₂, wherein Rₓ, respectively independently, means —C₁₋₈-aliphatic or —C₃₋₁₂-cycloaliphatic;
wherein
—C₁₋₈-aliphatic is preferably —(CH₂)ₘ—CH₃, wherein m is 0, 1, 2, 3, 4, 5, 6, 7, 8 or 9, in each case unsubstituted or mono- or disubstituted with —OH; and/or
—C₃₋₁₂-cycloaliphatic is preferably cyclopropyl, cyclobutyl, cyclopentyl or cyclohexyl, in each case unsubstituted; or
wherein R₄ means —H or —(CH₂)ₘ—CH₃, wherein m is 0, 1, 2, 3, 4, 5, 6, 7, 8 or 9, in each case unsubstituted or mono- or disubstituted with —OH.

9. The compound of claim 1, wherein
B and C, respectively independently, mean —O—, N, —CH or C-Rₓ; wherein Rₓ, respectively independently, means C₁₋₈-aliphatic which is preferably unsubstituted —(CH₂)ₘ—CH₃, wherein m is 0, 1, 2, 3, 4, 5, 6, 7, 8 or 9; and/or
A₁ means —O—, —S—, N or —CH.

10. The compound of claim 1, wherein
R₁, R₂, R₃, respectively independently, mean —H, —F, —Cl, —Br, —Rₓ, —C(═O)Rₓ, —C(═O)ORₓ, —C(═O)NHRₓ, —OH, ORₓ, —S(═O)₁₋₂NH₂, —S(═O)₁₋₂NHRₓ, —CH₂—S(═O)₁₋₂NHRₓ, —NH₂, —NHRₓ, -NH—S(═O)₁₋₂-Rₓ, —NHC(═O)Rₓ, —N(S(═O)₂—CH₃)(C(═O)Rₓ) or —N(S(═O)₂—CH₃)₂; or
R₁ and R₂ together form a ring which is unsubstituted —(CH₂)~-, wherein n is 1, 2, 3, 4, 5, 6, 7, 8 or 9; or
R₁ and R₄ together form a ring which is unsubstituted —(CH₂)~-, wherein n is 1, 2, 3, 4, 5, 6, 7, 8 or 9; or
unsubstituted —(CH₂)ₒ—NH—C(═O)—, wherein o is 1, 2, 3, 4, 5, 6, 7, 8 or 9; or unsubstituted —(CH₂)ₚ—C(CH₃)(OH)—(CH₂)_{q}—O—, wherein p and q, respectively independently, are 1, 2, 3, 4, 5, 6, 7, 8 or 9.

11. The compound of claim 1, wherein, unless defined otherwise,
with respect to “aliphatic” and “cycloaliphatic”, “mono- or polysubstituted”, respectively independently, means the mono- or polysubstitution of one or more hydrogen atoms by —F, —Cl, —Br, —I, —CN, —NO₂, —CHO, ═O, —Rₓ, —OH, —ORₓ, —NH₂, —NHRₓ or -N(Rₓ)₂, wherein Rₓ, respectively independently, means —C₁₋₈-aliphatic or —C₃₋₁₂-cycloaliphatic; and/or
with respect to “aryl” and “heteroaryl”, “mono- or polysubstituted”, respectively independently, means the mono- or polysubstitution of one or more hydrogen atoms of the ring system by substituents selected from the group comprising —F, —Cl, —Br, —I, —CN, —NO₂, —CHO, ═O or —OH; and/or
Rₓ, respectively independently, means
—C₁₋₈-aliphatic, -C₃₋₁₂-cycloaliphatic, -aryl or heteroaryl,
preferably C₁₋₈-aliphatic or —C₃₋₁₂-cycloaliphatic, preferably in each case unsubstituted.

12. The compound of claim 1,
wherein with respect to general Formula (A) none of A₁, B and C is —S—; and/or wherein with respect to general Formula (A) D is not —C—CN; and/or
which is not

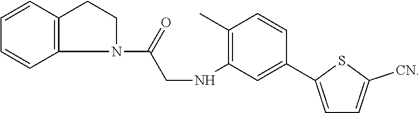 [SMILES: Cc1ccc(-c2ccc(C#N)s2)cc1NCC(=O)N1CCc2ccccc21]

13. The compound of claim 1, wherein
H is Tritium (3H), and/or
F is ¹⁸F, and/or
C is ¹¹C or ¹³C or ¹⁴C, and/or
O is 150, and/or
N is ¹³N, and/or
S is ³⁵S, and/or
Cl is ³⁶Cl, and/or
I is ¹²⁵I or ¹³¹I.

14. The compound of claim 1, which is selected from the group consisting of
1-(4-methoxyindolin-1-yl)-2-((2-methyl-5-(3-methyl-1,2,4-thiadiazol-5-yl)phenyl)amino)ethan-1-one (1);
1-(4-methoxyindolin-1-yl)-2-((2-methyl-5-(3-methyl-1,2,4-oxadiazol-5-yl)phenyl)amino)ethan-1-one (2);
2-((5-(3-ethyl-1,2,4-oxadiazol-5-yl)-2-fluorophenyl)amino)-1-(6-fluoro-5-methoxyindolin-1-yl)ethan-1-one (3);
2-((2-methoxy-5-(3-methyl-1,2,4-thiadiazol-5-yl)phenyl)amino)-1-(4-methoxyindolin-1-yl)ethan-1-one (4);
2-((5-(2-ethyl-2H-tetrazol-5-yl)-2-methoxyphenyl)amino)-1-(4-methoxyindolin-1-yl)ethan-1-one (5);
1-(5-fluoro-1H-indol-1-yl)-2-((2-methoxy-5-(3-methyl-1,2,4-thiadiazol-5-yl)phenyl)amino)ethan-1-one (6);
2-(5-(3-ethyl-1,2,4-oxadiazol-5-yl)-2-methoxyphenylamino)-1-(6-(trifluoromethyl)indolin-1-yl)ethanone (7);
2-((3-fluoro-2-methoxy-5-(3-methyl-1,2,4-thiadiazol-5-yl)phenyl)amino)-1-(4-methoxyindolin-1-yl)ethan-1-one (8);
1-(6-bromoindolin-1-yl)-2-((5-(3-ethyl-1,2,4-oxadiazol-5-yl)-2-methoxyphenyl)amino)ethan-1-one (9);
1-(5-chloro-2,3-dihydro-1H-pyrrolo[3,2-b]pyridin-1-yl)-2-((2-methyl-5-(3-methyl-1,2,4-thiadiazol-5-yl)phenyl)amino)ethan-1-one (10);
2-((5-(3-ethyl-1,2,4-oxadiazol-5-yl)-2-methoxyphenyl)amino)-1-(5-methoxy-2,3-dihydro-1H-pyrrolo[3,2-b]pyridin-1-yl)ethan-1-one (11);
1-(4-(2-hydroxy-2-methylpropoxy)indolin-1-yl)-2-((2-methyl-5-(3-methyl-1,2,4-thiadiazol-5-yl)phenyl)amino) (12);
2-(1-((2-methyl-5-(3-methyl-1,2,4-thiadiazol-5-yl)phenyl)glycyl)indolin-4-yl)isothiazolidin-3-one 1,1-dioxide (13);
2-((5-(3-bromo-1,2,4-thiadiazol-5-yl)-2-methylphenyl)amino)-1-(4-(2-hydroxy-2-methylpropoxy)indolin-1-yl)ethan-1-one (14);
2-((5-(3-ethynyl-1,2,4-thiadiazol-5-yl)-2-methylphenyl)amino)-1-(4-methoxyindolin-1-yl)ethan-1-one (15);
1-(4-(3-hydroxypropoxy)indolin-1-yl)-2-((2-methyl-5-(3-methyl-1,2,4-thiadiazol-5-yl)phenyl)amino)ethan-1-one (16);
1-(1-((2-methyl-5-(3-methyl-1,2,4-thiadiazol-5-yl)phenyl)glycyl)indolin-4-yl)pyrrolidine-2,5-dione (17);
1-(4-(2-(2-hydroxyethoxy)ethoxy)indolin-1-yl)-2-((2-methyl-5-(3-vinyl-1,2,4-thiadiazol-5-yl)phenyl)amino)ethan-1-one (18);
2-((5-(3-ethyl-1,2,4-thiadiazol-5-yl)-2-methylphenyl)amino)-1-(4-(2-(2-hydroxyethoxy)ethoxy)indolin-1-yl)ethan-1-one (19);
N-(1-((5-(3-ethyl-1,2,4-thiadiazol-5-yl)-2-methylphenyl)glycyl)indolin-4-yl)methanesulfonamide (20);
2-((5-(3-ethyl-1,2,4-thiadiazol-5-yl)-2-fluorophenyl)amino)-1-(4-(1-hydroxyethyl)indolin-1-yl)ethan-1-one (21);
1-(4-(2-hydroxypropan-2-yl)indolin-1-yl)-2-((2-methyl-5-(3-methyl-1,2,4-thiadiazol-5-yl)phenyl)amino)ethan-1-one (22);
1-(4-(2-oxa-7-azaspiro[3.5]nonan-7-yl)indolin-1-yl)-2-((2-methyl-5-(3-methyl-1,2,4-thiadiazol-5-yl)phenyl)amino)ethan-1-one (23);
1-(4-(2-hydroxy-2-methylpropoxy)indolin-1-yl)-2-((5-(3-methyl-1,2,4-thiadiazol-5-yl)-2-(pyrrolidin-1-yl)phenyl)amino)ethan-1-one (24);
1-(4-(3-hydroxypyrrolidin-1-yl)indolin-1-yl)-2-((2-methyl-5-(3-methyl-1,2,4-oxadiazol-5-yl)phenyl)amino)ethan-1-one (25);
1-((5-(3-ethyl-1,2,4-thiadiazol-5-yl)-2-methylphenyl)glycyl)-N-methylindoline-4-sulfonamide (26);
1-(4-(2,6-dimethylmorpholino)indolin-1-yl)-2-((5-(3-ethyl-1,2,4-thiadiazol-5-yl)-2-methylphenyl)amino)ethan-1-one (27);
1-(4-(hydroxymethyl)indolin-1-yl)-2-((2-methyl-5-(5-methylfuran-2-yl)phenyl)amino)ethan-1-one (28);
1-(4-(1-hydroxyethyl)indolin-1-yl)-2-((2-methyl-5-(3-methyl-1,2,4-thiadiazol-5-yl)phenyl)amino)ethan-1-one (29);
1-(4-(3-hydroxy-3-methylbutoxy)indolin-1-yl)-2-((2-methyl-5-(3-methyl-1,2,4-thiadiazol-5-yl)phenyl)amino)ethan-1-one (30);
1-(4-(2-(2-hydroxyethoxy)ethoxy)indolin-1-yl)-2-((2-methyl-5-(3-methyl-1,2,4-thiadiazol-5-yl)phenyl)amino)ethan-1-one (31);
2-((2-methyl-5-(3-methyl-1,2,4-thiadiazol-5-yl)phenyl)amino)-1-(4-(pyridin-3-yl)indolin-1-yl)ethan-1-one (32);
2-((2-methyl-5-(3-methyl-1,2,4-thiadiazol-5-yl)phenyl)amino)-1-(4-(2,2,2-trifluoro-1-hydroxyethyl)indolin-1-yl)ethan-1-one (33);
2-((2-fluoro-5-(3-methyl-1,2,4-thiadiazol-5-yl)phenyl)amino)-1-(4-methoxyindolin-1-yl)ethan-1-one (34);
1-(4-(1H-imidazol-1-yl)indolin-1-yl)-2-((5-(3-ethyl-1,2,4-thiadiazol-5-yl)-2-methylphenyl)amino)ethan-1-one (35);
1-(4-(3-hydroxy-8-azabicyclo[3.2.1]octan-8-yl)indolin-1-yl)-2-((2-methyl-5-(5-methylfuran-2-yl)phenyl)amino)ethan-1-one (36);
1-(4-acetylindolin-1-yl)-2-((2-methyl-5-(3-methyl-1,2,4-thiadiazol-5-yl)phenyl)amino)ethan-1-one (37);
2-((5-(3-cyclopropyl-1,2,4-thiadiazol-5-yl)-2-methylphenyl)amino)-1-(4-(2-hydroxy-2-methylpropoxy)indolin-1-yl)ethan-1-one (38);
2-((2-chloro-5-(3-chloro-1,2,4-thiadiazol-5-yl)phenyl)amino)-1-(4-hydroxyindolin-1-yl)ethan-1-one (39);
2-((5-(3-(difluoromethyl)-1,2,4-thiadiazol-5-yl)-2-methylphenyl)amino)-1-(4-(2-hydroxypropan-2-yl)indolin-1-yl)ethan-1-one (40);
2-((2-fluoro-5-(3-methyl-1,2,4-oxadiazol-5-yl)phenyl)amino)-1-(4-(2,2,2-trifluoro-1-hydroxyethyl)indolin-1-yl)ethan-1-one (41);
1-(5-methoxy-2,3-dihydro-1H-pyrrolo[3,2-b]pyridin-1-yl)-2-((2-methyl-5-(3-methyl-1,2,4-thiadiazol-5-yl)phenyl)amino)ethan-1-one (42);
2-((2-fluoro-5-(5-methylfuran-2-yl)phenyl)amino)-1-(4-(2-hydroxy-2-methylpropoxy)indolin-1-yl)ethan-1-one (43);
1-(4-((2-hydroxy-2-methylpropyl)amino)indolin-1-yl)-2-((2-methyl-5-(3-methyl-1,2,4-thiadiazol-5-yl)phenyl)amino)ethan-1-one (44);
2-((5-(3-ethyl-1,2,4-thiadiazol-5-yl)-2-methylphenyl)amino)-1-(4-(4-methylpiperazine-1-carbonyl)indolin-1-yl)ethan-1-one (45);
N-(1-((2-methyl-5-(3-methyl-1,2,4-thiadiazol-5-yl)phenyl)glycyl)indolin-4-yl)methanesulfonamide (46);
1-(4-(3-hydroxy-3-methylbutoxy)indolin-1-yl)-2-((2-methyl-5-(3-methyl-1,2,4-oxadiazol-5-yl)phenyl)amino)ethan-1-one (47);
1-(4-(1H-imidazol-1-yl)indolin-1-yl)-2-((2-methyl-5-(3-methyl-1,2,4-thiadiazol-5-yl)phenyl)amino)ethan-1-one (48);
1-(4-(2-hydroxy-2-methylpropoxy)indolin-1-yl)-2-((2-methyl-5-(3-methyl-1,2,4-oxadiazol-5-yl)phenyl)amino)ethan-1-one (49);
2-((5-(3-cyclopropyl-1,2,4-oxadiazol-5-yl)-2-methylphenyl)amino)-1-(4-(2-hydroxy-2-methylpropoxy)indolin-1-yl)ethan-1-one (50);
1-((2-methyl-5-(3-methyl-1,2,4-thiadiazol-5-yl)phenyl)glycyl)indoline-4-sulfonamide (51);
1-(indolin-1-yl)-2-((2-methyl-5-(3-methyl-1,2,4-thiadiazol-5-yl)phenyl)amino)ethan-1-one (52);
2-((2-fluoro-5-(3-methyl-1,2,4-oxadiazol-5-yl)phenyl)amino)-1-(4-(2-hydroxypropan-2-yl)indolin-1-yl)ethan-1-one (53);
1-(1-((2-methyl-5-(3-methyl-1,2,4-thiadiazol-5-yl)phenyl)glycyl)indolin-4-yl)imidazolidine-2,4-dione (54);
1-(4-(3-hydroxyoxetan-3-yl)indolin-1-yl)-2-((2-methyl-5-(3-methyl-1,2,4-thiadiazol-5-yl)phenyl)amino)ethan-1-one (55);
2-((5-(3-ethyl-1,2,4-thiadiazol-5-yl)-2-fluorophenyl)amino)-1-(4-(3-hydroxyazetidin-1-yl)indolin-1-yl)ethan-1-one (56);
methyl 1-((2-methyl-5-(3-methyl-1,2,4-thiadiazol-5-yl)phenyl)glycyl)indoline-3-carboxylate (57);
1-(3-(2-hydroxyethyl)indolin-1-yl)-2-((2-methyl-5-(3-methyl-1,2,4-thiadiazol-5-yl)phenyl)amino)ethan-1-one (58);
N-methyl-1-((2-methyl-5-(3-methyl-1,2,4-thiadiazol-5-yl)phenyl)glycyl)indoline-3-carboxamide (59);
1-(4-(1,2-dihydroxypropan-2-yl)indolin-1-yl)-2-((2-fluoro-5-(3-methyl-1,2,4-oxadiazol-5-yl)phenyl)amino)ethan-1-one (60);
2-((5-(3-(difluoromethyl)-1,2,4-thiadiazol-5-yl)-2-methylphenyl)amino)-1-(4-(1,2-dihydroxypropan-2-yl)indolin-1-yl)ethan-1-one (61);
1-(4-(1,2-dihydroxypropan-2-yl)indolin-1-yl)-2-((2-methyl-5-(3-methyl-1,2,4-thiadiazol-5-yl)phenyl)amino)ethan-1-one (62);
1-(4-(2-fluoro-1-hydroxypropan-2-yl)indolin-1-yl)-2-((2-methyl-5-(3-methyl-1,2,4-thiadiazol-5-yl)phenyl)amino)ethan-1-one (63);
rel-(R)-1-(4-(1,2-dihydroxypropan-2-yl)indolin-1-yl)-2-((2-methyl-5-(3-methyl-1,2,4-thiadiazol-5-yl)phenyl)amino)ethan-1-one (64);
rel-(R)-1-(4-(1,2-dihydroxypropan-2-yl)indolin-1-yl)-2-((2-methyl-5-(3-methyl-1,2,4-thiadiazol-5-yl)phenyl)amino)ethan-1-one (65);
2-((5-(3-ethyl-1,2,4-thiadiazol-5-yl)-2-methylphenyl)amino)-1-(4-(3-hydroxyazetidin-1-yl)indolin-1-yl)ethan-1-one (66);
2-(1-((2-methyl-5-(3-methyl-1,2,4-thiadiazol-5-yl)phenyl)glycyl)indolin-4-yl)propan-2-yl 2,3-dihydroxypropanoate (67);
1-(4-(3-hydroxypyrrolidin-1-yl)indolin-1-yl)-2-((2-methyl-5-(3-methyl-1,2,4-thiadiazol-5-yl)phenyl)amino)ethan-1-one (68);
imino(methyl)((1-((2-methyl-5-(3-methyl-1,2,4-thiadiazol-5-yl)phenyl) glycyl)indolin-4-yl)methyl)sulfanone (69);
1-(4-(1H-pyrazol-3-yl)indolin-1-yl)-2-((2-methyl-5-(3-methyl-1,2,4-thiadiazol-5-yl)phenyl)amino)ethan-1-one (70);
2-((5-(3-ethyl-1,2,4-thiadiazol-5-yl)-2-fluorophenyl)amino)-1-(4-(3-hydroxypyrrolidin-1-yl)indolin-1-yl)ethan-1-one (71);
2-((5-(3-ethyl-1,2,4-thiadiazol-5-yl)-2-methylphenyl)amino)-1-(4-(piperazin-1-yl)indolin-1-yl)ethan-1-one (72);
1-(4-(1H-imidazol-4-yl)indolin-1-yl)-2-((2-methyl-5-(5-methylfuran-2-yl)phenyl)amino)ethan-1-one (73);
1-(4-aminoindolin-1-yl)-2-((2-methyl-5-(3-methyl-1,2,4-thiadiazol-5-yl)phenyl)amino)ethan-1-one (74);
1-(4-aminoindolin-1-yl)-2-((2-fluoro-5-(3-methyl-1,2,4-oxadiazol-5-yl)phenyl)amino)ethan-1-one (75);
N-(1-((2-methyl-5-(3-methyl-1,2,4-thiadiazol-5-yl)phenyl)glycyl)indolin-4-yl)acetamide (76);
N-(1-((2-methyl-5-(3-methyl-1,2,4-thiadiazol-5-yl)phenyl)glycyl)indolin-4-yl)-N-(methylsulfonyl)acetamide (77);
N-(1-((2-methyl-5-(3-methyl-1,2,4-thiadiazol-5-yl)phenyl)glycyl)indolin-4-yl)-N-(methylsulfonyl)methanesulfonamide (78);
1-(1-((2-methyl-5-(3-methyl-1,2,4-thiadiazol-5-yl)phenyl)glycyl)indolin-4-yl)-1H-pyrrole-2,5-dione (79);
3-amino-1-(1-((2-methyl-5-(3-methyl-1,2,4-thiadiazol-5-yl)phenyl)glycyl)indolin-4-yl)pyrrolidine-2,5-dione (80);
(R)-N-(1-((2-methyl-5-(3-methyl-1,2,4-thiadiazol-5-yl)phenyl)glycyl)indolin-4-yl)pyrrolidine-2-carboxamide (81);
(2R,3R)-2,3-dihydroxy-4-((1-((2-methyl-5-(3-methyl-1,2,4-thiadiazol-5-yl)phenyl)glycyl)indolin-4-yl)amino)-4-oxobutanoic acid (82);
3-(1-((2-methyl-5-(3-methyl-1,2,4-oxadiazol-5-yl)phenyl)glycyl)indolin-4-yl)imidazolidine-2,4-dione (83);
2-((5-(3-ethyl-1,2,4-thiadiazol-5-yl)-2-methylphenyl)amino)-1-(4-hydroxyindolin-1-yl)ethan-1-one (84);
1-(4-hydroxyindolin-1-yl)-2-((2-methyl-5-(3-methyl-1,2,4-thiadiazol-5-yl)phenyl)amino)ethan-1-one (85);
1-(4-hydroxyindolin-1-yl)-2-((2-methyl-5-(3-methyl-1,2,4-oxadiazol-5-yl)phenyl)amino)ethan-1-one (86);
2-((1-((5-(3-ethyl-1,2,4-thiadiazol-5-yl)-2-methylphenyl)glycyl)indolin-4-yl)oxy)-2,2-difluoroacetic acid (87);
1-(4-(1-amino-2,2,2-trifluoroethyl)indolin-1-yl)-2-((2-methyl-5-(3-methyl-1,2,4-thiadiazol-5-yl)phenyl)amino)ethan-1-one (88);
1-(4-(1-amino-2,2,2-trifluoroethyl)indolin-1-yl)-2-((2-methyl-5-(3-methyl-1,2,4-oxadiazol-5-yl)phenyl)amino)ethan-1-one (89);
1-(4-(2-hydroxypropan-2-yl)-2,3-dihydro-1H-pyrrolo[3,2-c]pyridin-1-yl)-2-((2-methyl-5-(3-methyl-1,2,4-thiadiazol-5-yl)phenyl)amino)ethan-1-one (90);
1-(4-(2-hydroxypropan-2-yl)-2,3-dihydro-1H-pyrrolo[2,3-c]pyridin-1-yl)-2-((2-methyl-5-(3-methyl-1,2,4-thiadiazol-5-yl)phenyl)amino)ethan-1-one (91);
1-(4-(i-hydroxyethyl)-2,3-dihydro-1H-pyrrolo[3,2-c]pyridin-1-yl)-2-((2-methyl-5-(3-methyl-1,2,4-thiadiazol-5-yl)phenyl)amino)ethan-1-one (92);
1-(7-methoxy-1H-indol-3-yl)-2-((2-methoxy-5-(3-methyl-1,2,4-thiadiazol-5-yl)phenyl)amino)ethan-1-one (93);
1-(5-fluoro-6-methoxy-1H-indol-3-yl)-2-((2-methoxy-5-(3-methyl-1,2,4-thiadiazol-5-yl)phenyl)amino)ethan-1-one (94);
1-(6-methoxy-1H-pyrrolo[2,3-b]pyridin-3-yl)-2-((2-methyl-5-(3-methyl-1,2,4-thiadiazol-5-yl)phenyl)amino)ethan-1-one (95);
2-((5-(3-ethyl-1,2,4-oxadiazol-5-yl)-2-methoxyphenyl)amino)-1-(6-methoxy-1H-indol-3-yl)ethan-1-one (96);
2-((5-(3-ethyl-1,2,4-oxadiazol-5-yl)-2-methoxyphenyl)amino)-1-(5-fluoro-6-methoxy-1H-indol-3-yl)ethan-1-one (97);
2-((5-(3-ethyl-1,2,4-oxadiazol-5-yl)-2-methoxyphenyl)amino)-1-(5-fluoro-6-methoxy-1-methyl-1H-indol-3-yl)ethan-1-one (98);
7-((2-methyl-5-(3-methyl-1,2,4-thiadiazol-5-yl)phenyl)glycyl)-3,4-dihydro-[1,4]diazepino[6,7,1-hi]indol-1(2H)-one (99);
2-((2-methyl-5-(3-methyl-1,2,4-thiadiazol-5-yl)phenyl)amino)-1-(1H-pyrrolo[2,3-b]pyridin-3-yl)ethan-1-one (100);
2-((3-fluoro-2-methoxy-5-(3-methyl-1,2,4-thiadiazol-5-yl)phenyl)amino)-1-(5-fluoro-6-methoxy-1H-indol-3-yl)ethan-1-one (101);
1-(1H-indol-3-yl)-2-((2-methyl-5-(3-methyl-1,2,4-thiadiazol-5-yl)phenyl)amino)ethan-1-one (102)
1-(5,6-dihydro-4H-pyrrolo[3,2,1-ij]quinolin-1-yl)-2-((2-methyl-5-(3-methyl-1,2,4-thiadiazol-5-yl)phenyl)amino)ethan-1-one (103);
2-((2-methoxy-5-(3-methyl-1,2,4-thiadiazol-5-yl)phenyl)amino)-1-(1,6,7,8-tetrahydrocyclopenta[g]indol-3-yl)ethan-1-one (104);
1-(3-hydroxy-3-methyl-3,4-dihydro-2H-[1,4]oxazepino[2,3,4-hi]indol-7-yl)-2-((2-methyl-5-(3-methyl-1,2,4-thiadiazol-5-yl)phenyl)amino)ethan-1-one (105);
1-(1H-indol-3-yl)-2-((2-methyl-5-(5-methyl-1,2,4-oxadiazol-3-yl)phenyl)amino)ethan-1-one (106);
2-((2-methyl-5-(3-methyl-1,2,4-oxadiazol-5-yl)phenyl)amino)-1-(1H-pyrrolo[2,3-b]pyridin-3-yl)ethan-1-one (107);
2-(4-bromo-5-(3-ethyl-1,2,4-oxadiazol-5-yl)-2-methoxyphenylamino)-1-(5-bromo-6-methoxy-1H-indol-3-yl)ethanone (108);
2-((4-bromo-5-(3-ethyl-1,2,4-oxadiazol-5-yl)-2-methoxyphenyl)amino)-1-(6-methoxy-1H-indol-3-yl)ethan-1-one (109);
1-(7-methoxy-1H-pyrrolo[2,3-c]pyridin-3-yl)-2-((2-methyl-5-(3-methyl-1,2,4-thiadiazol-5-yl)phenyl)amino)ethan-1-one (110);
2-((5-(3-ethyl-1,2,4-oxadiazol-5-yl)-2-methylphenyl)amino)-1-(1-methyl-1H-indol-3-yl)ethan-1-one (111);
1-(7-(1-hydroxyethyl)-1H-indol-3-yl)-2-((2-methyl-5-(3-methyl-1,2,4-thiadiazol-5-yl)phenyl)amino)ethan-1-one (112);
1-(4-(2-(2-hydroxyethoxy)ethoxy)indolin-1-yl)-2-((5-(5-isopropylpyridin-2-yl)-2-methylphenyl)amino)ethan-1-one (113);
2-((5-(5-cyclopropylpyridin-2-yl)-2-methylphenyl)amino)-1-(4-(2-hydroxy-2-methylpropoxy)indolin-1-yl)ethan-1-one (114);
2-((5-(5-chloropyridin-2-yl)-2-methylphenyl)amino)-1-(4-(2-hydroxy-2-methylpropoxy)indolin-1-yl)ethan-1-one (115);
1-(4-(hydroxymethyl)indolin-1-yl)-2-((2-methyl-5-(5-methylpyridin-2-yl)phenyl)amino)ethan-1-one (116);
1-(4-(2-hydroxy-2-methylpropoxy)indolin-1-yl)-2-((2-methyl-5-(5-methylpyridin-2-yl)phenyl)amino)ethan-1-one (117);
1-(5,6-difluoroindolin-1-yl)-2-((2-methoxy-5-(5-methylpyridin-2-yl)phenyl)amino)ethan-1-one (118);
methyl 1-((2-methoxy-5-(5-methylpyridin-2-yl)phenyl)glycyl)indoline-4-carboxylate (119);
2-((2-methoxy-5-(5-methylpyridin-2-yl)phenyl)amino)-1-(4-methoxyindolin-1-yl)ethan-1-one (120);
2-((5-(4,5-dimethylpyridin-2-yl)-2-methoxyphenyl)amino)-1-(4-hydroxyindolin-1-yl)ethan-1-one (121);
1-(5,6-difluoro-1H-indol-3-yl)-2-((3-(pyridin-2-yl)phenyl)amino)ethan-1-one (122);
2-((4-bromo-2-methyl-5-(3-methyl-1,2,4-thiadiazol-5-yl)phenyl)amino)-1-(6-bromo-4-(1,2-dihydroxypropan-2-yl)indolin-1-yl)ethan-1-one (123);
and a physiologically acceptable salt thereof or a physiologically acceptable solvate thereof.

15. A medicament comprising a compound according to claim 1.

16. The compound of claim 1, for use as medicament.

17. The compound according to claim 1 for use in the treatment of a disease or disorder selected from the group consisting of
pain, inflammation and cancer, preferably colon cancer; or
neuropathic pain, spinal cord injury, epilepsy, stroke, acute brain injury, multiple sclerosis, neurodegenerative diseases such as Parkinson's and Alzheimer's and amyotropic lateral sclerosis (ALS), obesity, obesity-dependent inflammation, artherosclerosis, allergen-induced airway inflammation, allergic asthma, airway remodeling, rheumatoid arthritis, colitis, alcohol-induced liver inflammation, steatohepatitis, inflammatory and fibrotic diseases, itch and cutaneous pain, muscle pain, chronic pain, diabetic neuropathy, trigeminal neuralgia, other types of pain, pain and depression comorbidity, liver fibrosis, hepatitits virus-induced hepatocellular carcinoma, prostate cancer, gastric cancer, breast cancer, glioma, colon cancer, and disorders of the central nervous system.

18. The medicament according to claim 15 for use in the treatment of a disease or disorder selected from the group consisting of
pain, inflammation and cancer, preferably colon cancer; or
neuropathic pain, spinal cord injury, epilepsy, stroke, acute brain injury, multiple sclerosis, neurodegenerative diseases such as Parkinson's and Alzheimer's and amyotropic lateral sclerosis (ALS), obesity, obesity-dependent inflammation, artherosclerosis, allergen-induced airway inflammation, allergic asthma, airway remodeling, rheumatoid arthritis, colitis, alcohol-induced liver inflammation, steatohepatitis, inflammatory and fibrotic diseases, itch and cutaneous pain, muscle pain, chronic pain, diabetic neuropathy, trigeminal neuralgia, other types of pain, pain and depression comorbidity, liver fibrosis, hepatitits virus-induced hepatocellular carcinoma, prostate cancer, gastric cancer, breast cancer, glioma, colon cancer, and disorders of the central nervous system.
